SEQUENCE LISTING (27 sequences)

SEQ ID NO: 1 - protein, 39 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 39: note = The C-terminal amino acid is amidated as a C-terminal primary amide
YXEGTFTSDYSIXLDKIAQKAFVQWLIAGGPSSGAPPPS

SEQ ID NO: 2 - protein, 35 aa, synthetic construct
  SITE 9: note = X at position 9 is Aib
  MOD_RES 16: note = K at position 16 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 35: note = The C-terminal amino acid is amidated as a C-terminal primary amide
TFTSDYSIXLDKIAQKAFVQWLIAGGPSSGAPPPS

SEQ ID NO: 3 - protein, 4 aa, synthetic construct
  MOD_RES 2: note = A at position 2 is Aib
YAEG

SEQ ID NO: 4 - protein, 8 aa, synthetic construct
  SITE 2: note = X at position 2 is Aib
YXEGTFTS

SEQ ID NO: 5 - protein, 31 aa, synthetic construct
  SITE 5: note = X at position 5 is Aib
  MOD_RES 12: note = K at position 12 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 31: note = The C-terminal amino acid is amidated as a C-terminal primary amide
DYSIXLDKIAQKAFVQWLIAGGPSSGAPPPS

SEQ ID NO: 6 - protein, 30 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
YXEGTFTSDYSIXLDKIAQKAFVQWLIAGG

SEQ ID NO: 7 - protein, 34 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
YXEGTFTSDYSIXLDKIAQKAFVQWLIAGGPSSG

SEQ ID NO: 8 - protein, 6 aa, synthetic construct
  MOD_RES 6: note = The C-terminal amino acid is amidated as a C-terminal primary amide
GAPPPS

SEQ ID NO: 9 - protein, 29 aa, synthetic construct
  SITE 2: note = X at position 2 is Aib
  SITE 13: note = X at position 13 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
YXEGTFTSDYSIXLDKIAQKAFVQWLIAG

SEQ ID NO: 10 - protein, 9 aa, synthetic construct
  SITE 2: note = X at position 2 is Aib
YXEGTFTSD

SEQ ID NO: 11 - protein, 30 aa, synthetic construct
  SITE 4: note = X at position 4 is Aib
  MOD_RES 11: note = K at position 11 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 30: note = The C-terminal amino acid is amidated as a C-terminal primary amide
YSIXLDKIAQKAFVQWLIAGGPSSGAPPPS

SEQ ID NO: 12 - protein, 39 aa, synthetic construct
  SITE 2: note = X at position 2 is Aib
  SITE 13: note = X at position 13 is alpha-methyl-Leu
  MOD_RES 17: note = K at position 17 is chemically modified through conjugation to the epsilon-amino group of the K side-chain with (2-[2-(2-Amino-ethoxy)-ethoxy]-acetyl)-(gamma-Glu)-CO -(CH2)18-CO2H
  MOD_RES 39: note = The C-terminal amino acid is amidated as a C-terminal primary amide
  SITE 20: note = X at position 20 is Aib
YXQGTFTSDYSIXLDKKAQXAFIEYLLEGGPSSGAPPPS

SEQ ID NO: 13 - protein, 35 aa, synthetic construct
  SITE 9: note = X at position 9 is alpha-methyl-Leu
  MOD_RES 13: note = K at position 13 is chemically modified through conjugation to the epsilon-amino group of the K side-chain with (2-[2-(2-Amino-ethoxy)-ethoxy]-acetyl)-(gamma-Glu)-CO -(CH2)18-CO2H
  SITE 16: note = X at position 16 is Aib
  MOD_RES 35: note = The C-terminal amino acid is amidated as a C-terminal primary amide
TFTSDYSIXLDKKAQXAFIEYLLEGGPSSGAPPPS

SEQ ID NO: 14 - protein, 20 aa, synthetic construct
  SITE 2: note = X at position 2 is Aib
  SITE 13: note = X at position 13 is alpha-methyl-Leu
  MOD_RES 17: note = K at position 17 is chemically modified through conjugation to the epsilon-amino group of the K side-chain with (2-[2-(2-Amino-ethoxy)-ethoxy]-acetyl)-(gamma-Glu)-CO -(CH2)18-CO2H
  SITE 20: note = X at position 20 is Aib
YXQGTFTSDYSIXLDKKAQX

SEQ ID NO: 15 - protein, 16 aa, synthetic construct
  SITE 9: note = X at position 9 is alpha-methyl-Leu
  MOD_RES 13: note = K at position 13 is chemically modified through conjugation to the epsilon-amino group of the K side-chain with (2-[2-(2-Amino-ethoxy)-ethoxy]-acetyl)-(gamma-Glu)-CO -(CH2)18-CO2H
  SITE 16: note = X at position 16 is Aib
TFTSDYSIXLDKKAQX

SEQ ID NO: 16 - protein, 34 aa, synthetic construct
  SITE 2: note = X at position 2 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 34: note = The C-terminal amino acid is amidated as a C-terminal primary amide
HXQGTFTSDYSKYLDEKKAKEFVEWLLEGGPSSG

SEQ ID NO: 17 - protein, 30 aa, synthetic construct
  MOD_RES 16: note = K at position 16 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 30: note = The C-terminal amino acid is amidated as a C-terminal primary amide
TFTSDYSKYLDEKKAKEFVEWLLEGGPSSG

SEQ ID NO: 18 - protein, 22 aa, synthetic construct
  MOD_RES 8: note = K at position 8 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 22: note = The C-terminal amino acid is amidated as a C-terminal primary amide
YLDEKKAKEFVEWLLEGGPSSG

SEQ ID NO: 19 - protein, 8 aa, synthetic construct
  SITE 2: note = X at position 2 is Aib
HXQGTFTS

SEQ ID NO: 20 - protein, 21 aa, synthetic construct
  MOD_RES 7: note = K at position 7 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 21: note = The C-terminal amino acid is amidated as a C-terminal primary amide
LDEKKAKEFVEWLLEGGPSSG

SEQ ID NO: 21 - protein, 9 aa, synthetic construct
  SITE 2: note = X at position 2 is Aib
HXQGTFTSD

SEQ ID NO: 22 - protein, 5 aa, synthetic construct
  MOD_RES 5: note = The C-terminal amino acid is amidated as a C-terminal primary amide
APPPS

SEQ ID NO: 23 - protein, 29 aa, synthetic construct
  SITE 2: note = X at position 2 is Aib
  SITE 13: note = X at position 13 is alpha-methyl-Leu
  MOD_RES 17: note = K at position 17 is chemically modified through conjugation to the epsilon-amino group of the K side-chain with (2-[2-(2-Amino-ethoxy)-ethoxy]-acetyl)-(gamma-Glu)-CO -(CH2)18-CO2H
YXQGTFTSDYSIXLDKKAQXAFIEYLLEG

SEQ ID NO: 24 - protein, 10 aa, synthetic construct
  MOD_RES 10: note = The C-terminal amino acid is amidated as a C-terminal primary amide
GPSSGAPPPS

SEQ ID NO: 25 - protein, 29 aa, synthetic construct
  SITE 2: note = X at position 2 is Aib
  MOD_RES 20: note = K at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
HXQGTFTSDYSKYLDEKKAKEFVEWLLEG

SEQ ID NO: 26 - protein, 13 aa, synthetic construct
  SITE 2: note = X at position 2 is Aib
  SITE 13: note = X at position 13 is Aib
YXEGTFTSDYSIX

SEQ ID NO: 27 - protein, 10 aa, synthetic construct
  SITE 9: note = X at position 9 is Aib
TFTSDYSIXL

(19) United States
(12) Patent Application Publication
(10) Pub. No.: US 2026/0200981 A1
(43) Pub. Date: Jul. 16, 2026

(54) PEPTIDES FOR INCRETIN SYNTHESIS

(71) Applicant: ELI LILLY AND COMPANY, Indianapolis, IN (US)

(72) Inventors: David Andrew COATES, New Palestine, IN (US); Emily Jeannette GUINN, Indianapolis, IN (US); Ankur JALAN, Indianapolis, IN (US); Youngsun KIM, Carmel, IN (US); Michael Edward KOBIERSKI, Greenwood, IN (US); Michael Eugene KOPACH, Greenwood, IN (US); Jeremy Miles MERRITT, Indianapolis, IN (US); Emily Suzanne MURZINSKI, Zionsville, IN (US); Jon Gordon SELBO, Noblesville, IN (US); Jing TENG, West Lafayette, IN (US); Jingyao WANG, Carmel, IN (US)

(21) Appl. No.: 19/348,544

(22) Filed: Oct. 2, 2025

Related U.S. Application Data
(63) Continuation of application No. 19/118,209, filed on Oct. 5, 2023.
(60) Provisional application No. 63/378,397, filed on Oct. 5, 2022.
(60) Provisional application No. 63/477,742, filed on Dec. 29, 2022.

(51) Int. Cl. C07K 7/06; C07K 5/103; C07K 5/107

(52) U.S. Cl. CPC C07K 7/06; C07K 5/1008; C07K 5/1016

(57) ABSTRACT

Provided are crystalline forms of peptide fragments, method of preparation thereof, and use thereof for preparing peptides. The present crystalline compounds may be employed as intermediates with improved purity and physical properties for peptide syntheses.

### RELATED APPLICATIONS

This application is a continuation application of U.S. patent application Ser. No. 19/118,209, filed on Apr. 3, 2025, which is a National Stage Application of International Application No. PCT/US2023/076098, filed on Oct. 5, 2023, which claims priority to U.S. Provisional Patent Application No. 63/378,397, filed Oct. 5, 2022, and U.S. Provisional Patent Application No. 63/477,742, filed Dec. 29, 2022. The entire contents of these applications are incorporated herein by reference.

### SEQUENCE LISTING

The present application is being filed along with a Sequence Listing in ST 0.26 XML format. The Sequence Listing is provided as a file titled “30267A_US.xml” created Nov. 11, 2024, and is 51 KB kilobytes in size. The Sequence Listing information in the ST.26 XML format is incorporated herein by reference in its entirety.

### DISCLOSURE

The present disclosure relates to peptides useful in the preparation of biopharmaceutical products and methods of their preparation and use.

Diabetes mellitus is a chronic disorder characterized by hyperglycemia resulting from defects in insulin secretion, insulin action, or both. In type 2 diabetes mellitus (“T2D”), the combined effects of impaired insulin secretion and insulin resistance are associated with elevated blood glucose levels.

The GIP/GLP1 dual agonist tirzepatide (“TZP”) is described and claimed in U.S. Pat. No. 9,474,780. Tirzepatide can be useful in the treatment of type 2 diabetes mellitus (“T2D”). Further processes for preparing tirzepatide and intermediates in those processes are described in United States patent application publication 2022/0135639 A1. Retatrutide (“GGG”), a GLP-1/GIP/glucagon triple receptor agonist is described in international patent publication nos. WO 2019/125938 and WO 2021/034815. Mazdutide (“OXM”), a GLP-1/glucagon dual agonist, is described and claimed in U.S. Pat. No. 9,935,335 and international patent publication no. WO 2021/252829. Both retatrutide and mazdutide can also be useful in treating diabetes and boosting weight loss. These patents and patent application publications are incorporated by reference herein, in their entireties.

There is a need for further processes and intermediates to enable improved technology for streamlined production of peptides, such as tirzepatide, retatrutide, and mazdutide, with commercially desirable purity and volume. Similarly, there is a need for efficient and environmentally “green” processes, including stable intermediates to provide such peptides with fewer purification steps. Improved environmentally friendly processes are also desired to provide peptide manufacturing processes producing minimal waste streams for both environmental and operator enhanced safety. The preparation of large-scale, pharmaceutically elegant peptides presents a number of technical challenges that may affect the overall yield and purity. There is a need for processes to avoid the use of transition metals and/or harsh reaction conditions that are incompatible with peptide synthesis.

The present disclosure seeks to meet these needs by providing novel intermediates and processes useful in the manufacture of peptides, such as tirzepatide, retatrutide, and mazdutide.

Tirzepatide (“TZP”) has the structure of SEQ ID NO: 1, as shown below:

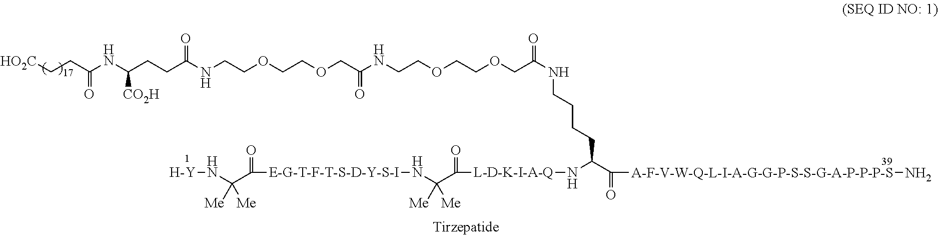

Retatrutide (“GGG”) has the structure of SEQ ID NO: 12, as shown below:

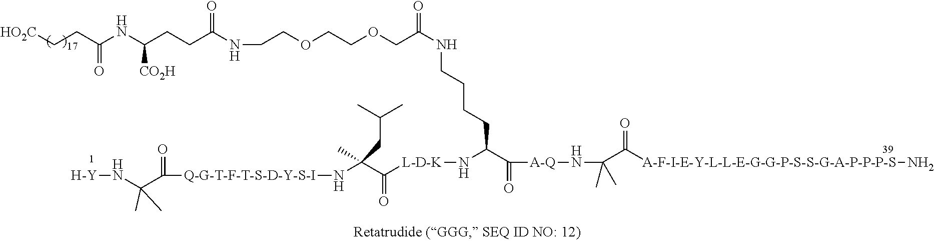

Mazdutide (“OXM”) has the structure of SEQ ID NO: 16, as shown below:

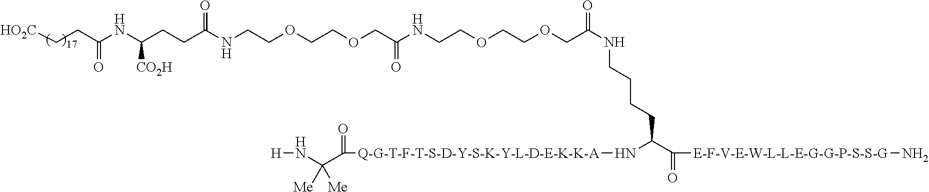

### SUMMARY

The present disclosure describes peptide compounds that are useful for preparing tirzepatide (SEQ ID NO: 1), retatrutide (SEQ ID NO: 12), mazdutide (SEQ ID NO: 16), and other peptides with improved efficiency.

In some embodiments, the present disclosure describes compounds of a Formula (I), or a salt, solvate, or hydrate thereof,

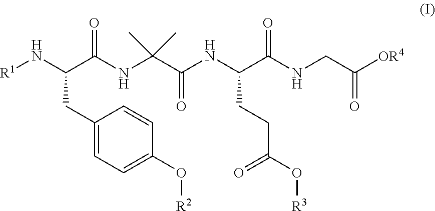

wherein R¹ is H or a protecting group; R² is H or a protecting group; R³ is H or a protecting group; and R⁴ is H or a protecting group; and wherein at least one of R¹, R², R³, and R⁴ is a protecting group. In some embodiments, R¹ is H, Fmoc, or Boc. In some embodiments, R² is H or t-butyl. In some embodiments, R⁴ is H or benzyl. In some embodiments, R³ is H, t-butyl, benzyl, carboxybenzyl, methyl, allyl, cyclohexyl, or trityl. In some embodiments, at least two of R¹, R², R³, and R⁴ are H. In some embodiments, at least two of R¹, R², R³, and R⁴ are protecting groups.

In some embodiments, the compound of Formula (I) is a formula:

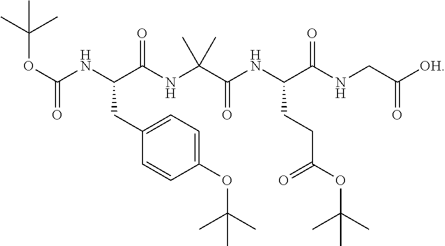 [SMILES: CC(C)(C)OC(=O)CC[C@H](NC(=O)C(C)(C)NC(=O)[C@H](Cc1ccc(OC(C)(C)C)cc1)NC(=O)OC(C)(C)C)C(=O)NCC(=O)O]

In some embodiments, the compound of Formula (I) is a formula:

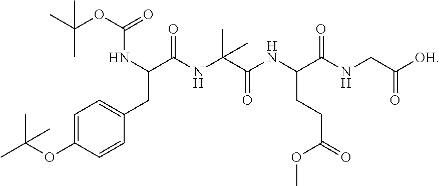 [SMILES: COC(=O)CCC(NC(=O)C(C)(C)NC(=O)C(Cc1ccc(OC(C)(C)C)cc1)NC(=O)OC(C)(C)C)C(=O)NCC(=O)O]

In some embodiments, the compound of Formula (I) is a formula:

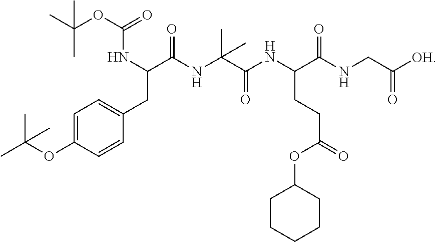 [SMILES: CC(C)(C)OC(=O)NC(Cc1ccc(OC(C)(C)C)cc1)C(=O)NC(C)(C)C(=O)NC(CCC(=O)OC1CCCCC1)C(=O)NCC(=O)O]

In some embodiments, the compound of Formula (I) is a formula:

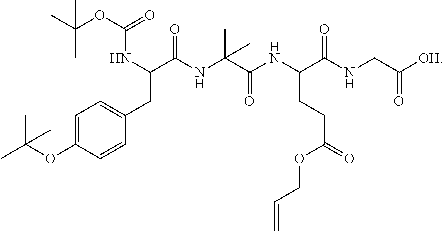 [SMILES: C=CCOC(=O)CCC(NC(=O)C(C)(C)NC(=O)C(Cc1ccc(OC(C)(C)C)cc1)NC(=O)OC(C)(C)C)C(=O)NCC(=O)O]

In some embodiments, the compound of Formula (I) is a formula:

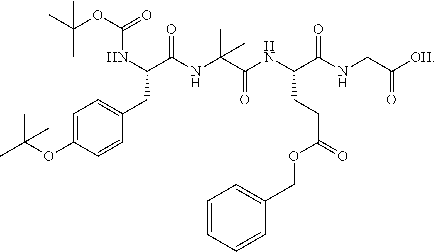 [SMILES: CC(C)(C)OC(=O)N[C@@H](Cc1ccc(OC(C)(C)C)cc1)C(=O)NC(C)(C)C(=O)N[C@@H](CCC(=O)OCc1ccccc1)C(=O)NCC(=O)O]

In some embodiments, the compound of Formula (I) is a solvate. In some embodiments, the solvate is produced from methyl t-butyl ether (MTBE) or a mixture comprising MTBE.

In some embodiments, the compound of Formula (I) is crystalline. In some embodiments, the compound is in the form of a crystalline solid characterized by a peak in the X-ray powder diffraction pattern at diffraction angle 2θ±0.2 degrees of 5.2° in combination of one or more peaks select from 8.4°, 8.8°, 10.4°, 15.5°, 17.1°, and 17.7°. In some embodiments, the compound is in the form of a crystalline solid characterized by a peak in the X-ray powder diffraction pattern at diffraction angle 2θ±0.2 degrees of 6.1° in combination of one or more peaks select from 10.3°, 14.9°, 16.8°, 18.1°, and 18.2°. In some embodiments, the compound is in the form of a crystalline solid characterized by a peak in the X-ray powder diffraction pattern at diffraction angle 2θ±0.2 degrees of 6.2° in combination of one or more peaks select from 8.9°, 12.3°, 14.9°, 15.4°, and 21.8°.

In some embodiments, the present disclosure describes a method of synthesizing the compound of Formula (I), the method comprising:
reacting a compound of formula (Yₚᵣₒₜ)

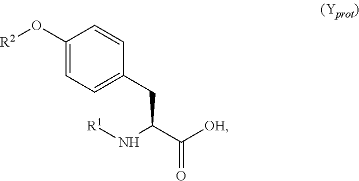

wherein R¹ and R² are protecting groups, and a compound of formula (Aibₚᵣₒₜ)

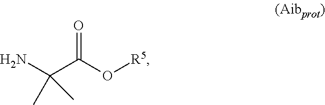

wherein R⁵ is a protecting group, forming a compound of formula (Yₚᵣₒₜ-Aibₚᵣₒₜ),

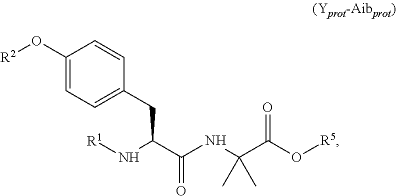

removing the protecting group of R⁵, and forming a compound of formula (Yₚᵣₒₜ-Aib)

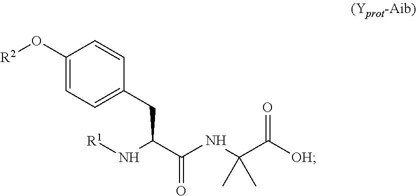

reacting the compound of formula (Yₚᵣₒₜ-Aib) and a compound of formula (Eₚᵣₒₜ)

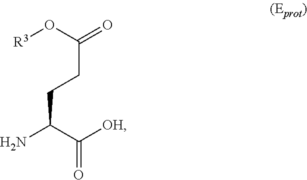

wherein R³ is a protecting group, forming a compound of a formula (Yₚᵣₒₜ-Aib-Eₚᵣₒₜ)

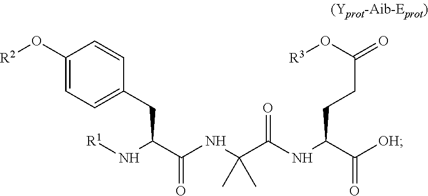

reacting the compound of formula (Yₚᵣₒₜ-Aib-Eₚᵣₒₜ) and a compound of formula (Gₚᵣₒₜ)

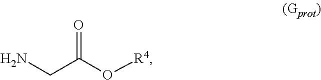

wherein R⁴ is a tin group, forming a compound of formula Yₚᵣₒₜ-Aib-Eₚᵣₒₜ-Gₚᵣₒₜ)

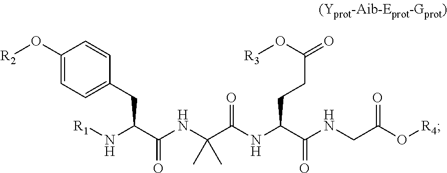

removing one or more of protecting groups R¹, R², R³, and R⁴.

In some embodiments, the present disclosure describes a method of synthesizing a polypeptide of SEQ ID NO: 1, the method comprising conjugating a compound of Formula (I) via the C-terminus of the compound to the N-terminus of a polypeptide of SEQ ID NO: 2,

In some embodiments, the present disclosure describes a method of synthesizing a polypeptide of SEQ ID NO: 26, the method comprising conjugating a compound of Formula (I) via the C-terminus of the compound to the N-terminus of a polypeptide of SEQ ID NO: 27.

In some embodiments, the present disclosure describes compounds of a Formula (II), or a salt, solvate, or hydrate thereof,

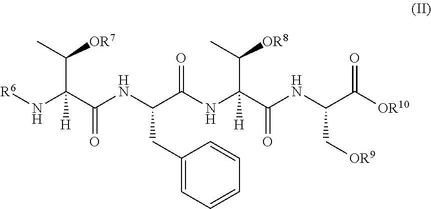

wherein R⁶ is H or a protecting group; R⁷ is H or a protecting group; R⁸ is H or a protecting group; R⁹ is H or a protecting group; and R¹⁰ is H or a protecting group. In some embodiments, each protecting group is independently chosen from Boc, Fmoc, tert-butyl, and trityl groups. In some embodiments, R⁶ is H, Fmoc, or Boc. In some embodiments, R⁷ is H or t-butyl. In some embodiments, R⁸ is H or t-butyl. In some embodiments, R⁹ is H or t-butyl. In some embodiments, R¹⁰ is H or benzyl. In some embodiments, at least one of R⁶, R⁷, R⁸, R⁹, and R¹⁰ is a protecting group. In some embodiments, R⁶, R⁷, R⁸, R⁹, and R¹⁰ are H.

In some embodiments, the compound of Formula (II) is of a formula:

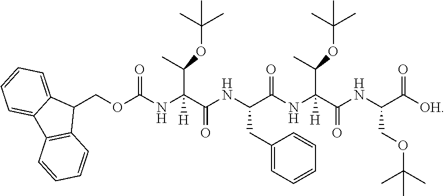 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)O)[C@@H](C)OC(C)(C)C]

In some embodiments, the compound of Formula (I) is a solvate. In some embodiments, the solvate is produced from a solvent comprising heptane.

In some embodiments, the compound of Formula (I) is crystalline. In some embodiments, the compound is in the form of a crystalline solid characterized by a peak in the X-ray powder diffraction pattern at diffraction angle 2θ±0.2 degrees of 7.7-7.9° in combination of one or more peaks select from 5.8°, 10.0°, 10.8-10.9°, 11.3-11.4°, 12.0-12.1°, 12.8°, 14.2-14.4°, and 16.8-17.0°. In some embodiments, the compound is in the form of a crystalline solid characterized by a peak in the X-ray powder diffraction pattern at diffraction angle 2θ±0.2 degrees of 7.3° in combination of one or more peaks select from 5.1°, 5.7°, 7.6°, 9.5°, and 12.4°. In some embodiments, the compound is in the form of a crystalline solid characterized by a peak in the X-ray powder diffraction pattern at diffraction angle 2θ±0.2 degrees of 7.8° in combination of one or more peaks select from 8.5°, 11.5°, 12.0°, 12.8°, 14.30, 15.5°, 20.2° and 23.30. In some embodiments, compound is in the form of a crystalline solid characterized by a peak in the X-ray powder diffraction pattern at diffraction angle 2θ±0.2 degrees of 8.2-8.3° in combination of one or more peaks select from 5.9°, 7.7°, 9.2°, 10.2°, 11.3°, 13.8-13.9°, 15.5-15.7°, 17.10, and 18.5°.

In some embodiments, the present disclosure describes a method of synthesizing a polypeptide of SEQ ID NO: 1, the method comprising (i) conjugating a compound of Formula (II) via the N-terminus of the compound to the C-terminus of a polypeptide of SEQ ID NO: 3 to form a polypeptide of SEQ ID NO: 4; and (ii) conjugating the polypeptide of SEQ ID NO: 4 via its C-terminus to the N-terminus of a polypeptide of SEQ ID NO: 5.

In some embodiments, the present disclosure describes compounds of Formula (III), or a salt, solvate, or hydrate thereof,

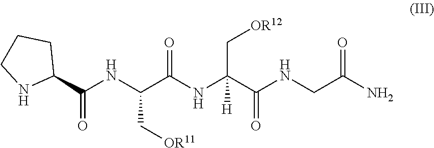

wherein R¹¹ is H or a protecting group; and R¹² is H or a protecting group. In some embodiments, each protecting group is independently chosen from Boc, Fmoc, tert-butyl, and trityl groups. In some embodiments, R¹¹ is H or t-butyl. In some embodiments, R¹² is H or t-butyl. In some embodiments, at least one of R¹¹ and R¹² is a protecting group. In some embodiments, R¹¹ and R¹² are H.

In some embodiments, the compound of Formula (III) is a compound of a formula:

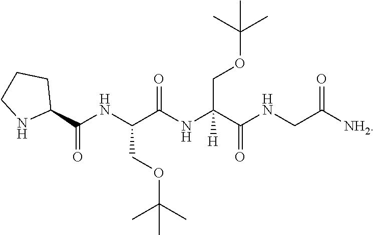 [SMILES: CC(C)(C)OC[C@H](NC(=O)[C@H](COC(C)(C)C)NC(=O)[C@@H]1CCCN1)C(=O)NCC(N)=O]

In some embodiments, the compound of Formula (III) is a solvate.

In some embodiments, the compound of Formula (III) is crystalline. In some embodiments, the compound is in the form of a crystalline solid characterized by a peak in the X-ray powder diffraction pattern at diffraction angle 2θ±0.2 degrees of 8.0° in combination of one or more peaks select from 7.0°, 10.3°, 14.1°, 15.2°, 16.7°, 18.0°, 19.0°, 19.7°, 20.8°, and 21.9°. In some embodiments, the compound is in the form of a crystalline solid characterized by a peak in the X-ray powder diffraction pattern at diffraction angle 2θ±0.2 degrees of 9.0° in combination of one or more peaks select from 5.7°, 9.9°, 16.2°, 17.1°, 17.9°, 18.1°, 18.4°, 18.8°, 19.9°, 20.1°, and 22.5°. In some embodiments, the compound is in the form of a crystalline solid characterized by a peak in the X-ray powder diffraction pattern at diffraction angle 2θ±0.2 degrees of 10.5° in combination of one or more peaks select from 5.6°, 11.9°, 13.3°, 15.4°, 15.6°, 18.1°, 19.9°, and 21.1°. In some embodiments, the compound is in the form of a crystalline solid characterized by a peak in the X-ray powder diffraction pattern at diffraction angle 2θ±0.2 degrees of 10.7° in combination of one or more peaks select from 4.9°, 14.8°, 20.3°, and 21.5°. In some embodiments, the compound is in the form of a crystalline solid characterized by a peak in the X-ray powder diffraction pattern at diffraction angle 2θ±0.2 degrees of 10.5° in combination of one or more peaks select from 5.9°, 10.5°, 10.9°, 12.1°, 13.1°, 15.9°, 17.5°, 20.9°, 21.1°, and 21.9°. In some embodiments, the compound is in the form of a crystalline solid characterized by a peak in the X-ray powder diffraction pattern at diffraction angle 2θ±0.2 degrees of 7.8° in combination of one or more peaks select from 11.3°, 11.5°, 15.4°, 15.6°, and 21.5°. In some embodiments, the compound is in the form of a crystalline solid characterized by a peak in the X-ray powder diffraction pattern at diffraction angle 2θ±0.2 degrees of 10.0° in combination of one or more peaks select from 8.1°, 12.5°, 13.5°, 14.7°, 17.8°, 18.8°, 20.0°, and 22.4°. In some embodiments, the compound is in the form of a crystalline solid characterized by a peak in the X-ray powder diffraction pattern at diffraction angle 2θ±0.2 degrees of 21.1° in combination of one or more peaks select from 5.6°, 10.5°, 10.8°, 11.9°, 15.4°, and 23.8°.

In some embodiments, the present disclosure describes a method of synthesizing a polypeptide of SEQ ID NO: 1, the method comprising (i) conjugating a compound of Formula (III) via the N-terminus of the compound to the C-terminus of a polypeptide of SEQ ID NO: 6 to form a polypeptide of SEQ ID NO: 7; and (ii) conjugating the polypeptide of SEQ ID NO: 7 via its C-terminus to the N-terminus of a polypeptide of SEQ ID NO: 8.

In some embodiments, the present disclosure describes compounds of a Formula (IV), or a salt, solvate, or hydrate thereof,

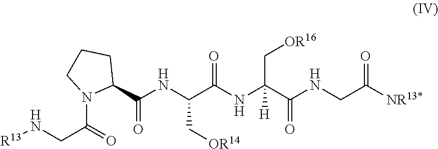

wherein R¹³ is H or a protecting group; R^{13*}is H or a protecting group; R¹⁴ is H or a protecting group; and R¹⁵ is H or a protecting group. In some embodiments, each protecting group is independently chosen from Boc, Fmoc, tert-butyl, and trityl groups. In some embodiments, R¹³ is H, Fmoc, or Boc. In some embodiments, R^{13*} is H. In some embodiments, R¹⁴ is H or t-butyl. In some embodiments, R¹⁵ is H or t-butyl. In some embodiments, at least one of R¹³, R^{13*}, R¹⁴ and R¹⁵ is a protecting group. In some embodiments, R¹³, R^{13*}, R¹⁴, and R¹⁵ are H.

In some embodiments, the compound of Formula (IV) is of a formula:

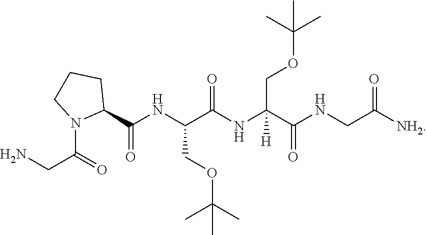 [SMILES: CC(C)(C)OC[C@H](NC(=O)[C@H](COC(C)(C)C)NC(=O)[C@@H]1CCCN1C(=O)CN)C(=O)NCC(N)=O]

In some embodiments, the compound of Formula (IV) is a solvate.

In some embodiments, the compound of Formula (IV) is crystalline. In some embodiments, the compound is in the form of a crystalline solid characterized by a peak in the X-ray powder diffraction pattern at diffraction angle 2θ±0.2 degrees of 5.1° in combination of one or more peaks select from 4.3°, 6.1°, 8.0°, 10.1°, and 18.7°. In some embodiments, the compound is in the form of a crystalline solid characterized by a peak in the X-ray powder diffraction pattern at diffraction angle 2θ±0.2 degrees of 5.2° in combination of one or more peaks select from 6.0°, 6.7°, 10.0°, 10.3°, 16.4°, 17.8°, 18.3° 19.4°, and 22.4°.

In some embodiments, the present disclosure describes a method of synthesizing a polypeptide of SEQ ID NO: 1, the method comprising (i) conjugating a compound of Formula (IV) via the N-terminus of the compound to the C-terminus of a polypeptide of SEQ ID NO: 9 to form a polypeptide of SEQ ID NO: 7; and (ii) conjugating the polypeptide of SEQ ID NO: 7 via its C-terminus to the N-terminus of a polypeptide of SEQ ID NO: 8.

In some embodiments, the compound of Formula (IV) is of a formula,

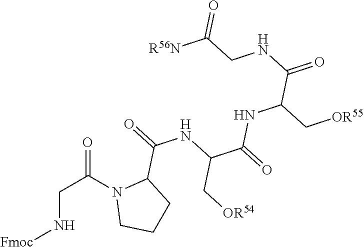

wherein R⁵⁴ is H or a protecting group; R⁵⁵ is H or a protecting group; and R⁵⁶ is H or a protecting group. In some embodiments, each protecting group is independently chosen from Boc, Fmoc, tert-butyl, and trityl groups. In some embodiments, R⁵⁴ is H or tert-butyl. In some embodiments, R⁵⁵ is H or tert-butyl. In some embodiments, R⁵⁶ is H or Boc. In some embodiments, at least one of R⁵⁴, R⁵⁵, and R⁵⁶ is H. In some embodiments, at least one of R⁵⁴, R⁵⁵, and R⁵⁶ is a protecting group.

In some embodiments, the compound of Formula (IV) has a structure of Formula (IV-b):

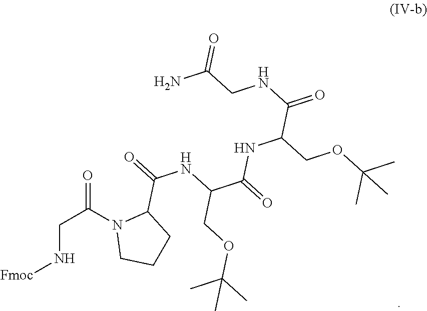

In some embodiments, the compound of Formula (IV-b) is a solvate. In some embodiments, the compound is a solvate produced from acetone. In some embodiments, the compound is a de-solvate.

In some embodiments, the compound of Formula (IV-b) is crystalline. In some embodiments, the compound is the compound is in the form of a crystalline solid characterized by peaks in the X-ray powder diffraction pattern at diffraction angle 2-theta±0.2 degrees of 5.8° and 18.5° in combination of one or more peaks chosen from 8.6°, 9.4°, 12.9°, 13.8°, 17.2°, and 19.4°. In some embodiments, the compound is in the form of a crystalline solid characterized by peaks in the X-ray powder diffraction pattern at diffraction angle 2-theta±0.2 degrees of 7.0-7.1° and 7.5-7.7° in combination of one or more peaks chosen from 5.3-5.4°, 9.7-9.9°, and 14.7-14.9. In some embodiments, the compound is in the form of a crystalline solid characterized by a peak in the X-ray powder diffraction pattern at diffraction angle 2-theta±0.2 degrees of 8.3° in combination of one or more peaks chosen from 6.3°, 11.4°, 14.3°, and 16.6°. In some embodiments, the compound is in the form of a crystalline solid characterized by a peak in the X-ray powder diffraction pattern at diffraction angle 2-theta±0.2 degrees of 7.2° in combination of one or more peaks chosen from 6.8°, 8.6°, 15.8°, and 18.9°. In some embodiments, the compound is in the form of a crystalline solid characterized by a peak in the X-ray powder diffraction pattern at diffraction angle 2-theta±0.2 degrees of 6.1° in combination of one or more peaks chosen from 6.3°, 7.8°, 10.0°, and 12.4°.

In some embodiments, the present disclosure describes a method of synthesizing the compound of Formula (IV-b), the method comprising:
reacting a compound of formula (Fmoc-G) and a compound of formula (Pₚᵣₒₜ) to form a compound of formula (Fmoc-G-Pₚᵣₒₜ)

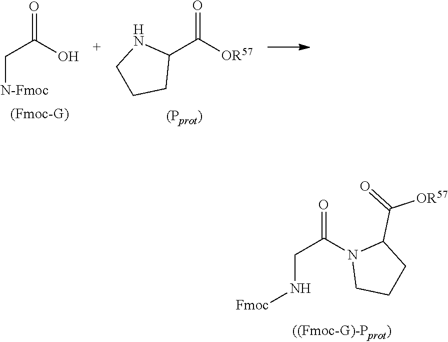

wherein R⁵⁷ is a protecting group;
removing the protecting group R⁵⁷ to form a compound of (Fmoc-G-P)

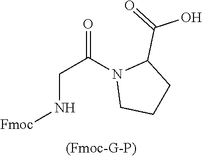

reacting the compound of formula (Fmoc-G-P) and a compound of formula (Sₚᵣₒₜ₂) to form a compound of a formula (Fmoc-G-P-Sₚᵣₒₜ₂)

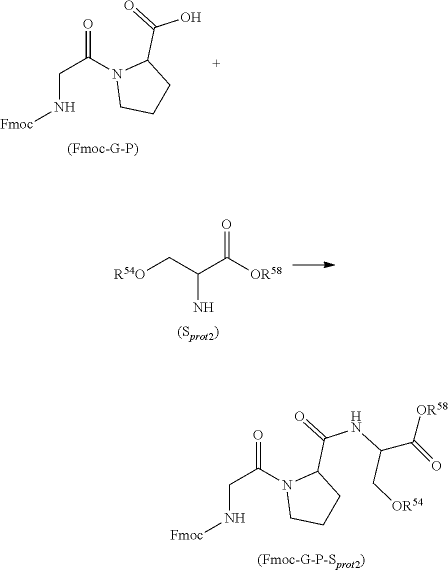

wherein R⁵⁴ and R⁵⁸ are protecting groups;
removing the protecting group R⁵⁸ to form a compound of (Fmoc-G-P-Sₚᵣₒₜ₁)

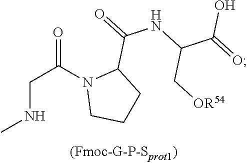

reacting the compound of formula (Fmoc-G-P-Sₚᵣₒₜ₁) and a compound of formula (Sₚᵣₒₜ₂) to form a compound of formula (Fmoc-G-P-Sₚᵣₒₜ₁-Sₚᵣₒₜ₂)

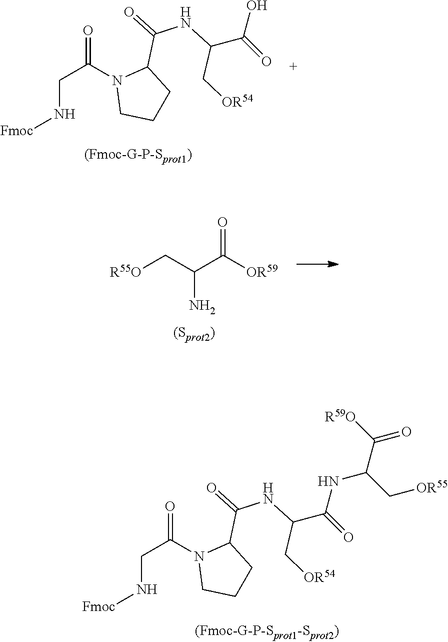

wherein R⁵⁵ and R⁵⁹ are protecting groups; and
removing the protecting group R⁵⁹ to form a compound of (Fmoc-F-Gₚᵣₒₜ₁-Sₚᵣₒₜ₁-Sₚᵣₒₜ₁)

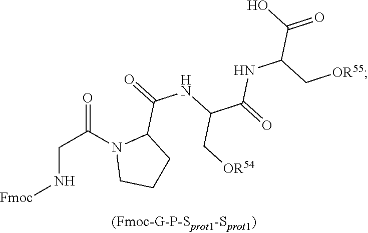

reacting the compound of formula (Fmoc-G-P-Sₚᵣₒₜ₁-Sₚᵣₒₜ₁) and a compound of formula (Gₚᵣₒₜ) to form a compound of formula (Fmoc-G-P-Sₚᵣₒₜ₁-Sₚᵣₒₜ₁-Gₚᵣₒₜ)

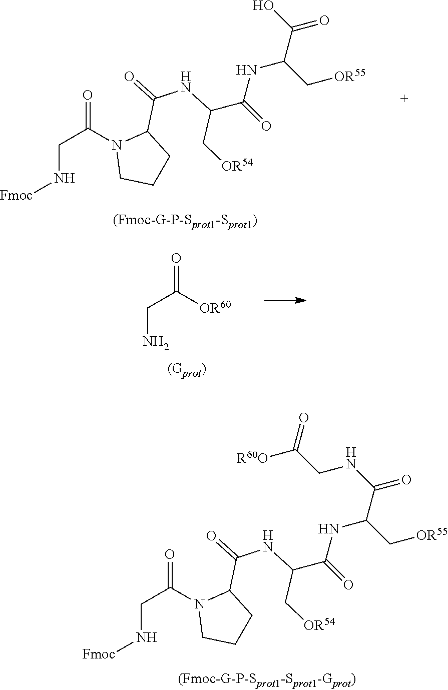

wherein R⁶⁰ is a protecting group;
converting the compound of formula (Fmoc-G-P-Sₚᵣₒₜ₁-Sₚᵣₒₜ₁-Gₚᵣₒₜ) to the compound of claim 36

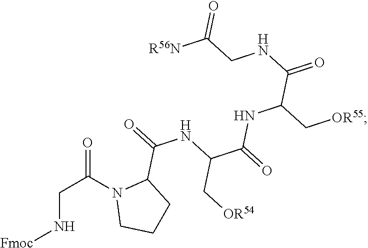

and
optionally removing one or more protecting groups R⁵⁴, R⁵⁵, and R⁵⁶.

In some embodiments, each protecting group is independently chosen from Boc, Fmoc, tert-butyl and trityl groups.

In some embodiments, the present disclosure describes a method of synthesizing the polypeptide of SEQ ID NO: 12, the method comprising: (i) conjugating a compound of Formula (IV-b) via the C-terminus of the compound to the N-terminus of a polypeptide of SEQ ID NO: 22, and (ii) conjugating the resulting compound via its N-terminus to the C-terminus of a polypeptide of SEQ ID NO: 23.

In some embodiments, the present disclosure describes a method of synthesizing the polypeptide of SEQ ID NO: 24, the method comprising conjugating a compound of Formula (IV-b) via the C-terminus of the compound to the N-terminus of a polypeptide of SEQ ID NO: 22.

In some embodiments, the present disclosure describes a method of synthesizing the polypeptide of SEQ ID NO: 16, the method comprising conjugating a compound of Formula (IV-b) via the N-terminus of the compound to the C-terminus of a polypeptide of SEQ ID NO: 25.

In some embodiments, the present disclosure describes a method of synthesizing the polypeptide of SEQ ID NO: 1, the method comprising: (i) conjugating a compound of Formula (IV-b) via the C-terminus of the compound to the N-terminus of a polypeptide of SEQ ID NO: 8, and (ii) conjugating the resulting compound via its N-terminus to the C-terminus of a polypeptide of SEQ ID NO: 9.

In some embodiments, the present disclosure describes a compound of a Formula (V), or a salt, solvate, or hydrate thereof,

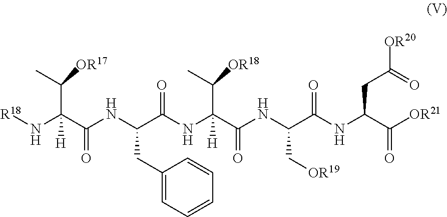

wherein R¹⁶ is H or a protecting group; R¹⁷ is H or a protecting group; R¹⁸ is H or a protecting group; R¹⁹ is H or a protecting group; R²⁰ is H or a protecting group; and R²¹ is H or a protecting group. In some embodiments, each protecting group is independently chosen from Boc, Fmoc, tert-butyl, and trityl groups. In some embodiments, R¹⁶ is H, Fmoc, or Boc. In some embodiments, R¹⁷ is H or t-butyl. In some embodiments, R¹⁸ is H or t-butyl. In some embodiments, R¹⁹ is H or t-butyl. In some embodiments, R²⁰ is H or t-butyl. In some embodiments, R²¹ is H or benzyl. In some embodiments, at least one of R¹⁶, R¹⁷, R¹⁸, R¹⁹, R²⁰, and R²¹ is a protecting group. In some embodiments, R¹⁶, R¹⁷, R¹⁸, R¹⁹, R²⁰, and R²¹ are H.

In some embodiments, the compound of Formula (V) is of a formula:

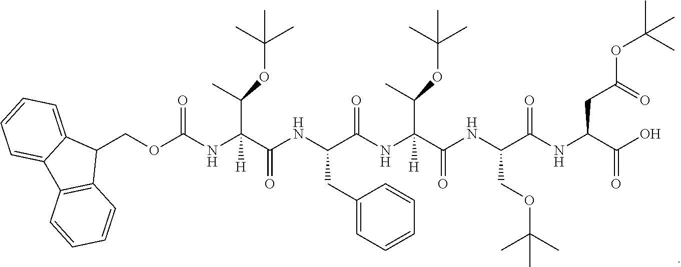 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)N[C@@H](CC(=O)OC(C)(C)C)C(=O)O)[C@@H](C)OC(C)(C)C]

In some embodiments, the compound of Formula (V) is a solvate.

In some embodiments, the compound of Formula (V) is crystalline. In some embodiments, the compound is in the form of a crystalline solid characterized by a peak in the X-ray powder diffraction pattern at diffraction angle 2θ±0.2 degrees of 5.8-6.1° in combination of one or more peaks select from 6.7-7.1° and 8.8-9.0°. In some embodiments, the compound is in the form of a crystalline solid characterized by a peak in the X-ray powder diffraction pattern at diffraction angle 2θ±0.2 degrees of 5.0-5.2° in combination of one or more peaks select from 5.3-5.4°, 5.7-6.0°, 6.1-6.2°, 7.6-7.9°, and 8.7-9.1°.

In some embodiments, the present disclosure describes a method of synthesizing a polypeptide of SEQ ID NO: 1, the method comprising (i) conjugating a compound of Formula (V) via the N-terminus of the compound to the C-terminus of a polypeptide of SEQ ID NO: 3 to form a polypeptide of SEQ ID NO: 10; and (ii) conjugating the polypeptide of SEQ ID NO: 10 via its C-terminus to the N-terminus of a polypeptide of SEQ ID NO: 11.

In some embodiments, the present disclosure describes a compound of Formula (VI), or a salt, solvate, or hydrate thereof,

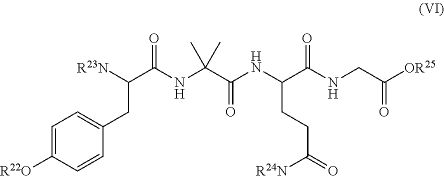

wherein R²² is H or a protecting group; R²³ is H or a protecting group; R²⁴ is H or a protecting group, and R²⁵ is H or a protecting group. In some embodiments, each protecting group is independently chosen from Boc, Fmoc, tert-butyl, and trityl groups. In some embodiments, R²² is H or Boc. In some embodiments, R²³ is H or tert-butyl. In some embodiments, R²⁴ is H or trityl. In some embodiments, R²⁵ is H or tert-butyl. In some embodiments, at least one of R²², R²³, R²⁴, and R²⁵ is H. In some embodiments, at least one of R²², R²³, R²⁴, and R²⁵ is a protecting group.

In some embodiments, the compound of Formula (VI) is a compound of Formula (VI-a):

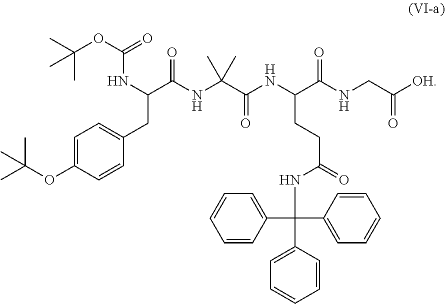 [SMILES: CC(C)(C)OC(=O)NC(Cc1ccc(OC(C)(C)C)cc1)C(=O)NC(C)(C)C(=O)NC(CCC(=O)NC(c1ccccc1)(c1ccccc1)c1ccccc1)C(=O)NCC(=O)O]

In some embodiments, the compound of Formula (VI) is a solvate. In some embodiments, the compound is a solvate produced from pentyl acetate, mixtures comprising pentyl acetate, ethyl acetate, or mixtures comprising 2-methyl tetrahydrofuran and t-amyl methyl ether. In some embodiments, the mixtures comprising pentyl acetate are chosen from mixtures comprising pentyl acetate and t-butyl ethyl ether, mixtures comprising pentyl acetate and t-amyl methyl ether, or mixtures comprising pentyl acetate and heptane. In some embodiments, the compound is a de-solvate or a partial de-solvate.

In some embodiments, the compound of Formula (VI) is crystalline. In some embodiments, the compound is in the form of a crystalline solid characterized by a peak in the X-ray powder diffraction pattern at diffraction angle 2-theta±0.2 degrees of 6.3-6.4° in combination of one or more peaks chosen from 4.5°, 7.1°, 13.0-13.1°, 15.9-16.0°, and 18.4-18.6°. In some embodiments, the compound is in the form of a crystalline solid characterized by a peak in the X-ray powder diffraction pattern at diffraction angle 2-theta±0.2 degrees of 7.0-7.2° in combination of one or more peaks chosen from 5.0-5.4°, 7.6-7.7°, 8.8-8.9°, 9.4-9.5°, and 12.5-12.7°.

In some embodiments, the present disclosure describes a method of synthesizing a compound of Formula (VI), the method comprising:
reacting a compound of formula (Yₚᵣₒₜ) and a compound of formula (Aibₚᵣₒₜ) to form a compound of formula (Yₚᵣₒₜ-Aibₚᵣₒₜ)

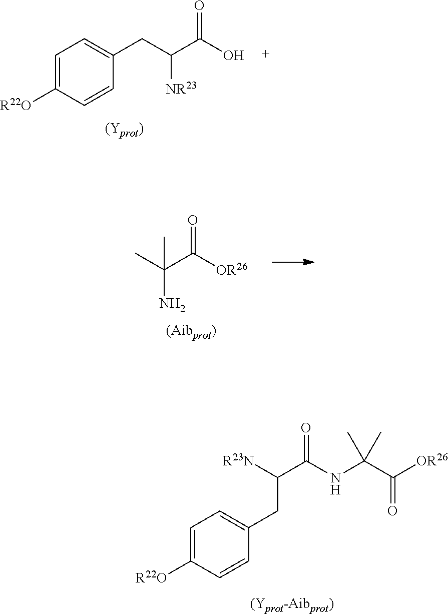

wherein R²², R²³, and R²⁶ are protecting groups;
removing the protecting group R²⁶ to form a compound of (Yₚᵣₒₜ-Aib)

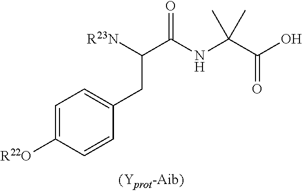

reacting the compound of formula (Yₚᵣₒₜ-Aib) and a compound of formula (Qₚᵣₒₜ₂) to form a compound of a formula (Yₚᵣₒₜ-Aib-Qₚᵣₒₜ₂)

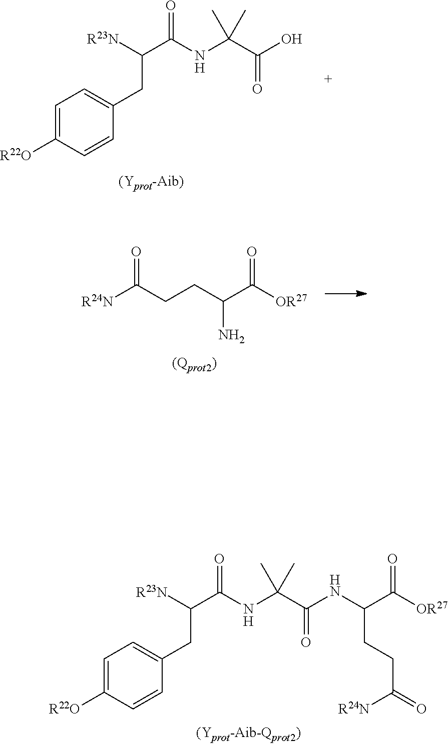

wherein R²⁴ and R²⁷ are protecting groups;
removing the protecting group R²⁷ to form a compound of (Yₚᵣₒₜ-Aib-Qₚᵣₒₜ₁)

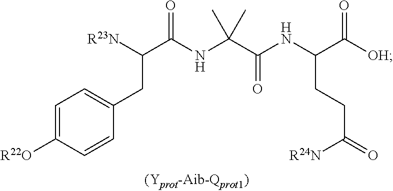

reacting the compound of formula (Yₚᵣₒₜ-Aib-Qₚᵣₒₜ₁) and a compound of formula (Gₚᵣₒₜ) to form a compound of Formula (VI)

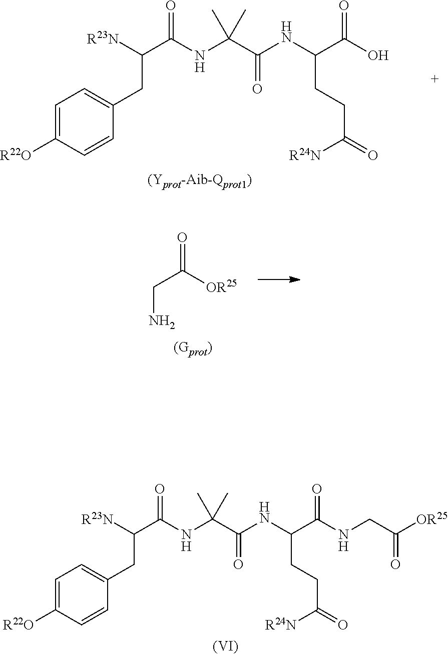

wherein R²⁵ is a protecting group; and
optionally removing one or more protecting groups R²², R²³, R²⁴, and R²⁵.

In some embodiments, each protecting group is independently chosen from Boc, Fmoc, tert-butyl, and trityl groups.

In some embodiments, the present disclosure describes a method of synthesizing a polypeptide of SEQ ID NO: 12, the method comprising conjugating a compound of Formula (VI) via the C-terminus of the compound to the N-terminus of a polypeptide of SEQ ID NO: 13.

In some embodiments, the present disclosure describes a method of synthesizing a polypeptide of SEQ ID NO: 14, the method comprising conjugating a compound of Formula (VI) via the C-terminus of the compound to the N-terminus of a polypeptide of SEQ ID NO: 15.

In some embodiments, the present disclosure describes a compound of Formula (VII), or a salt, solvate, or hydrate thereof,

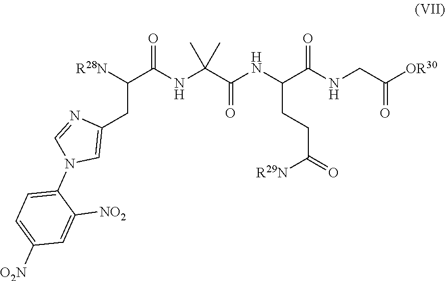

wherein R²⁸ is H or a protecting group; R²⁹ is H or a protecting group, and R³⁰ is H or a protecting group. In some embodiments, each protecting group is independently chosen from Boc, Fmoc, tert-butyl, and trityl groups. In some embodiments, R²⁸ is H or Boc. In some embodiments, R²⁹ is H or trityl. In some embodiments, R³⁰ is H or tert-butyl. In some embodiments, at least one of R²⁸, R²⁹, and R³⁰ is H. In some embodiments, at least one of R²⁸, R²⁹, and R³⁰ is a protecting group.

In some embodiments, the compound is Formula (VII) is a compound of Formula (VII-a):

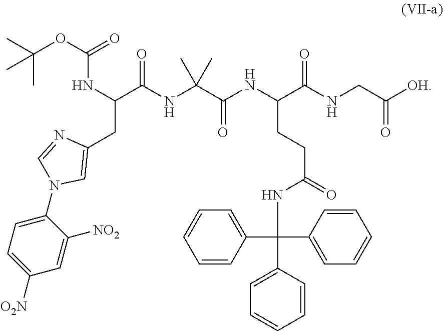 [SMILES: CC(C)(C)OC(=O)NC(Cc1cn(-c2ccc([N+](=O)[O-])cc2[N+](=O)[O-])cn1)C(=O)NC(C)(C)C(=O)NC(CCC(=O)NC(c1ccccc1)(c1ccccc1)c1ccccc1)C(=O)NCC(=O)O]

In some embodiments, the compound is Formula (VII) is a solvate. In some embodiments, the compound is a solvate produced from any one of a mixture of acetonitrile and methyl tert-butyl ether, a mixture of nitromethane and methyl tert-butyl ether, a mixture of tetrahydrofuran and methyl tert-butyl ether, methyl acetate, and ethyl acetate.

In some embodiments, the compound of Formula (VII) is crystalline. In some embodiments, the compound is in the form of a crystalline solid characterized by a peak in the X-ray powder diffraction pattern at diffraction angle 2-theta±0.2 degrees of 4.8° in combination of one or more peaks chosen from 5.6°, 6.2°, 14.8°, and 15.6°. In some embodiments, the compound is in the form of a crystalline solid characterized by a peak in the X-ray powder diffraction pattern at diffraction angle 2-theta±0.2 degrees of 5.3° in combination of one or more peaks chosen from 7.7°, 10.5°, 11.3°, 11.6°, and 14.4°. In some embodiments, the compound is in the form of a crystalline solid characterized by peaks in the X-ray powder diffraction pattern at diffraction angle 2-theta±0.2 degrees of 6.20 and 6.90.

In some embodiments, the present disclosure describes a method of synthesizing a compound is Formula (VII), the method comprising:
reacting a compound of formula (H(dnp)ₚᵣₒₜ) and a compound of formula (Aibₚᵣₒₜ) to form a compound of formula (H(dnp)ₚᵣₒₜ-Aibₚᵣₒₜ)

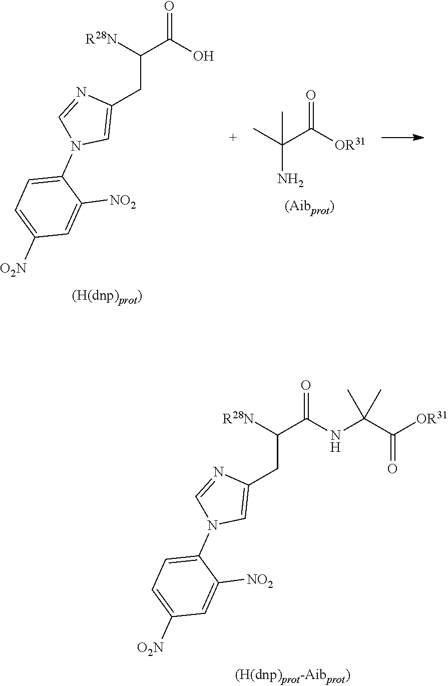

wherein R²⁸ and R³¹ are protecting groups;
removing the protecting group R³¹ to form a compound of (H(dnp)ₚᵣₒₜ-Aib)

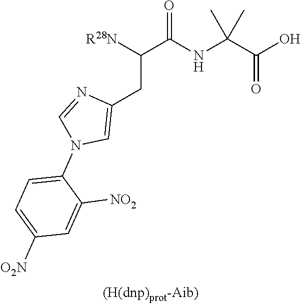

reacting the compound of formula (H(dnp)ₚᵣₒₜ-Aib) and a compound of formula (Qₚᵣₒₜ₂) to form a compound of a formula (H(dnp)ₚᵣₒₜ-Aib-Qₚᵣₒₜ₂)

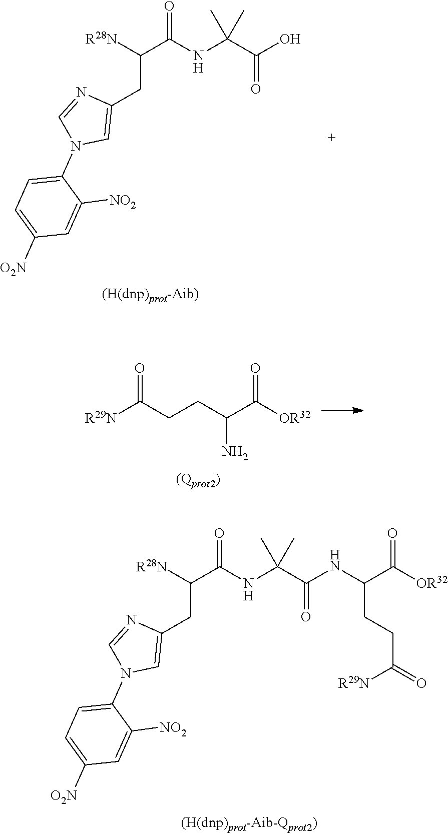

wherein R²⁹ and R³² are protecting groups;
removing the protecting group R³² to form a compound of (H(dnp)ₚᵣₒₜ-Aib-Qₚᵣₒₜ₁)

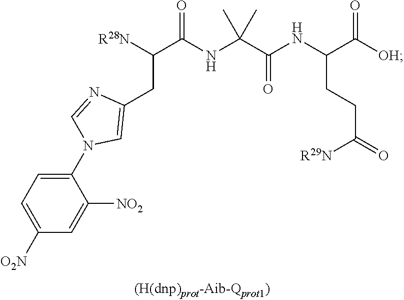

reacting the compound of formula (H(dnp)ₚᵣₒₜ-Aib-Qₚᵣₒₜ₁) and a compound of formula (Gₚᵣₒₜ) to form a compound of Formula (VII)

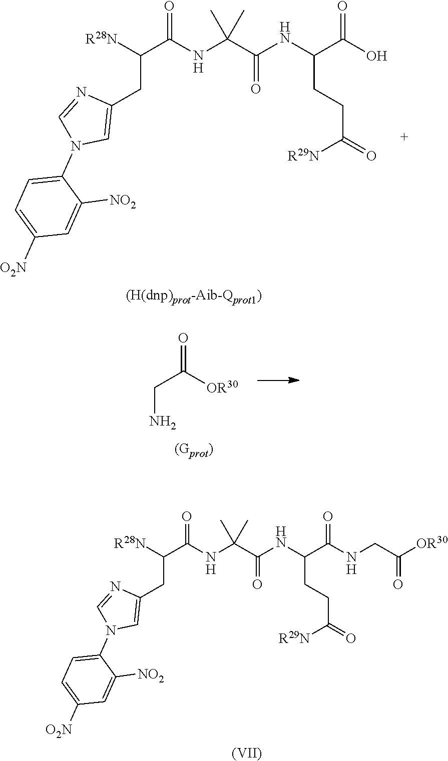

wherein R³⁰ is a protecting group; and
optionally removing one or more protecting groups R²⁸, R²⁹, and R³⁰.

In some embodiments, each protecting group is independently chosen from Boc, Fmoc, tert-butyl, and trityl groups.

In some embodiments, the present disclosure describes a method of synthesizing a polypeptide of SEQ ID NO: 16, the method comprising conjugating a compound of Formula (VII) via the C-terminus of the compound to the N-terminus of a polypeptide of SEQ ID NO: 17.

In some embodiments, the present disclosure describes a compound of Formula (VIII), or a salt, solvate, or hydrate thereof,

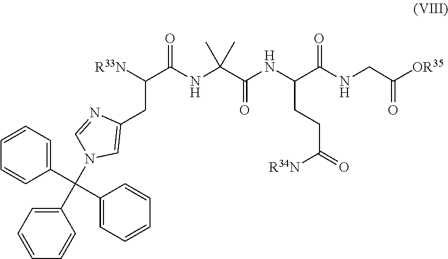

wherein R³³ is H or a protecting group; R³⁴ is H or a protecting group; and R³⁵ is H or a protecting group. In some embodiments, each protecting group is independently chosen from Boc, Fmoc, tert-butyl, and trityl groups. In some embodiments, R³³ is H or Boc. In some embodiments, R³⁴ is H or trityl. In some embodiments, R³⁵ is H or tert-butyl. In some embodiments, at least one of R³³, R³⁴, and R³⁵ is H. In some embodiments, at least one of R³³, R³⁴, and R³⁵ is a protecting group.

In some embodiments, the compound of Formula (VIII) is a compound of Formula (VIII-a):

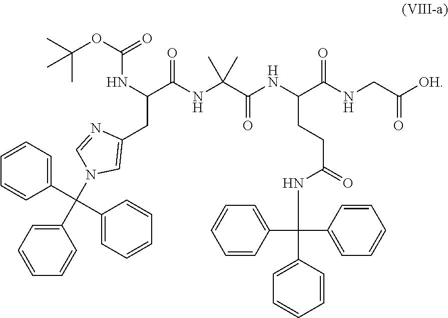 [SMILES: CC(C)(C)OC(=O)NC(Cc1cn(C(c2ccccc2)(c2ccccc2)c2ccccc2)cn1)C(=O)NC(C)(C)C(=O)NC(CCC(=O)NC(c1ccccc1)(c1ccccc1)c1ccccc1)C(=O)NCC(=O)O]

In some embodiments, the compound of Formula (VIII) is a solvate. In some embodiments, the compound is a solvate produced from any one of a mixture of tetrahydrofuran and methyl tert-butyl ether, a mixture of tetrahydrofuran and heptane, a mixture of 1,4-dioxane and water, a mixture of ethyl acetate and methyl tert-butyl ether, and a mixture of acetonitrile and methyl tert-butyl ether.

In some embodiments, the compound of Formula (VIII) is crystalline. In some embodiments, the compound is in the form of a crystalline solid characterized by a peak in the X-ray powder diffraction pattern at diffraction angle 2-theta±0.2 degrees of 4.7° in combination of one or more peaks chosen from 5.5°, 8.2°, 10.1°, 11.8°, 13.3°, 13.6°, and 18.9°. In some embodiments, the compound is in the form of a crystalline solid characterized by a peak in the X-ray powder diffraction pattern at diffraction angle 2-theta±0.2 degrees of 5.8° in combination of one or more peaks chosen from 5.3°, 8.9°, 9.2°, 15.2°, 18.6°, and 19.5°.

In some embodiments, the present disclosure describes a method of synthesizing a compound of Formula (VIII), the method comprising:
reacting a compound of formula (H(trt)ₚᵣₒₜ) and a compound of formula (Aibₚᵣₒₜ) to form a compound of formula (H(trt)ₚᵣₒₜ-Aibₚᵣₒₜ)

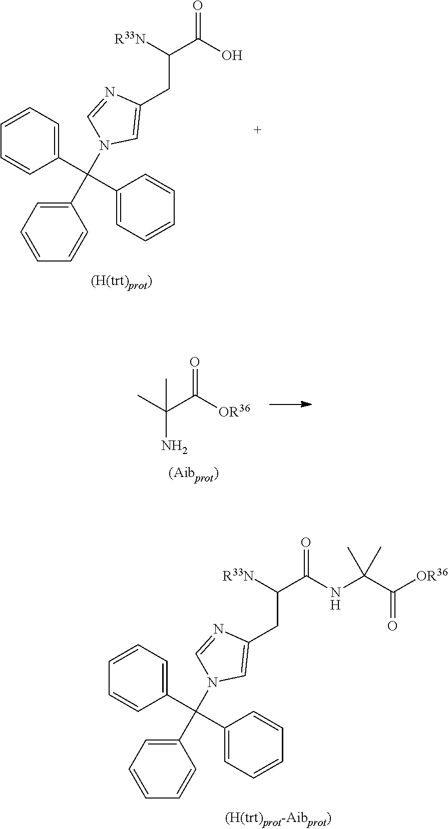

wherein R³³ and R³⁶ are protecting groups;
removing the protecting group R³⁶ to form a compound of (H(trt)ₚᵣₒₜ-Aib)

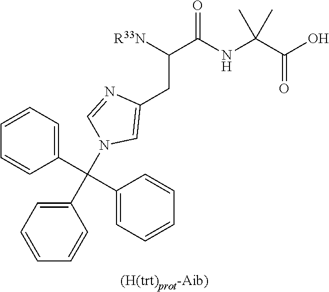

reacting the compound of formula (H(trt)ₚᵣₒₜ-Aib) and a compound of formula (Qₚᵣₒₜ₂) to form a compound of a formula (H(trt)ₚᵣₒₜ-Aib-Qₚᵣₒₜ₂)

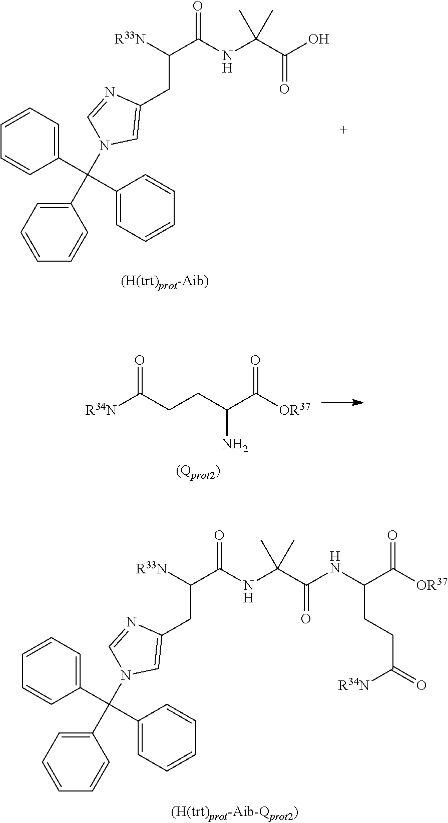

wherein R³³ and R³⁷ are protecting groups;
removing the protecting group R³ to form a compound of (H(trt)ₚᵣₒₜ-Aib-Qₚᵣₒₜ₁)

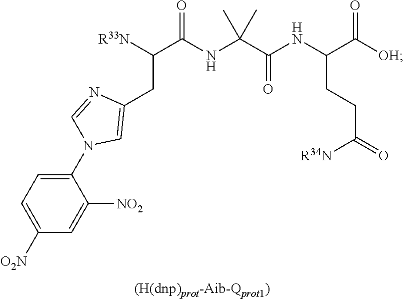

reacting the compound of formula (H(trt)ₚᵣₒₜ-Aib-Qₚᵣₒₜ₁) and a compound of formula (Gₚᵣₒₜ) to form a compound of Formula (VIII)

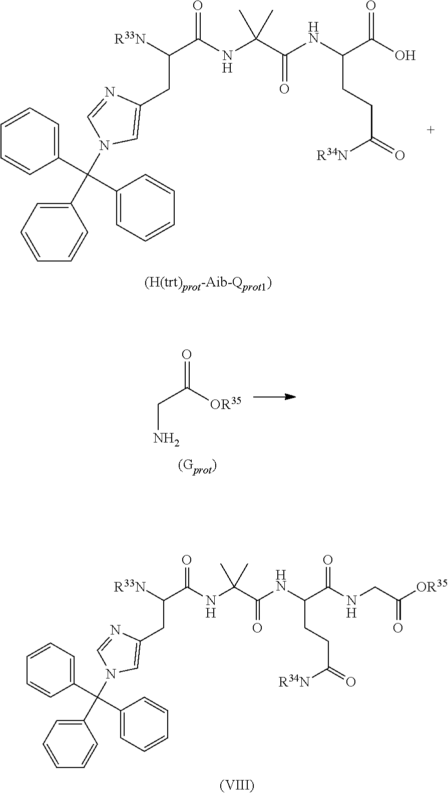

wherein R³⁵ is a protecting group; and
optionally removing one or more protecting groups R³³, R³⁴, and R³⁵.

In some embodiments, each protecting group is independently chosen from Boc, Fmoc, tert-butyl, and trityl groups.

In some embodiments, the present disclosure describes a method of synthesizing a polypeptide of SEQ ID NO: 16, the method comprising conjugating a compound of Formula (VIII) via the C-terminus of the compound to the N-terminus of a polypeptide of SEQ ID NO: 17.

In some embodiments, the present disclosure describes a compound of Formula (IX), or a salt, solvate, or hydrate thereof,

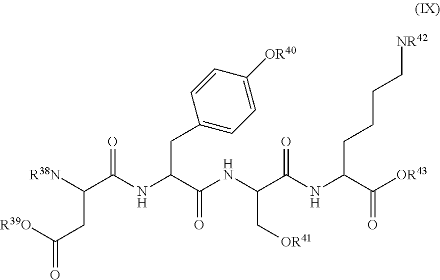

wherein R³⁸ is H or a protecting group; R³⁹ is H or a protecting group; R⁴⁰ is H or a protecting group; R⁴¹ is H or a protecting group; R⁴² is H or a protecting group; and R⁴³ is H or a protecting group. In some embodiments, each protecting group is independently chosen from Boc, Fmoc, tert-butyl, and trityl groups. In some embodiments, R³⁸ is H or Fmoc. In some embodiments, R³⁹ is H or tert-butyl. In some embodiments, R⁴⁰ is H or tert-butyl. In some embodiments, R⁴¹ is H or tert-butyl. In some embodiments, R⁴² is H or Boc. In some embodiments, R⁴³ is H or tert-butyl. In some embodiments, at least one of R³⁸, R³⁹, R⁴⁰, R⁴¹, R⁴², and R⁴³ is H. In some embodiments, at least one of R³⁸, R³⁹, R⁴⁰, R⁴¹, R⁴², and R⁴³ is a protecting group.

In some embodiments, the compound is Formula (IX) is a compound of Formula (IX-a):

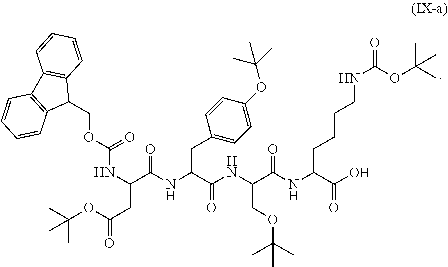 [SMILES: CC(C)(C)OCC(NC(=O)C(Cc1ccc(OC(C)(C)C)cc1)NC(=O)C(CC(=O)OC(C)(C)C)NC(=O)OCC1c2ccccc2-c2ccccc21)C(=O)NC(CCCCNC(=O)OC(C)(C)C)C(=O)O]

In some embodiments, the compound is Formula (IX) is a solvate. In some embodiments, the compound is a solvate produced from any one of a mixture of methyl acetate and dibutyl ether, a mixture of acetone and dibutyl ether, a mixture of acetonitrile and dibutyl ether, a mixture of ethyl acetate and dibutyl ether, a mixture of methyl acetate and heptane, and a mixture of methyl ethyl ketone and dibutyl ether. In some embodiments, the compound is a de-solvate or is anhydrous.

In some embodiments, the compound is Formula (IX) is crystalline. In some embodiments, the compound is in the form of a crystalline solid characterized by a peak in the X-ray powder diffraction pattern at diffraction angle 2-theta±0.2 degrees of 5.3° in combination of one or more peaks chosen from 6.0°, 6.9°, 7.2°, 8.0°, 12.2°, and 15.6°. In some embodiments, the compound is in the form of a crystalline solid characterized by a peak in the X-ray powder diffraction pattern at diffraction angle 2-theta±0.2 degrees of 5.8° in combination of one or more peaks chosen from 4.4°, 6.6°, 10.1°, 11.4°, 13.4°, and 15.5°. In some embodiments, the compound is in the form of a crystalline solid characterized by peaks in the X-ray powder diffraction pattern at diffraction angle 2-theta±0.2 degrees of 4.5° and 5.5° in combination of one or more peaks chosen from 6.0° and 7.3°.

In some embodiments, the present disclosure describes a method of synthesizing a compound is Formula (IX), the method comprising:
reacting a compound of formula (Dₚᵣₒₜ) and a compound of formula (Yₚᵣₒₜ₂) to form a compound of formula (Dₚᵣₒₜ-Yₚᵣₒₜ₂)

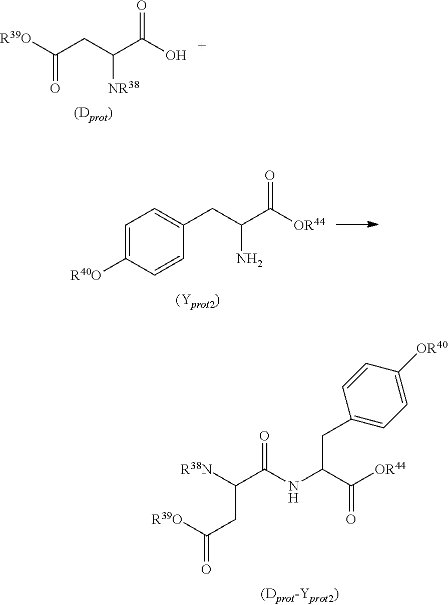

wherein R³⁸, R³⁹, R⁴⁰, and R⁴⁴ are protecting groups;
removing the protecting group R⁴ to form a compound of (Dₚᵣₒₜ-Yₚᵣₒₜ₁)

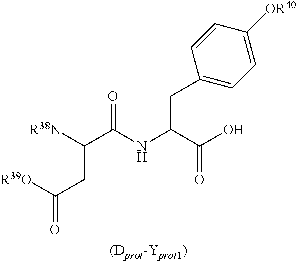

reacting the compound of formula (Dₚᵣₒₜ-Yₚᵣₒₜ₁) and a compound of formula (Sₚᵣₒₜ₂) to form a compound of a formula (Dₚᵣₒₜ-Yₚᵣₒₜ₁-Sₚᵣₒₜ₂)

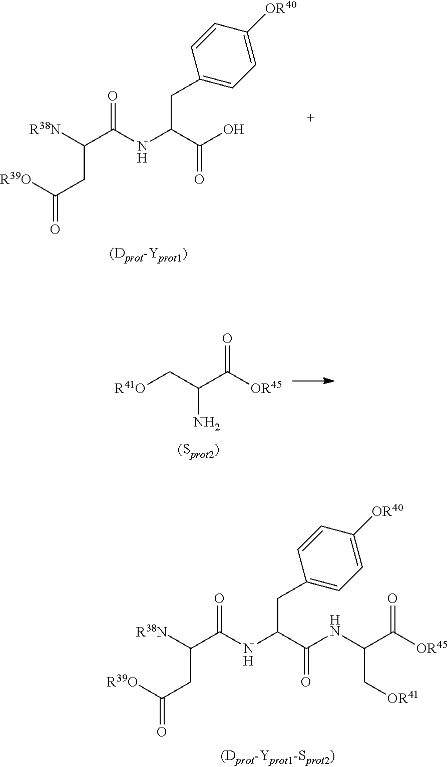

wherein R⁴¹ and R⁴⁵ are protecting groups;
removing the protecting group R⁴⁵ to form a compound of (Dₚᵣₒₜ-Yₚᵣₒₜ₁-Sₚᵣₒₜ₁)

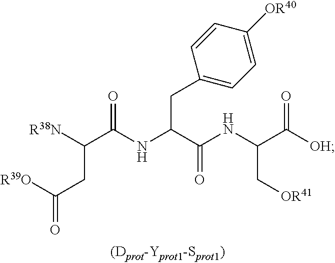

reacting the compound of formula (Dₚᵣₒₜ-Yₚᵣₒₜ₁-Sₚᵣₒₜ₁) and a compound of formula (Kₚᵣₒₜ₂) to form a compound of Formula (IX)

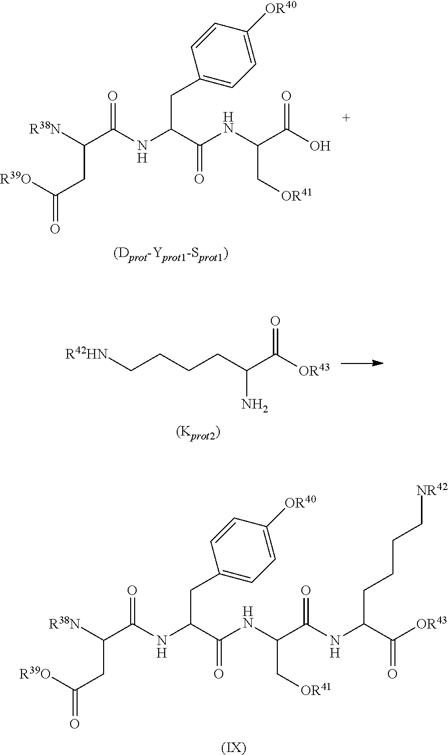

wherein R⁴² and R⁴³ are protecting groups; and
optionally removing one or more protecting groups R³⁸, R³⁹, R⁴⁰, R⁴¹, R⁴², and R⁴³.

In some embodiments, the protecting groups are chosen from Boc, Fmoc, tert-butyl and trityl groups.

In some embodiments, the present disclosure describes a method of synthesizing a polypeptide of SEQ ID NO: 16, the method comprising: (i) conjugating a compound of Formula (IX) via the C-terminus of the compound to the N-terminus of a polypeptide of SEQ ID NO: 18, and (ii) conjugating the compound via its N-terminus to the C-terminus of a polypeptide of SEQ ID NO: 19.

In some embodiments, the present disclosure describes a compound of Formula (X), or a salt, solvate, or hydrate thereof,

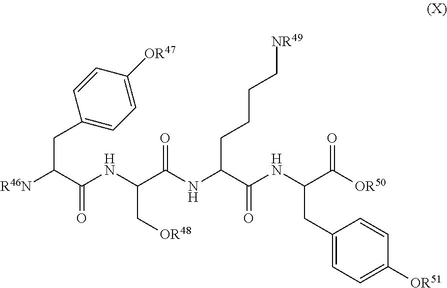

wherein R⁴⁶ is H or a protecting group; R⁴⁷ is H or a protecting group; R⁴⁸ is H or a protecting group; R⁴⁹ is H or a protecting group; R⁵⁰ is H or a protecting group; and R⁵¹ is H or a protecting group. In some embodiments, each protecting group is independently chosen from Boc, Fmoc, tert-butyl, and trityl groups. In some embodiments, R⁴⁶ is H or tert-butyl. In some embodiments, R⁴⁷ is H or tert-butyl. In some embodiments, R⁴⁸ is H or Boc. In some embodiments, R⁴⁹ is H or Fmoc. In some embodiments, R⁵⁰ is H or tert-butyl. In some embodiments, R⁵¹ is H, or tert-butyl. In some embodiments, at least one of R⁴⁶, R⁴⁷, R⁴⁸, R⁴⁹, R⁵⁰, and R⁵¹ is H. In some embodiments, at least one of R⁴⁶, R⁴⁷, R⁴⁸, R⁴⁹, R⁵⁰, and R⁵¹ is a protecting group.

In some embodiments, the compound is Formula (X) is a compound of Formula (X-a):

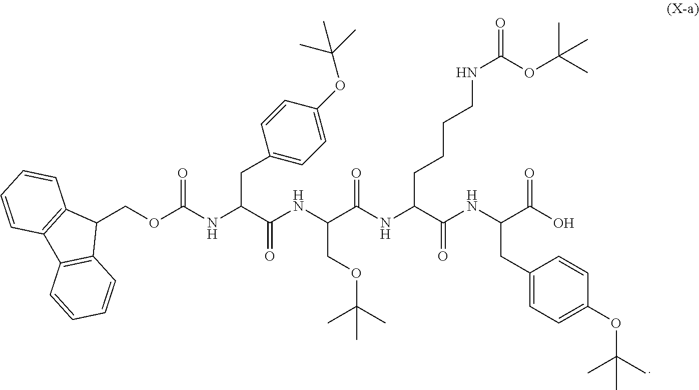 [SMILES: CC(C)(C)OCC(NC(=O)C(Cc1ccc(OC(C)(C)C)cc1)NC(=O)OCC1c2ccccc2-c2ccccc21)C(=O)NC(CCCCNC(=O)OC(C)(C)C)C(=O)NC(Cc1ccc(OC(C)(C)C)cc1)C(=O)O]

In some embodiments, the compound is Formula (X) is a solvate. In some embodiments, the compound is a solvate produced from ethanol or isopropyl alcohol. In some embodiments, the compound is a de-solvate.

In some embodiments, the compound is Formula (X) is crystalline. In some embodiments, the compound is in the form of a crystalline solid characterized by peaks in the X-ray powder diffraction pattern at diffraction angle 2-theta±0.2 degrees of 18.1° and 18.7° in combination of one or more peaks chosen from 5.7°, 8.7°, 13.7°, 14.3°, 15.9°, and 16.2°. In some embodiments, the compound is in the form of a crystalline solid characterized by peaks in the X-ray powder diffraction pattern at diffraction angle 2-theta±0.2 degrees of 5.9° and 10.5° in combination of one or more peaks chosen from 7.1°, 8.9°, 14.6°, and 16.6°. In some embodiments, the compound is in the form of a crystalline solid characterized by peaks in the X-ray powder diffraction pattern at diffraction angle 2-theta±0.2 degrees of 7.8° and 20.3° in combination of one or more peaks chosen from 5.8°, 15.5°, and 19.5°. In some embodiments, the compound is in the form of a crystalline solid characterized by peaks in the X-ray powder diffraction pattern at diffraction angle 2-theta±0.2 degrees of 5.9° and 7.4° in combination of one or more peaks chosen from 6.5°, 6.9°, and 14.8°.

In some embodiments, the present disclosure describes a method of synthesizing a compound is Formula (X), the method comprising:
reacting a compound of formula (Yₚᵣₒₜ) and a compound of formula (Sₚᵣₒₜ₂) to form a compound of formula (YₚᵣₒₜSₚᵣₒₜ₂)

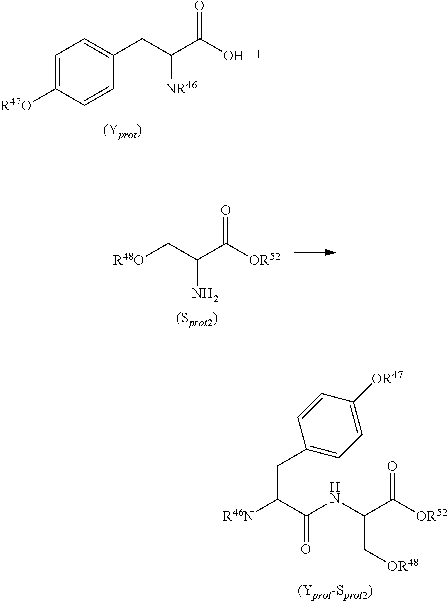

wherein R⁴⁶, R⁴⁷, R⁴⁸, and R⁵² are protecting groups;
removing the protecting group R⁵² to form a compound of (Yₚᵣₒₜ-Sₚᵣₒₜ₁)

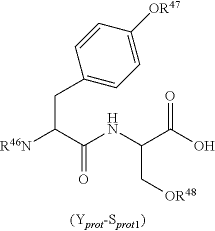

reacting the compound of formula (Yₚᵣₒₜ-Sₚᵣₒₜ₁) and a compound of formula (Kₚᵣₒₜ₂) to form a compound of a formula Yₚᵣₒₜ-Sₚᵣₒₜ₁-Kₚᵣₒₜ₂)

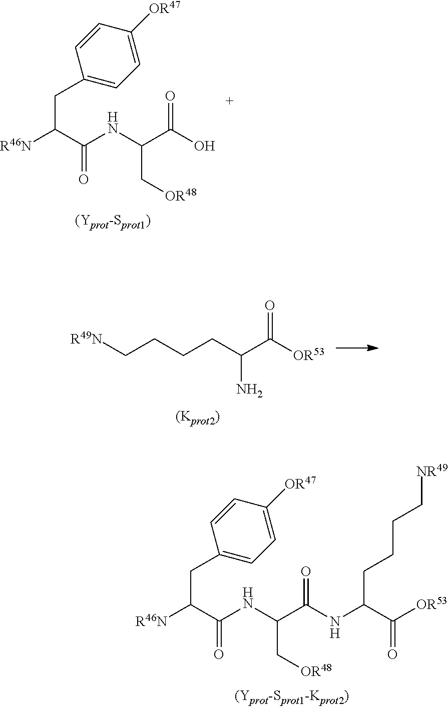

wherein R⁴⁹ and R⁵³ are protecting groups;
removing the protecting group R⁵³ to form a compound of (Yₚᵣₒₜ-Sₚᵣₒₜ₁-Kₚᵣₒₜ₁)

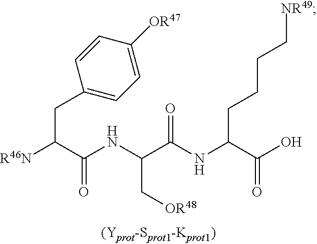

reacting the compound of formula (Yₚᵣₒₜ-Sₚᵣₒₜ₁-Kₚᵣₒₜ₁) and a compound of formula (Yₚᵣₒₜ₂) to form a compound of Formula (X)

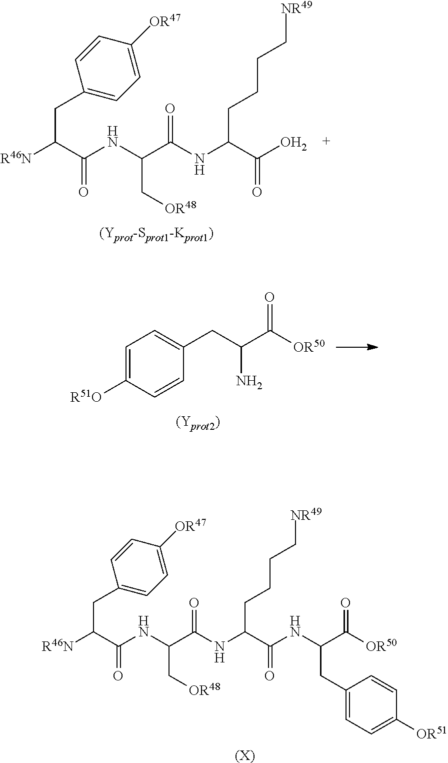

wherein R⁵⁰ and R⁵¹ are protecting groups; and
optionally removing one or more protecting groups R⁴⁶, R⁴⁷, R⁴⁸, R⁴⁹, R⁵⁰, and R⁵¹.

In some embodiments, the protecting groups are chosen from Boc, Fmoc, tert-butyl, and trityl groups.

In some embodiments, the present disclosure describes a method of synthesizing a polypeptide of SEQ ID NO: 16, the method comprising: (i) conjugating a compound is Formula (X) via the C-terminus of the compound to the N-terminus of a polypeptide of SEQ ID NO: 20, and (ii) conjugating the resulting compound via its N-terminus to the C-terminus of a polypeptide of SEQ ID NO: 21.

In some embodiments, the present disclosure describes a compound of Formula (XI), or a salt, solvate, or hydrate thereof,

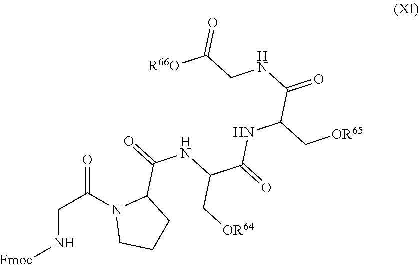

wherein R⁶⁴ is H or a protecting group; R⁶⁵ is H or a protecting group; and R⁶⁶ is H or a protecting group. In some embodiments, each protecting group is independently chosen from Boc, Fmoc, tert-butyl, and trityl groups. In some embodiments, at least one of R⁶⁴, R⁶⁵, and R⁶⁶ is H. In some embodiments, at least one of R⁶⁴, R⁶⁵, and R⁶⁶ is a protecting group.

In some embodiments, the compound of Formula (XI) is a compound of the Formula:

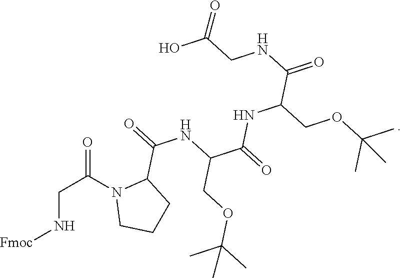

In some embodiments, the compound of Formula (XI) is a solvate.

In some embodiments, the compound of Formula (XI) is crystalline. In some embodiments, the compound is in the form of a crystalline solid characterized by peaks in the X-ray powder diffraction pattern at diffraction angle 2-theta±0.2 degrees of 6.1° and 8.5° in combination of one or more peaks chosen from 5.8°, 16.9°, 18.5°, 18.8°, 19.3°, and 20.9.

In some embodiments, the present disclosure describes a compound of Formula (XII), or a salt, solvate, or hydrate thereof,

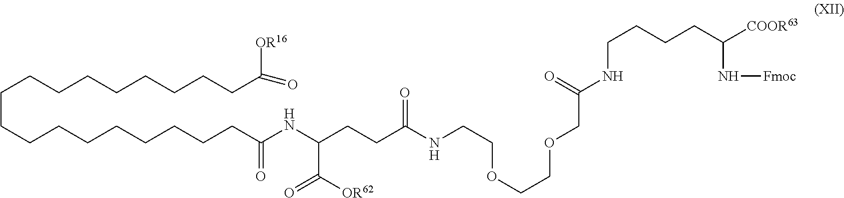

wherein R⁶¹ is H or a protecting group; R⁶² is H or a protecting group; and R⁶³ is H or a protecting group. In some embodiments, each protecting group is independently chosen from Boc, Fmoc, tert-butyl, and trityl groups. In some embodiments, R⁶¹ is H or tert-butyl. In some embodiments, R⁶² is H or tert-butyl. In some embodiments, R⁶³ is H or tert-butyl. In some embodiments, at least one of R⁶¹, R⁶², and R⁶³ is H. In some embodiments, at least one of R⁶¹, R⁶², and R⁶³ is a protecting group.

In some embodiments, the compound of Formula (XII) is a compound of Formula (XII-a):

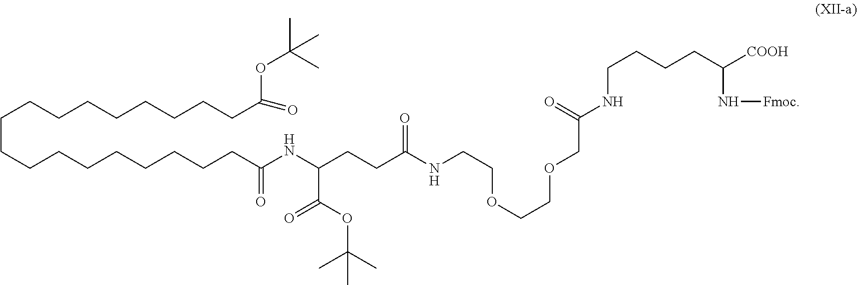

In some embodiments, the compound of Formula (XII) is a solvate. In some embodiments, the compound is a de-solvate or is anhydrous.

In some embodiments, the compound of Formula (XII) is crystalline. In some embodiments, the compound is in the form of a crystalline solid characterized by peaks in the X-ray powder diffraction pattern at diffraction angle 2-theta±0.2 degrees of 11.40 in combination of one or more peaks chosen from 6.0°, 8.9°, 12.7°, 13.6°, 14.6°, 17.0°, and 18.8°. In some embodiments, the compound is in the form of a crystalline solid characterized by peaks in the X-ray powder diffraction pattern at diffraction angle 2-theta±0.2 degrees of 10.6° in combination of one or more peaks chosen from 7.1°, 12.1°, 13.6°, 14.2°, 15.2°, 16.0°, and 16.8°. In some embodiments, the compound is in the form of a crystalline solid characterized by peaks in the X-ray powder diffraction pattern at diffraction angle 2-theta±0.2 degrees of 10.10 and 15.5° in combination of one or more peaks chosen from 6.1°, 8.7°, 11.4°, 16.6°, and 19.2°.

In some embodiments, the present disclosure describes a compound of Formula (XIII), or a salt, solvate, or hydrate thereof,

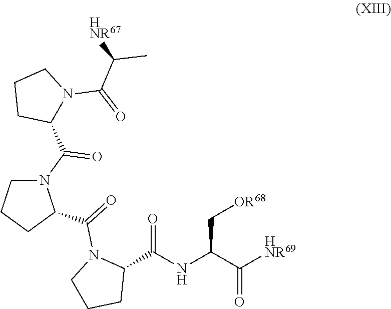

wherein R⁶⁷ is H or a protecting group; R⁶⁸ is H or a protecting group; and R⁶⁹ is H or a protecting group. In some embodiments, each protecting group is independently chosen from Boc, Fmoc, tert-butyl, and trityl groups. In some embodiments, R⁶⁷ is H or tert-butyl. In some embodiments, R⁶⁸ is H or tert-butyl. In some embodiments, R⁶⁹ is H or tert-butyl. In some embodiments, at least one of R⁶⁷, R⁶⁸, and R⁶⁹ is H. In some embodiments, at least one of R⁶⁷, R⁶⁸, and R⁶⁶ is a protecting group.

In some embodiments, the compound of Formula (XIII) is a compound of the Formula:

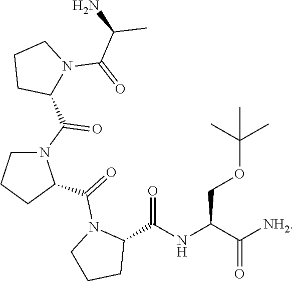 [SMILES: C[C@H](N)C(=O)N1CCC[C@H]1C(=O)N1CCC[C@H]1C(=O)N1CCC[C@H]1C(=O)N[C@@H](COC(C)(C)C)C(N)=O]

In some embodiments, the compound of Formula (XIII) is a solvate. In some embodiments, the compound is a de-solvate or is anhydrous.

In some embodiments, the compound of Formula (XIII) is crystalline. In some embodiments, the compound is in the form of a crystalline solid characterized by peaks in the X-ray powder diffraction pattern at diffraction angle 2-theta±0.2 degrees of 5.0° in combination of one or more peaks chosen from 8.3, 9.7, and 11.2°. In some embodiments, the compound is in the form of a crystalline solid characterized by peaks in the X-ray powder diffraction pattern at diffraction angle 2-theta±0.2 degrees of 7.2° in combination of one or more peaks chosen from 5.3, 8.1, 14.4, and 16.2°.

In some embodiments, the present disclosure describes a compound of Formula (XIV), or a salt, solvate, or hydrate thereof,

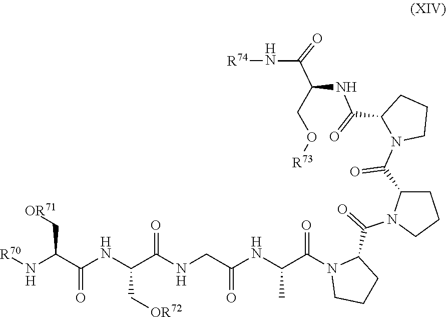

wherein R⁷⁰ is H or a protecting group; R⁷¹ is H or a protecting group; R⁷² is H or a protecting group; R⁷³ is H or a protecting group; and R⁷⁴ is H or a protecting group. In some embodiments, each protecting group is independently chosen from Boc, Fmoc, tert-butyl, and trityl groups. In some embodiments, R⁷⁰ is H, Fmoc, or tert-butyl. In some embodiments, R⁷¹ is H or tert-butyl. In some embodiments, R⁷² is H or tert-butyl. In some embodiments, R⁷³ is H or tert-butyl. In some embodiments, R⁷⁴ is H, Fmoc, or tert-butyl. In some embodiments, at least one of R⁷⁰, R⁷¹, R⁷², R⁷³, and R⁷⁴ is H. In some embodiments, at least one of R⁷⁰, R⁷¹, R⁷², R⁷³, and R⁷⁴ is a protecting group.

In some embodiments, the compound of Formula (XIV) is a compound of the Formula:

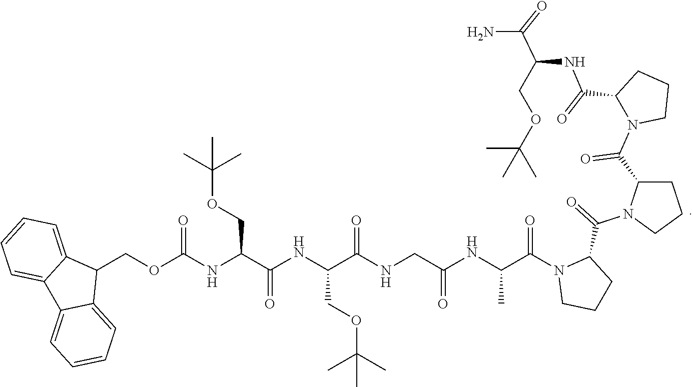 [SMILES: C[C@H](NC(=O)CNC(=O)[C@H](COC(C)(C)C)NC(=O)[C@H](COC(C)(C)C)NC(=O)OCC1c2ccccc2-c2ccccc21)C(=O)N1CCC[C@H]1C(=O)N1CCC[C@H]1C(=O)N1CCC[C@H]1C(=O)N[C@@H](COC(C)(C)C)C(N)=O]

In some embodiments, the compound of Formula (XIV) is a solvate. In some embodiments, the compound is a de-solvate or is anhydrous

In some embodiments, the compound of Formula (XIV) is crystalline. In some embodiments, the compound is in the form of a crystalline solid characterized by peaks in the X-ray powder diffraction pattern at diffraction angle 2-theta±0.2 degrees of 7.5° in combination of one or more peaks chosen from 6.1, 8.7, 10.6, 15.0, 16.1, and 18.6°.

### BRIEF DESCRIPTION OF THE DRAWINGS

FIG. 1A, FIG. 1B, and FIG. 1C show representative x-ray powder diffraction (“XRPD”) patterns of Y-Aib-E-G tetramer Form A, Form B, and Form C, respectively.

FIG. 2A, FIG. 2B, FIG. 2C, and FIG. 2D show representative XRPD patterns of T-F-T-S tetramer Form A, Form B, Form C, and Form D, respectively.

FIG. 3 shows a polymorph map for the relationship of various crystalline solid forms of P-S-S-G-NH₂.

FIG. 4A, FIG. 4B, FIG. 4C, FIG. 4D, FIG. 4E, FIG. 4F, FIG. 4G, and FIG. 4H show representative XRPD patterns of P-S-S-G-NH₂ tetramer Form A, Form B, Form C, Form D, Form E, Form F, Form G, and Form H, respectively.

FIG. 5A and FIG. 5B show representative XRPD patterns of G-P-S-S-G-NH₂ pentamer Form A and Form B, respectively.

FIG. 6A and FIG. 6B show representative XRPD patterns of T-F-T-S-D pentamer Form A and Form B, respectively. The traces in FIG. 6A and FIG. 6B show results of products obtained in various experiments.

FIG. 7A and FIG. 7B show representative XRPD patterns of Y-Aib-Q-G tetramer Form A and Form B, respectively.

FIG. 8A, FIG. 8B, and FIG. 8C show representative XRPD patterns of H(dnp)-Aib-Q-G tetramer Form A, Form B, and Form C, respectively.

FIG. 9A and FIG. 9B show representative XRPD patterns for H(trt)-Aib-Q-G tetramer Form A and Form B, respectively.

FIG. 10A, FIG. 10B, and FIG. 10C show representative XRPD patterns for D-Y-S-K tetramer Form A, Form B, and Form C, respectively.

FIG. 11 shows a polymorph map for the relationship of various crystalline solid forms of Y-S-K-Y.

FIG. 12A, FIG. 12B, FIG. 12C, and FIG. 12D show representative XRPD patterns for Y-S-K-Y Form A, Form B, Form C, and Form D, respectively.

FIG. 13 shows a polymorph map for the relationship of various crystalline solid forms of Fmoc-G-P-S-S-G-NH₂.

FIG. 14A, FIG. 14B, FIG. 14C, FIG. 14D, and FIG. 14E show representative XRPD patterns for Fmoc-G-P-S-S-G-NH₂ pentamer Form A, Form B, Form C, Form D, and Form E, respectively.

FIG. 15A and FIG. 15B show representative XRPD patterns for Fmoc-G-P-S-S-G-OH tetramer Form A and Form B, respectively.

FIG. 16A, FIG. 16B, and FIG. 16C show representative XRPD patterns for GGG side chain Form A, Form B, and Form C, respectively.

FIG. 17A and FIG. 17C show representative XRPD patterns for H-A-P-P-P-S-NH₂ HCl tetramer Form A and Form B, respectively. FIG. 17B show representative XRPD patterns for H-A-P-P-P-S-NH₂ HCl tetramer Form A in dried form.

FIG. 18 shows a representative XRPD patterns for H-A Fmoc-S-S-G-A-P-P-P-S-NH₂ tetramer Form A.

FIG. 19 shows LCMS analytical results for a coupling agent study.

FIG. 20 shows UPLC-MS results for Fmoc-Y-Aib-E-G compounds.

### DETAILED DESCRIPTION

The present disclosure relates to crystalline forms of peptide compounds with high purity and desirable physical properties, such as hygroscopicity, and are useful for preparing biopharmaceutical compounds. Crystalline peptide compounds provided herein can be useful for preparing biopharmaceutical peptides such as tirzepatide (SEQ ID NO: 1), retatrutide (SEQ ID NO: 12), and mazdutide (SEQ ID NO: 16) with improved efficiency.

### Formula (I)

In one aspect, the present disclosure provides a compound of Formula (I), or a salt, solvate, or hydrate thereof,

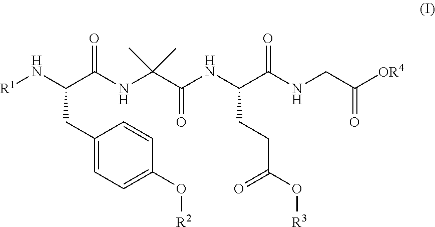

wherein R¹ is H or a protecting group; R² is H or a protecting group; R³ is H or a protecting group; and R⁴ is H or a protecting group.

In some embodiments, each protecting group is independently chosen from Boc, Fmoc, tert-butyl, benzyl, carboxybenzyl, methyl, allyl, cyclohexyl, and trityl groups.

In another aspect, the present disclosure provides a crystalline compound of Formula (I), or a salt, solvate, or hydrate thereof,

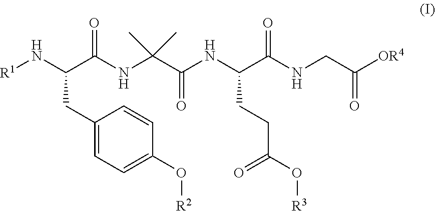

wherein R¹ is H or a protecting group; R² is H or a protecting group; R³ is H or a protecting group; and R⁴ is H or a protecting group.

In some embodiments, R¹ is H, Fmoc, or Boc. In some embodiments, R² is H or t-butyl. In some embodiments, R³ is H or t-butyl. In some embodiments, R⁴ is H or benzyl.

In some embodiments, at least one of R¹, R², R³, and R⁴ is a protecting group. In some embodiments, R¹, R², R³, and R are H.

In some embodiments, R³ is tert-butyl. In some embodiments, R³ is benzyl. In some embodiments, R³ is carboxybenzyl. In some embodiments, R³ is methyl. In some embodiments, R³ is allyl. In some embodiments, R³ is cyclohexyl. In some embodiments, R³ is trityl.

In some embodiments, a crystalline form of Formula (I) is Boc-l-Tyr (tBu)-Aib-Glu(OtBu)-Gly-OH having a structure of Formula (I-a)

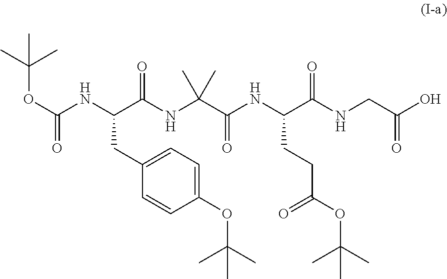 [SMILES: CC(C)(C)OC(=O)CC[C@H](NC(=O)C(C)(C)NC(=O)[C@H](Cc1ccc(OC(C)(C)C)cc1)NC(=O)OC(C)(C)C)C(=O)NCC(=O)O]

In some embodiments, a compound of Formula (I) or (I-a) is crystalline. In some embodiments, a compound of Formula (I) or (I-a) is a solvate. In some embodiments, a solvated compound of Formula (I) or (I-a) is crystalline. In some embodiments, a compound of Formula (I) or (I-a) is a solvate produced from methyl t-butyl ether (MTBE) or a mixture comprising MTBE.

In some embodiments, a compound of Formula (I-a) is in the form of a crystalline solid characterized by a peak in the X-ray powder diffraction pattern at diffraction angle 2θ±0.2 degrees of 5.2° in combination of one or more peaks select from the group consisting of 8.4°, 8.8°, 10.4°, 15.5°, 17.1°, and 17.7°.

In some embodiments, a compound of Formula (I-a) is in the form of a crystalline solid characterized by a peak in the X-ray powder diffraction pattern at diffraction angle 2θ±0.2 degrees of 6.1° in combination of one or more peaks select from the group consisting of 10.3°, 14.9°, 16.8°, 18.10, and 18.2°.

In some embodiments, a compound of Formula (I-a) is in the form of a crystalline solid characterized by a peak in the X-ray powder diffraction pattern at diffraction angle 2θ±0.2 degrees of 6.2° in combination of one or more peaks select from the group consisting of 8.9°, 12.3°, 14.9°, 15.4°, and 21.8°.

In some embodiments, a compound of Formula (I-a) is in the form of a crystalline solid characterized by a peak in the X-ray powder diffraction pattern at diffraction angle 2 theta±0.2 degrees of 5.2°, 8.4°, 8.8°, 10.4°, 15.5°, 17.1°, and 17.7.

In some embodiments, a compound of Formula (I) is

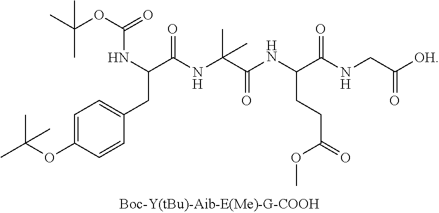 [SMILES: COC(=O)CCC(NC(=O)C(C)(C)NC(=O)C(Cc1ccc(OC(C)(C)C)cc1)NC(=O)OC(C)(C)C)C(=O)NCC(=O)O]

In some embodiments, a compound of Formula (I) is

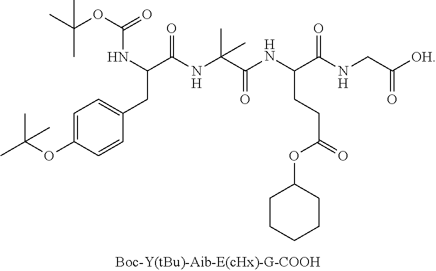 [SMILES: CC(C)(C)OC(=O)NC(Cc1ccc(OC(C)(C)C)cc1)C(=O)NC(C)(C)C(=O)NC(CCC(=O)OC1CCCCC1)C(=O)NCC(=O)O]

In some embodiments, a compound of Formula (I) is

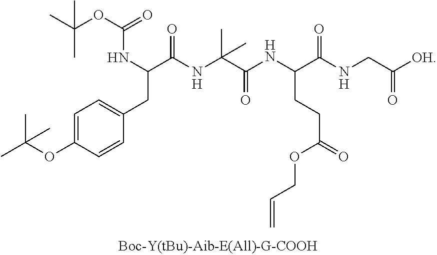 [SMILES: C=CCOC(=O)CCC(NC(=O)C(C)(C)NC(=O)C(Cc1ccc(OC(C)(C)C)cc1)NC(=O)OC(C)(C)C)C(=O)NCC(=O)O]

In some embodiments, a compound of Formula (I) is

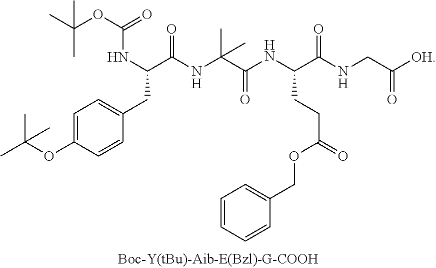 [SMILES: CC(C)(C)OC(=O)N[C@@H](Cc1ccc(OC(C)(C)C)cc1)C(=O)NC(C)(C)C(=O)N[C@@H](CCC(=O)OCc1ccccc1)C(=O)NCC(=O)O]

The present disclosure also provides a method of synthesizing the compound of Formula (I), the method comprising:
(i) reacting a compound of formula (Yₚᵣₒₜ)

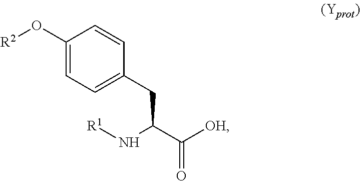

wherein R¹ and R² are protecting groups, and a compound of formula (Aibₚᵣₒₜ)

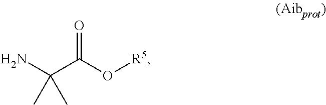

wherein R⁵ is a protecting group, forming a compound of formula (Yₚᵣₒₜ-Aibₚᵣₒₜ),

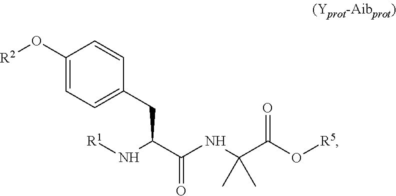

removing the protecting group of R⁵, and forming a compound of formula (Yₚᵣₒₜ-Aib)

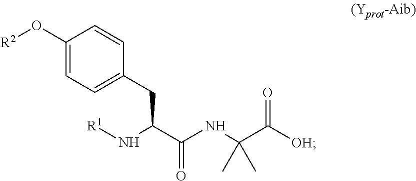

(ii) reacting the compound of formula (Yₚᵣₒₜ-Aib) and a compound of formula (Eₚᵣₒₜ)

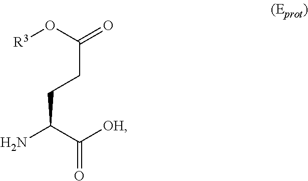

wherein R³ is a protecting group, forming a compound of a formula (Yₚᵣₒₜ-Aib-Eₚᵣₒₜ)

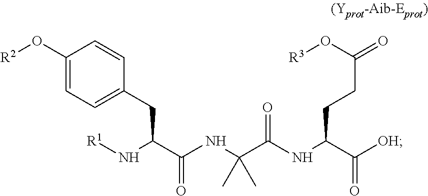

(iii) reacting the compound of formula (Yₚᵣₒₜ-Aib-Eₚᵣₒₜ) and a compound of formula (Gₚᵣₒₜ)

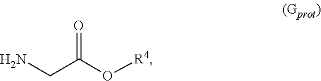

wherein R⁴ is a protecting group, forming a compound of formula (Yₚᵣₒₜ-Aib-Eₚᵣₒₜ-Gₚᵣₒₜ)

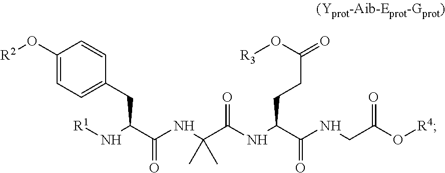

and optionally (iv) removing one or more of protecting groups R¹, R², R³, and R⁴.

In some embodiments, R⁵ is t-butyl.

In some embodiments, R¹ is Fmoc, R² is t-butyl, R³ is t-butyl, R⁴ is t-butyl, and R⁵ is t-butyl.

### Formula (III)

In another aspect, the present disclosure provides a compound of Formula (II), or a salt, solvate, or hydrate thereof

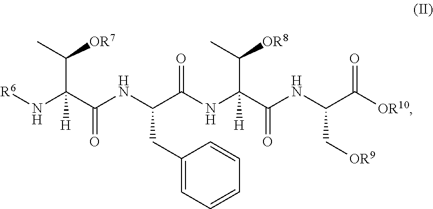

wherein R⁶ is H or a protecting group; R⁷ is H or a protecting group; R⁸ is H or a protecting group; R⁹ is H or a protecting group; and R¹⁰ is H or a protecting group.

In some embodiments, each protecting group is independently chosen from Boc, Fmoc, tert-butyl, benzyl, carboxybenzyl, methyl, allyl, cyclohexyl, and trityl groups. In some embodiments, protecting groups are Boc or Fmoc.

In some embodiments, R⁶ is H, Fmoc, or Boc. In some embodiments, R⁷ is H or t-butyl. In some embodiments, R⁸ is H or t-butyl. In some embodiments, R⁹ is H or t-butyl. In some embodiments, R¹⁰ is H or benzyl. In some embodiments, at least one of R⁶, R⁷, R⁸, R⁹, and R¹⁰ is a protecting group. In some embodiments, R⁶, R⁷, R⁸, R⁹, and R¹⁰ are H.

In some embodiments, Formula (II) is Fmoc-Thr(tBu)-Phe-Thr(tBu)-Ser(tBu)-OH having a structure of Formula (II-a)

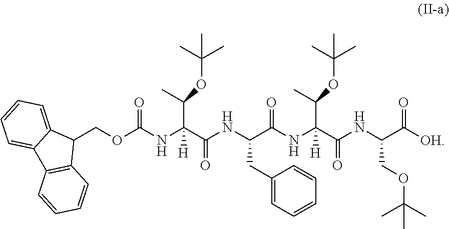 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)O)[C@@H](C)OC(C)(C)C]

In some embodiments, a compound of Formula (II) or (II-a) is crystalline. In some embodiments, a compound of Formula (II) or (II-a) is a solvate. In some embodiments, a compound of Formula (II) or (II-a) is a solvate produced from a solvent comprising heptane. In some embodiments, a solvate of compound of Formula (II) or (II-a) is crystalline.

In some embodiments, a compound of Formula (II-a) is in the form of a crystalline solid characterized by a peak in the X-ray powder diffraction pattern at diffraction angle 2θ±0.2 degrees of 7.7-7.9° in combination of one or more peaks select from the group consisting of 5.8°, 10.0°, 10.8-10.9°, 11.3-11.4°, 12.0-12.1°, 12.8°, 14.2-14.4°, and 16.8-17.0°.

In some embodiments, a compound of Formula (II-a) is in the form of a crystalline solid characterized by a peak in the X-ray powder diffraction pattern at diffraction angle 2θ±0.2 degrees of 7.3° in combination of one or more peaks select from the group consisting of 5.1°, 5.7°, 7.6°, 9.5°, and 12.4°.

In some embodiments, a compound of Formula (II-a) is in the form of a crystalline solid characterized by a peak in the X-ray powder diffraction pattern at diffraction angle 2θ±0.2 degrees of 7.8° in combination of one or more peaks select from the group consisting of 8.5°, 11.5°, 12.0°, 12.8°, 14.3°, 15.5°, 20.2° and 23.3°.

In some embodiments, a compound of Formula (II-a) is in the form of a crystalline solid characterized by a peak in the X-ray powder diffraction pattern at diffraction angle 2θ±0.2 degrees of 8.2-8.3° in combination of one or more peaks select from the group consisting of 5.9°, 7.7°, 9.2°, 10.2°, 11.3°, 13.8-13.9°, 15.5-15.7°, 17.1°, and 18.5°.

### Formula (III)

In another aspect, the present disclosure provides a compound of Formula (III), or a salt, solvate, or hydrate thereof,

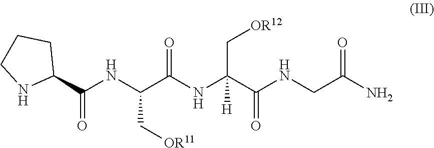

wherein R¹¹ is H or a protecting group; and R¹² is H or a protecting group.

In some embodiments, each protecting group is independently chosen from Boc, Fmoc, tert-butyl, benzyl, carboxybenzyl, methyl, allyl, cyclohexyl, and trityl groups.

In some embodiments, R¹¹ is H or t-butyl. In some embodiments, R¹² is H or t-butyl. In some embodiments, at least one of R¹¹ and R¹² is a protecting group. In some embodiments, R¹¹ and R¹² are H.

In some embodiments, Formula (III) is Pro-Ser(tBu)-Ser(tBu)-Gly-NH₂ having a structure of Formula (III-a).

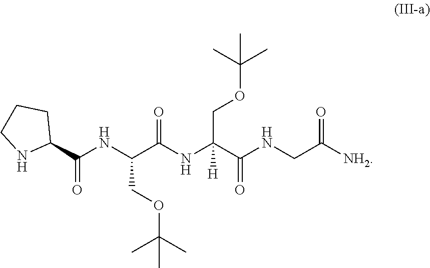 [SMILES: CC(C)(C)OC[C@H](NC(=O)[C@H](COC(C)(C)C)NC(=O)[C@@H]1CCCN1)C(=O)NCC(N)=O]

In some embodiments, a compound of Formula (III) or (III-a) is crystalline. In some embodiments, a compound of Formula (III) or (III-a) is a solvate. In some embodiments, a solvated form of Formula (III) or (III-a) is crystalline.

In some embodiments, a compound of Formula (III-a) is in the form of a crystalline solid characterized by a peak in the X-ray powder diffraction pattern at diffraction angle 2θ±0.2 degrees of 8.0° in combination of one or more peaks select from the group consisting of 7.0°, 10.30, 14.1°, 15.2°, 16.7°, 18.0°, 19.0°, 19.7°, 20.8°, and 21.9°.

In some embodiments, a compound of Formula (III-a) is in the form of a crystalline solid characterized by a peak in the X-ray powder diffraction pattern at diffraction angle 2θ±0.2 degrees of 9.0° in combination of one or more peaks select from the group consisting of 5.7°, 9.9°, 16.2°, 17.1°, 17.9°, 18.1°, 18.4°, 18.8°, 19.9°, 20.1°, and 22.5°.

In some embodiments, a compound of Formula (III-a) is in the form of a crystalline solid characterized by a peak in the X-ray powder diffraction pattern at diffraction angle 2θ±0.2 degrees of 10.5° in combination of one or more peaks select from the group consisting of 5.6°, 11.9°, 13.3°, 15.4°, 15.6°, 18.1°, 19.9°, and 21.1°.

In some embodiments, a compound of Formula (III-a) is in the form of a crystalline solid characterized by a peak in the X-ray powder diffraction pattern at diffraction angle 2θ±0.2 degrees of 10.7° in combination of one or more peaks select from the group consisting of 4.9°, 14.8°, 20.3°, and 21.5°.

In some embodiments, a compound of Formula (III-a) is in the form of a crystalline solid characterized by a peak in the X-ray powder diffraction pattern at diffraction angle 2θ±0.2 degrees of 10.5° in combination of one or more peaks select from the group consisting of 5.9°, 10.5°, 10.9°, 12.1°, 13.1°, 15.9°, 17.5°, 20.9°, 21.1°, and 21.9°.

In some embodiments, a compound of Formula (III-a) is in the form of a crystalline solid characterized by a peak in the X-ray powder diffraction pattern at diffraction angle 2θ±0.2 degrees of 7.8° in combination of one or more peaks select from the group consisting of 11.3°, 11.5°, 15.4°, 15.6°, and 21.5°.

In some embodiments, a compound of Formula (III-a) is in the form of a crystalline solid characterized by a peak in the X-ray powder diffraction pattern at diffraction angle 2θ±0.2 degrees of 10.0° in combination of one or more peaks select from the group consisting of 8.1°, 12.5°, 13.5°, 14.7°, 17.8°, 18.8°, 20.0°, and 22.4°.

In some embodiments, a compound of Formula (III-a) is in the form of a crystalline solid characterized by a peak in the X-ray powder diffraction pattern at diffraction angle 2θ±0.2 degrees of 21.1° in combination of one or more peaks select from the group consisting of 5.6°, 10.5°, 10.8°, 11.9°, 15.4°, and 23.8°.

### Formula (IV)

In another aspect, the present disclosure provides a compound of Formula (IV), or a salt, solvate, or hydrate thereof,

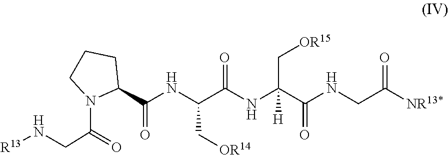

wherein R¹³ is H or a protecting group; R^{13*} is H or a protecting group; R¹⁴ is H or a protecting group; and R¹⁵ is H or a protecting group.

In some embodiments, each protecting group is independently chosen from Boc, Fmoc, tert-butyl, benzyl, carboxybenzyl, methyl, allyl, cyclohexyl, and trityl groups.

In some embodiments, R¹³ is H, Fmoc, or Boc. In some embodiments, R^{13*} is H. In some embodiments, R¹⁴ is H or t-butyl. In some embodiments, R¹⁵ is H or t-butyl. In some embodiments, at least one of R¹³, R¹⁴, and R¹⁵ is a protecting group. In some embodiments, R¹³, R^{13*}, R¹⁴, and R¹⁵ are H.

In some embodiments, Formula (IV) is Gly-Pro-Ser(tBu)-Ser(tBu)-Gly-NH₂ having a structure of Formula (IV-a)

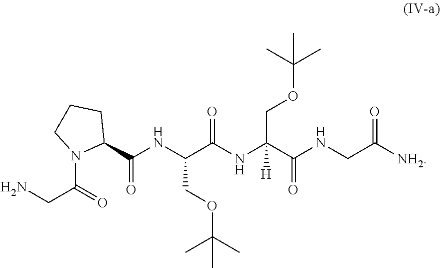 [SMILES: CC(C)(C)OC[C@H](NC(=O)[C@H](COC(C)(C)C)NC(=O)[C@@H]1CCCN1C(=O)CN)C(=O)NCC(N)=O]

In some embodiments, a compound of Formula (IV) or (IV-a) is crystalline. In some embodiments, a compound of Formula (IV) or (IV-a) is a solvate. In some embodiments, a solvated compound of Formula (IV) or (IV-a) is crystalline.

In some embodiments, a compound of Formula (IV-a) is in the form of a crystalline solid characterized by a peak in the X-ray powder diffraction pattern at diffraction angle 2θ±0.2 degrees of 5.1° in combination of one or more peaks select from the group consisting of 4.3°, 6.1°, 8.0°, 10.1°, and 18.7°.

In some embodiments, a compound of Formula (IV-a) is in the form of a crystalline solid characterized by a peak in the X-ray powder diffraction pattern at diffraction angle 2θ±0.2 degrees of 5.2° in combination of one or more peaks select from the group consisting of 6.0°, 6.7°, 10.0°, 10.3°, 16.4°, 17.8°, 18.3°, 19.4°, and 22.4°.

In some embodiments, Formula (IV) has a structure of Formula (IV-b):

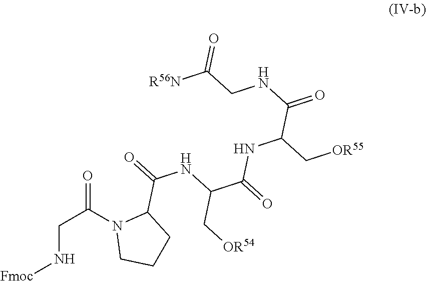

wherein R⁵⁴ is H or a protecting group; R⁵⁵ is H or a protecting group; and R⁵⁶ is H or a protecting group.

In some embodiments, the protecting groups are chosen from Boc, Fmoc, tert-butyl, and trityl groups. In some embodiments, R⁵⁴ is H or tert-butyl. In some embodiments, R⁵⁵ is H or tert-butyl. In some embodiments, R⁵⁶ is H or Boc. In some embodiments, at least one of R⁵⁴, R⁵⁵, and R⁵⁶ is H. In some embodiments, at least one of R⁵⁴, R⁵⁵, and R⁵⁶ is a protecting group.

In some embodiments, Formula (IV) is Fmoc-Gly-Pro-Ser(tBu)-Ser(tBu)-Gly-NH₂ having a structure of Formula (IV-c):

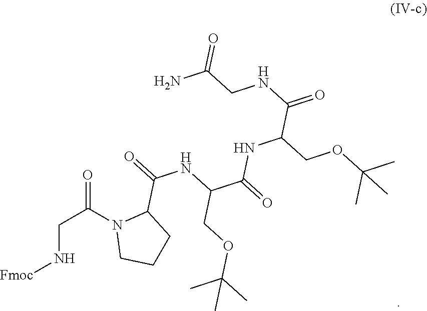

In some embodiments, the compound of Formula (IV-c) is a solvate.

In some embodiments, the solvate is produced from acetone.

In some embodiments, the compound of Formula (IV-c) is a de-solvate.

In some embodiments, the compound of Formula (IV-c) is crystalline.

In some embodiments, the compound of Formula (IV-c) is in the form of a crystalline solid characterized by peaks in the X-ray powder diffraction pattern at diffraction angle 2-theta±0.2 degrees of 5.8° and 18.5° in combination of one or more peaks chosen from 8.6°, 9.4°, 12.9°, 13.8°, 17.2°, and 19.4°.

In some embodiments, the compound of Formula (IV-c) is in the form of a crystalline solid characterized by peaks in the X-ray powder diffraction pattern at diffraction angle 2-theta±0.2 degrees of 7.0-7.1° and 7.5-7.7° in combination of one or more peaks chosen from 5.3-5.4°, 9.7-9.9°, and 14.7-14.9.

In some embodiments, the compound of Formula (IV-c) is in the form of a crystalline solid characterized by a peak in the X-ray powder diffraction pattern at diffraction angle 2-theta±0.2 degrees of 8.3° in combination of one or more peaks chosen from 6.3°, 11.4°, 14.3°, and 16.6°.

In some embodiments, the compound of Formula (IV-c) is in the form of a crystalline solid characterized by a peak in the X-ray powder diffraction pattern at diffraction angle 2-theta±0.2 degrees of 7.2° in combination of one or more peaks chosen from 6.8°, 8.6°, 15.8°, and 18.9°.

In some embodiments, the compound of Formula (IV-c) is in the form of a crystalline solid characterized by a peak in the X-ray powder diffraction pattern at diffraction angle 2-theta±0.2 degrees of 6.1° in combination of one or more peaks chosen from 6.3°, 7.8°, 10.0°, and 12.4°.

The present disclosure also provides a method of synthesizing the compound of Formula (IV-b), the method comprising:
(i) reacting a compound of formula (Fmoc-G) and a compound of formula (Pₚᵣₒₜ) to form a compound of formula (Fmoc-G-Pₚᵣₒₜ)

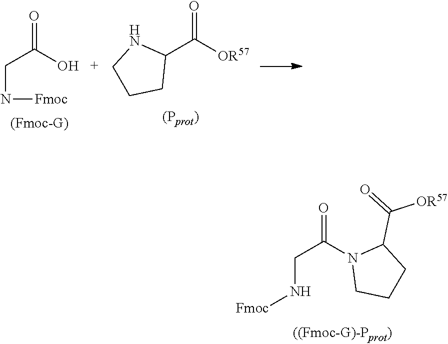

wherein R⁵⁷ is a protecting group;
(ii) removing the protecting group R⁵⁷ to form a compound of (Fmoc-G-P)

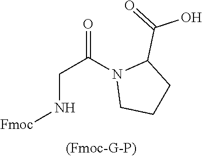

(iii) reacting the compound of formula (Fmoc-G-P) and a compound of formula (Sₚᵣₒₜ₂) to form a compound of a formula (Fmoc-G-P-Sₚᵣₒₜ₂)

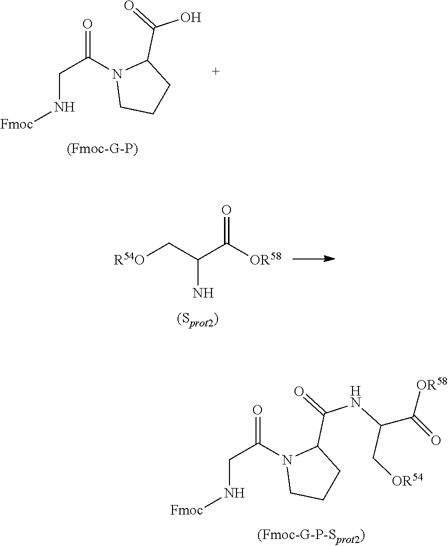

wherein R⁵⁴ and R⁵⁸ are protecting groups;
(iv) removing the protecting group R⁵⁸ to form a compound of (Fmoc-G-P-Sₚᵣₒₜ₁)

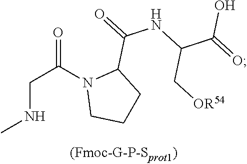

(v) reacting the compound of formula (Fmoc-G-P-Sₚᵣₒₜ₁) and a compound of formula (Sₚᵣₒₜ₂) to form a compound of formula (Fmoc-G-P-Sₚᵣₒₜ₁-Sₚᵣₒₜ₂)

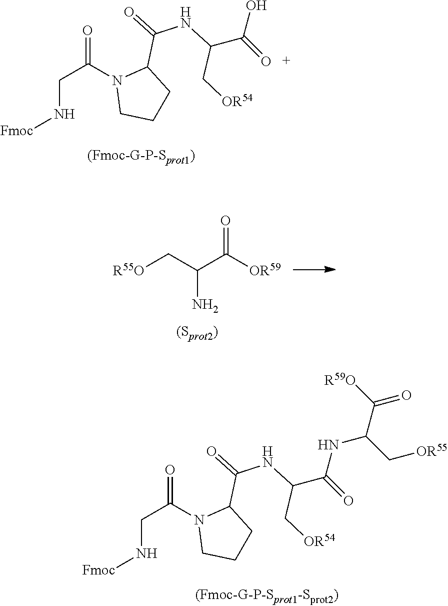

wherein R⁵⁵ and R⁵⁹ are protecting groups; and
(vi) removing the protecting group R⁵⁹ to form a compound of (Fmoc-F-Gₚᵣₒₜ₁-Sₚᵣₒₜ₁-Sₚᵣₒₜ₁)

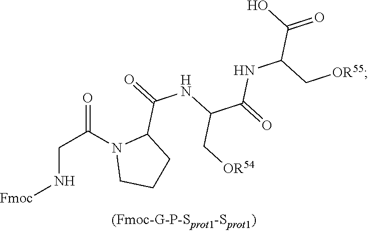

(vii) reacting the compound of formula (Fmoc-G-P-Sₚᵣₒₜ₁-Sₚᵣₒₜ₁) and a compound of formula (Gₚᵣₒₜ) to form a compound of formula (Fmoc-G-P-Sₚᵣₒₜ₁-Sₚᵣₒₜ₁-Gₚᵣₒₜ)

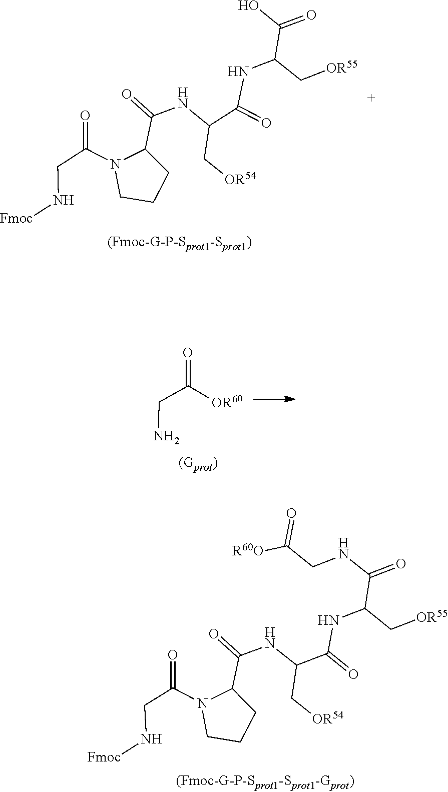

wherein R⁶⁰ is a protecting group;
(viii) converting the compound of formula (Fmoc-G-P-Sₚᵣₒₜ₁-Sₚᵣₒₜ₁-Gₚᵣₒₜ₁) to a compound of Formula (IV-b)

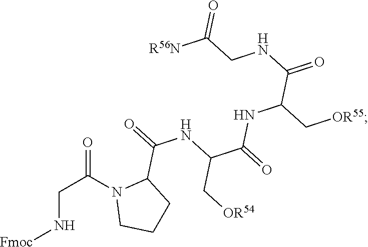

(ix) optionally removing one or more protecting groups R⁵⁴, R⁵⁵ and R⁵⁶.

In some embodiments, the protecting groups are chosen from Boc, Fmoc, tert-butyl, and trityl groups.

The present disclosure also provides a method of synthesizing a polypeptide of SEQ ID NO: 12, the method comprising conjugating the compound of Formula (IV-b) via its C-terminus to the N-terminus of a polypeptide of SEQ ID NO: 22, and conjugating the compound of Formula (IV-b) via its N-terminus to the C-terminus of a polypeptide of SEQ ID NO: 23.

The present disclosure also provides a method of synthesizing a polypeptide of SEQ ID NO: 24, the method comprising conjugating the compound of Formula (IV-b) via its C-terminus to the N-terminus of a polypeptide of SEQ ID NO: 22.

The present disclosure also provides a method of synthesizing a polypeptide of SEQ ID NO: 16, the method comprising conjugating the compound of Formula (IV-b) via its N-terminus to the C-terminus of a polypeptide of SEQ ID NO: 25.

The present disclosure also provides a method of synthesizing a polypeptide of SEQ ID NO: 1, the method comprising conjugating the compound of Formula (IV-b) via its C-terminus to the N-terminus of a polypeptide of SEQ ID NO: 8, and conjugating the compound of Formula (IV-b) via its N-terminus to the C-terminus of a polypeptide of SEQ ID NO: 9.

### Formula (V)

In yet another aspect, the present disclosure provides a compound of Formula (V), or a salt, solvate, or hydrate thereof,

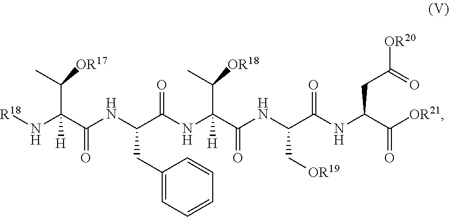

wherein R¹⁶ is H or a protecting group; R¹⁷ is H or a protecting group; R¹⁸ is H or a protecting group; R¹⁹ is H or a protecting group; R²⁰ is H or a protecting group; and R²¹ is H or a protecting group.

In some embodiments, each protecting group is independently chosen from Boc, Fmoc, tert-butyl, benzyl, carboxybenzyl, methyl, allyl, cyclohexyl, and trityl groups.

In some embodiments, R¹⁶ is H, Fmoc, or Boc. In some embodiments, R¹⁷ is H or t-butyl. In some embodiments, R¹⁸ is H or t-butyl. In some embodiments, R¹⁹ is H or t-butyl. In some embodiments, R²⁰ is H or t-butyl. In some embodiments, R²¹ is H or benzyl. In some embodiments, at least one of R¹⁶, R¹⁷, R¹⁸, R¹⁹, R²⁰, and R²¹ is a protecting group. In some embodiments, R¹⁶, R¹⁷, R¹⁸, R¹⁹, R²⁰, and R²¹ are H.

In some embodiments, Formula (V) is Fmoc-Thr(tBu)-Phe-Thr(tBu)-Ser(tBu)-Asp(OtBu)-OH having a structure of Formula (V-a)

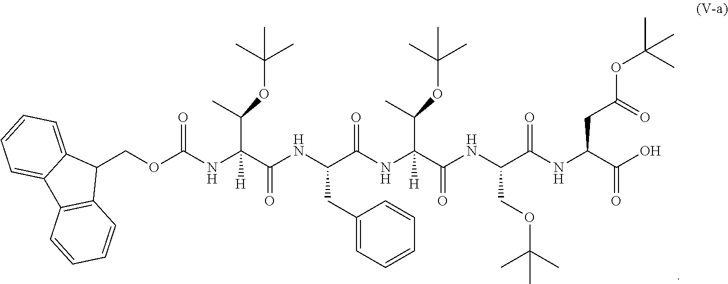 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)N[C@@H](CC(=O)OC(C)(C)C)C(=O)O)[C@@H](C)OC(C)(C)C]

In some embodiments, a compound of Formula (V) or (V-a) is crystalline.

In some embodiments, a compound of Formula (V-a) is in the form of a crystalline solid characterized by a peak in the X-ray powder diffraction pattern at diffraction angle 2θ±0.2 degrees of 5.8-6.1° in combination of one or more peaks select from the group consisting of 6.7-7.1° and 8.8-9.0°.

In some embodiments, a compound of Formula (V-a) is in the form of a crystalline solid characterized by a peak in the X-ray powder diffraction pattern at diffraction angle 2θ±0.2 degrees of 5.0-5.2° in combination of one or more peaks select from the group consisting of 5.3-5.4°, 5.7-6.0°, 6.1-6.2°, 7.6-7.9°, and 8.7-9.1°.

In some embodiments, polypeptide compounds as described herein may be used for synthesizing the active polypeptide TZP (SEQ ID NO: 1, 39 amino acids). In some embodiments, some of the compounds disclosed herein are conjugated to other polypeptide fragments to form the full-length TZP. In some embodiments, the present crystalline compounds (such as Y-Aib-E-G tetramer of Formula (I-a)) may be employed as pure, stable, easy to use intermediates for TZP synthesis at high yield. In some embodiments, crystallization of the present compounds may help to reject impurities from the crude material. Compared to amorphous crude materials, the crystalline compounds may show improved purity (e.g., as measured by UPLC-MS analysis). In some embodiments, crystallization of the crude compounds reduce the necessary solvent flow, providing a “greener,” more ecologically desired process. In some embodiments, crystallization of the crude compounds spiked with 1% dimer (impurity) may yield crystalline compound with almost complete rejection of the dimer. In some embodiments, crystallization also improves physical properties, for example, the hygroscopicity. In some embodiments, the crystalline compounds (such as the Y-Aib-E-G tetramer of Formula (I-a)) may display significantly reduced weight gain from moisture absorption compared to the amorphous compounds.

Various synthesis routes may be selected using peptide synthesis technologies known in the art. In any given synthesis route, one or more compounds as described in the present disclosure may be employed.

In some embodiments, disclosed is a method of synthesizing a polypeptide of SEQ ID NO: 1 (TZP), the method comprising conjugating the compound of Formula (I) via its C-terminus to the N-terminus of a polypeptide of SEQ ID NO: 2 (TZP amino acids 5-39).

In some embodiments, disclosed is a method of synthesizing a polypeptide of SEQ ID NO: 26 (TZP 1-14 aa, which is Tyr Xaa Glu Gly Thr Phe Thr Ser Asp Tyr Ser Ile Xaa Leu where Xaa ia Aib), the method comprising conjugating the compound of Formula (I) via its C-terminus to the N-terminus of a polypeptide of SEQ ID NO: 27 (TZP 5-14, which is Thr Phe Thr Ser Asp Tyr Ser Ile Xaa Leu where Xaa is Aib).

### Formula (VI)

In another aspect, the present disclosure provides a compound of Formula (VI), or a salt, solvate, or hydrate thereof,

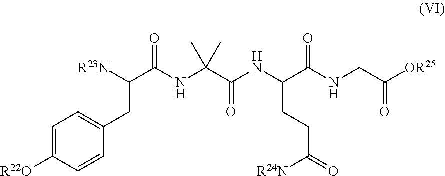

wherein R²² is H or a protecting group; R²³ is H or a protecting group; R²⁴ is H or a protecting group, and R²⁵ is H or a protecting group.

In some embodiments, each protecting group is independently chosen from Boc, Fmoc, tert-butyl, benzyl, carboxybenzyl, methyl, allyl, cyclohexyl, and trityl groups. In some embodiments, R²² is H or Boc. In some embodiments, wherein R²³ is H or tert-butyl. In some embodiments, R²⁴ is H or trityl. In some embodiments, R²⁵ is H or tert-butyl. In some embodiments, at least one of R²², R²³, R²⁴, and R²⁵ is H. In some embodiments, at least one of R²², R²³, R²⁴, and R²⁵ is a protecting group.

In some embodiments, Formula (VI) is Boc-Tyr(tBu)-Aib-Gln(trt)-Gly-OH having a structure of Formula (VI-a):

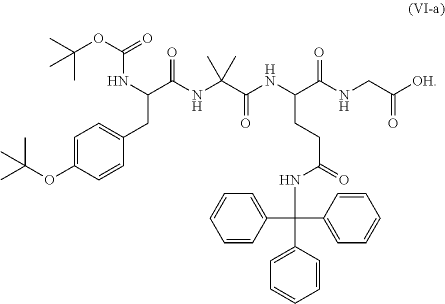 [SMILES: CC(C)(C)OC(=O)NC(Cc1ccc(OC(C)(C)C)cc1)C(=O)NC(C)(C)C(=O)NC(CCC(=O)NC(c1ccccc1)(c1ccccc1)c1ccccc1)C(=O)NCC(=O)O]

In some embodiments, the compound of Formula (VI) or (VI-a) is a solvate. In some embodiments, the solvate produced from pentyl acetate, mixtures comprising pentyl acetate, ethyl acetate, or mixtures comprising 2-methyl tetrahydrofuran and t-amyl methyl ether.

In some embodiments, the mixtures comprising pentyl acetate are chosen from mixtures comprising pentyl acetate and t-butyl ethyl ether, mixtures comprising pentyl acetate and t-amyl methyl ether, or mixtures comprising pentyl acetate and heptane.

In some embodiments, the compound of Formula (VI) or (VI-a) is a de-solvate or a partial de-solvate.

In some embodiments, the compound of Formula (VI) or (VI-a) is crystalline.

In some embodiments, the compound of Formula (VI-a) is in the form of a crystalline solid characterized by a peak in the X-ray powder diffraction pattern at diffraction angle 2-theta±0.2 degrees of 6.3-6.4° in combination of one or more peaks chosen from 4.5°, 7.1°, 13.0-13.1°, 15.9-16.0°, and 18.4-18.6°.

In some embodiments, the compound of Formula (VI-a) is in the form of a crystalline solid characterized by a peak in the X-ray powder diffraction pattern at diffraction angle 2-theta±0.2 degrees of 7.0-7.2° in combination of one or more peaks chosen from 5.0-5.4°, 7.6-7.7°, 8.8-8.9°, 9.4-9.5°, and 12.5-12.7°.

The present disclosure also provides a method of synthesizing the compound of Formula (VI), the method comprising:
(i) reacting a compound of formula (Yₚᵣₒₜ) and a compound of formula (Aibₚᵣₒₜ) to form a compound of formula (Yₚᵣₒₜ-Aibₚᵣₒₜ)

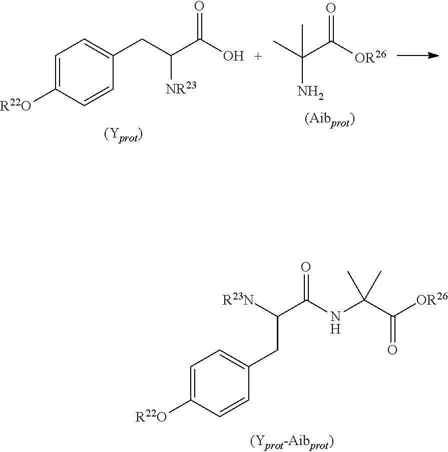

wherein R²², R²³, and R²⁶ are protecting groups;
(ii) removing the protecting group R²⁶ to form a compound of (Yₚᵣₒₜ-Aib)

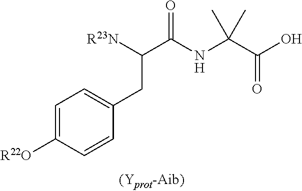

(iii) reacting the compound of formula (Yₚᵣₒₜ-Aib) and a compound of formula (Qₚᵣₒₜ₂) to form a compound of a formula (Yₚᵣₒₜ-Aib-Qₚᵣₒₜ₂)

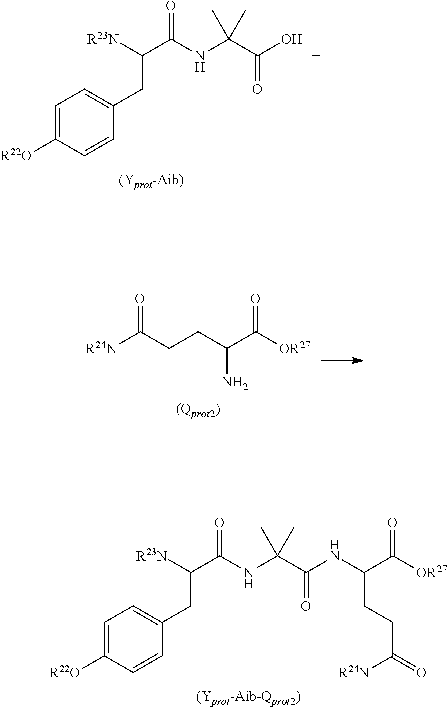

wherein R²⁴ and R²⁷ are protecting groups;
(iv) removing the protecting group R²⁷ to form a compound of (Yₚᵣₒₜ-Aib-Qₚᵣₒₜ₁)

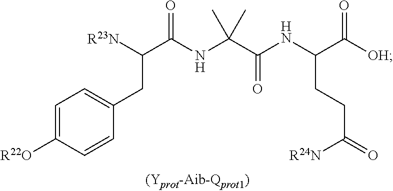

(v) reacting the compound of formula (Yₚᵣₒₜ-Aib-Qₚᵣₒₜ₁) and a compound of formula (Gₚᵣₒₜ) to form a compound of Formula (VI)

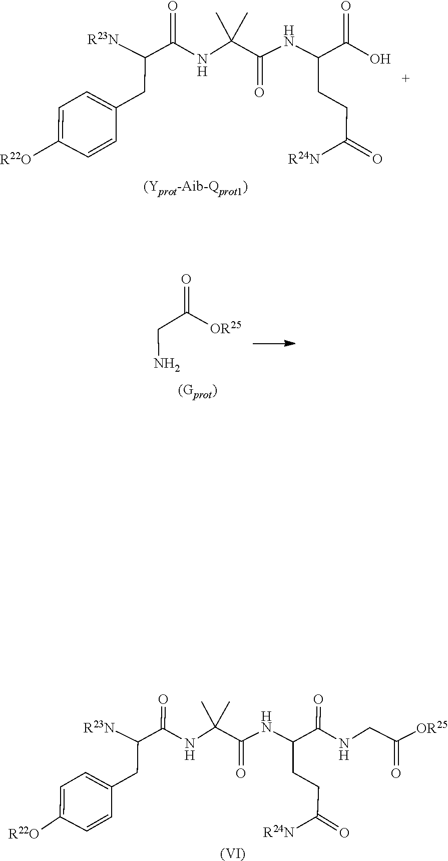

wherein R²⁵ is a protecting group; and
(vi) optionally removing one or more protecting groups R²², R²³, R²⁴, and R²⁵.

In some embodiments, the protecting groups are chosen from Boc, Fmoc, tert-butyl, and trityl groups.

The present disclosure further provides a method of synthesizing a polypeptide of SEQ ID NO: 12, the method comprising conjugating the C-terminus of the compound of Formula (VI-a) to the N-terminus of a polypeptide of SEQ ID NO: 13.

The present disclosure also provides a method of synthesizing a polypeptide of SEQ ID NO: 14, the method comprising conjugating the C-terminus of the compound of Formula (VI-a) to the N-terminus of a polypeptide of SEQ ID NO: 15.

### Formula (VII)

In another aspect, the present disclosure provides a compound of Formula (VII), or a salt, solvate, or hydrate thereof,

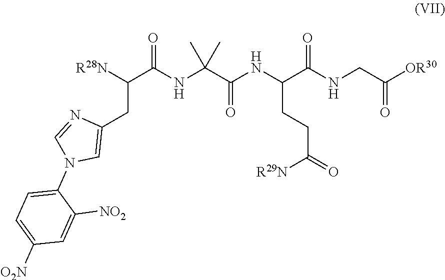

wherein R²⁸ is H or a protecting group; R²⁹ is H or a protecting group, and R³⁰ is H or a protecting group.

In some embodiments, each protecting group is independently chosen from Boc, Fmoc, tert-butyl, benzyl, carboxybenzyl, methyl, allyl, cyclohexyl, and trityl groups. In some embodiments, R²⁸ is H or Boc. In some embodiments, R²⁹ is H or trityl. In some embodiments, R³⁰ is H or tert-butyl. In some embodiments, at least one of R²⁸, R²⁹, and R³⁰ is H. In some embodiments, at least one of R²⁸, R²⁹, and R³⁰ is a protecting group.

In some embodiments, Formula (VII) is Boc-His(dnp)-Aib-Gln(trt)-Gly-OH having a structure of Formula (VII-a):

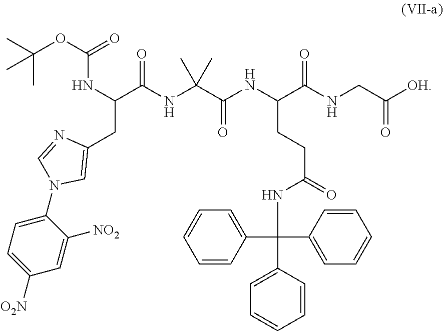 [SMILES: CC(C)(C)OC(=O)NC(Cc1cn(-c2ccc([N+](=O)[O-])cc2[N+](=O)[O-])cn1)C(=O)NC(C)(C)C(=O)NC(CCC(=O)NC(c1ccccc1)(c1ccccc1)c1ccccc1)C(=O)NCC(=O)O]

In some embodiments, the compound of Formula (VII) or (VII-a) is a solvate. In some embodiments, the solvate is produced from any one of a mixture of acetonitrile and methyl tert-butyl ether, a mixture of nitromethane and methyl tert-butyl ether, a mixture of tetrahydrofuran and methyl tert-butyl ether, methyl acetate, and ethyl acetate.

In some embodiments, the compound of Formula (VII) or (VII-a) is crystalline.

In some embodiments, the compound of Formula (VII) or (VII-a) is in the form of a crystalline solid characterized by a peak in the X-ray powder diffraction pattern at diffraction angle 2-theta±0.2 degrees of 4.8° in combination of one or more peaks chosen from 5.6°, 6.2°, 14.8°, and 15.6°.

In some embodiments, the compound of Formula (VII-a) is in the form of a crystalline solid characterized by a peak in the X-ray powder diffraction pattern at diffraction angle 2-theta±0.2 degrees of 5.3° in combination of one or more peaks chosen from 7.7°, 10.5°, 11.3°, 11.6°, and 14.4°.

In some embodiments, the compound of Formula (VII-a) is in the form of a crystalline solid characterized by peaks in the X-ray powder diffraction pattern at diffraction angle 2-theta±0.2 degrees of 6.2° and 6.9°.

The present disclosure also provides a method of synthesizing a compound of Formula (VII), the method comprising:
(i) reacting a compound of formula (H(dnp)ₚᵣₒₜ) and a compound of formula (Aibₚᵣₒₜ) to form a compound of formula (H(dnp)ₚᵣₒₜ-Aibₚᵣₒₜ)

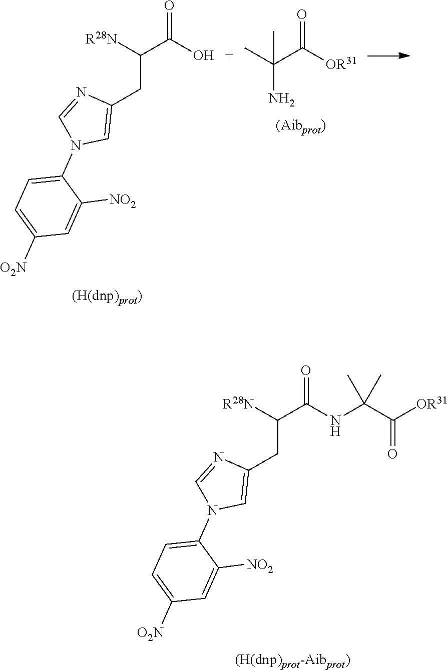

wherein R²⁸ and R³¹ are protecting groups;
(ii) removing the protecting group R³¹ to form a compound of (H(dnp)ₚᵣₒₜ-Aib)

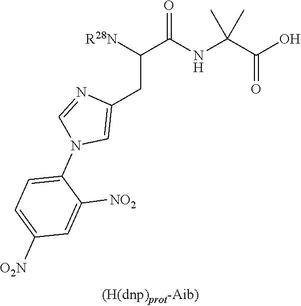

(iii) reacting the compound of formula (H(dnp)ₚᵣₒₜ-Aib) and a compound of formula (Qₚᵣₒₜ₂) to form a compound of a formula (H(dnp)ₚᵣₒₜ-Aib-Qₚᵣₒₜ₂)

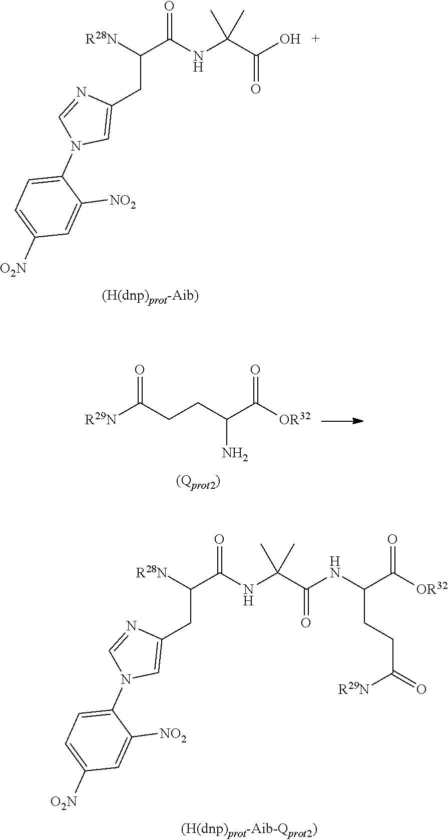

wherein R²⁹ and R³² are protecting groups;
(iv) removing the protecting group R³² to form a compound of (H(dnp)ₚᵣₒₜ-Aib-Qₚᵣₒₜ₁)

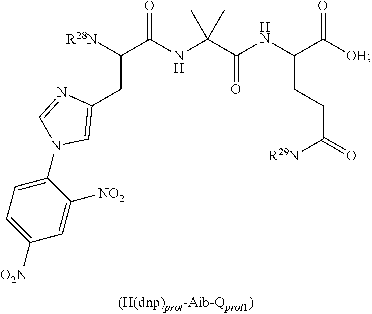

(v) reacting the compound of formula (H(dnp)ₚᵣₒₜ-Aib-Qₚᵣₒₜ₁) and a compound of formula (Gₚᵣₒₜ) to form a compound of Formula (VII)

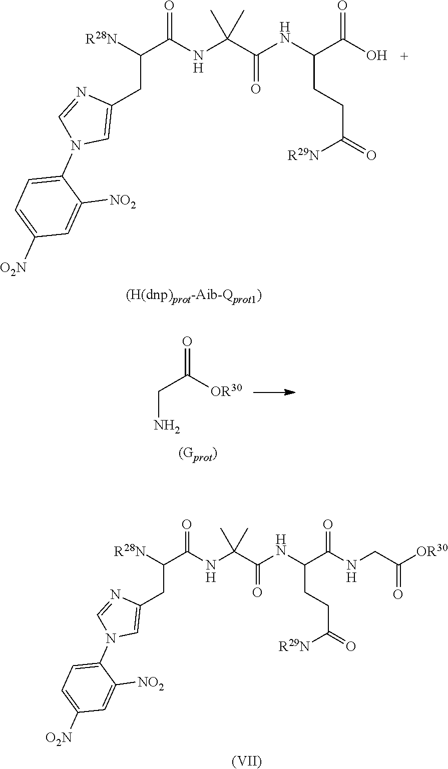

wherein R⁰ is a protecting group; and
(vi) optionally removing one or more protecting groups R²⁸, R²⁹, and R³⁰.

The present disclosure also provides a method of synthesizing a polypeptide of SEQ ID NO: 16, the method comprising conjugating the compound of Formula (VII) or Formula (VII-a) via its C-terminus to the N-terminus of a polypeptide of SEQ ID NO: 17.

### Formula (VIII)

In another aspect, the present disclosure provides a compound of Formula (VIII), or a salt, solvate, or hydrate thereof,

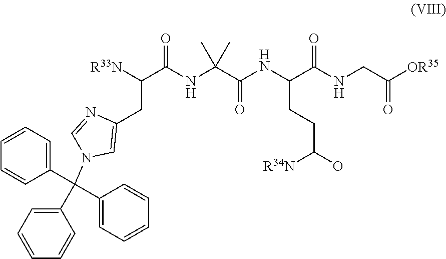

wherein R³³ is H or a protecting group; R³⁴ is H or a protecting group; and R³⁵ is H or a protecting group.

In some embodiments, each protecting group is independently chosen from Boc, Fmoc, tert-butyl, benzyl, carboxybenzyl, methyl, allyl, cyclohexyl, and trityl groups. In some embodiments, R³³ is H, or Boc. In some embodiments, R³⁴ is H or trityl. In some embodiments, R³⁵ is H, or tert-butyl. In some embodiments, at least one of R³³, R³⁴, and R³⁵ is H. In some embodiments, at least one of R³³, R³⁴, and R³⁵ is a protecting group.

In some embodiments, Formula (VIII) is Boc-His(trt)-Aib-Gln(trt)-Gly-OH having a structure of Formula (VIII-a):

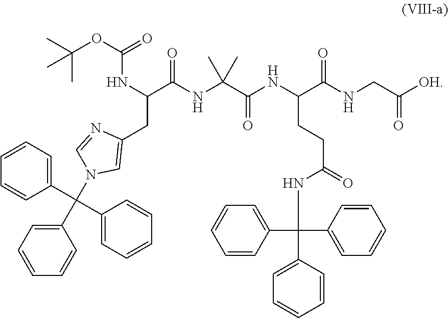 [SMILES: CC(C)(C)OC(=O)NC(Cc1cn(C(c2ccccc2)(c2ccccc2)c2ccccc2)cn1)C(=O)NC(C)(C)C(=O)NC(CCC(=O)NC(c1ccccc1)(c1ccccc1)c1ccccc1)C(=O)NCC(=O)O]

In some embodiments, the compound of Formula (VIII) or (VIII-a) is a solvate. In some embodiments, the solvate is produced from any one of a mixture of tetrahydrofuran and methyl tert-butyl ether, a mixture of tetrahydrofuran and heptane, a mixture of 1,4-dioxane and water, a mixture of ethyl acetate and methyl tert-butyl ether, and a mixture of acetonitrile and methyl tert-butyl ether.

In some embodiments, the compound of Formula (VIII) or (VIII-a) is crystalline.

In some embodiments, the compound of Formula (VIII-a) is in the form of a crystalline solid characterized by a peak in the X-ray powder diffraction pattern at diffraction angle 2-theta±0.2 degrees of 4.7° in combination of one or more peaks chosen from 5.5°, 8.2°, 10.1°, 11.8°, 13.3°, 13.6°, and 18.9°.

In some embodiments, the compound of Formula (VIII-a) is in the form of a crystalline solid characterized by a peak in the X-ray powder diffraction pattern at diffraction angle 2-theta±0.2 degrees of 5.8° in combination of one or more peaks chosen from 5.3°, 8.9°, 9.2°, 15.2°, 18.6°, and 19.5°.

The present disclosure also provides a method of synthesizing the compound of Formula (VIII), the method comprising:
(i) reacting a compound of formula (H(trt)ₚᵣₒₜ) and a compound of formula (Aibₚᵣₒₜ) to form a compound of formula (H(trt)ₚᵣₒₜ-Aibₚᵣₒₜ)

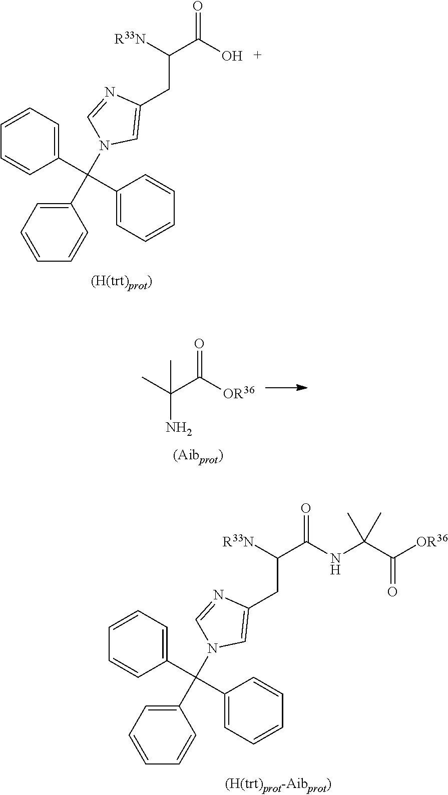

wherein R³³ and R³⁶ are protecting groups;
(ii) removing the protecting group R³⁶ to form a compound of (H(trt)ₚᵣₒₜ-Aib)

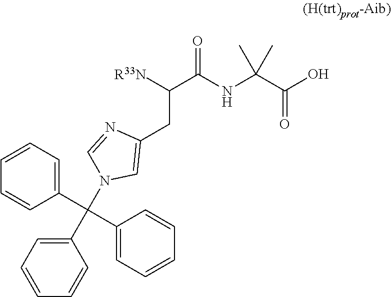

(iii) reacting the compound of formula (H(trt)ₚᵣₒₜ-Aib) and a compound of formula (Qₚᵣₒₜ₂) to form a compound of a formula (H(trt)ₚᵣₒₜ-Aib-Qₚᵣₒₜ₂)

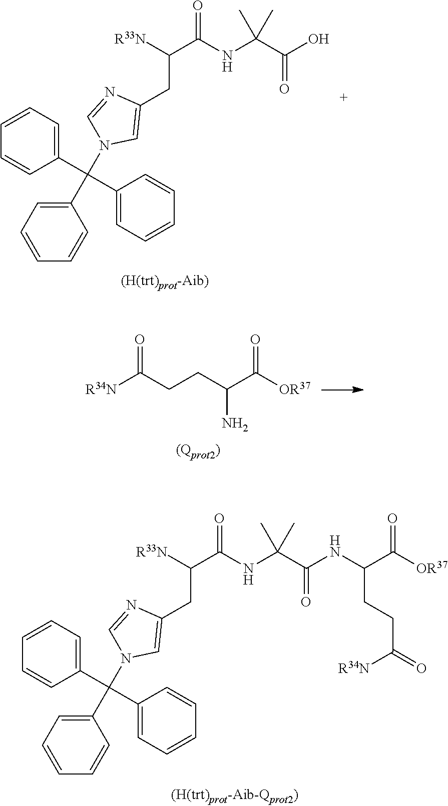

wherein R³³ and R³⁷ are protecting groups;
(iv) removing the protecting group R³⁷ to form a compound of (H(trt)ₚᵣₒₜ-Aib-Qₚᵣₒₜ₁)

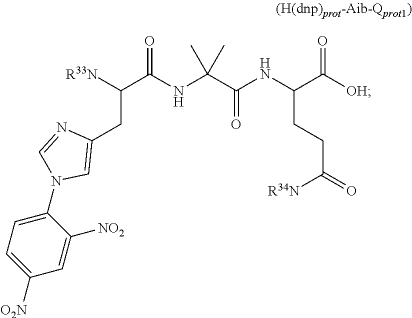

(v) reacting the compound of formula (H(trt)ₚᵣₒₜ-Aib-Qₚᵣₒₜ₁) and a compound of formula (Gₚᵣₒₜ) to form a compound of Formula (VIII)

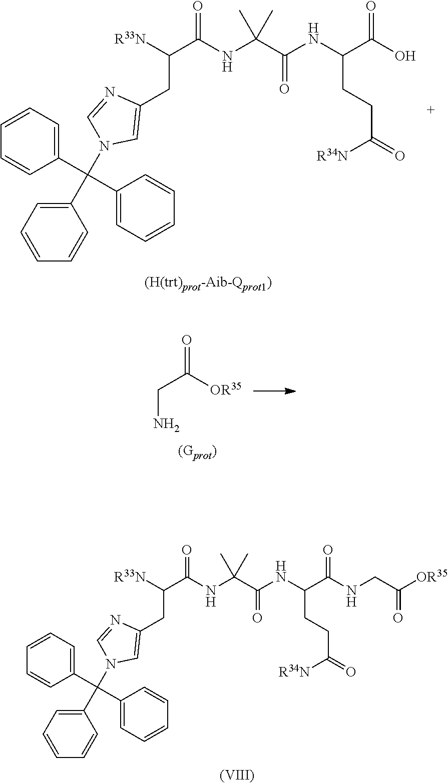

wherein R³⁵ is a protecting group; and
(vi) optionally removing one or more protecting groups R³³, R³⁴, and R³⁵.

In some embodiments, the protecting groups are chosen from Boc, Fmoc, tert-butyl, and trityl groups.

The present disclosure also provides a method of synthesizing a polypeptide of SEQ ID NO: 16, the method comprising conjugating the compound of Formula (VIII-a) via its C-terminus to the N-terminus of a polypeptide of SEQ ID NO: 17.

### Formula (IX)

In another aspect, the present disclosure provides a compound of Formula (IX), or a salt, solvate, or hydrate thereof,

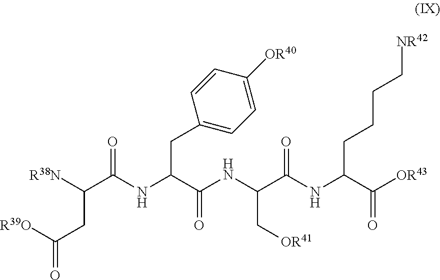

wherein R³⁸ is H or a protecting group; R³⁹ is H or a protecting group; R⁴⁰ is H or a protecting group; R⁴¹ is H or a protecting group; R⁴² is H or a protecting group; and R⁴³ is H or a protecting group.

In some embodiments, each protecting group is independently chosen from Boc, Fmoc, tert-butyl, benzyl, carboxybenzyl, methyl, allyl, cyclohexyl, and trityl groups.

In some embodiments, R³⁸ is H, or Fmoc. In some embodiments, R³⁹ is H or tert-butyl. In some embodiments, R⁴⁰ is H, or tert-butyl. In some embodiments, R⁴¹ is H or tert-butyl. In some embodiments, R⁴² is H, or Boc. In some embodiments, R⁴³ is H, or tert-butyl In some embodiments, at least one of R³⁸, R³⁹, R⁴⁰, R⁴¹, R⁴², and R⁴³ is H. In some embodiments, at least one of R³⁸, R³⁹, R⁴⁰, R⁴¹, R⁴², and R⁴³ is a protecting group.

In some embodiments, Formula (IX) is Fmoc-Asp(tBu)-Tyr(tBu)-Ser(TBu)-(TBu)Lys-OH having a structure of Formula (IX-a):

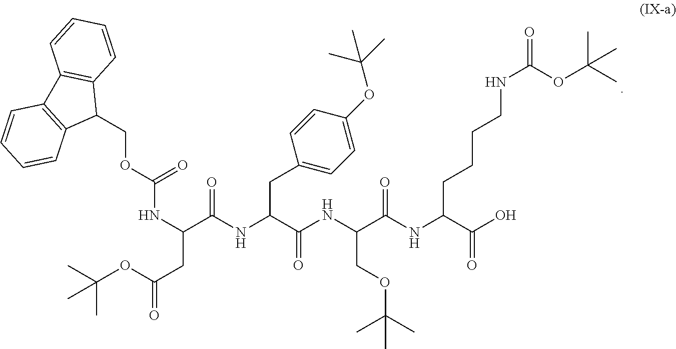 [SMILES: CC(C)(C)OCC(NC(=O)C(Cc1ccc(OC(C)(C)C)cc1)NC(=O)C(CC(=O)OC(C)(C)C)NC(=O)OCC1c2ccccc2-c2ccccc21)C(=O)NC(CCCCNC(=O)OC(C)(C)C)C(=O)O]

In some embodiments, the compound of Formula (IX) or (IX-a) is a solvate.

In some embodiments, the solvate is produced from any one of a mixture of methyl acetate and dibutyl ether, a mixture of acetone and dibutyl ether, a mixture of acetonitrile and dibutyl ether, a mixture of ethyl acetate and dibutyl ether, a mixture of methyl acetate and heptane, and a mixture of methyl ethyl ketone and dibutyl ether.

In some embodiments, the compound of Formula (IX) or (IX-a) is a de-solvate or is anhydrous.

In some embodiments, the compound of Formula (IX-a) is crystalline.

In some embodiments, the compound of Formula (IX-a) is in the form of a crystalline solid characterized by a peak in the X-ray powder diffraction pattern at diffraction angle 2-theta±0.2 degrees of 5.3° in combination of one or more peaks chosen from 6.0°, 6.9°, 7.2°, 8.0°, 12.2°, and 15.6°.

In some embodiments, the compound of Formula (IX-a) is in the form of a crystalline solid characterized by a peak in the X-ray powder diffraction pattern at diffraction angle 2-theta±0.2 degrees of 5.8° in combination of one or more peaks chosen from 4.4°, 6.6°, 10.1°, 11.4°, 13.4°, and 15.5°.

In some embodiments, the compound of Formula (IX-a) is in the form of a crystalline solid characterized by peaks in the X-ray powder diffraction pattern at diffraction angle 2-theta±0.2 degrees of 4.5° and 5.5° in combination of one or more peaks chosen from 6.0° and 7.3°.

The present disclosure also provides a method of synthesizing the compound of Formula (IX), the method comprising:
(i) reacting a compound of formula (Dₚᵣₒₜ) and a compound of formula (Yₚᵣₒₜ₂) to form a compound of formula (Dₚᵣₒₜ-Yₚᵣₒₜ₂)

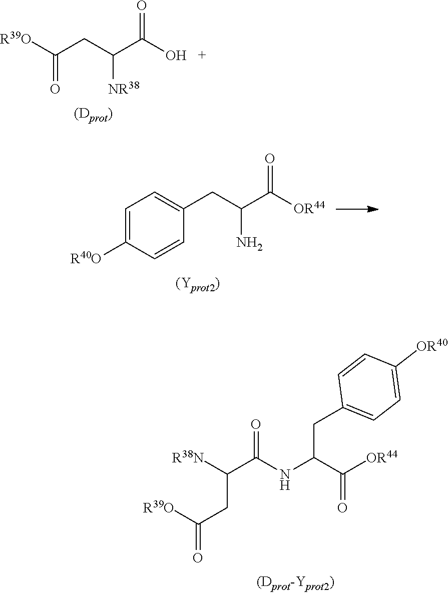

wherein R³⁸, R³⁹, R⁴⁰, and R⁴⁴ are protecting groups;
(ii) removing the protecting group R⁴⁴ to form a compound of (Dₚᵣₒₜ-Yₚᵣₒₜ₁)

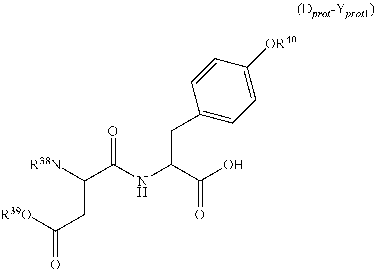

(iii) reacting the compound of formula (Dₚᵣₒₜ-Yₚᵣₒₜ₁) and a compound of formula (Sₚᵣₒₜ₂) to form a compound of a formula (Dₚᵣₒₜ-Yₚᵣₒₜ₁-Sₚᵣₒₜ₂)

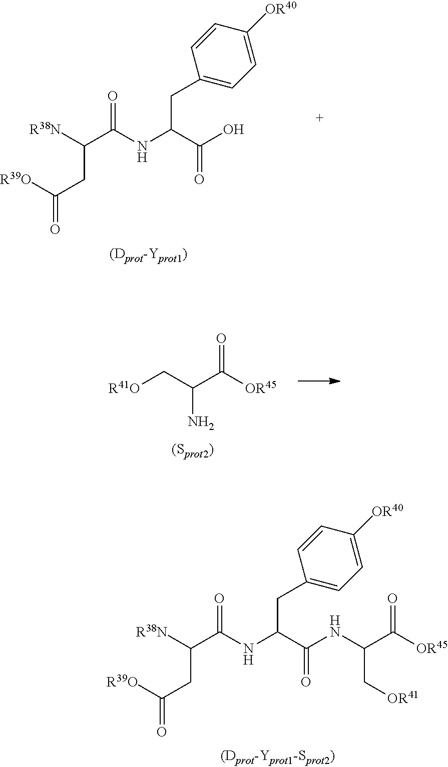

wherein R⁴¹ and R^{4S} are protecting groups;
(iv) removing the protecting group R⁴⁵ to form a compound of (Dₚᵣₒₜ-Yₚᵣₒₜ₁-Sₚᵣₒₜ₁)

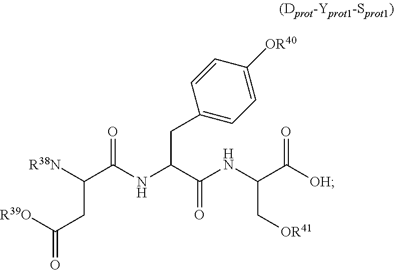

(v) reacting the compound of formula (Dₚᵣₒₜ-Yₚᵣₒₜ₁-Sₚᵣₒₜ₁) and a compound of formula (Kₚᵣₒₜ₂) to form a compound of Formula (IX)

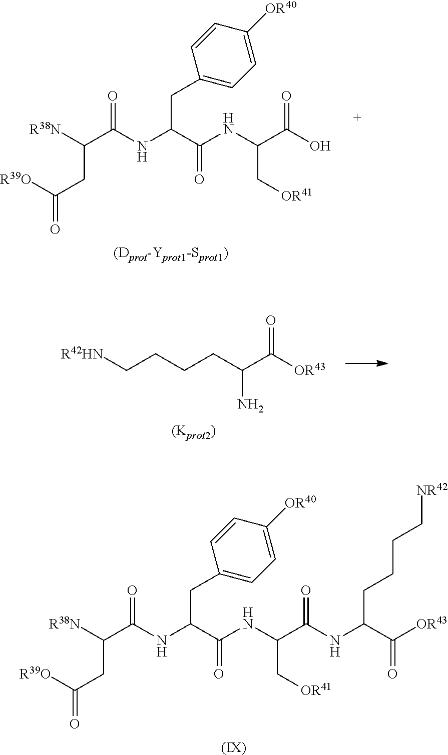

wherein R⁴² and R⁴³ are protecting groups; and
(vi) optionally removing one or more protecting groups R³⁸, R³⁹, R⁴⁰, R⁴¹, R⁴², and R⁴³.

In some embodiments, the protecting groups are chosen from Boc, Fmoc, tert-butyl, and trityl groups.

The present disclosure also provides a method of synthesizing a polypeptide of SEQ ID NO: 16, the method comprising conjugating the compound of Formula (IX-a) via its C-terminus to the N-terminus of a polypeptide of SEQ ID NO: 18, and conjugating the compound of Formula (IX-a) via its N-terminus to the C-terminus of a polypeptide of SEQ ID NO: 19.

### Formula (X)

In another aspect, the present disclosure provides a compound of Formula (X), or a salt, solvate, or hydrate thereof,

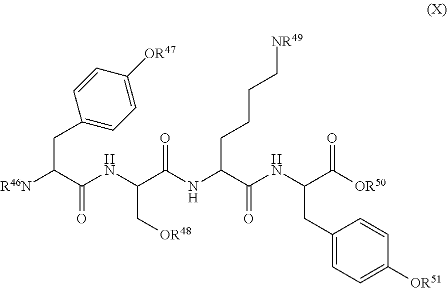

wherein R⁴⁶ is H or a protecting group; R⁴⁷ is H or a protecting group; R⁴⁸ is H or a protecting group; R⁴⁹ is H or a protecting group; R⁵⁰ is H or a protecting group; and R⁵¹ is H or a protecting group.

In some embodiments, each protecting group is independently chosen from Boc, Fmoc, tert-butyl, benzyl, carboxybenzyl, methyl, allyl, cyclohexyl, and trityl groups.

In some embodiments, R⁴⁶ is H or tert-butyl. In some embodiments, R⁴⁷ is H or tert-butyl. In some embodiments, R⁴⁸ is H or Boc. In some embodiments, R⁴⁹ is H or Fmoc. In some embodiments, R⁵⁰ is H or tert-butyl. In some embodiments, R⁵¹ is H or tert-butyl. In some embodiments, at least one of R⁴⁶, R⁴⁷, R⁴⁸, R⁴⁹, R⁵⁰, and R⁵¹ is H. In some embodiments, at least one of R⁴⁶, R⁴⁷, R⁴⁸, R⁴⁹, R⁵⁰, and R⁵¹ is a protecting group.

In some embodiments, Formula (X) is Fmoc-Tyr(tBu)-Ser(tBu)-Lys(Boc)-(tBu)Tyr-OH having a structure of Formula (X-a):

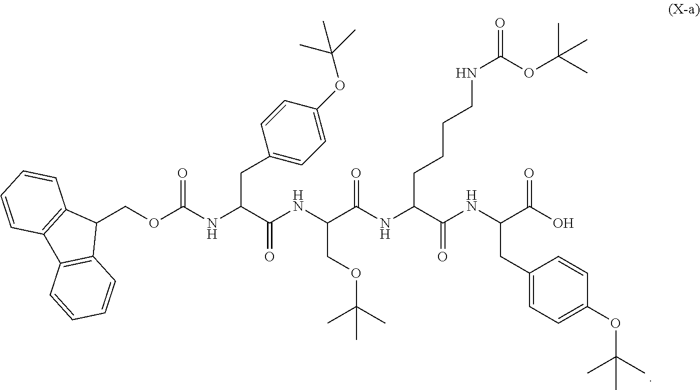 [SMILES: CC(C)(C)OCC(NC(=O)C(Cc1ccc(OC(C)(C)C)cc1)NC(=O)OCC1c2ccccc2-c2ccccc21)C(=O)NC(CCCCNC(=O)OC(C)(C)C)C(=O)NC(Cc1ccc(OC(C)(C)C)cc1)C(=O)O]

In some embodiments, the compound of Formula (X) or (X-a) is a solvate.

In some embodiments, the solvate is produced from ethanol or isopropyl alcohol.

In some embodiments, the compound of Formula (X) or (X-a) is a de-solvate.

In some embodiments, the compound of Formula (X) or (X-a) is crystalline.

In some embodiments, the compound of Formula (X-a) is in the form of a crystalline solid characterized by peaks in the X-ray powder diffraction pattern at diffraction angle 2-theta±0.2 degrees of 18.1° and 18.7° in combination of one or more peaks chosen from 5.7°, 8.7°, 13.7°, 14.3°, 15.9°, and 16.2°.

In some embodiments, the compound of Formula (X-a) is in the form of a crystalline solid characterized by peaks in the X-ray powder diffraction pattern at diffraction angle 2-theta±0.2 degrees of 5.9° and 10.5° in combination of one or more peaks chosen from 7.1°, 8.9°, 14.6°, and 16.6°.

In some embodiments, the compound of Formula (X-a) is in the form of a crystalline solid characterized by peaks in the X-ray powder diffraction pattern at diffraction angle 2-theta±0.2 degrees of 7.8° and 20.3° in combination of one or more peaks chosen from 5.8°, 15.5°, and 19.5°.

In some embodiments, the compound of Formula (X-a) is in the form of a crystalline solid characterized by peaks in the X-ray powder diffraction pattern at diffraction angle 2-theta±0.2 degrees of 5.9° and 7.4° in combination of one or more peaks chosen from 6.5°, 6.9°, and 14.8°.

The present disclosure also provides a method of synthesizing the compound of Formula (X), the method comprising:
(i) reacting a compound of formula (Yₚᵣₒₜ) and a compound of formula (Sₚᵣₒₜ₂) to form a compound of formula (Yₚᵣₒₜ-Sₚᵣₒₜ₂)

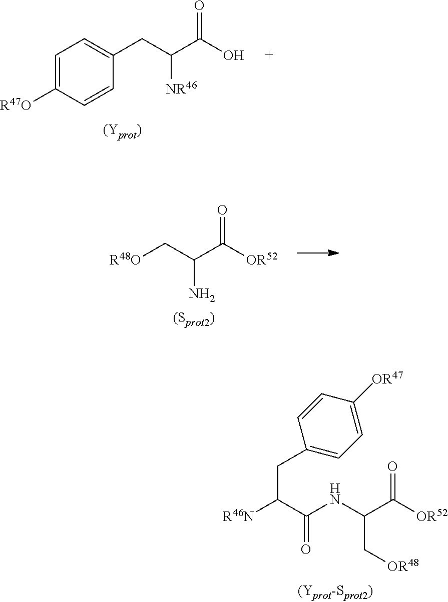

wherein R⁴⁶, R⁴⁷, R⁴⁸, and R⁵² are protecting groups;
(ii) removing the protecting group R⁵² to form a compound of (Yₚᵣₒₜ-Sₚᵣₒₜ₁)

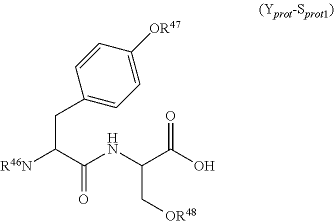

(iii) reacting the compound of formula (Yₚᵣₒₜ-Sₚᵣₒₜ₁) and a compound of formula (Kₚᵣₒₜ₂) to form a compound of a formula (Yₚᵣₒₜ-Sₚᵣₒₜ₁-Kₚᵣₒₜ₂)

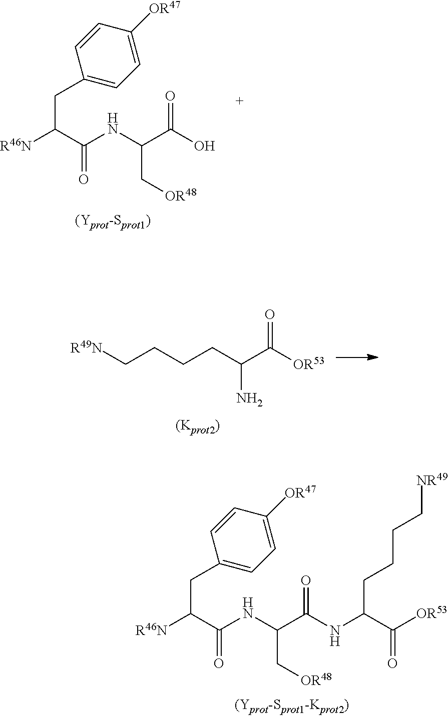

wherein R⁴⁹ and R⁵³ are protecting groups;
(iv) removing the protecting group R⁵³ to form a compound of (Yₚᵣₒₜ-Sₚᵣₒₜ₁-Kₚᵣₒₜ₁)

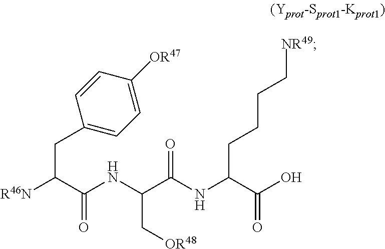

(v) reacting the compound of formula (Yₚᵣₒₜ-Sₚᵣₒₜ₁-Kₚᵣₒₜ₁) and a compound of formula (Yₚᵣₒₜ₂) to form a compound of Formula (X)

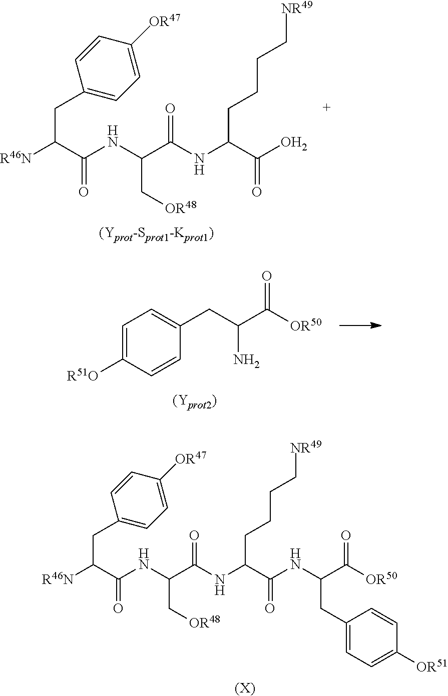

wherein R⁵⁰ and R⁵¹ are protecting groups; and
(vi) optionally removing one or more protecting groups R⁴⁶, R⁴⁷, R⁴⁸, R⁴⁹, R⁵⁰, and R⁵¹.

In some embodiments, the protecting groups are chosen from Boc, Fmoc, tert-butyl, and trityl groups.

The present disclosure also provides a method of synthesizing a polypeptide of SEQ ID NO: 16, the method comprising conjugating the compound of Formula (X-a) via its C-terminus to the N-terminus of a polypeptide of SEQ ID NO: 20, and conjugating the compound of Formula (X-a) via its N-terminus to the C-terminus of a polypeptide of SEQ ID NO: 21.

### Formula (XI)

In another aspect, the present disclosure provides a compound of Formula (XI), or a salt, solvate, or hydrate thereof,

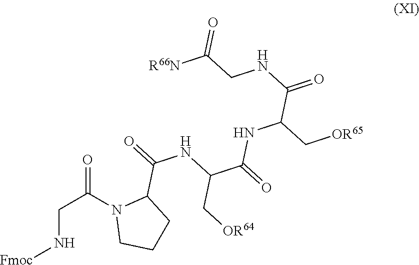

wherein R⁶⁴ is H or a protecting group; R⁶⁵ is H or a protecting group; and R⁶⁶ is H or a protecting group. In some embodiments, R⁶⁴ and R⁶⁵ are protecting groups; and R⁶⁶ is H or a protecting group. In some embodiments, R⁶⁴ and R⁶⁵ are protecting groups; and R⁶⁶ is H.

In some embodiments, each protecting group is independently chosen from Boc, Fmoc, tert-butyl, benzyl, carboxybenzyl, methyl, allyl, cyclohexyl, and trityl groups.

In some embodiments, Formula (XI) is Fmoc-Gly-Pro-Ser(tBu)-Ser(tBu)-Gly-OH having a structure of Formula (XI-a):

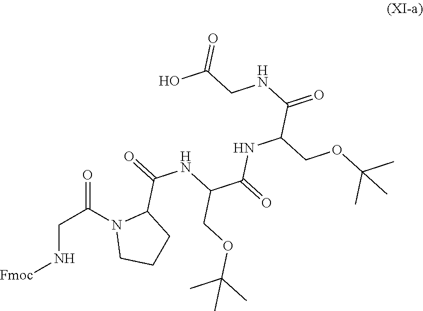

In some embodiments, the compound of Formula (XI-a) is crystalline.

In some embodiments, the compound of Formula (XI-a) is in the form of a crystalline solid characterized by peaks in the X-ray powder diffraction pattern at diffraction angle 2-theta±0.2 degrees of 6.1° and 8.5° in combination of one or more peaks chosen from 5.8°, 16.9°, 18.5°, 18.8°, 19.3°, and 20.9°.

### Formula (X)

In another aspect, the present disclosure provides a compound of Formula (XII), or a salt, solvate, or hydrate thereof,

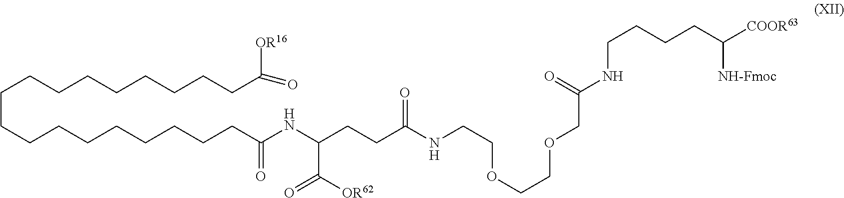

wherein R⁶¹ is H or a protecting group; R⁶² is H or a protecting group; and R⁶³ is H or a protecting group.

In some embodiments, each protecting group is independently chosen from Boc, Fmoc, tert-butyl, carboxybenzyl, methyl, allyl, cyclohexyl, and trityl groups. In some embodiments, R⁶¹ is H or tert-butyl. In some embodiments, R⁶² is H or tert-butyl. In some embodiments, R⁶³ is H or tert-butyl. In some embodiments, at least one of R⁶¹, R⁶², and R⁶³ is H. In some embodiments, at least one of R⁶¹, R⁶², and R⁶³ is a protecting group.

In some embodiments, Formula (XII) is a GGG side chain having a structure of Formula (XII-a):

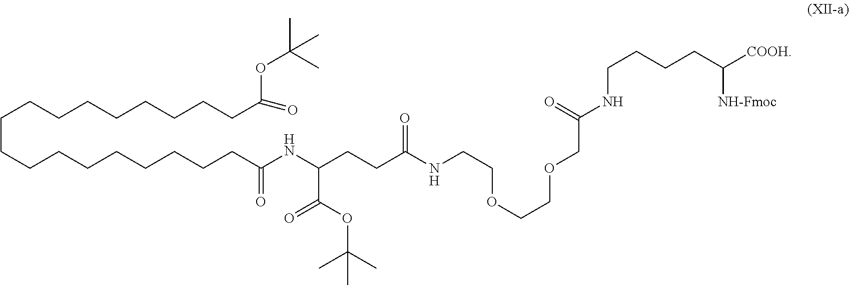

In some embodiments, the compound of Formula (XII) or (XII-a) is a de-solvate or is anhydrous.

In some embodiments, the compound of Formula (XII) or (XII-a) is crystalline.

In some embodiments, the compound of Formula (XII-a) is in the form of a crystalline solid characterized by a peak in the X-ray powder diffraction pattern at diffraction angle 2-theta±0.2 degrees of 11.4° in combination of one or more peaks chosen from 6.0°, 8.9°, 12.7°, 13.6°, 14.6°, 17.0°, and 18.8°.

In some embodiments, the compound of Formula (XII-a) is in the form of a crystalline solid characterized by a peak in the X-ray powder diffraction pattern at diffraction angle 2-theta±0.2 degrees of 10.6° in combination of one or more peaks chosen from 7.1°, 12.1°, 13.6°, 14.2°, 15.2°, 16.0°, and 16.8°.

In some embodiments, the compound of Formula (XII-a) is in the form of a crystalline solid characterized by peaks in the X-ray powder diffraction pattern at diffraction angle 2-theta±0.2 degrees of 10.1° and 15.5° in combination of one or more peaks chosen from 6.1°, 8.7°, 11.4°, 16.6°, and 19.2°.

### Formula (XIII)

In another aspect, the present disclosure provides a compound of Formula (XIII), or a salt, solvate, or hydrate thereof,

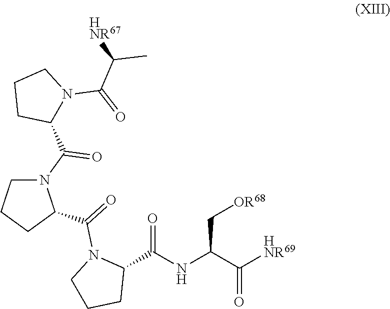

wherein R⁶⁷ is H or a protecting group; R⁶⁸ is H or a protecting group; and R⁶⁹ is H or a protecting group.

In some embodiments, each protecting group is independently chosen from Boc, Fmoc, tert-butyl, carboxybenzyl, methyl, allyl, cyclohexyl, and trityl groups.

In some embodiments, R⁶⁷ is H or tert-butyl. In some embodiments, R⁶⁸ is H or tert-butyl. In some embodiments, R⁶⁹ is H or tert-butyl. In some embodiments, at least one of R⁶⁷, R⁶⁸, and R⁶⁹ is H. In some embodiments, at least one of R⁶⁷, R⁶⁸, and R⁶⁹ is a protecting group. In some embodiments, R⁶⁷ and R⁶⁹ are H, and R⁶⁸ is a protecting group.

In some embodiments, Formula (XIII) is a H-Ala-Pro-Pro-Pro-Ser(tBu)-NH₂ (H-A-P-P-P-S-NH₂) having a structure of Formula (XIII-a):

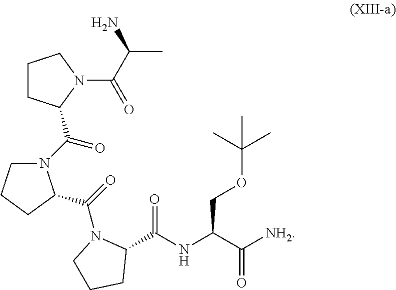 [SMILES: C[C@H](N)C(=O)N1CCC[C@H]1C(=O)N1CCC[C@H]1C(=O)N1CCC[C@H]1C(=O)N[C@@H](COC(C)(C)C)C(N)=O]

In some embodiments, the compound of Formula (XIII) or (XIII-a) is a de-solvate or is anhydrous.

In some embodiments, the compound of Formula (XIII) or (XIII-a) is crystalline.

In some embodiments, the compound of Formula (XIII-a) is in the form of a crystalline solid characterized by a peak in the X-ray powder diffraction pattern at diffraction angle 2-theta±0.2 degrees of 5.0° in combination of one or more peaks chosen from 8.3, 9.7, and 11.2°.

In some embodiments, the compound of Formula (XIII-a) is in the form of a crystalline solid characterized by a peak in the X-ray powder diffraction pattern at diffraction angle 2-theta±0.2 degrees of 7.2° in combination of one or more peaks chosen from 5.3, 8.1, 14.4, and 16.2°.

### Formula (XIV)

In another aspect, the present disclosure provides a compound of Formula (XIV), or a salt, solvate, or hydrate thereof,

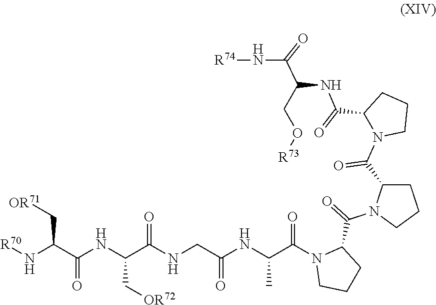

wherein R⁷⁰ is H or a protecting group; R⁷¹ is H or a protecting group; R⁷² is H or a protecting group; R⁷³ is H or a protecting group; and R⁷⁴ is H or a protecting group.

In some embodiments, each protecting group is independently chosen from Boc, Fmoc, tert-butyl, carboxybenzyl, methyl, allyl, cyclohexyl, and trityl groups.

In some embodiments, R⁷⁰ is H, Fmoc, or tert-butyl. In some embodiments, R⁷¹ is H or tert-butyl. In some embodiments, R⁷² is H or tert-butyl. In some embodiments, R⁷³ is H or tert-butyl. In some embodiments, R⁷⁴ is H, Fmoc, or tert-butyl. In some embodiments, at least one of R⁷⁰, R⁷¹, R⁷², R⁷³, and R⁷⁴ is H. In some embodiments, at least one of R⁷⁰, R⁷¹, R⁷², R⁷³, and R⁷⁴ is a protecting group. In some embodiments, R⁷¹, R⁷², and R⁷³ are tert-butyl. In some embodiments, R⁷⁴ is H. In some embodiments, R⁷⁰ is Fmoc.

In some embodiments, Formula (XIV) is a Fmoc-Ser(tBu)-Ser(tBu)-Gly-Ala-Pro-Pro-Pro-Ser(tBu)-NH₂ (Fmoc-S-S-G-A-P-P-P-S-NH₂) having a structure of Formula (XIV-a):

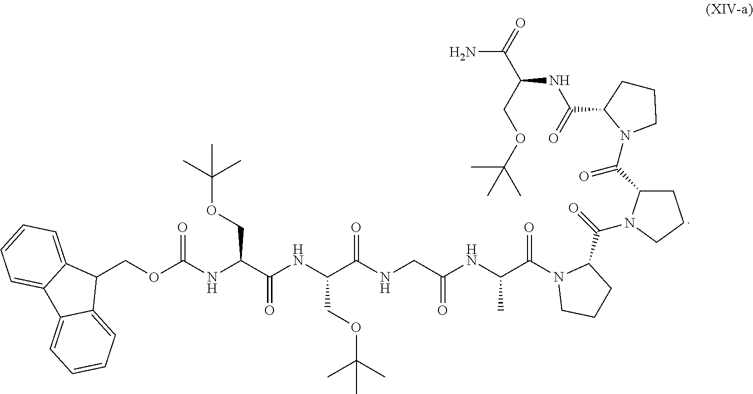 [SMILES: C[C@H](NC(=O)CNC(=O)[C@H](COC(C)(C)C)NC(=O)[C@H](COC(C)(C)C)NC(=O)OCC1c2ccccc2-c2ccccc21)C(=O)N1CCC[C@H]1C(=O)N1CCC[C@H]1C(=O)N1CCC[C@H]1C(=O)N[C@@H](COC(C)(C)C)C(N)=O]

In some embodiments, the compound of Formula (XIV) or (XIV-a) is a de-solvate or is anhydrous.

In some embodiments, the compound of Formula (XIV) or (XIV-a) is crystalline.

In some embodiments, the compound of Formula (XIV-a) is in the form of a crystalline solid characterized by a peak in the X-ray powder diffraction pattern at diffraction angle 2-theta±0.2 degrees of 7.5° in combination of one or more peaks chosen from 6.1, 8.7, 10.6, 15.0, 16.1, and 18.6°.

### Use

In some embodiments, polypeptide compounds as described herein may be used for synthesizing the active polypeptide GGG (SEQ ID NO: 12, 39 amino acids). In some embodiments, some of the compounds disclosed herein are conjugated to other polypeptide fragments to form the full-length GGG. In some embodiments, the present crystalline compounds (such as Y-Aib-Q-G tetramer of Formula (VI-a) and Fmoc-G-P-S-S-G-NH₂ pentamer of Formula (XI-a)) may be employed as pure, stable, easy to use intermediates for GGG synthesis at high yield. In some embodiments, crystallization of the present compounds may help to reject impurities from the crude material. Compared to amorphous crude materials, the crystalline compounds may show improved purity (e.g., as measured by UPLC-MS analysis). In some embodiments, crystallization of the crude compounds reduce the necessary solvent flow, providing a “greener,” more ecologically desired process. In some embodiments, crystallization of the crude compounds spiked with 1% dimer (impurity) may yield crystalline compound with almost complete rejection of the dimer. In some embodiments, crystallization also improves physical properties, for example, the hygroscopicity. In some embodiments, the crystalline compounds (such as Y-Aib-Q-G tetramer of Formula (VI-a) and Fmoc-G-P-S-S-G-NH₂ pentamer of Formula (XI-a)) may display significantly reduced weight gain from moisture absorption compared to the amorphous compounds.

In some embodiments, polypeptide compounds as described herein may be used for synthesizing the active polypeptide OXM (SEQ ID NO: 16, 34 amino acids). In some embodiments, some of the compounds disclosed herein are conjugated to other polypeptide fragments to form the full-length OXM. In some embodiments, the present crystalline compounds (such as H(dnp)-Aib-Q-G tetramer of Formula (VII-a), H(trt)-Aib-Q-G tetramer of Formula (VIII-a), D-Y-S-K tetramer of Formula (IX-a), Y-S-K-Y tetramer of Formula (X-a) and Fmoc-G-P-S-S-G-NH₂ pentamer of Formula (XI-a)) may be employed as pure, stable, easy to use intermediates for OXM synthesis at high yield. In some embodiments, crystallization of the present compounds may help to reject impurities from the crude material. Compared to amorphous crude materials, the crystalline compounds may show improved purity (e.g., as measured by UPLC-MS analysis). In some embodiments, crystallization of the crude compounds reduce the necessary solvent flow, providing a “greener,” more ecologically desired process. In some embodiments, crystallization of the crude compounds spiked with 1% dimer (impurity) may yield crystalline compound with almost complete rejection of the dimer. In some embodiments, crystallization also improves physical properties, for example, the hygroscopicity. In some embodiments, the crystalline compounds (such as H(dnp)-Aib-Q-G tetramer of Formula (VII-a), H(trt)-Aib-Q-G tetramer of Formula (VIII-a), D-Y-S-K tetramer of Formula (IX-a), Y-S-K-Y tetramer of Formula (X-a) and Fmoc-G-P-S-S-G-NH₂ pentamer of Formula (XI-a)) may display significantly reduced weight gain from moisture absorption compared to the amorphous compounds.

In some embodiments, the compounds of Formula (I), (II), (III), (IV), (V), (VI), (VII), (VIII), (IX), (X), (XI), and (XII) comprise or more protecting groups. In some embodiments, the one or more protecting groups are chosen from Fmoc, Boc, tert-butyl, benzyl, carboxybenzyl, methyl, allyl, cyclohexyl, and trityl groups, or combinations thereof. In some embodiments, the one or more protecting groups is Fmoc. In some embodiments, the one or more protecting groups is Boc. In some embodiments, the one or more protecting groups is tert-butyl. In some embodiments, the one or more protecting groups is trityl. In some embodiments, the one or more protecting groups is benzyl. In some embodiments, the one or more protecting groups is carboxybenzyl. In some embodiments, the one or more protecting groups is methyl. In some embodiments, the one or more protecting groups is allyl. In some embodiments, the one or more protecting groups is cyclohexyl. In some embodiments, the compounds are protected with Fmoc, Boc, tert-butyl, benzyl, carboxybenzyl, methyl, allyl, cyclohexyl, and trityl groups. In some embodiments, the compounds are protected with Fmoc groups. In some embodiments, the compounds are protected with Fmoc and Boc groups. In some embodiments, the compounds are protected with Fmoc and tert-butyl groups. In some embodiments, the compounds are protected with Fmoc and trityl groups. In some embodiments, the compounds are protected with Fmoc and benzyl groups. In some embodiments, the compounds are protected with Boc groups. In some embodiments, the compounds are protected with Boc and tert-butyl groups. In some embodiments, the compounds are protected with Boc and trityl groups. In some embodiments, the compounds are protected with Boc and benzyl groups. In some embodiments, the compounds are protected with tert-butyl groups. In some embodiments, the compounds are protected with tert-butyl and trityl groups. In some embodiments, the compounds are protected with tert-butyl and benzyl groups. In some embodiments, the compounds are protected with trityl groups. In some embodiments, the compounds are protected with trityl and benzyl groups. In some embodiments, the compounds are protected with benzyl groups. In some embodiments, the compounds are protected with Fmoc, Boc, and tert-butyl groups. In some embodiments, the compounds are protected with Fmoc, Boc, and trityl groups. In some embodiments, the compounds are protected with Fmoc, Boc, and benzyl groups. In some embodiments, the compounds are protected with Fmoc, tert-butyl, and trityl groups. In some embodiments, the compounds are protected with Fmoc, tert-butyl, and benzyl groups. In some embodiments, the compounds are protected with Fmoc, trityl and benzyl groups. In some embodiments, the compounds are protected with Boc, tert-butyl, and trityl groups. In some embodiments, the compounds are protected with Boc, tert-butyl, and benzyl groups. In some embodiments, the compounds are protected with Fmoc, tert-butyl, and trityl groups.

As used herein, the following abbreviations have the meanings as set forth herein: “API” means active pharmaceutical ingredient, “DCM” means dichloromethane, “DIC” means diisopropylcarbodiimide, “Oxyma” means ethyl cyanohydroxyiminoacetate, “DTT” means dithiothreitol, “Fmoc” means fluorenylmethyloxycarbonyl chloride, “GGG” means retatrutide, “IPA” means isopropanol, “MTBE” means methyl-tert-butyl ether, “OXM” means mazdutide, “Pip” means piperidine, “PyBOP” means (benzotriazol-1-yloxy)tripyrrolidinophosphonium hexafluorophosphate), “SPPS” means solid phase peptide synthesis, “TFA” means trifluoroacetic acid, “TNTU” means 2-(5-norbornene-2,3-dicarboximido)-1,1,3,3-tetramethyluronium tetrafluoroborates, “TZP” means tirzepatide, and “UPLC” means ultra high performance liquid chromatography.

In some embodiments, amino acid one letter abbreviations are presented in bold print, while atoms are presented as unbolded text, to distinguish from one letter amino acid abbreviations. In some embodiments, amino acids are represented by their three-letter abbreviations. As used herein, when an amino acid abbreviation appears with a number above the amino acid, the number refers to the corresponding amino acid position in the final product. The numbers are provided for convenience and the appearance or absence of such numbers in a sequence does not influence the amino acid sequence or the peptide indicated in such sequence.

As used herein, the term “protected” means that a protecting group is attached at the indicated position.

The term “crystalline” as used herein includes all crystalline forms that have a discernable X-ray pattern.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

A “peptide” or “polypeptide” is a linked sequence of two or more amino acids linked by peptide bonds. The polypeptide can be natural, synthetic, or a modification or combination of natural and synthetic.

The term “protecting group” or “amino acid protecting group” as used herein refers to a group that protects an acid or amine moiety of the amino acid or a reactive moiety on the side chain of an amino acid. The “acid moiety” includes, for example, carboxylic acid group (—COOH). The “amine moiety” includes, for example, a primary amine group (—NH₂), a secondary amine group (—NH—), amide group (—C(O)—NH₂), and a guanidinium group ([—NHC(NH₂)—NH₂]⁺). The acid moiety or amine moiety may be a moiety of a terminal amino acid in a peptide or polypeptide or a moiety on a side chain of a non-terminal amino acid in a peptide or polypeptide. Other reactive moieties on the side chain of an amino acid include, for example, hydroxy (—OH) and thiol (—SH) groups.

A protecting group may be a removable group that is known in the art to (i) protect a reactive group (such an amine group or a carboxylic acid group) against undesirable reaction during synthetic procedures, e.g., to block or protect the functionality of the reactive group while the reactions involving other functional sites of the compound are carried out, and (ii) be selectively deprotected in a multiply-protected structure without affecting other protecting groups. Suitable protecting groups and the methods of introducing and removing such groups include those known in the art, such as those described in T. W. Green and P. G. M. Wuts, Greene's Protective Groups in Organic Synthesis, John Wiley and Sons, 2007 and Isidro-Llobet et al., Amino Acid-Protecting Groups, Chem. Rev, 2009, 109(6), 2455-2504, which are incorporated herein in their entirety.

Suitable protecting groups for aspartic acid (Asp) include, but are not limited to, tert-butyl (t-Bu), 3-methyl-3-pentyl (mpe), allyl, and 4-{N-[1-(4,4-dimethyl-2,6-dioxocyclohexylidene)-3-methylbutyl]amino}benzyl (DMAB). In some embodiments, the protecting group for aspartic acid (Asp) is t-Bu or mpe.

Suitable protecting groups for serine (Ser), threonine (Thr), or tyrosine (Tyr) include, but are not limited to, t-Bu and triphenylmethyl (trityl or trt). In some embodiments, the protecting group for serine (Ser), threonine (Thr), or tyrosine (Tyr) is t-Bu or TRT.

Suitable protecting groups for glutamic acid (Glu) include, but are not limited to, t-Bu, trt, allyl, and DMAB. In some embodiments, the protecting group for glutamic acid (Glu) is t-Bu or trt.

Suitable protecting groups for glutamine (Gln) include, but are not limited to, trt, 4-methoxytrityl (4-methyltrityl, or MTT), acetamidomethyl (ACM), and trimethoxybenzyl (TMOB). In some embodiments, the protecting group for glutamine (Gln) is TRT.

Suitable protecting groups for lysine (Lys) include, but are not limited to, t-butoxycarbonyl (Boc), allyloxycarbony (Alloc), 4-phenylacetoxybenzyloxycarbonyl (PhAc), MTT, 1-(4,4-dimethyl-2,6-dioxocyclohex-1-ylidene)ethyl (ivDde), and 2-(4,4-dimethyl-2,6-dioxocyclohexylidene)ethyl (Dde). In some embodiments, the protecting group for lysine (Lys) is Boc, MTT, or Alloc.

Suitable protecting groups for tryptophan (Trp) include, but are not limited to, Boc and formyl. In some embodiments, the protecting group for tryptophan (Trp) is Boc.

Suitable protecting groups for histidine (His) include, but are not limited to, Boc, trt, and 2,4-dinitrophenyl (dnp). In some embodiments, the protecting group for histidine (His) is Boc, trt, or dnp.

Exemplary acid protecting groups include esters such as substituted and unsubstituted C₁-C₈ lower alkyl (e.g., methyl, ethyl, t-butyl), methoxymethyl, methylthiomethyl, 2,2,2-trichloroethyl, tetrahydropyranyl, substituted and unsubstituted phenylalkyl (e.g., benzyl) and substituted derivatives thereof (e.g., alkoxybenzyl, nitrobenzyl), cinnamyl, dialkylaminoalkyl (e.g., dimethylaminoethyl), trimethylsilyl, substituted and unsubstituted amides and hydrazides (e.g., amides and hydrazides of N,N-dimethylamine), 7-nitroindole, hydrazine, N-phenylhydrazine, acyloxyalkyl (e.g., pivaloyloxymethyl, propionyloxymethyl), aroyloxyalkyl (e.g., benzoyloxyethyl), alkoxycarbonylalkyl (e.g., methoxycarbonylmethyl), cyclohexyloxycarbonylmethyl, alkoxycarbonyloxyalkyl (e.g., t-butyloxycarbonyloxymethyl), alkoxycarbonylaminoalkyl (e.g., t-butyloxycarbonylaminomethyl), alkylaminocarbonylaminoalkyl (e.g., methylaminocarbonylaminomethyl), acylaminoalkyl (e.g., acetylaminomethyl), heterocyclylcarbonyloxyalkyl (e.g., 4-methylpiperazinyl-carbonyloxymethyl), dialkylaminocarbonylalkyl (e.g., dimethylaminocarbonyl-methyl), (5-(lower alkyl)-2-oxo-1,3-dioxolen-4-yl)alkyl (e.g., (5-t-butyl-2-oxo-1,3-dioxolen-4-yl)methyl), and (5-phenyl-2-oxo-1,3-dioxolen-4-yl)alkyl (e.g., (5-phenyl-2-oxo-1,3-dioxolen-4-yl)methyl).

Exemplary amine and/or amide protecting groups include, but are not limited to, acyl (e.g., formyl, acetyl, chloroacetyl, trichloroacetyl, o-nitrophenylacetyl, o-nitrophenoxy-acetyl, trifluoroacetyl, acetoacetyl, 4-chlorobutyryl, isobutyryl, o-nitrocinnamoyl, picolinoyl, acylisothiocyanate, aminocaproyl, benzoyl), acyloxy (e.g., methoxy-carbonyl, 9-fluorenylmethoxycarbonyl, 2,2,2-trifluoroethoxycarbonyl, 2-trimethylsilylethoxy-carbonyl, vinyloxycarbonyl, allyloxycarbonyl, t-butyloxycarbonyl (Boc), 1,1-dimethyl-propynyloxycarbonyl, benzyloxycarbonyl (Cbz), p-nitrobenzyloxycarbonyl, 2,4-dichloro-benzyloxycarbonyl), 9-xanthenyl, and trityl. Additional exemplary amide protecting groups include, but are not limited to, o-nitrocinnamoyl, picolinoyl, aminocaproyl, benzoyl, acyloxy (e.g., methoxy-carbonyl, 9-fluorenylmethoxycarbonyl, 2,2,2-trifluoroethoxycarbonyl, 2-trimethylsilylethoxy-carbonyl, vinyloxycarbonyl, allyloxycarbonyl, t-butyloxycarbonyl (Boc), 1,1-dimethyl-propynyloxycarbonyl, benzyloxycarbonyl (Cbz), p-nitrobenzyloxycarbonyl, and 2,4-dichloro-benzyloxycarbonyl). Exemplary indole protecting groups include, but are not limited to, formyl (For) and t-butyloxycarbonyl (Boc). Exemplary imidazole protecting groups include, but are not limited to, tosyl (Tos), benzyloxymethyl (Bom), trityl (Trt), and t-butyloxycarbonyl (Boc). Exemplary guanidinium protecting groups include, but are not limited to, 2,2,4,6,7-pentamethyl-2,3-dihydrobenzofuran-5-sulfonyl (Pbf) and t-butyloxycarbonyl (Boc).

Exemplary hydroxyl protecting groups include, but are not limited to, unsubstituted or substituted alkyls (e.g., t-butyl, allyl, benzyl, methoxymethyl, tetrahydropyranyl, o-nitrobenzyl), silyl (e.g. t-butyldimethylsilyl (TBDMS), t-butyldiphenylsilyl (TBDPS)), acyl (e.g. acetyl, benzoyl, pivaloyl). Exemplary thiol protecting groups include, but are not limited to, p-methylbenzyl (Meb), acetamidomethyl (Acm), and trityl (Trt).

### ENUMERATED EMBODIMENTS

Embodiment 1. A compound of a Formula (I), or a salt, solvate, or hydrate thereof,

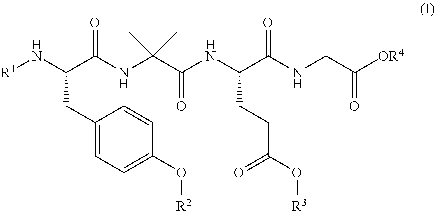

wherein R¹ is H or a protecting group; R² is H or a protecting group; R³ is H or a protecting group; and R⁴ is H or a protecting group; and wherein at least one of R¹, R², R³, and R⁴ is a protecting group.

Embodiment 2. The compound of Embodiment 1, wherein R¹ is H, Fmoc, or Boc.

Embodiment 3. The compound of any one of Embodiments 1-2, wherein R² is H or t-butyl.

Embodiment 4. The compound of any one of Embodiments 1-3, wherein R⁴ is H or benzyl.

Embodiment 5. The compound of any one of Embodiments 1-4, wherein R³ is H, t-butyl, benzyl, carboxybenzyl, methyl, allyl, cyclohexyl, or trityl.

Embodiment 6. The compound of any one of Embodiments 1-5, wherein at least two of R¹, R², R³, and R⁴ are H.

Embodiment 7. The compound of any one of Embodiments 1-6, wherein at least two of R¹, R², R³, and R⁴ are protecting groups.

Embodiment 8. The compound of Embodiment 1, wherein the compound is a formula:

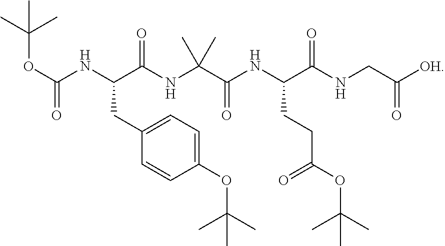 [SMILES: CC(C)(C)OC(=O)CC[C@H](NC(=O)C(C)(C)NC(=O)[C@H](Cc1ccc(OC(C)(C)C)cc1)NC(=O)OC(C)(C)C)C(=O)NCC(=O)O]

Embodiment 9. The compound of Embodiment 1, wherein the compound is a formula:

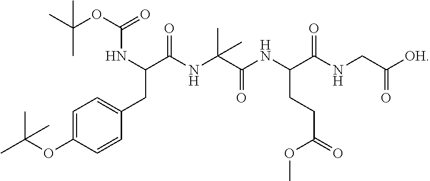 [SMILES: COC(=O)CCC(NC(=O)C(C)(C)NC(=O)C(Cc1ccc(OC(C)(C)C)cc1)NC(=O)OC(C)(C)C)C(=O)NCC(=O)O]

Embodiment 10. The compound of Embodiment 1, wherein the compound is a formula:

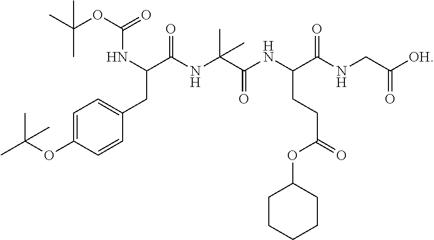 [SMILES: CC(C)(C)OC(=O)NC(Cc1ccc(OC(C)(C)C)cc1)C(=O)NC(C)(C)C(=O)NC(CCC(=O)OC1CCCCC1)C(=O)NCC(=O)O]

Embodiment 11. The compound of Embodiment 1, wherein the compound is a formula:

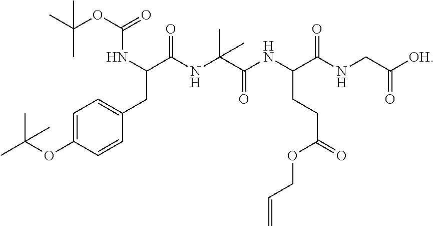 [SMILES: C=CCOC(=O)CCC(NC(=O)C(C)(C)NC(=O)C(Cc1ccc(OC(C)(C)C)cc1)NC(=O)OC(C)(C)C)C(=O)NCC(=O)O]

Embodiment 12. The compound of Embodiment 1, wherein the compound is a formula:

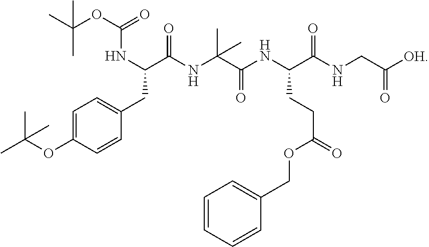 [SMILES: CC(C)(C)OC(=O)N[C@@H](Cc1ccc(OC(C)(C)C)cc1)C(=O)NC(C)(C)C(=O)N[C@@H](CCC(=O)OCc1ccccc1)C(=O)NCC(=O)O]

Embodiment 13. The compound of any one of Embodiments 1-12, wherein the compound is a solvate.

Embodiment 14. The compound of Embodiment 13, wherein the solvate is produced from methyl t-butyl ether (MTBE) or a mixture comprising MTBE.

Embodiment 15. The compound of any one of Embodiments 1-12, wherein the compound is crystalline.

Embodiment 16. The compound Embodiment 15, wherein the compound is in the form of a crystalline solid characterized by a peak in the X-ray powder diffraction pattern at diffraction angle 2θ±0.2 degrees of 5.2° in combination of one or more peaks select from 8.4°, 8.8°, 10.4°, 15.5°, 17.1°, and 17.7°.

Embodiment 17. The compound Embodiment 15, wherein the compound is in the form of a crystalline solid characterized by a peak in the X-ray powder diffraction pattern at diffraction angle 2θ±0.2 degrees of 6.1° in combination of one or more peaks select from 10.3°, 14.9°, 16.8°, 18.1°, and 18.2°.

Embodiment 18. The compound Embodiment 15, wherein the compound is in the form of a crystalline solid characterized by a peak in the X-ray powder diffraction pattern at diffraction angle 2θ±0.2 degrees of 6.2° in combination of one or more peaks select from 8.9°, 12.3°, 14.9°, 15.4°, and 21.8°.

Embodiment 19. A method of synthesizing the compound of Embodiment 1, the method comprising:
reacting a compound of formula (Yₚᵣₒₜ)

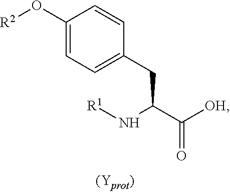

wherein R¹ and R² are protecting groups, and a compound of formula (Aibₚᵣₒₜ)

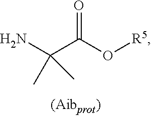

wherein R⁵ is a protecting group, forming a compound of formula (Yₚᵣₒₜ-Aibₚᵣₒₜ),

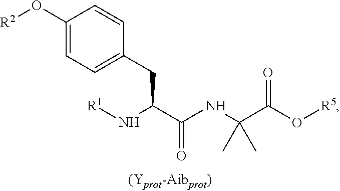

removing the protecting group of R⁵, and forming a compound of formula (Yₚᵣₒₜ-Aib)

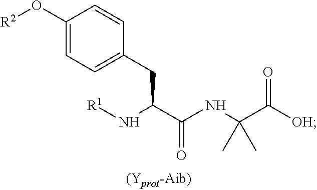

reacting the compound of formula (Yₚᵣₒₜ-Aib) and a compound of formula (Eₚᵣₒₜ)

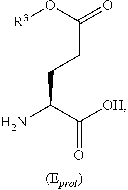

wherein R³ is a protecting group, forming a compound of a formula (Yₚᵣₒₜ-Aib-Eₚᵣₒₜ)

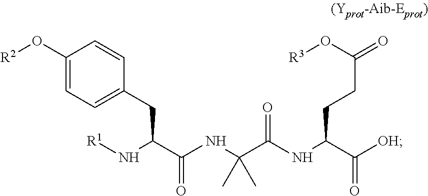

reacting the compound of formula (Yₚᵣₒₜ-Aib-Eₚᵣₒₜ) and a compound of formula (Gₚᵣₒₜ)

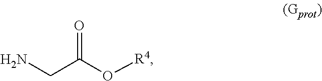

wherein R⁴ is a protecting group, forming a compound of formula (Yₚᵣₒₜ-Aib-Eₚᵣₒₜ-Gₚᵣₒₜ)

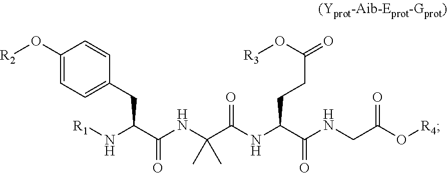

and optionally
removing one or more of protecting groups R¹, R², R³, and R⁴.

Embodiment 20. A method of synthesizing a polypeptide of SEQ ID NO: 1, the method comprising conjugating the compound of any one of Embodiments 1-12 via the C-terminus of the compound to the N-terminus of a polypeptide of SEQ ID NO: 2,

Embodiment 21. A method of synthesizing a polypeptide of SEQ ID NO: 26, the method comprising conjugating the compound of any one of Embodiments 1-12 via the C-terminus of the compound to the N-terminus of a polypeptide of SEQ ID NO: 27.

Embodiment 22. A compound of a Formula (II), or a salt, solvate, or hydrate thereof,

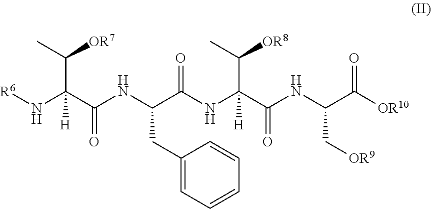

wherein R⁶ is H or a protecting group; R⁷ is H or a protecting group; R⁸ is H or a protecting group; R⁹ is H or a protecting group; and R¹⁰ is H or a protecting group.

Embodiment 23. The compound Embodiment 22, wherein each protecting group is independently chosen from Boc, Fmoc, tert-butyl, and trityl groups.

Embodiment 24. The compound of any one of Embodiments 22-23, wherein R⁶ is H, Fmoc, or Boc.

Embodiment 25. The compound of any one of Embodiments 22-24, wherein R⁷ is H or t-butyl.

Embodiment 26. The compound of any one of Embodiments 22-25, wherein R⁸ is H or t-butyl.

Embodiment 27. The compound of any one of Embodiments 22-26, wherein R⁹ is H or t-butyl.

Embodiment 28. The compound of any one of Embodiments 22-27, wherein R¹⁰ is H or benzyl.

Embodiment 29. The compound of any one of Embodiments 22-28, wherein at least one of R⁶, R⁷, R⁸, R⁹, and R¹⁰ is a protecting group.

Embodiment 30. The compound of any one of Embodiments 22-29, wherein R⁶, R⁷, R⁸, R⁹, and R¹⁰ are H.

Embodiment 31. The compound of Embodiment 22, wherein the compound is of a formula:

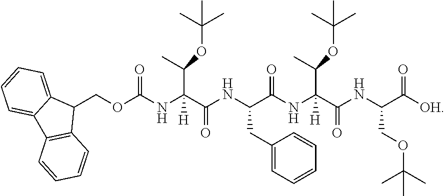 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)O)[C@@H](C)OC(C)(C)C]

Embodiment 32. The compound of any one of Embodiments 22-31, wherein the compound is a solvate.

Embodiment 33. The compound of Embodiment 32, wherein the solvate is produced from a solvent comprising heptane.

Embodiment 34. The compound of any one of Embodiments 22-31, wherein the compound is crystalline.

Embodiment 35. The compound of Embodiment 34, wherein the compound is in the form of a crystalline solid characterized by a peak in the X-ray powder diffraction pattern at diffraction angle 2θ±0.2 degrees of 7.7-7.9° in combination of one or more peaks select from 5.8°, 10.0°, 10.8-10.9°, 11.3-11.4°, 12.0-12.1°, 12.8°, 14.2-14.4°, and 16.8-17.0°,

Embodiment 36. The compound of Embodiment 34, wherein the compound is in the form of a crystalline solid characterized by a peak in the X-ray powder diffraction pattern at diffraction angle 2θ±0.2 degrees of 7.3° in combination of one or more peaks select from 5.1°, 5.7° 7.6°, 9.5°, and 12.4°.

Embodiment 37. The compound of Embodiment 34, wherein the compound is in the form of a crystalline solid characterized by a peak in the X-ray powder diffraction pattern at diffraction angle 2θ±0.2 degrees of 7.8° in combination of one or more peaks select from 8.5°, 11.5°, 12.0°, 12.8°, 14.3°, 15.5°, 20.2° and 23.3°.

Embodiment 38. The compound of Embodiment 34, wherein the compound is in the form of a crystalline solid characterized by a peak in the X-ray powder diffraction pattern at diffraction angle 2θ±0.2 degrees of 8.2-8.3° in combination of one or more peaks select from 5.9°, 7.7°, 9.2°, 10.2°, 11.3°, 13.8-13.9°, 15.5-15.7°, 17.1°, and 18.5°.

Embodiment 39. A method of synthesizing a polypeptide of SEQ ID NO: 1, the method comprising (i) conjugating the compound of any one of Embodiments 22-31 via the N-terminus of the compound to the C-terminus of a polypeptide of SEQ ID NO: 3 to form a polypeptide of SEQ ID NO: 4; and (ii) conjugating the polypeptide of SEQ ID NO: 4 via its C-terminus to the N-terminus of a polypeptide of SEQ ID NO: 5.

Embodiment 40. A compound of Formula (III), or a salt, solvate, or hydrate thereof,

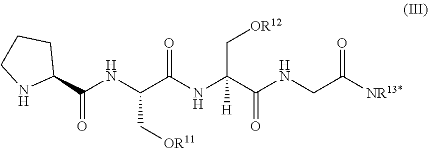

wherein R¹¹ is H or a protecting group; and R¹² is H or a protecting group.

Embodiment 41. The compound of Embodiment 40, wherein each protecting group is independently chosen from Boc, Fmoc, tert-butyl, and trityl groups.

Embodiment 42. The compound of any one of Embodiments 40-41, wherein R¹¹ is H or t-butyl.

Embodiment 43. The compound of any one of Embodiments 40-42, wherein R¹² is H or t-butyl.

Embodiment 44. The compound of any one of Embodiments 40-43, wherein at least one of R¹¹ and R¹² is a protecting group.

Embodiment 45. The compound of any one of Embodiments 40-43, wherein R¹¹ and R¹² are H.

Embodiment 46. The compound of Embodiment 40, wherein the compound is a compound of a formula:

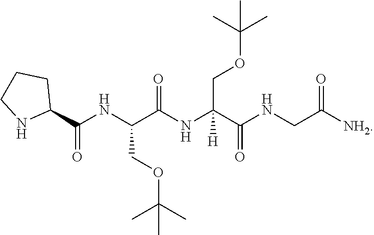 [SMILES: CC(C)(C)OC[C@H](NC(=O)[C@H](COC(C)(C)C)NC(=O)[C@@H]1CCCN1)C(=O)NCC(N)=O]

Embodiment 47. The compound of any one of Embodiments 40-46, wherein the compound is a solvate.

Embodiment 48. The compound of any one of Embodiments 40-46, wherein the compound is crystalline.

Embodiment 49. The compound of Embodiment 48, wherein the compound is in the form of a crystalline solid characterized by a peak in the X-ray powder diffraction pattern at diffraction angle 2θ±0.2 degrees of 8.0° in combination of one or more peaks select from 7.0°, 10.30, 14.1°, 15.2°, 16.7°, 18.0°, 19.0°, 19.7°, 20.8°, and 21.9°.

Embodiment 50. The compound of Embodiment 48, wherein the compound is in the form of a crystalline solid characterized by a peak in the X-ray powder diffraction pattern at diffraction angle 2θ±0.2 degrees of 9.0° in combination of one or more peaks select from 5.7°, 9.9°, 16.2°, 17.1°, 17.9°, 18.1°, 18.4°, 18.8°, 19.9°, 20.1°, and 22.5°.

Embodiment 51. The compound of Embodiment 48, wherein the compound is in the form of a crystalline solid characterized by a peak in the X-ray powder diffraction pattern at diffraction angle 2θ±0.2 degrees of 10.5° in combination of one or more peaks select from 5.6°, 11.9°, 13.30, 15.4°, 15.6°, 18.1°, 19.9°, and 21.1°.

Embodiment 52. The compound of Embodiment 48, wherein the compound is in the form of a crystalline solid characterized by a peak in the X-ray powder diffraction pattern at diffraction angle 2θ±0.2 degrees of 10.7° in combination of one or more peaks select from 4.9°, 14.8°, 20.3°, and 21.5°.

Embodiment 53. The compound of Embodiment 48, wherein the compound is in the form of a crystalline solid characterized by a peak in the X-ray powder diffraction pattern at diffraction angle 2θ±0.2 degrees of 10.5° in combination of one or more peaks select from 5.9°, 10.5°, 10.9°, 12.1°, 13.1°, 15.9°, 17.5°, 20.9°, 21.1°, and 21.9°.

Embodiment 54. The compound of Embodiment 48, wherein the compound is in the form of a crystalline solid characterized by a peak in the X-ray powder diffraction pattern at diffraction angle 2θ±0.2 degrees of 7.8° in combination of one or more peaks select from 11.3°, 11.5°, 15.4°, 15.6°, and 21.5°.

Embodiment 55. The compound of Embodiment 48, wherein the compound is in the form of a crystalline solid characterized by a peak in the X-ray powder diffraction pattern at diffraction angle 2θ±0.2 degrees of 10.0° in combination of one or more peaks select from 8.1°, 12.5°, 13.5°, 14.7°, 17.8°, 18.8°, 20.0°, and 22.4°.

Embodiment 56. The compound of Embodiment 48, wherein the compound is in the form of a crystalline solid characterized by a peak in the X-ray powder diffraction pattern at diffraction angle 2θ±0.2 degrees of 21.1° in combination of one or more peaks select from 5.6°, 10.5°, 10.8°, 11.9°, 15.4°, and 23.8°.

Embodiment 57. A method of synthesizing a polypeptide of SEQ ID NO: 1, the method comprising (i) conjugating the compound of any one of Embodiments 40-46 via the N-terminus of the compound to the C-terminus of a polypeptide of SEQ ID NO: 6 to form a polypeptide of SEQ ID NO: 7; and (ii) conjugating the polypeptide of SEQ ID NO: 7 via its C-terminus to the N-terminus of a polypeptide of SEQ ID NO: 8.

Embodiment 58. A compound of a Formula (IV), or a salt, solvate, or hydrate thereof,

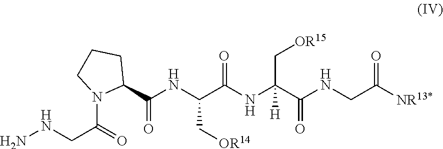

wherein R¹³ is H or a protecting group; R^{13*} is H or a protecting group; R¹⁴ is H or a protecting group; and R¹⁵ is H or a protecting group

Embodiment 59. The compound of Embodiment 58, wherein each protecting group is independently chosen from Boc, Fmoc, tert-butyl, and trityl groups.

Embodiment 60. The compound of any one of Embodiments 58-59, wherein R¹³ is H, Fmoc, or Boc.

Embodiment 61. The compound of any one of Embodiments 58-60, wherein R^{13*} is H.

Embodiment 62. The compound of any one of Embodiments 58-61, wherein R¹⁴ is H or t-butyl.

Embodiment 63. The compound of any one of Embodiments 58-62, wherein R¹⁵ is H or t-butyl.

Embodiment 64. The compound of any one of Embodiments 58-63, wherein at least one of R¹³, R^{13*}, R¹⁴ and R¹⁵ is a protecting group

Embodiment 65. The compound of any one of Embodiments 58-63, wherein R¹³, R^{13*}, R¹⁴, and R¹⁵ are H.

Embodiment 66. The compound of Embodiment 58, wherein the compound is of a formula:

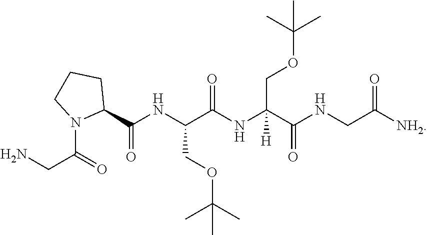 [SMILES: CC(C)(C)OC[C@H](NC(=O)[C@H](COC(C)(C)C)NC(=O)[C@@H]1CCCN1C(=O)CN)C(=O)NCC(N)=O]

Embodiment 67. The compound of any one of Embodiments 58-66, wherein the compound is a solvate.

Embodiment 68. The compound of any one of Embodiments 58-66, wherein the compound is crystalline.

Embodiment 69. The compound of Embodiment 68, wherein the compound is in the form of a crystalline solid characterized by a peak in the X-ray powder diffraction pattern at diffraction angle 2θ±0.2 degrees of 5.1° in combination of one or more peaks select from 4.3°, 6.1°, 8.0°, 10.1°, and 18.7°.

Embodiment 70. The compound of Embodiment 68, wherein the compound is in the form of a crystalline solid characterized by a peak in the X-ray powder diffraction pattern at diffraction angle 2θ±0.2 degrees of 5.2° in combination of one or more peaks select from 6.0°, 6.7°, 10.0°, 10.3°, 16.4°, 17.8°, 18.3°, 19.4°, and 22.4°.

Embodiment 71. A method of synthesizing a polypeptide of SEQ ID NO: 1, the method comprising (i) conjugating the compound of any one of claims 58-66 via the N-terminus of the compound to the C-terminus of a polypeptide of SEQ ID NO: 9 to form a polypeptide of SEQ ID NO: 7; and (ii) conjugating the polypeptide of SEQ ID NO: 7 via its C-terminus to the N-terminus of a polypeptide of SEQ ID NO: 8.

Embodiment 72. The compound of Embodiment 58, wherein the compound is of a formula,

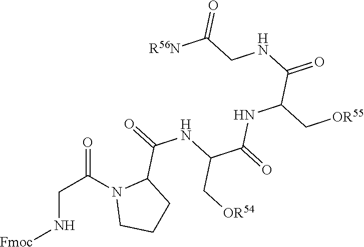

wherein R⁵⁴ is H or a protecting group; R⁵⁵ is H or a protecting group; and R⁵⁶ is H or a protecting group.

Embodiment 73. The compound of Embodiment 72, wherein each protecting group is independently chosen from Boc, Fmoc, tert-butyl, and trityl groups.

Embodiment 74. The compound of any one of Embodiments 72-73, wherein R⁵⁴ is H or tert-butyl.

Embodiment 75. The compound of any one of Embodiments 72-74, wherein R⁵⁵ is H or tert-butyl.

Embodiment 76. The compound of any one of Embodiments 72-75, wherein R⁵⁶ is H or Boc.

Embodiment 77. The compound of any one of Embodiments 72-76, wherein at least one of R⁵⁴, R⁵⁵, and R⁵⁶ is H.

Embodiment 78. The compound of any one of Embodiments 72-77, wherein at least one of R⁵⁴, R⁵⁵, and R⁵⁶ is a protecting group.

Embodiment 79. The compound of Embodiment 72, wherein the compound is of a formula:

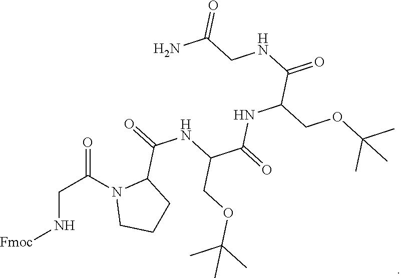

Embodiment 80. The compound of any one of Embodiments 72-79, wherein the compound is a solvate.

Embodiment 81. The compound of Embodiment 80, wherein the compound is a solvate produced from acetone.

Embodiment 82. The compound of any one of Embodiments 72-79, wherein the compound is a de-solvate.

Embodiment 83. The compound of any one of Embodiments 72-79, wherein the compound is crystalline.

Embodiment 84. The compound of Embodiment 83, wherein the compound is the compound is in the form of a crystalline solid characterized by peaks in the X-ray powder diffraction pattern at diffraction angle 2-theta±0.2 degrees of 5.8° and 18.5° in combination of one or more peaks chosen from 8.6°, 9.4°, 12.9°, 13.8°, 17.2°, and 19.4°.

Embodiment 85. The compound of Embodiment 83, wherein the compound is in the form of a crystalline solid characterized by peaks in the X-ray powder diffraction pattern at diffraction angle 2-theta±0.2 degrees of 7.0-7.1° and 7.5-7.7° in combination of one or more peaks chosen from 5.3-5.4°, 9.7-9.9°, and 14.7-14.9.

Embodiment 86. The compound of Embodiment 83, wherein the compound is in the form of a crystalline solid characterized by a peak in the X-ray powder diffraction pattern at diffraction angle 2-theta±0.2 degrees of 8.3° in combination of one or more peaks chosen from 6.3°, 11.4°, 14.3°, and 16.6°.

Embodiment 87. The compound of Embodiment 83, wherein the compound is in the form of a crystalline solid characterized by a peak in the X-ray powder diffraction pattern at diffraction angle 2-theta±0.2 degrees of 7.2° in combination of one or more peaks chosen from 6.8°, 8.6°. 15.8°, and 18.9°.

Embodiment 88. The compound of Embodiment 83, wherein the compound is in the form of a crystalline solid characterized by a peak in the X-ray powder diffraction pattern at diffraction angle 2-theta±0.2 degrees of 6.1° in combination of one or more peaks chosen from 6.3°, 7.8°, 10.0°, and 12.4°.

Embodiment 89. A method of synthesizing the compound of Embodiment 72, the method comprising:
reacting a compound of formula (Fmoc-G) and a compound of formula (Pₚᵣₒₜ) to form a compound of formula (Fmoc-G-Pₚᵣₒₜ)

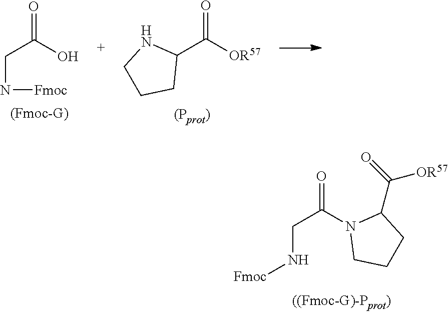

wherein R⁵⁷ is a protecting group;
removing the protecting group R⁵⁷ to form a compound of (Fmoc-G-P)

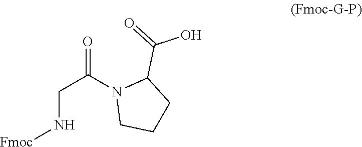

reacting the compound of formula (Fmoc-G-P) and a compound of formula (Sₚᵣₒₜ₂) to form a compound of a formula (Fmoc-G-P-Sₚᵣₒₜ₂)

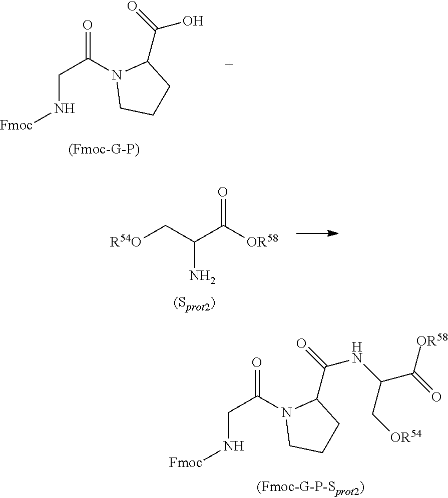

wherein R⁵⁴ and R⁵⁸ are protecting groups;
removing the protecting group R⁵⁸ to form a compound of (Fmoc-G-P-Sₚᵣₒₜ₁)

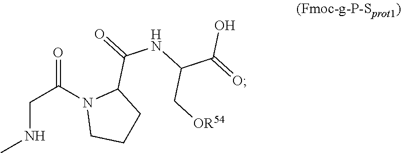

reacting the compound of formula (Fmoc-G-P-Sₚᵣₒₜ₁) and a compound of formula (Sₚᵣₒₜ₂) to form a compound of formula (Fmoc-G-P-Sₚᵣₒₜ₁-Sₚᵣₒₜ₂)

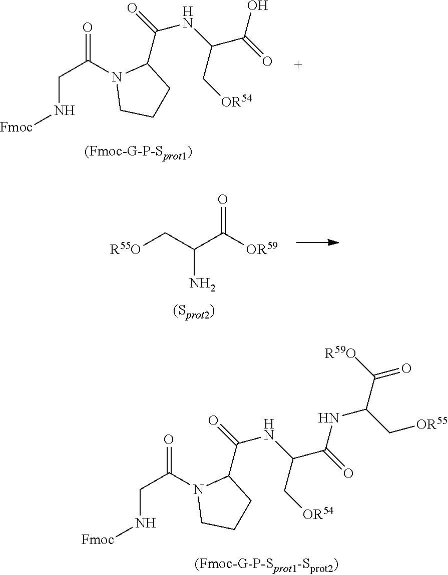

wherein R⁵⁵ and R⁵⁹ are protecting groups; and
removing the protecting group R⁵⁹ to form a compound of (Fmoc-F-Gₚᵣₒₜ₁-Sₚᵣₒₜ₁-Sₚᵣₒₜ₁)

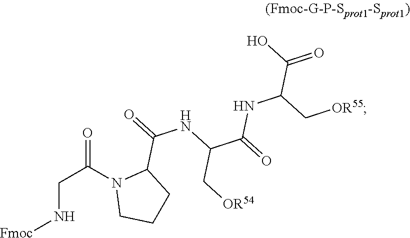

reacting the compound of formula (Fmoc-G-P-Sₚᵣₒₜ₁-Sₚᵣₒₜ₁) and a compound of formula (Gₚᵣₒₜ) to form a compound of formula (Fmoc-G-P-Sₚᵣₒₜ₁-Sₚᵣₒₜ₁-Gₚᵣₒₜ)

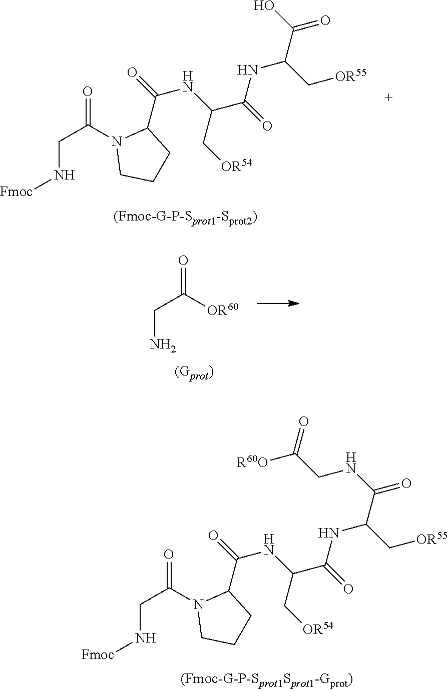

wherein R⁶⁰ is a protecting group;
converting the compound of formula (Fmoc-G-P-Sₚᵣₒₜ₁-Sₚᵣₒₜ₁-Gₚᵣₒₜ) to the compound of claim 36

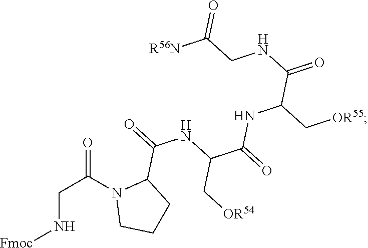

and
optionally removing one or more protecting groups R⁵⁴, R⁵⁵, and R⁵⁶.

Embodiment 90. The method of Embodiment 89, wherein each protecting group is independently chosen from Boc, Fmoc, tert-butyl and trityl groups.

Embodiment 91. A method of synthesizing the polypeptide of SEQ ID NO: 12, the method comprising: (i) conjugating the compound of any one of Embodiments 72-79 via the C-terminus of the compound to the N-terminus of a polypeptide of SEQ ID NO: 22, and (ii) conjugating the resulting compound via its N-terminus to the C-terminus of a polypeptide of SEQ ID NO: 23.

Embodiment 92. A method of synthesizing the polypeptide of SEQ ID NO: 24, the method comprising conjugating the compound of any one of Embodiments 72-79 via the C-terminus of the compound to the N-terminus of a polypeptide of SEQ ID NO: 22.

Embodiment 93. A method of synthesizing the polypeptide of SEQ ID NO: 16, the method comprising conjugating the compound of any one of Embodiments 72-79 via the N-terminus of the compound to the C-terminus of a polypeptide of SEQ ID NO: 25.

Embodiment 94. A method of synthesizing the polypeptide of SEQ ID NO: 1, the method comprising: (i) conjugating the compound of any one of Embodiments 72-79 via the C-terminus of the compound to the N-terminus of a polypeptide of SEQ ID NO: 8, and (ii) conjugating the resulting compound via its N-terminus to the C-terminus of a polypeptide of SEQ ID NO: 9.

Embodiment 95. A compound of a Formula (V), or a salt, solvate, or hydrate thereof,

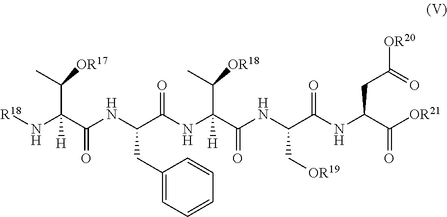

wherein R¹⁶ is H or a protecting group; R¹⁷ is H or a protecting group; R¹⁸ is H or a protecting group; R¹⁹ is H or a protecting group; R²⁰ is H or a protecting group; and R²¹ is H or a protecting group.

Embodiment 96. The compound of Embodiment 95, wherein each protecting group is independently chosen from Boc, Fmoc, tert-butyl, and trityl groups.

Embodiment 97. The compound of any one of Embodiments 95-96, wherein R¹⁶ is H, Fmoc, or Boc.

Embodiment 98. The compound of any one of Embodiments 95-97, wherein R¹⁷ is H or t-butyl.

Embodiment 99. The compound of any one of Embodiments 95-98, wherein R¹⁸ is H or t-butyl.

Embodiment 100. The compound of any one of Embodiments 95-99, wherein R¹⁹ is H or t-butyl.

Embodiment 101. The compound of any one of Embodiments 95-100, wherein R²⁰ is H or t-butyl.

Embodiment 102. The compound of any one of Embodiments 95-101, wherein R²¹ is H or benzyl.

Embodiment 103. The compound of any one of Embodiments 95-102, wherein at least one of R¹⁶, R¹⁷, R¹⁸, R¹⁹, R²⁰, and R²¹ is a protecting group.

Embodiment 104. The compound of any one of Embodiments 95-102, wherein R¹⁶, R¹⁷, R¹⁸, R¹⁹, R²⁰, and R²¹ are H.

Embodiment 105. The compound of Embodiment 95, wherein the compound is of a formula:

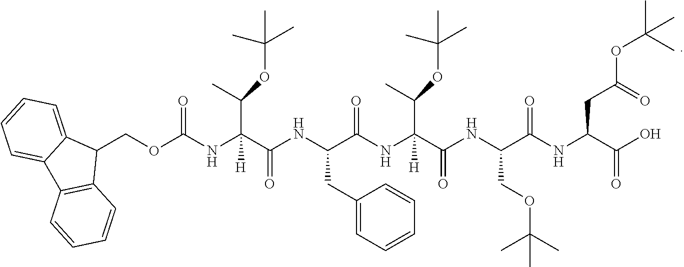 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)N[C@@H](CC(=O)OC(C)(C)C)C(=O)O)[C@@H](C)OC(C)(C)C]

Embodiment 106. The compound of any one of Embodiments 95-105, wherein the compound is a solvate.

Embodiment 107. The compound of any one of Embodiments 95-105, wherein the compound is crystalline.

Embodiment 108. The compound of Embodiment 107, wherein the compound is in the form of a crystalline solid characterized by a peak in the X-ray powder diffraction pattern at diffraction angle 2θ±0.2 degrees of 5.8-6.1° in combination of one or more peaks select from 6.7-7.1° and 8.8-9.0°.

Embodiment 109. The compound of Embodiment 107, wherein the compound is in the form of a crystalline solid characterized by a peak in the X-ray powder diffraction pattern at diffraction angle 2θ±0.2 degrees of 5.0-5.2° in combination of one or more peaks select from 5.3-5.4°, 5.7-6.0°, 6.1-6.2°, 7.6-7.9°, and 8.7-9.1°.

Embodiment 110. A method of synthesizing a polypeptide of SEQ ID NO: 1, the method comprising (i) conjugating the compound of any one of Embodiments 95-105 via the N-terminus of the compound to the C-terminus of a polypeptide of SEQ ID NO: 3 to form a polypeptide of SEQ ID NO: 10; and (ii) conjugating the polypeptide of SEQ ID NO: 10 via its C-terminus to the N-terminus of a polypeptide of SEQ ID NO: 11.

Embodiment 111. A compound of Formula (VI), or a salt, solvate, or hydrate thereof,

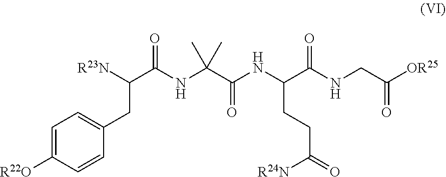

wherein R²² is H or a protecting group; R²³ is H or a protecting group; R²⁴ is H or a protecting group, and R²⁵ is H or a protecting group.

Embodiment 112. The compound of Embodiment 111, wherein each protecting group is independently chosen from Boc, Fmoc, tert-butyl, and trityl groups.

Embodiment 113. The compound of any one of Embodiments 111-112, wherein R²² is H or Boc.

Embodiment 114. The compound of any one of Embodiments 111-113, wherein R²³ is H or tert-butyl.

Embodiment 115. The compound of any one of Embodiments 111-114, wherein R²⁴ is H or trityl.

Embodiment 116. The compound of any one of Embodiments 111-115, wherein R²⁵ is H or tert-butyl.

Embodiment 117. The compound of any one of Embodiments 111-116, wherein at least one of R²², R²³, R²⁴, and R²⁵ is H.

Embodiment 118. The compound of any one of Embodiments 111-117, wherein at least one of R²², R²³, R²⁴, and R²⁵ is a protecting group.

Embodiment 119. The compound of Embodiment 111, wherein the compound is Formula (VI-a):

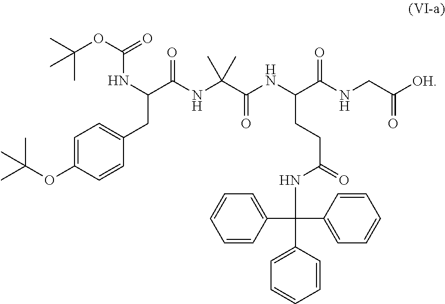 [SMILES: CC(C)(C)OC(=O)NC(Cc1ccc(OC(C)(C)C)cc1)C(=O)NC(C)(C)C(=O)NC(CCC(=O)NC(c1ccccc1)(c1ccccc1)c1ccccc1)C(=O)NCC(=O)O]

Embodiment 120. The compound of any one of Embodiments 111-119, wherein the compound is a solvate.

Embodiment 121. The compound of Embodiment 120, wherein the compound is a solvate produced from pentyl acetate, mixtures comprising pentyl acetate, ethyl acetate, or mixtures comprising 2-methyl tetrahydrofuran and t-amyl methyl ether.

Embodiment 122. The compound of Embodiment 121, wherein the mixtures comprising pentyl acetate are chosen from mixtures comprising pentyl acetate and t-butyl ethyl ether, mixtures comprising pentyl acetate and t-amyl methyl ether, or mixtures comprising pentyl acetate and heptane.

Embodiment 123. The compound of any one of Embodiments 111-119, wherein the compound is a de-solvate or a partial de-solvate.

Embodiment 124. The compound of any one of Embodiments 111-119, wherein the compound is crystalline.

Embodiment 125. The compound of Embodiment 124, wherein the compound is in the form of a crystalline solid characterized by a peak in the X-ray powder diffraction pattern at diffraction angle 2-theta±0.2 degrees of 6.3-6.4° in combination of one or more peaks chosen from 4.5°, 7.1°, 13.0-13.1°, 15.9-16.0°, and 18.4-18.6°.

Embodiment 126. The compound of Embodiment 124, wherein the compound is in the form of a crystalline solid characterized by a peak in the X-ray powder diffraction pattern at diffraction angle 2-theta±0.2 degrees of 7.0-7.2° in combination of one or more peaks chosen from 5.0-5.4°, 7.6-7.7°, 8.8-8.9°, 9.4-9.5°, and 12.5-12.7°.

Embodiment 127. A method of synthesizing the compound of Embodiment 111, the method comprising:
reacting a compound of formula (Yₚᵣₒₜ) and a compound of formula (Aibₚᵣₒₜ) to form a compound of formula (Yₚᵣₒₜ-Aibₚᵣₒₜ)

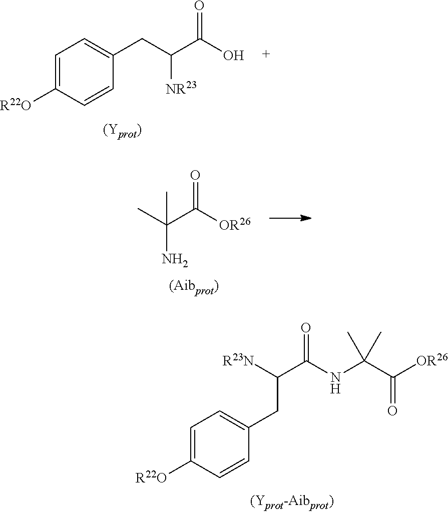

wherein R²², R²³, and R²⁶ are protecting groups;
removing the protecting group R²⁶ to form a compound of (Yₚᵣₒₜ-Aib)

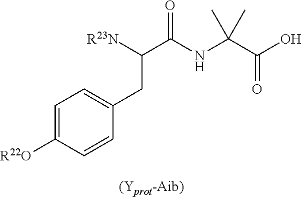

reacting the compound of formula (Yₚᵣₒₜ-Aib) and a compound of formula (Qₚᵣₒₜ₂) to form a compound of a formula (Yₚᵣₒₜ-Aib-Qₚᵣₒₜ₂)

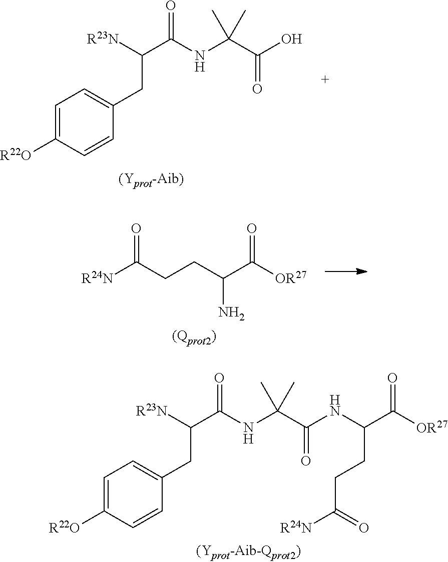

wherein R²⁴ and R²⁷ are protecting groups;
removing the protecting group R²⁷ to form a compound of (Yₚᵣₒₜ-Aib-Qₚᵣₒₜ₁)

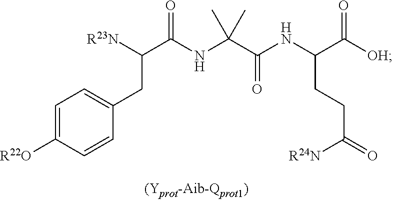

reacting the compound of formula (Yₚᵣₒₜ-Aib-Qₚᵣₒₜ₁) and a compound of formula (Gₚᵣₒₜ) to form a compound of Formula (VI)

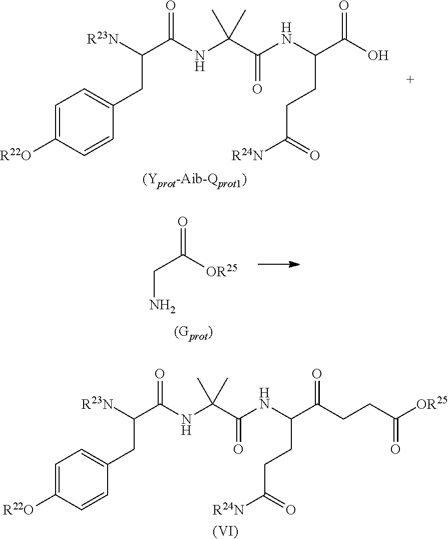

wherein R²⁵ is a protecting group; and
optionally removing one or more protecting groups R²², R²³, R²⁴, and R²⁵.

Embodiment 128. The method of Embodiment 127, wherein each protecting group is independently chosen from Boc, Fmoc, tert-butyl, and trityl groups.

Embodiment 129. A method of synthesizing a polypeptide of SEQ ID NO: 12, the method comprising conjugating a compound of any one of Embodiments 111-119 via the C-terminus of the compound to the N-terminus of a polypeptide of SEQ ID NO: 13.

Embodiment 130. A method of synthesizing a polypeptide of SEQ ID NO: 14, the method comprising conjugating a compound of any one of Embodiments 111-119 via the C-terminus of the compound to the N-terminus of a polypeptide of SEQ ID NO: 15.

Embodiment 131. A compound of Formula (VII), or a salt, solvate, or hydrate thereof,

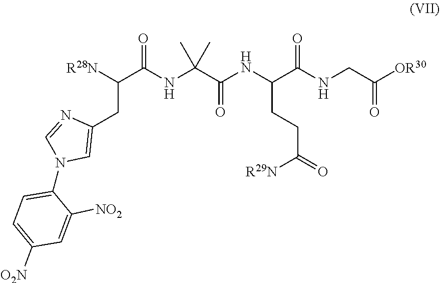

wherein R²⁸ is H or a protecting group; R²⁹ is H or a protecting group, and R³⁰ is H or a protecting group.

Embodiment 132. The compound of Embodiment 131, wherein each protecting group is independently chosen from Boc, Fmoc, tert-butyl, and trityl groups.

Embodiment 133. The compound of any one of Embodiments 131-132, wherein R²⁸ is H or Boc.

Embodiment 134. The compound of any one of Embodiments 131-133, wherein R²⁹ is H or trityl.

Embodiment 135. The compound of any one of Embodiments 131-134, wherein R³⁰ is H or tert-butyl.

Embodiment 136. The compound of any one of Embodiments 131-135, wherein at least one of R²⁸, R²⁹, and R³⁰ is H.

Embodiment 137. The compound of any one of Embodiments 131-136, wherein at least one of R²⁸, R²⁹, and R³⁰ is a protecting group.

Embodiment 138. The compound of Embodiment 131, wherein the compound is Formula (VII-a):

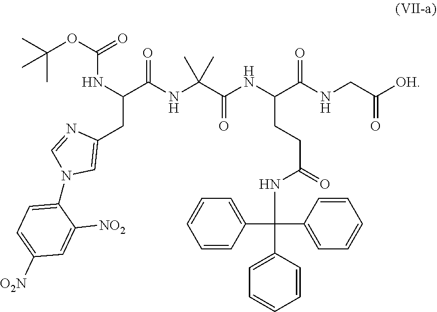 [SMILES: CC(C)(C)OC(=O)NC(Cc1cn(-c2ccc([N+](=O)[O-])cc2[N+](=O)[O-])cn1)C(=O)NC(C)(C)C(=O)NC(CCC(=O)NC(c1ccccc1)(c1ccccc1)c1ccccc1)C(=O)NCC(=O)O]

Embodiment 139. The compound of any one of Embodiments 131-137, wherein the compound is a solvate.

Embodiment 140. The compound of Embodiment 139, wherein the compound is a solvate produced from any one of a mixture of acetonitrile and methyl tert-butyl ether, a mixture of nitromethane and methyl tert-butyl ether, a mixture of tetrahydrofuran and methyl tert-butyl ether, methyl acetate, and ethyl acetate.

Embodiment 141. The compound of any one of Embodiments 131-137, wherein the compound is crystalline.

Embodiment 142. The compound of Embodiment 141, wherein the compound is in the form of a crystalline solid characterized by a peak in the X-ray powder diffraction pattern at diffraction angle 2-theta±0.2 degrees of 4.8° in combination of one or more peaks chosen from 5.6°, 6.2°, 14.8°, and 15.6°.

Embodiment 143. The compound of Embodiment 141, wherein the compound is in the form of a crystalline solid characterized by a peak in the X-ray powder diffraction pattern at diffraction angle 2-theta±0.2 degrees of 5.3° in combination of one or more peaks chosen from 7.7°, 10.5°, 11.3°, 11.6°, and 14.4°.

Embodiment 144. The compound of Embodiment 141, wherein the compound is in the form of a crystalline solid characterized by peaks in the X-ray powder diffraction pattern at diffraction angle 2-theta±0.2 degrees of 6.2° and 6.9°.

Embodiment 145. A method of synthesizing the compound of Embodiment 131, the method comprising:
reacting a compound of formula (H(dnp)ₚᵣₒₜ) and a compound of formula (Aibₚᵣₒₜ) to form a compound of formula (H(dnp)ₚᵣₒₜ-Aibₚᵣₒₜ)

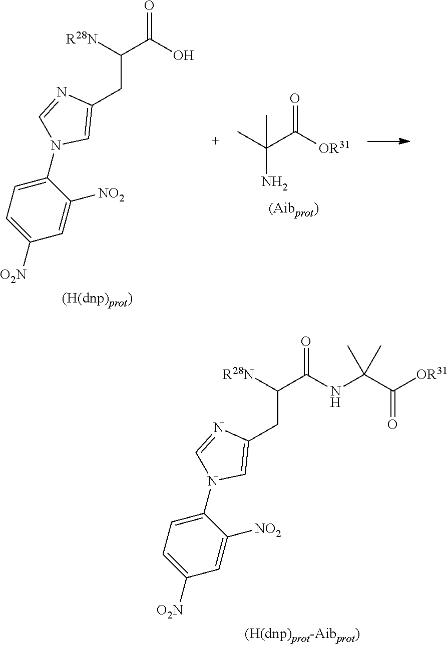

wherein R²⁸ and R³¹ are protecting groups;
removing the protecting group R³¹ to form a compound of (H(dnp)ₚᵣₒₜ-Aib)

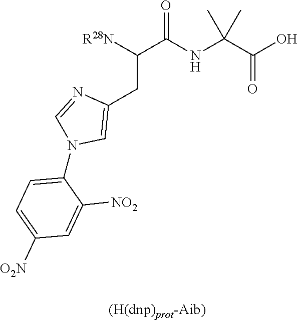

reacting the compound of formula (H(dnp)ₚᵣₒₜ-Aib) and a compound of formula (Qₚᵣₒₜ₂) to form a compound of a formula (H(dnp)ₚᵣₒₜ-Aib-Qₚᵣₒₜ₂)

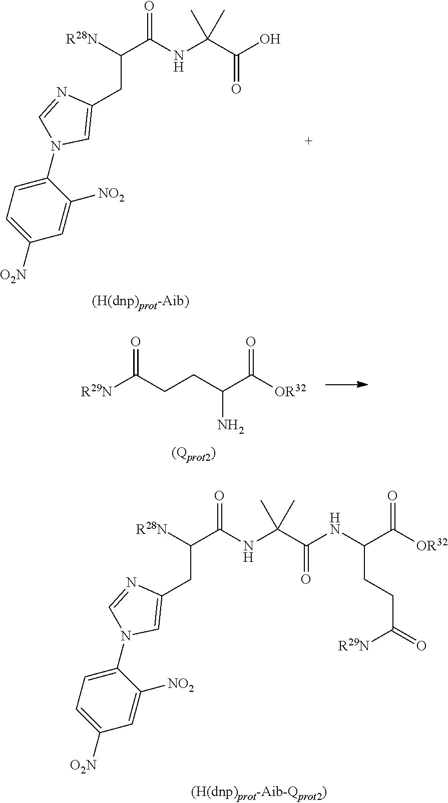

wherein R²⁹ and R³² are protecting groups;
removing the protecting group R³² to form a compound of (H(dnp)ₚᵣₒₜ-Aib-Qₚᵣₒₜ₁)

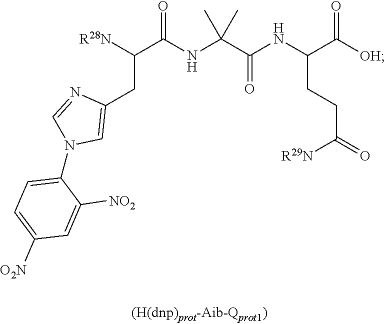

reacting the compound of formula (H(dnp)ₚᵣₒₜ-Aib-Qₚᵣₒₜ₁) and a compound of formula (Gₚᵣₒₜ) to form a compound of Formula (VII)

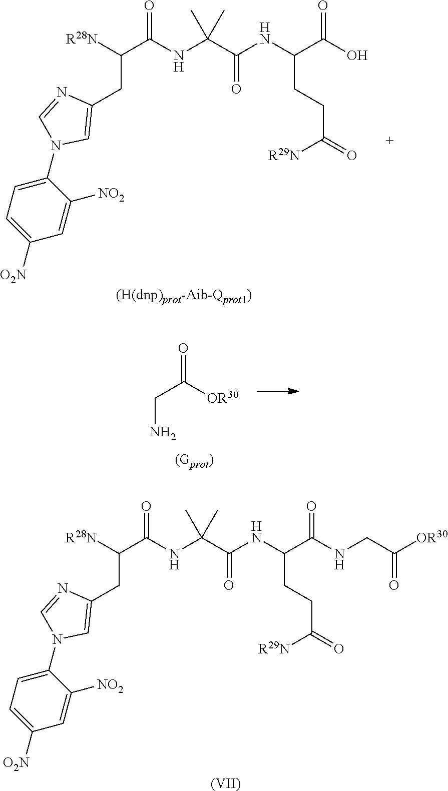

wherein R³⁰ is a protecting group; and
optionally removing one or more protecting groups R²⁸, R²⁹, and R³⁰.

Embodiment 146. The method of Embodiment 145, wherein each protecting group is independently chosen from Boc, Fmoc, tert-butyl, and trityl groups.

Embodiment 147. A method of synthesizing a polypeptide of SEQ ID NO: 16, the method comprising conjugating the compound of any one of Embodiments 131-137 via the C-terminus of the compound to the N-terminus of a polypeptide of SEQ ID NO: 17.

Embodiment 148. A compound of Formula (VIII), or a salt, solvate, or hydrate thereof,

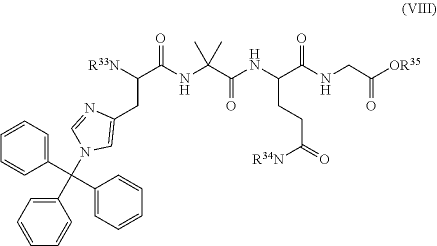

wherein R³³ is H or a protecting group; R³⁴ is H or a protecting group; and R³⁵ is H or a protecting group.

Embodiment 149. The compound of Embodiment 148, wherein each protecting group is independently chosen from Boc, Fmoc, tert-butyl, and trityl groups.

Embodiment 150. The compound of any one of Embodiments 148-149, wherein R³³ is H or Boc.

Embodiment 151. The compound of any one of Embodiments 148-150, wherein R³⁴ is H or trityl.

Embodiment 152. The compound of any one of Embodiments 148-151, wherein R³⁵ is H or tert-butyl.

Embodiment 153. The compound of any one of Embodiments 148-152, wherein at least one of R³³, R³⁴, and R³⁵ is H.

Embodiment 154. The compound of any one of Embodiments 148-153, wherein at least one of R³³, R³⁴, and R³⁵ is a protecting group.

Embodiment 155. The compound of Embodiment 148, wherein the compound is Formula (VIII-a):

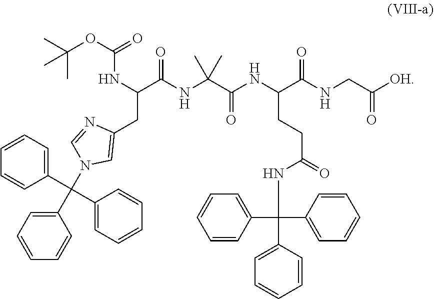 [SMILES: CC(C)(C)OC(=O)NC(Cc1cn(C(c2ccccc2)(c2ccccc2)c2ccccc2)cn1)C(=O)NC(C)(C)C(=O)NC(CCC(=O)NC(c1ccccc1)(c1ccccc1)c1ccccc1)C(=O)NCC(=O)O]

Embodiment 156. The compound of any one of Embodiments 148-155, wherein the compound is a solvate.

Embodiment 157. The compound of Embodiment 156, wherein the compound is a solvate produced from any one of a mixture of tetrahydrofuran and methyl tert-butyl ether, a mixture of tetrahydrofuran and heptane, a mixture of 1,4-dioxane and water, a mixture of ethyl acetate and methyl tert-butyl ether, and a mixture of acetonitrile and methyl tert-butyl ether.

Embodiment 158. The compound of any one of Embodiments 148-155, wherein the compound is crystalline.

Embodiment 159. The compound of Embodiment 158, wherein the compound is in the form of a crystalline solid characterized by a peak in the X-ray powder diffraction pattern at diffraction angle 2-theta±0.2 degrees of 4.7° in combination of one or more peaks chosen from 5.5°, 8.2°, 10.1°, 11.8°, 13.3°, 13.6°, and 18.9°; or

Embodiment 160. The compound of Embodiment 158, wherein the compound is in the form of a crystalline solid characterized by a peak in the X-ray powder diffraction pattern at diffraction angle 2-theta±0.2 degrees of 5.8° in combination of one or more peaks chosen from 5.3°, 8.9°, 9.2°, 15.2°, 18.60, and 19.5°.

Embodiment 161. A method of synthesizing the compound of Embodiment 148, the method comprising:
reacting a compound of formula (H(trt)ₚᵣₒₜ) and a compound of formula (Aibₚᵣₒₜ) to form a compound of formula (H(trt)ₚᵣₒₜ-Aibₚᵣₒₜ)

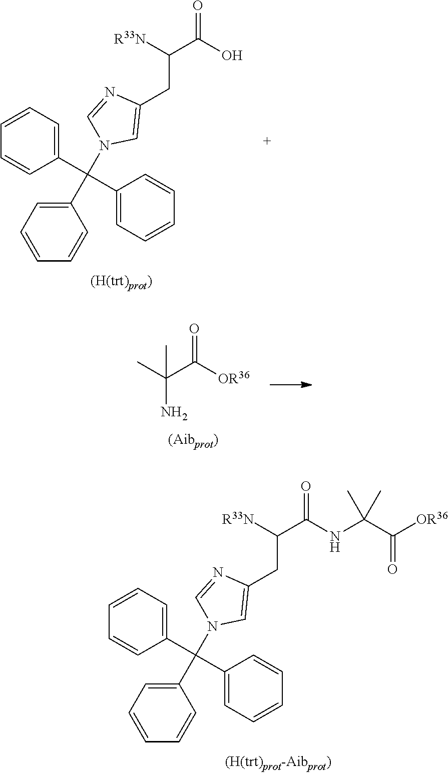

wherein R³³ and R³⁶ are protecting groups;
removing the protecting group R³⁶ to form a compound of (H(trt)ₚᵣₒₜ-Aib)

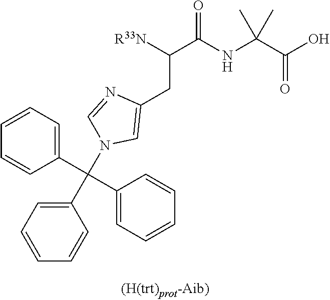

reacting the compound of formula (H(trt)ₚᵣₒₜ-Aib) and a compound of formula (Qₚᵣₒₜ₂) to form a compound of a formula (H(trt)ₚᵣₒₜ-Aib-Qₚᵣₒₜ₂)

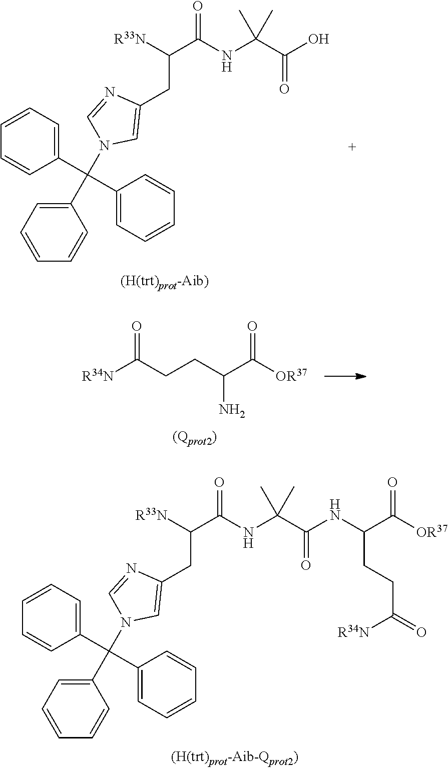

wherein R³³ and R³⁷ are protecting groups;
removing the protecting group R³⁷ to form a compound of (H(trt)ₚᵣₒₜ-Aib-Qₚᵣₒₜ₁)

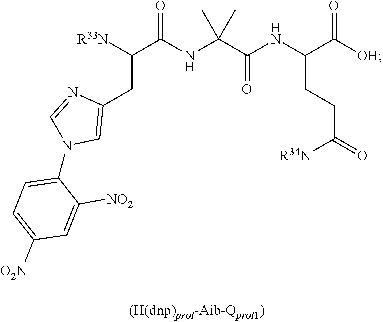

reacting the compound of formula (H(trt)ₚᵣₒₜ-Aib-Qₚᵣₒₜ₁) and a compound of formula (Gₚᵣₒₜ) to form a compound of Formula (VIII)

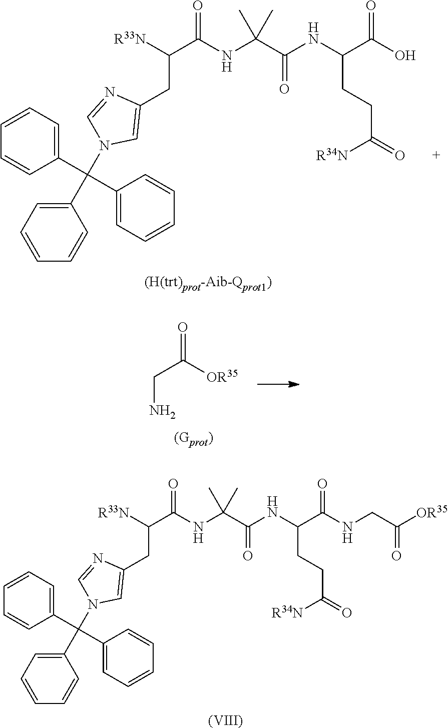

wherein R³⁵ is a protecting group; and
optionally removing one or more protecting groups R³³, R³⁴, and R³⁵.

Embodiment 162. The method of Embodiment 161, wherein each protecting group is independently chosen from Boc, Fmoc, tert-butyl, and trityl groups.

Embodiment 163. A method of synthesizing a polypeptide of SEQ ID NO: 16, the method comprising conjugating the compound of any one of Embodiments 148-155 via the C-terminus of the compound to the N-terminus of a polypeptide of SEQ ID NO: 17.

Embodiment 164. A compound of Formula (IX), or a salt, solvate, or hydrate thereof,

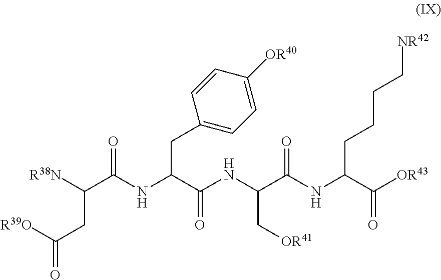

wherein R³⁸ is H or a protecting group; R³⁹ is H or a protecting group; R⁴⁰ is H or a protecting group; R⁴¹ is H or a protecting group; R⁴² is H or a protecting group; and R⁴³ is H or a protecting group.

Embodiment 165. The compound of Embodiment 164, wherein each protecting group is independently chosen from Boc, Fmoc, tert-butyl, and trityl groups.

Embodiment 166. The compound of any one of Embodiments 164-165, wherein R³⁸ is H or Fmoc.

Embodiment 167. The compound of any one of Embodiments 164-166, wherein R³⁹ is H or tert-butyl.

Embodiment 168. The compound of any one of Embodiments 164-167, wherein R⁴⁰ is H or tert-butyl.

Embodiment 169. The compound of any one of Embodiments 164-168, wherein R⁴¹ is H or tert-butyl.

Embodiment 170. The compound of any one of Embodiments 164-169, wherein R⁴² is H or Boc.

Embodiment 171. The compound of any one of Embodiments 164-170, wherein R⁴³ is H or tert-butyl.

Embodiment 172. The compound of any one of Embodiments 164-171, wherein at least one of R³⁸, R³⁹, R⁴⁰, R⁴¹, R⁴², and R⁴³ is H.

Embodiment 173. The compound of any one of Embodiments 164-172, wherein at least one of R³⁸, R³⁹, R⁴⁰, R⁴¹, R⁴², and R⁴³ is a protecting group.

Embodiment 174. The compound of Embodiment 164, wherein the compound is Formula (IX-a):

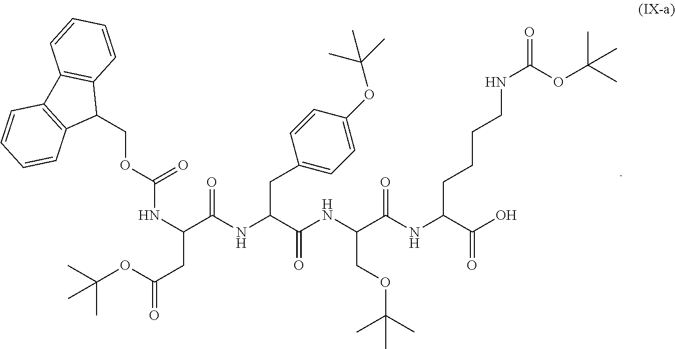 [SMILES: CC(C)(C)OCC(NC(=O)C(Cc1ccc(OC(C)(C)C)cc1)NC(=O)C(CC(=O)OC(C)(C)C)NC(=O)OCC1c2ccccc2-c2ccccc21)C(=O)NC(CCCCNC(=O)OC(C)(C)C)C(=O)O]

Embodiment 175. The compound of any one of Embodiments 164-174, wherein the compound is a solvate.

Embodiment 176. The compound of Embodiment 175, wherein the compound is a solvate produced from any one of a mixture of methyl acetate and dibutyl ether, a mixture of acetone and dibutyl ether, a mixture of acetonitrile and dibutyl ether, a mixture of ethyl acetate and dibutyl ether, a mixture of methyl acetate and heptane, and a mixture of methyl ethyl ketone and dibutyl ether.

Embodiment 177. The compound of any one of Embodiments 164-174, wherein the compound is a de-solvate or is anhydrous.

Embodiment 178. The compound of any one of Embodiments 164-174, wherein the compound is crystalline.

Embodiment 179. The compound of Embodiment 178, wherein the compound is in the form of a crystalline solid characterized by a peak in the X-ray powder diffraction pattern at diffraction angle 2-theta±0.2 degrees of 5.3° in combination of one or more peaks chosen from 6.0°, 6.9°, 7.2°, 8.0°, 12.2°, and 15.6°;

Embodiment 180. The compound of Embodiment 178, wherein the compound is in the form of a crystalline solid characterized by a peak in the X-ray powder diffraction pattern at diffraction angle 2-theta±0.2 degrees of 5.8° in combination of one or more peaks chosen from 4.4°, 6.6°, 10.1°, 11.4°, 13.4°, and 15.5°.

Embodiment 181. The compound of Embodiment 178, wherein the compound is in the form of a crystalline solid characterized by peaks in the X-ray powder diffraction pattern at diffraction angle 2-theta±0.2 degrees of 4.5° and 5.5° in combination of one or more peaks chosen from 6.0° and 7.3°.

Embodiment 182. A method of synthesizing the compound of Embodiment 164, the method comprising:
reacting a compound of formula (Dₚᵣₒₜ) and a compound of formula (Yₚᵣₒₜ₂) to form a compound of formula (Dₚᵣₒₜ-Yₚᵣₒₜ₂)

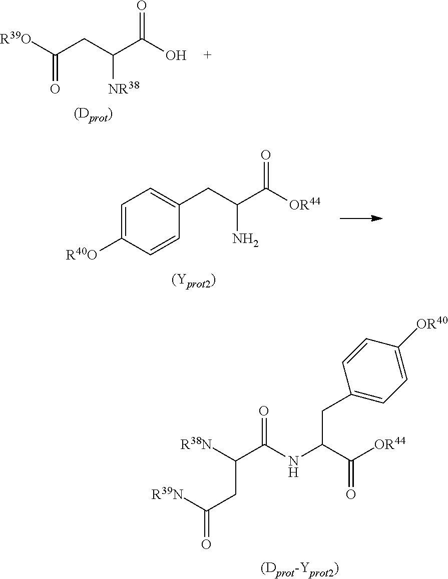

wherein R³⁸, R³⁹, R⁴⁰, and R⁴⁴ are protecting groups;
removing the protecting group R⁴⁴ to form a compound of (Dₚᵣₒₜ-Yₚᵣₒₜ₁)

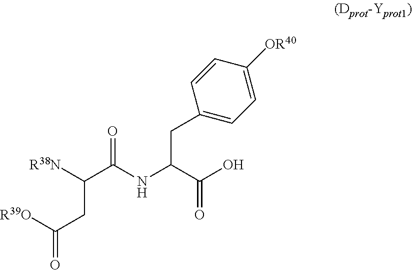

reacting the compound of formula (Dₚᵣₒₜ-Yₚᵣₒₜ₁) and a compound of formula (Sₚᵣₒₜ₂) to form a compound of a formula (Dₚᵣₒₜ-Yₚᵣₒₜ₁-Sₚᵣₒₜ₂)

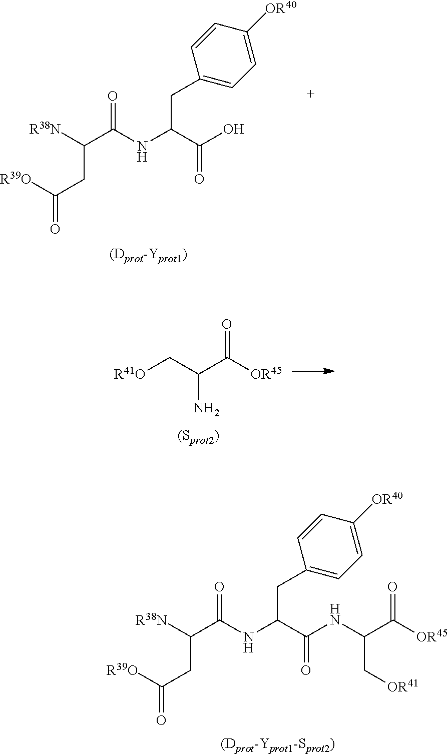

wherein R⁴¹ and R⁴⁵ are protecting groups;
removing the protecting group R⁴⁵ to form a compound of (Dₚᵣₒₜ-Yₚᵣₒₜ₁-Sₚᵣₒₜ₁)

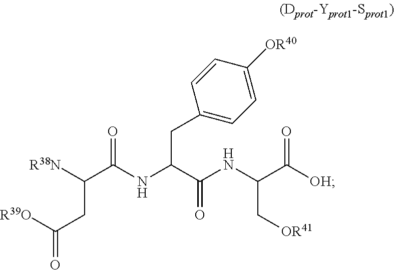

reacting the compound of formula (Dₚᵣₒₜ-Yₚᵣₒₜ₁-Sₚᵣₒₜ₁) and a compound of formula (Kₚᵣₒₜ₂) to form a compound of Formula (IX)

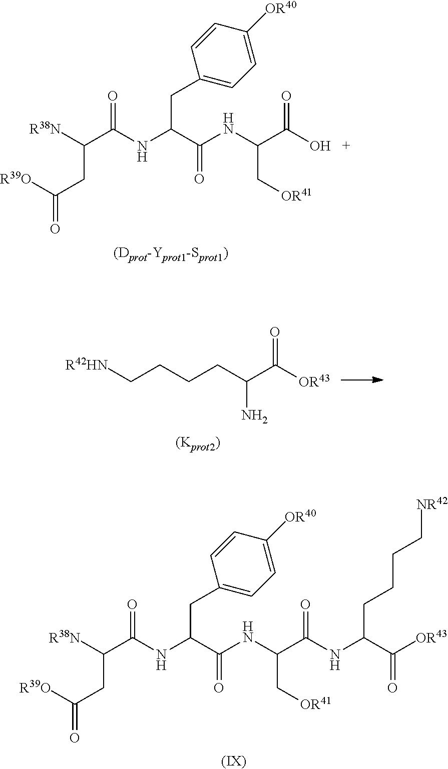

wherein R⁴² and R⁴³ are protecting groups; and
optionally removing one or more protecting groups R³⁸, R³⁹, R⁴⁰, R⁴¹, R⁴², and R⁴³.

Embodiment 183. The method of Embodiment 182, wherein the protecting groups are chosen from Boc, Fmoc, tert-butyl and trityl groups.

Embodiment 184. A method of synthesizing a polypeptide of SEQ ID NO: 16, the method comprising: (i) conjugating the compound of any one of Embodiments 164-174 via the C-terminus of the compound to the N-terminus of a polypeptide of SEQ ID NO: 18, and (ii) conjugating the compound via its N-terminus to the C-terminus of a polypeptide of SEQ ID NO: 19.

Embodiment 185. A compound of Formula (X), or a salt, solvate, or hydrate thereof,

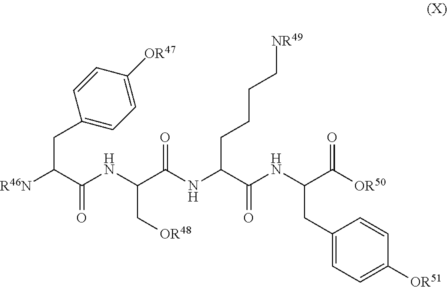

wherein R⁴⁶ is H or a protecting group; R⁴⁷ is H or a protecting group; R⁴⁸ is H or a protecting group; R⁴⁹ is H or a protecting group; R⁵⁰ is H or a protecting group; and R⁵¹ is H or a protecting group.

Embodiment 186. The compound of Embodiment 185, wherein each protecting group is independently chosen from Boc, Fmoc, tert-butyl, and trityl groups.

Embodiment 187. The compound of any one of Embodiments 185-186, wherein R⁴⁶ is H or tert-butyl.

Embodiment 188. The compound of any one of Embodiments 185-187, wherein R⁴⁷ is H or tert-butyl.

Embodiment 189. The compound of any one of Embodiments 185-188, wherein R⁴⁸ is H or Boc.

Embodiment 190. The compound of any one of Embodiments 185-189, wherein R⁴⁹ is H or Fmoc.

Embodiment 191. The compound of any one of Embodiments 185-190, wherein R⁵⁰ is H or tert-butyl.

Embodiment 192. The compound of any one of Embodiments 185-191, wherein R⁵¹ is H, or tert-butyl.

Embodiment 193. The compound of any one of Embodiments 185-192, wherein at least one of R⁴⁶, R⁴⁷, R⁴⁸, R⁴⁹, R⁵⁰, and R⁵¹ is H.

Embodiment 194. The compound of any one of Embodiments 185-193, wherein at least one of R⁴⁶, R⁴⁷, R⁴⁸, R⁴⁹, R⁵⁰, and R⁵¹ is a protecting group.

Embodiment 195. The compound of Embodiment 185, wherein the compound is Formula (X-a):

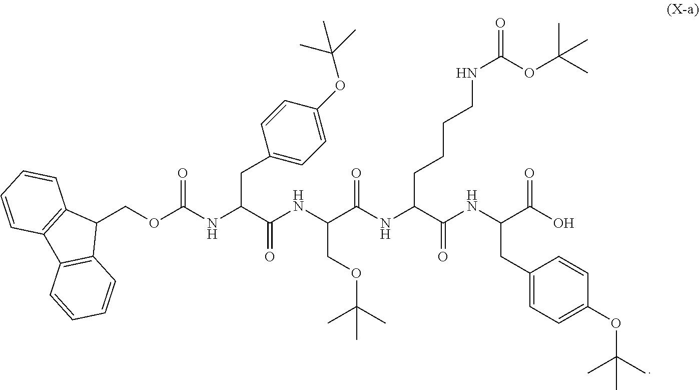 [SMILES: CC(C)(C)OCC(NC(=O)C(Cc1ccc(OC(C)(C)C)cc1)NC(=O)OCC1c2ccccc2-c2ccccc21)C(=O)NC(CCCCNC(=O)OC(C)(C)C)C(=O)NC(Cc1ccc(OC(C)(C)C)cc1)C(=O)O]

Embodiment 196. The compound of any one of Embodiments 185-195, wherein the compound is a solvate.

Embodiment 197. The compound of Embodiment 196, wherein the compound is a solvate produced from ethanol or isopropyl alcohol.

Embodiment 198. The compound of any one of Embodiments 185-195, wherein the compound is a de-solvate.

Embodiment 199. The compound of any one of Embodiments 185-195, wherein the compound is crystalline.

Embodiment 200. The compound of Embodiment 199, wherein the compound is in the form of a crystalline solid characterized by peaks in the X-ray powder diffraction pattern at diffraction angle 2-theta±0.2 degrees of 18.1° and 18.7° in combination of one or more peaks chosen from 5.7°, 8.7°, 13.7°, 14.3°, 15.9°, and 16.2°;

Embodiment 201. The compound of Embodiment 199, wherein the compound is in the form of a crystalline solid characterized by peaks in the X-ray powder diffraction pattern at diffraction angle 2-theta±0.2 degrees of 5.9° and 10.5° in combination of one or more peaks chosen from 7.1°, 8.9°, 14.6°, and 16.6°.

Embodiment 202. The compound of Embodiment 199, wherein the compound is in the form of a crystalline solid characterized by peaks in the X-ray powder diffraction pattern at diffraction angle 2-theta±0.2 degrees of 7.8° and 20.3° in combination of one or more peaks chosen from 5.8°, 15.5°, and 19.5°.

Embodiment 203. The compound of Embodiment 199, wherein the compound is in the form of a crystalline solid characterized by peaks in the X-ray powder diffraction pattern at diffraction angle 2-theta±0.2 degrees of 5.9° and 7.4° in combination of one or more peaks chosen from 6.5°, 6.9°, and 14.8°.

Embodiment 204. A method of synthesizing the compound of Embodiment 185, the method comprising:
reacting a compound of formula (Yₚᵣₒₜ) and a compound of formula (Sₚᵣₒₜ₂) to form a compound of formula (Yₚᵣₒₜ-Sₚᵣₒₜ₂)

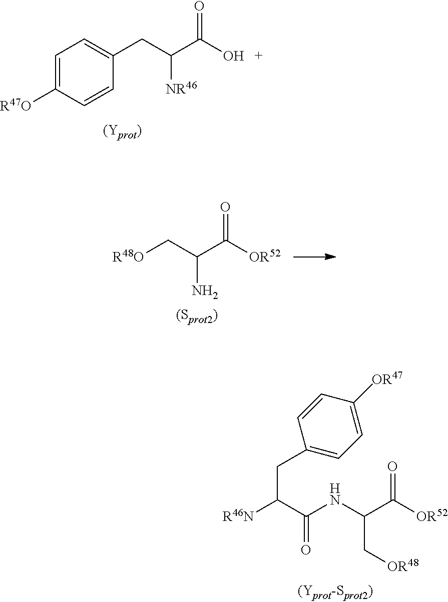

wherein R⁴⁶, R⁴⁷, R⁴⁸, and R⁵² are protecting groups;
removing the protecting group R⁵² to form a compound of (Yₚᵣₒₜ-Sₚᵣₒₜ₁)

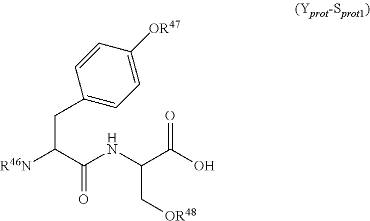

reacting the compound of formula (Yₚᵣₒₜ-Sₚᵣₒₜ₁) and a compound of formula (Kₚᵣₒₜ₂) to form a compound of a formula Yₚᵣₒₜ-Sₚᵣₒₜ₁-Kₚᵣₒₜ₂)

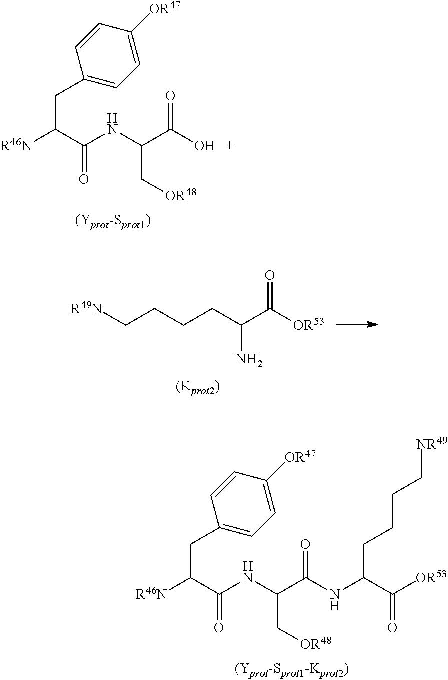

wherein R⁴⁹ and R⁵³ are protecting groups;
removing the protecting group R⁵³ to form a compound of (Yₚᵣₒₜ-Sₚᵣₒₜ₁-Kₚᵣₒₜ₁)

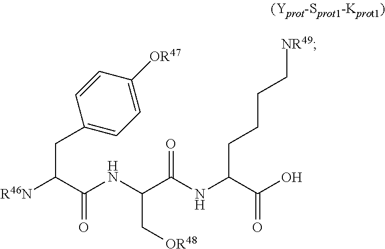

reacting the compound of formula (Yₚᵣₒₜ-Sₚᵣₒₜ₁-Kₚᵣₒₜ₁) and a compound of formula (Yₚᵣₒₜ₂) to form a compound of Formula (X)

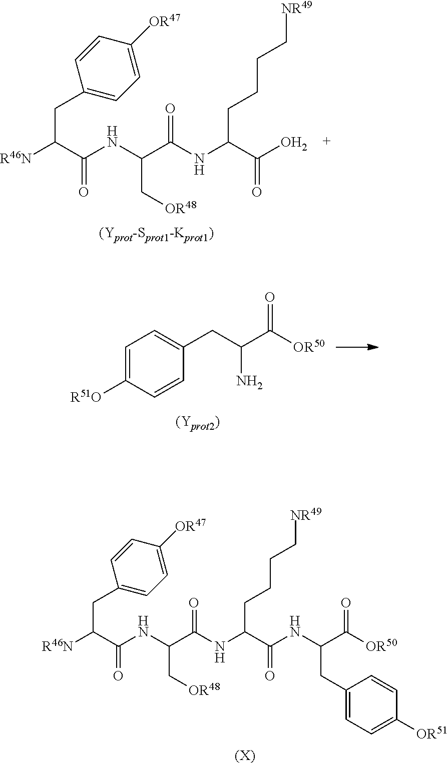

wherein R⁵⁰ and R⁵¹ are protecting groups; and
optionally removing one or more protecting groups R⁴⁶, R⁴⁷, R⁴⁸, R⁴⁹, R⁵⁰, and R⁵¹.

Embodiment 205. The method of Embodiment 204, wherein the protecting groups are chosen from Boc, Fmoc, tert-butyl, and trityl groups.

Embodiment 206. A method of synthesizing a polypeptide of SEQ ID NO: 16, the method comprising: (i) conjugating the compound of any one of Embodiments 185-195 via the C-terminus of the compound to the N-terminus of a polypeptide of SEQ ID NO: 20, and (ii) conjugating the resulting compound via its N-terminus to the C-terminus of a polypeptide of SEQ ID NO: 21.

Embodiment 207. A compound of Formula (XI), or a salt, solvate, or hydrate thereof,

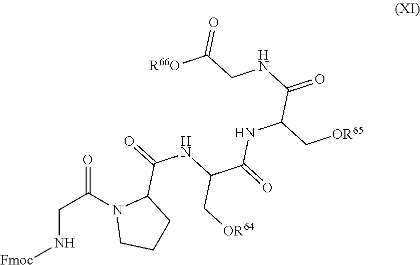

wherein R⁶⁴ is H or a protecting group; R⁶⁵ is H or a protecting group; and R⁶⁶ is H or a protecting group.

Embodiment 208. The compound of Embodiment 207, wherein each protecting group is independently chosen from Boc, Fmoc, tert-butyl, and trityl groups.

Embodiment 209. The compound of any one of Embodiments 207-208, wherein at least one of R⁶⁴, R⁶⁵, and R⁶⁶ is H.

Embodiment 210. The compound of any one of Embodiments 207-209, wherein at least one of R⁶⁴, R⁶⁵, and R⁶⁶ is a protecting group.

Embodiment 211. The compound of Embodiment 207, wherein the compound is of the Formula:

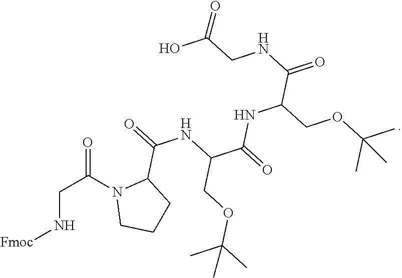

Embodiment 212. The compound of any one of Embodiments 207-211, wherein the compound is a solvate.

Embodiment 213. The compound of any one of Embodiments 207-211, wherein the compound is crystalline.

Embodiment 214. The compound of Embodiment 213, wherein the compound is in the form of a crystalline solid characterized by peaks in the X-ray powder diffraction pattern at diffraction angle 2-theta±0.2 degrees of 6.1° and 8.5° in combination of one or more peaks chosen from 5.8°, 16.9°, 18.5°, 18.8°, 19.3°, and 20.9.

Embodiment 215. A compound of Formula (XII), or a salt, solvate, or hydrate thereof,

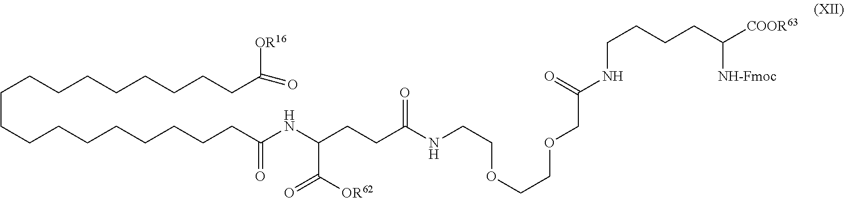

wherein R⁶¹ is H or a protecting group; R⁶² is H or a protecting group; and R⁶³ is H or a protecting group.

Embodiment 216. The compound of Embodiment 215, wherein each protecting group is independently chosen from Boc, Fmoc, tert-butyl, and trityl groups.

Embodiment 217. The compound of any one of Embodiments 215-216, wherein R⁶¹ is H or tert-butyl.

Embodiment 218. The compound of any one of Embodiments 215-217, wherein R⁶² is H or tert-butyl.

Embodiment 219. The compound of any one of Embodiments 215-218, wherein R⁶³ is H or tert-butyl.

Embodiment 220. The compound of any one of Embodiments 215-219, wherein at least one of R⁶¹, R⁶², and R⁶³ is H.

Embodiment 221. The compound of any one of Embodiments 215-220, wherein at least one of R⁶¹, R⁶², and R⁶³ is a protecting group.

Embodiment 222. The compound of Embodiment 215, wherein the compound is Formula (XII-a):

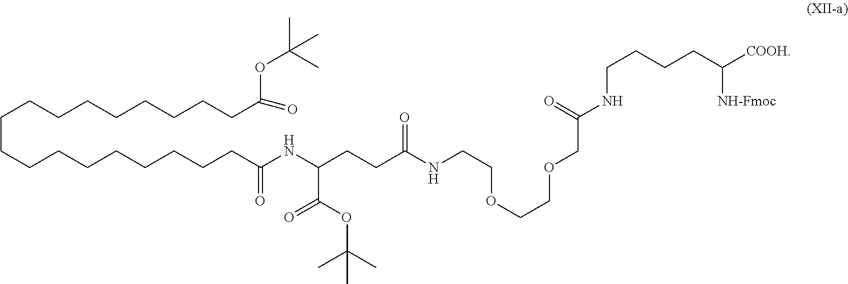

Embodiment 223. The compound of any one of Embodiments 215-222, wherein the compound is a solvate.

Embodiment 224. The compound of any one of Embodiments 215-222, wherein the compound is a de-solvate or is anhydrous.

Embodiment 225. The compound of any one of Embodiments 215-222, wherein the compound is crystalline.

Embodiment 226. The compound of Embodiment 225, wherein the compound is in the form of a crystalline solid characterized by peaks in the X-ray powder diffraction pattern at diffraction angle 2-theta±0.2 degrees of 11.4° in combination of one or more peaks chosen from 6.0°, 8.9°, 12.7°, 13.6°, 14.6°, 17.0°, and 18.8°.

Embodiment 227. The compound of Embodiment 225, wherein the compound is in the form of a crystalline solid characterized by peaks in the X-ray powder diffraction pattern at diffraction angle 2-theta±0.2 degrees of 10.6° in combination of one or more peaks chosen from 7.1°, 12.1°, 13.6°, 14.2°, 15.2°, 16.0°, and 16.8°.

Embodiment 228. The compound of Embodiment 225, wherein the compound is in the form of a crystalline solid characterized by peaks in the X-ray powder diffraction pattern at diffraction angle 2-theta±0.2 degrees of 10.1° and 15.5° in combination of one or more peaks chosen from 6.1°, 8.7°, 11.4°, 16.6°, and 19.2°.

Embodiment 229. A compound of Formula (XIII), or a salt, solvate, or hydrate thereof,

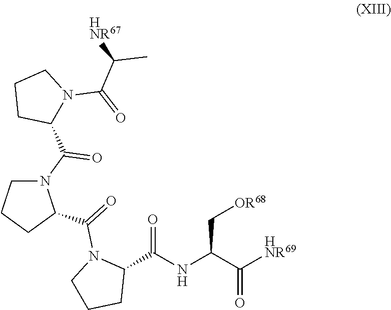

wherein R⁶⁷ is H or a protecting group; R⁶⁸ is H or a protecting group; and R⁶⁹ is H or a protecting group.

Embodiment 230. The compound of Embodiment 229, wherein each protecting group is independently chosen from Boc, Fmoc, tert-butyl, and trityl groups.

Embodiment 231. The compound of any one of Embodiments 229-230, wherein R⁶⁷ is H or tert-butyl.

Embodiment 232. The compound of any one of Embodiments 229-231, wherein R⁶⁸ is H or tert-butyl.

Embodiment 233. The compound of any one of Embodiments 229-232, wherein R⁶⁹ is H or tert-butyl.

Embodiment 234. The compound of any one of Embodiments 229-233, wherein at least one of R⁶⁷, R⁶⁸, and R⁶⁹ is H.

Embodiment 235. The compound of any one of Embodiments 229-234, wherein at least one of R⁶⁷, R⁶⁸, and R⁶⁹ is a protecting group.

Embodiment 236. The compound of Embodiment 229, wherein the compound is of the Formula:

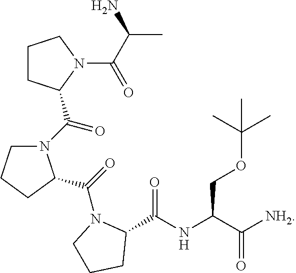 [SMILES: C[C@H](N)C(=O)N1CCC[C@H]1C(=O)N1CCC[C@H]1C(=O)N1CCC[C@H]1C(=O)N[C@@H](COC(C)(C)C)C(N)=O]

Embodiment 237. The compound of any one of Embodiments 229-236, wherein the compound is a solvate.

Embodiment 238. The compound of any one of Embodiments 229-236, wherein the compound is a de-solvate or is anhydrous.

Embodiment 239. The compound of any one of Embodiments 229-236, wherein the compound is crystalline.

Embodiment 240. The compound of Embodiment 239, wherein the compound is in the form of a crystalline solid characterized by peaks in the X-ray powder diffraction pattern at diffraction angle 2-theta±0.2 degrees of 5.0° in combination of one or more peaks chosen from 8.3, 9.7, and 11.2°.

Embodiment 241. The compound of Embodiment 239, wherein the compound is in the form of a crystalline solid characterized by peaks in the X-ray powder diffraction pattern at diffraction angle 2-theta±0.2 degrees of 7.2° in combination of one or more peaks chosen from 5.3, 8.1, 14.4, and 16.2°.

Embodiment 242. A compound of Formula (XIV), or a salt, solvate, or hydrate thereof,

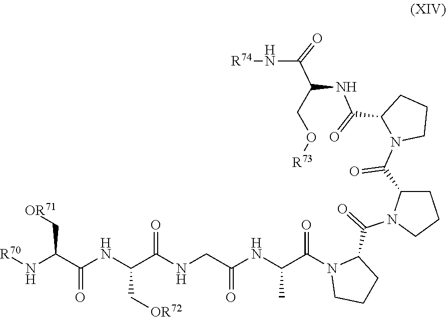

wherein R⁷⁰ is H or a protecting group; R⁷¹ is H or a protecting group; R⁷² is H or a protecting group; R⁷³ is H or a protecting group; and R⁷⁴ is H or a protecting group.

Embodiment 243. The compound of Embodiment 242, wherein each protecting group is independently chosen from Boc, Fmoc, tert-butyl, and trityl groups.

Embodiment 244. The compound of any one of Embodiments 242-243, wherein R⁷⁰ is H, Fmoc, or tert-butyl.

Embodiment 245. The compound of any one of Embodiments 242-244, wherein R⁷¹ is H or tert-butyl.

Embodiment 246. The compound of any one of Embodiments 242-245, wherein R⁷² is H or tert-butyl.

Embodiment 247. The compound of any one of Embodiments 242-246, wherein R⁷³ is H or tert-butyl.

Embodiment 248. The compound of any one of Embodiments 242-247, wherein R⁷⁴ is H, Fmoc, or tert-butyl.

Embodiment 249. The compound of any one of Embodiments 242-248, wherein at least one of R⁷⁰, R⁷¹, R⁷², R⁷³, and R⁷⁴ is H.

Embodiment 250. The compound of any one of Embodiments 242-249, wherein at least one of R⁷⁰, R⁷¹, R⁷², R⁷³, and R⁷⁴ is a protecting group.

Embodiment 251. The compound of Embodiment 242, wherein the compound is of the Formula:

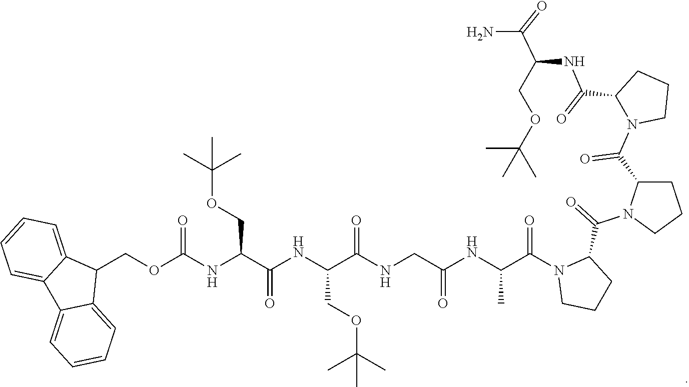 [SMILES: C[C@H](NC(=O)CNC(=O)[C@H](COC(C)(C)C)NC(=O)[C@H](COC(C)(C)C)NC(=O)OCC1c2ccccc2-c2ccccc21)C(=O)N1CCC[C@H]1C(=O)N1CCC[C@H]1C(=O)N1CCC[C@H]1C(=O)N[C@@H](COC(C)(C)C)C(N)=O]

Embodiment 252. The compound of any one of Embodiments 242-251, wherein the compound is a solvate.

Embodiment 253. The compound of any one of Embodiments 242-251, wherein the compound is a de-solvate or is anhydrous

Embodiment 254. The compound of any one of Embodiments 242-251, wherein the compound is crystalline.

Embodiment 255. The compound of Embodiment 254, wherein the compound is in the form of a crystalline solid characterized by peaks in the X-ray powder diffraction pattern at diffraction angle 2-theta±0.2 degrees of 7.5° in combination of one or more peaks chosen from 6.1, 8.7, 10.6, 15.0, 16.1, and 18.6°.

### EXAMPLES

### Example 1: Boc-l-Tyr (tBu)-Aib-Glu(OtBu)-Gly-OH

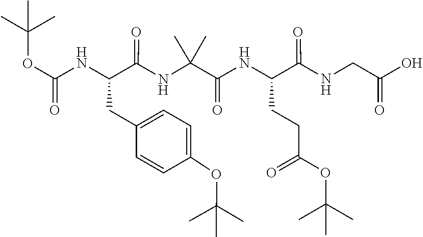 [SMILES: CC(C)(C)OC(=O)CC[C@H](NC(=O)C(C)(C)NC(=O)[C@H](Cc1ccc(OC(C)(C)C)cc1)NC(=O)OC(C)(C)C)C(=O)NCC(=O)O]

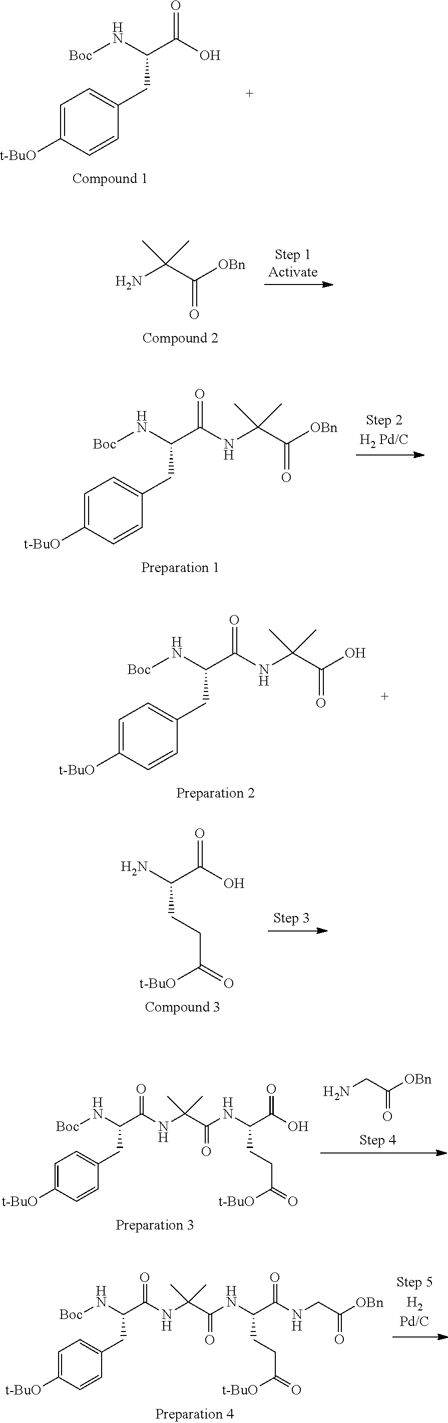

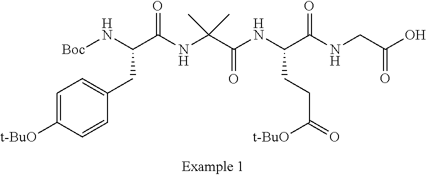 [SMILES: CC(C)(C)OC(=O)CC[C@H](NC(=O)C(C)(C)NC(=O)[C@H](Cc1ccc(OC(C)(C)C)cc1)NC(=O)OC(C)(C)C)C(=O)NCC(=O)O]

Compound 1 ((S)-3-(4-(tert-butoxy)phenyl)-2-((tert-butoxycarbonyl)amino)propanoic acid) was added to 10 volumes (vol) of EtOAc and the temperature was adjusted to −10 to 0° C. BOP (1.3 eq) was added in portions at −10 to 0° C. Compound 2 (benzyl 2-amino-2-methylpropanoate), 1.3 eq, was added at −10 to 0° C. DIPEA was added dropwise at −10 to 0° C. and the mixture was stirred for 16-20 hours at −10 to 0° C. The mixture was warmed to 0-20° C. and washed with 10 vol, 5 times, at 0-20° C. The aqueous layers were combined. The organic layer was washed with 0.5 mol/L Na₂CO₃ (5 vol), 2 times, at 0-20° C. The organic layer was washed with water (10 vol) at 0-20° C. and the aqueous layers were combined. The organic layer was concentrated to dryness below 40° C. EtOAc (2 vol) was added at 15-25° C., the mixture was stirred for 1 hour, and then filtered to obtain a wet cake. The filtrate was concentrated to dryness below 40° C. EtOH, 5 vol, was added to form a clear solution, and temperature was adjusted to 5-15° C. Water, 1-2 vol, was added into the mixture dropwise, seed (0.5% w/w) was charged into the mixture, and the mixture was stirred for 1-2 hours. Water, 3-4 vol, was added into the mixture dropwise, the mixture was stirred for 16-24 hours, and then filtered. The wet cake was washed with EtOH/water (1/1 vol/vol). The wet cake was dried in a vacuum at 35-45° C. for 20-24 hours to provide Preparation 1.

One equivalent of Preparation 1 was mixed with Pd/C (0.1×). MeOH (10.5×, 13.3 vol) was added to the mixture and argon was exchanged 3 times, then hydrogen was exchanged 3 times. The pressure was adjusted to 45 psi with hydrogen, and the mixture was heated to 40° C., and stirred for 18-24 hours. The mixture was cooled to 20-30° C., argon was exchanged 3 times, and the mixture was filtered. The wet cake was washed with MeOH (1 vol), and the filtrate was transferred into a vessel and concentrated to −2 vol below 45° C. EA was added (300 ml, 3 vol) and the mixture was concentrated to −2 vol below 45° C., EA was added (300 ml, 3 vol) and the mixture was concentrated to −2 vol below 45° C. Heptane (8 vol) was added dropwise, the mixture was stirred at 20-35° C. for 20-24 hours, and then the mixture was filtered. The wet cake was washed with heptane (1 vol) and then dried in a vacuum at 40 to 50° C. for 20-24 hours to provide Preparation 2.

THF was added to a vessel 1 and cooled to 0° C., IBCF (1.3 eq, 0.42×) was mixed with Preparation 2 (1.0×). Three vol of THF was added into another vessel 2 and NMM (1.35 eq, 0.325×) was added, then this mixture was added to the mixture containing Preparation 2 and stirred for 4-6 hours. Three vol of THF was added to vessel 2, followed by 3 vol of water, and the mixture was stirred for 30 min at 15-20° C. The contents of vessel 2 was added to vessel 1 at 0-5° C. Vessel 1 was heated to 20° C. over 3-4 hours and stirred for 16-20 hours. 15 vol of EA was added to vessel 1 and the organic layer was separated. 5% KHSO₄ aqueous (16 vol) was added to the organic layer at 15-25° C., the mixture was stirred for 1-2 hours and the organic layer was separated and washed with 5% NaCl (10 vol), 2 times. The mixture in vessel 1 was concentrated to −1 vol below 45° C., THF (5 vol) was added and the mixture was concentrated to −1 vol IPA/H₂O 1:2 (10.5 vol) was added and the mixture was stirred at 15-25° C. for 48 hours. The mixture was then filtered, the wet cake washed with IPA/H₂O 1:2 (1 vol) twice, and the wet cake dried in vacuum at 40-45° C. for 20-24 hours to produce Preparation 3.

Preparation 3 (1.0×) was added to benzyl glycinate (1.3 eq, 0.44×) and then 10 vol of ACN was added. 2,6-lutidine (3.0 eq, 0.53×) was added and the temperature was adjusted to −10° C. COMU (1.3 eq, 0.86×) was added at −10° C. and the mixture was stirred for 2-4 hours. The mixture was concentrated to −1 vol below 45° C., EA (10 vol) was added, and the mixture was stirred for 0.5-1 hour at 15-20° C. The mixture was then filtered and the wet cake was washed with EA (1 vol). The filtrate was added and concentrated to −1 vol below 45° C. MTBE (10 vol) was added and the mixture was stirred for 0.5-1 hour at 15-20° C. The mixture was then filtered and the wet cake was washed with MTBE (1 vol). The filtrate was washed with 0.5 ml/L NaCO₃ aqueous (10 vol) twice at 15-25° C., washed with 5% KHSO₄ aqueous (10 vol) twice at 15-25° C., and washed with water (10 vol) at 15-25° C. The organic layer was separated and concentrated to 1-2 vol below 45° C. MTBE (10 vol) was added and the mixture was heated to 40° C. and cooled to 10° C. over 2 hours. The mixture was filtered, and the cake was dried at 45° C. for 16-24 hours to produce Preparation 4.

Preparation 4 (1×, 1 eq) was added to IPA (7.92×, 10 vol), Pd/C was charged (0.1×), argon was exchanged 3 times, then hydrogen was exchanged 3 times, the pressure was adjusted to 45 psi with hydrogen, and the mixture was heated to 40° C. and stirred for 18-24 hours. The mixture was cooled to 20-30° C., argon was exchanged 3 times, the mixture filtered, the wet cake washed with IPA (1 vol), and the filtrate was concentrated to −1 vol below 45° C. MTBE (3 vol) was added, and the mixture was concentrated to −1 vol below 45° C. MTBE (10 vol) and IPA (0.5 vol) were added to the mixture, and the mixture was heated to 40° C., cooled to 20° C. over 2 hours, and stirred for 2-6 hours at 20° C. The solids were filtered, the wet cake washed with 1 vol MTBE, and the wet cake was dried under vacuum for 16-24 hours at 40-50° C. to produce Y-Aib-E-G.

### Preparation of Y-Aib-E-G Forms A, B, and C

Three solid forms of the Y-Aib-E-G tetramer were prepared: Form A, Form B, and Form C. Y-Aib-E-G Form A is a solvated form produced from MTBE or mixtures containing MTBE (such as MTBE/IPA, MTBE/heptane, etc.). Y-Aib-E-G Form B is a desolvated form from Form A by drying at elevated temperature/vacuum, heating to 110° C., or exposing to 75% RH, etc. Y-Aib-E-G Form C is a hydrate containing varied amounts of water in the unit cell including the case of zero water content.

Crystallization of Y-Aib-E-G helps to reject impurities from the crude material. Compared to the amorphous crude Y-Aib-E-G, the crystalline Y-Aib-E-G shows improved purity based on UPLC-MS analysis. Crystallization of the crude Y-Aib-E-G spiked with 1% dimer (impurity) yielded a crystalline Y-Aib-E-G with almost complete rejection of the dimer. Crystallization also improves physical properties, for example, the hygroscopicity. By dynamic vapor sorption (DVS) analysis, an amorphous Y-Aib-E-G displayed 4.1 wt % gain in sorption from 0% to 95% RH, while Y-Aib-E-G Form B only gained 1.4 wt % under the same conditions.

The XRPD patterns of crystalline TZP tetramers were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. In some cases, the sample was scanned between 4 and 30 2θ° with a scan rate of 0.25 seconds/step. The powder was packed on a quartz sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### Y-Aib-E-G Form A

0.2 mL MTBE was added to 110.9 mg of amorphous solids of Y-Aib-E-G and the sample was stirred at ambient conditions. After 4 days, an additional 0.4 mL of MTBE was added to the mixture and the sample was stirred for another day. A white slurry was obtained, which was transferred to 0.45 μm nylon centrifuge tube filters and centrifuged at ambient temperature for 5 minutes, and the resulting white solids were consistent with Y-Aib-E-G Form A.

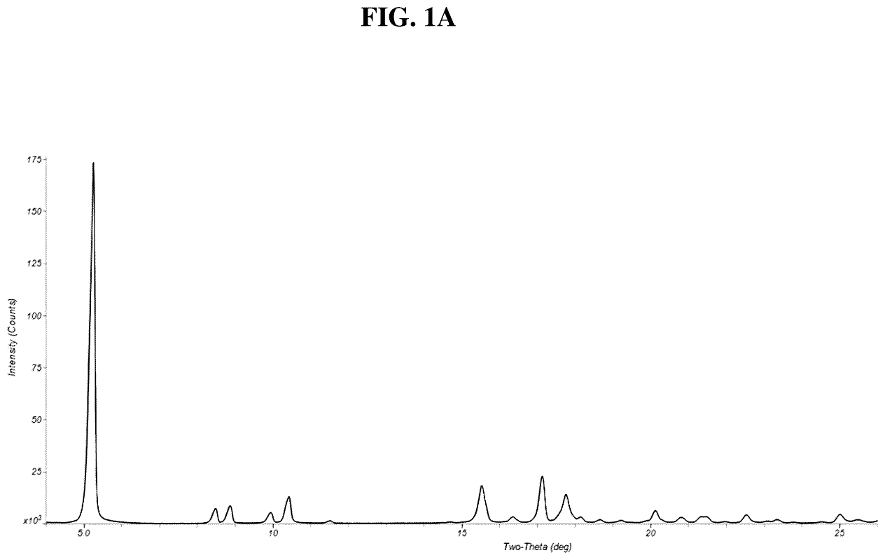

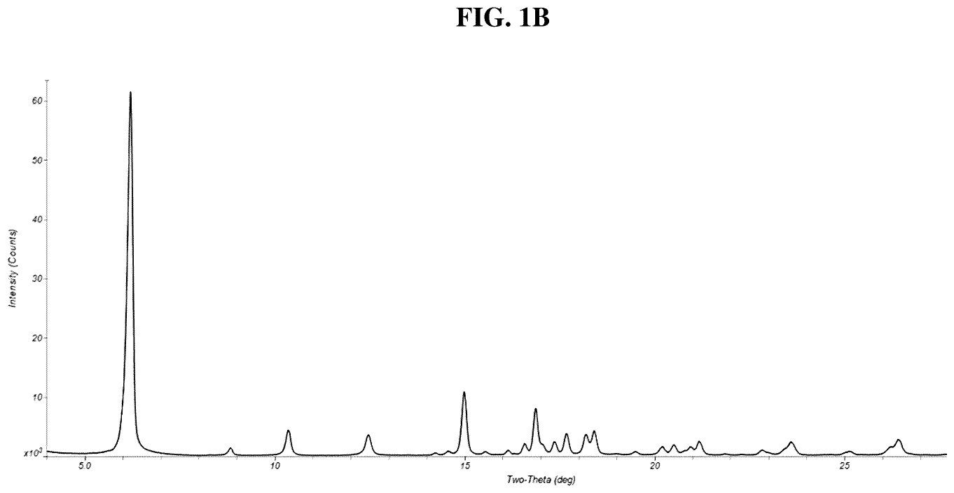

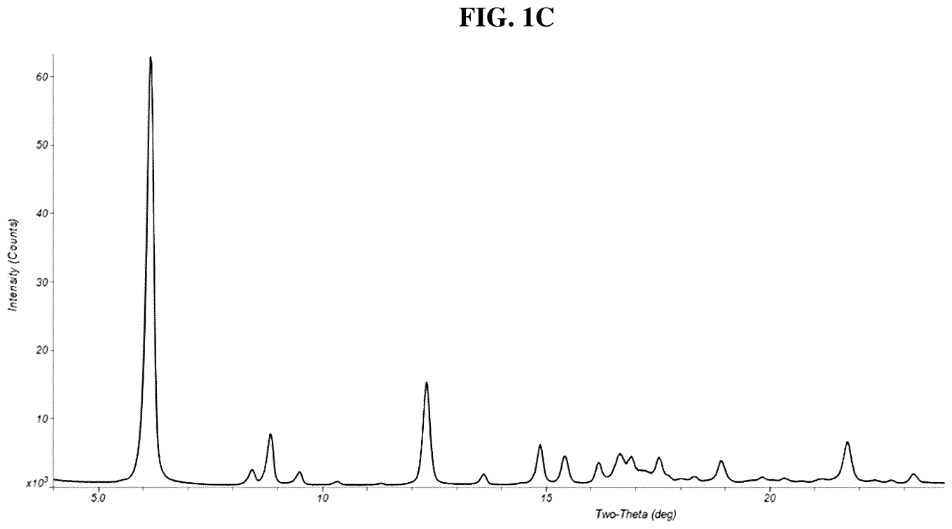

The prepared sample of Y-Aib-E-G Form A was characterized by an XRPD pattern using CuKα radiation as having diffraction peaks (2-theta values) as shown in Table 1 below, and in particular having a peak at 5.2 °2-Theta, in combination with one or more of the peaks selected from 9.9, 10.4, 15.5, and 17.1 °2-Theta; with a tolerance for the diffraction angles of 0.2 degrees. A representative XRPD pattern of Y-Aib-E-G Form A is shown in FIG. 1A.

TABLE 1
X-ray powder diffraction peaks of Y-Aib-E-G Form A
Y-Aib-E-G Form A

| | Angle | Relative Intensity |
|---|---|---|
| Peak | (°2-Theta) +/− 0.2° | (% of most intense peak) |
| 1 | 5.2 | 100.0% |
| 2 | 8.4 | 4.1% |
| 3 | 8.8 | 4.8% |
| 4 | 9.9 | 3.0% |
| 5 | 10.4 | 7.4% |
| 6 | 11.5 | 0.7% |
| 7 | 15.5 | 10.2% |
| 8 | 16.3 | 1.5% |
| 9 | 17.1 | 12.8% |
| 10 | 17.7 | 7.0% |
| 11 | 18.1 | 1.5% |
| 12 | 20.1 | 3.3% |
| 13 | 20.8 | 1.3% |
| 14 | 21.3 | 1.6% |
| 15 | 21.4 | 1.6% |
| 16 | 22.5 | 2.1% |
| 17 | 25.0 | 2.4% |

### Y-Aib-E-G Form B

6 mL MTBE was added to 1.01 gram of amorphous solids of Y-Aib-E-G and the sample was stirred at ambient conditions for 5 days, which yielded a white slurry. The slurry was transferred to 0.45 μm nylon centrifuge tube filters and centrifuged at ambient temperature for 5 minutes, and the resulting white solids (0.78 g) were primarily consistent with Y-Aib-E-G Form A. 81.6 mg of such white solids were dried at approximate 50° C. under vacuum for 1 day. The generated white solids were consistent with Y-Aib-E-G Form B.

The prepared sample of Y-Aib-E-G Form B was characterized by an XRPD pattern using CuKα radiation as having diffraction peaks (2-theta values) as described in Table 2 below, and in particular having a peak at 6.1 °2-Theta in combination with one or more of the peaks selected from 10.3, 16.8, and 18.1 °2-Theta; with a tolerance for the diffraction angles of 0.2 degrees. A representative XRPD pattern of Y-Aib-E-G Form B is shown in FIG. 1B.

TABLE 2
X-ray powder diffraction peaks of Y-Aib-E-G Form B
Y-Aib-E-G Form B

| | Angle | Relative Intensity |
|---|---|---|
| Peak | (°2-Theta) +/− 0.2° | (% of most intense peak) |
| 1 | 6.1 | 100.0% |
| 2 | 8.8 | 3.3% |
| 3 | 10.3 | 10.3% |
| 4 | 12.4 | 5.9% |
| 5 | 14.9 | 19.5% |
| 6 | 16.8 | 21.0% |
| 7 | 17.6 | 4.7% |
| 8 | 18.1 | 15.6% |
| 9 | 18.2 | 13.7% |
| 10 | 20.1 | 4.3% |
| 11 | 21.1 | 6.7% |
| 12 | 22.7 | 3.0% |
| 13 | 23.5 | 6.9% |
| 14 | 26.2 | 5.2% |

### Y-Aib-E-G Form C

285.8 mg of amorphous Y-Aib-E-G was stirred in 1 mL of water at ambient conditions overnight, resulting a thick slurry; additional amounts of water, 2×0.5 mL, and IPA, 2×0.1 mL were added to the slurry and the sample was stirred at ambient conditions for another 6 days. An off-white slurry was obtained, was transferred to 0.45 μm nylon centrifuge tube filters and centrifuged at ambient temperature for 5 minutes. The isolated solids were white and damp, were dried at 70-78° C. under vacuum for 2 hours. The resulting white solids were consistent with Y-Aib-E-G Form C.

The prepared sample of Y-Aib-E-G Form C was characterized by an XRPD pattern using CuKα radiation as having diffraction peaks (2-theta values) as described in Table 3 below, and in particular having a peak at 6.2 °2-Theta in combination with one or more of the peaks selected from 9.5, 13.6, and 21.8 °2-Theta; with a tolerance for the diffraction angles of 0.2 degrees. A representative XRPD pattern of Y-Aib-E-G Form C is shown in FIG. 1C.

TABLE 3
X-ray powder diffraction peaks of Y-Aib-E-G tetramer Form C
Y-Aib-E-G Form C

| | Angle | Relative Intensity |
|---|---|---|
| Peak | (°2-Theta) +/− 0.2° | (% of most intense peak) |
| 1 | 6.2 | 100.0% |
| 2 | 8.5 | 3.5% |
| 3 | 8.9 | 11.5% |
| 4 | 9.5 | 3.0% |
| 5 | 12.3 | 24.1% |
| 6 | 13.6 | 2.4% |
| 7 | 14.9 | 9.1% |
| 8 | 15.4 | 6.0% |
| 9 | 16.2 | 4.8% |
| 10 | 16.7 | 5.4% |
| 11 | 16.9 | 4.7% |
| 12 | 17.5 | 5.3% |
| 13 | 18.9 | 4.8% |
| 14 | 21.8 | 9.3% |
| 15 | 23.2 | 2.2% |

Coupling of Y-Aib-E-G Tetramer to 10-mer for TZP Synthesis

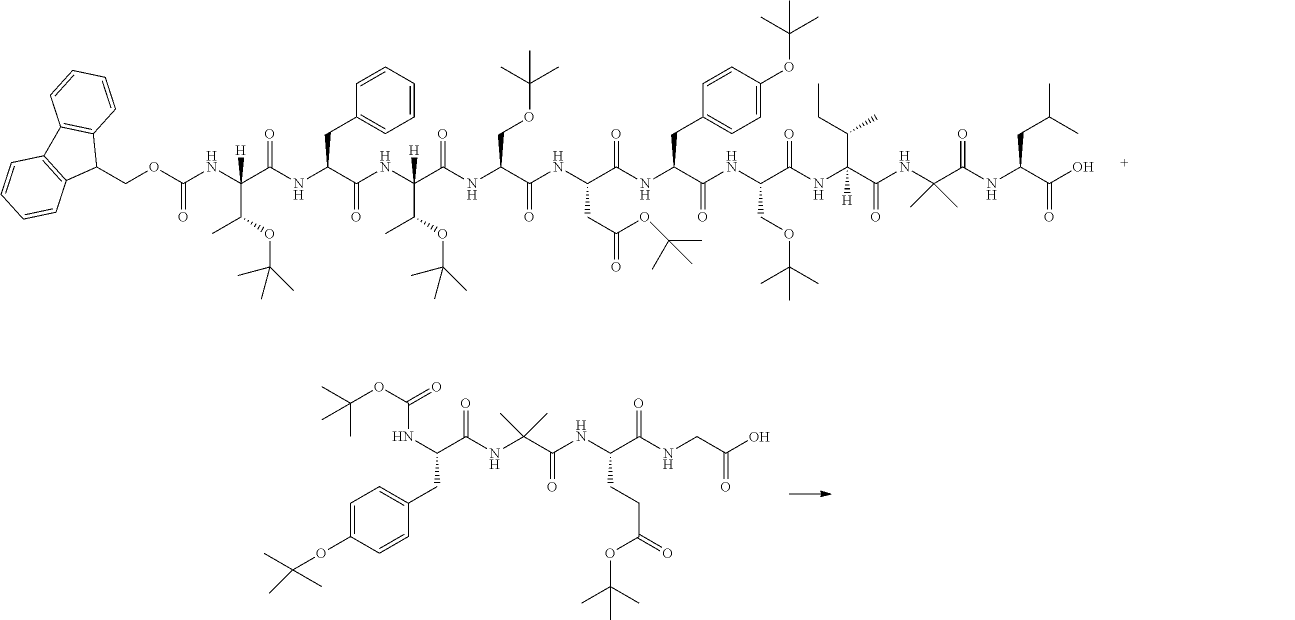

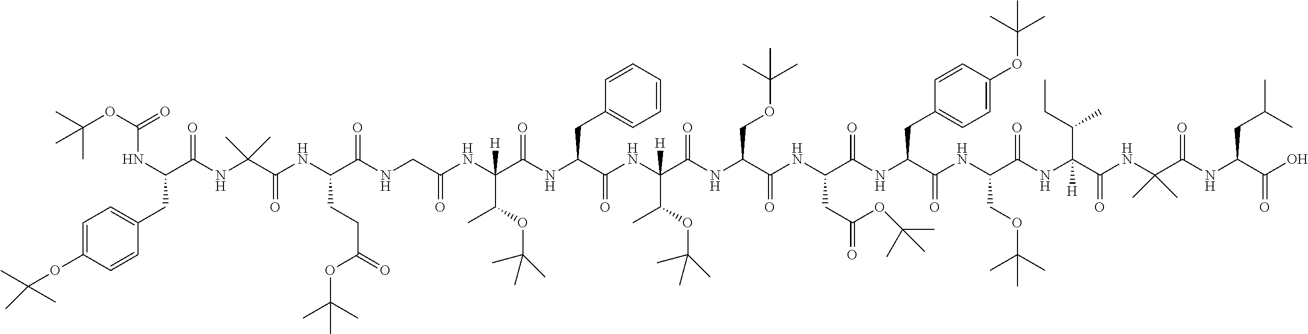 [SMILES: CC[C@H](C)[C@H](NC(=O)[C@H](COC(C)(C)C)NC(=O)[C@H](Cc1ccc(OC(C)(C)C)cc1)NC(=O)[C@H](CC(=O)OC(C)(C)C)NC(=O)[C@H](COC(C)(C)C)NC(=O)[C@@H](NC(=O)[C@H](Cc1ccccc1)NC(=O)[C@@H](NC(=O)CNC(=O)[C@H](CCC(=O)OC(C)(C)C)NC(=O)C(C)(C)NC(=O)[C@H](Cc1ccc(OC(C)(C)C)cc1)NC(=O)OC(C)(C)C)[C@@H](C)OC(C)(C)C)[C@@H](C)OC(C)(C)C)C(=O)NC(C)(C)C(=O)N[C@@H](CC(C)C)C(=O)O]

The crystalline Y-Aib-E-G can be used as a starting material to generate TZP fragment (AA 1-14) by a coupling reaction with a fragment 10-mer (AA 5-14). The TZP fragment (AA 1-14) may then couple with other fragments to produce protected TZP as an active pharmaceutical ingredient (API).

A TZP fragment 10-mer (AA 5-14), protected, on resin (1.2518 g, 0.500 mmol) in a 45 ml automated synthesizer reactor was swelled with 3×15 ml DMF for 15 minutes each, deprotected with 3×15 ml of 20% piperidine/DMF for 30 minutes each, and washed with 5×15 ml of DMF for one minute each. A solution was made of TZP tetramer (1.055 g, 1.500 mmol, 94.53 mass %) and ethyl cyanoglyoxylate-2-OXIME (214.3 mg, 1.500 mmol, 99 mass %) in 9 ml of DMF. N,N′-diisopropylcarbodiimide (258 μL, 1.650 mmol, 100 mass %) was added and the resulting yellow solution was allowed to stand for 30 minutes with occasional shaking, then the solution was added to the reactor containing the resin. The reaction was allowed to mix for 18 hours at ambient temperature and was then drained. The resin was washed with 5×15 ml of DMF for 1 minute each, 5×15 ml of DCM for 1 minute each, then drain dried for 4 hours to a constant weight of 1.4077 g.

For analysis, approximately 300 mg of resin was treated with 5 ml of a solution consisting of trifluoroacetic acid (4.64 mL, 61.4 mmol, 100 mass %), triisopropylsilane (125 μL, 0.609 mmol, 100 mass %), water (125 μL, 6.93874 mmol, 100 mass %), and DTI (125 mg, 0.810357 mmol, 100 mass %). The mixture was agitated on the rotary mixer for 2 hours, filtered, and washed with trifluoroacetic acid (2 mL, 26.45 mmol, 100 mass %). The combined filtrate and wash were added to 35 ml of cold MTBE in a centrifuge tube. After 30 minutes in the freezer, the mixture was centrifuged and the supernatant was decanted. The residual solid was washed with 2×30 ml of room temperature MTBE on the centrifuge and dried in the vacuum oven at 35° C. overnight. The yield of solid was 154.7 mg.

Analysis of the isolated solid by LC/MS showed complete coupling of the 10-mer to the tetramer to produce a TZP fragment 14-mer (AA 1-14).

### Example 2: Fmoc-Thr(tBu)-Phe-Thr(tBu)-Ser(tBu)-OH

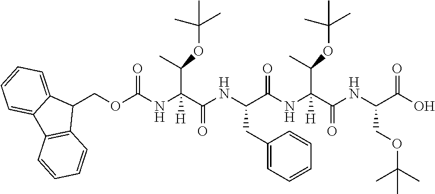 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)O)[C@@H](C)OC(C)(C)C]

Swelling of the resin: The resin (0.500 mmol) was added to the reactor and swelled with DMF (3×10 mL×20 min).

Washing after DeFmoc: Following deprotection, the resin was washed with DMF (5×10 mL×2 min).

Washing after coupling: Following coupling, the resin was washed with DMF (5×10 mL×2 min).

Washing and drying of the resin: Following final coupling or deprotection, the resin was washed with DMF (5×10 mL×2 min), followed by washing with DCM (5×10 mL×2 min), and then drain dried under a N₂ atmosphere to a constant weight.

Preparation 33 Procedure

| | | T(tBu)-F-T-(tBu)-S(tBu) | | | |
|---|---|---|---|---|---|
| | | DeFmoc | | | |
| | | reagents & | | | |
| | Fmoc- | DeFmoc | Coupling | Coupling | |
| Cycle | AA-OH | condition | reagents | Time | Comments |
| 1 | Fmoc-L- | N/A | CTC resin | 18 h | Loading of |
| | Ser(tBu)—OH | | AA eq/DIEA | | Fmoc-L- |
| | | | eq = 0.8/3.0 | | Ser(tBu)—OH on |
| | | | 7 vol DCM | | CTC resin, |
| | | | | | measure resin |
| | | | | | loading by |
| | | | | | qNMR |
| 2 | Fmoc-L- | 20% PIP/DMF | AA/Oxyma/DIC = | 4 h | Start with Fmoc- |
| | Thr(tBu)—OH | 3 × 30 min, | 3.0/3.0/3.3 | | L-Ser(tBu) on |
| | | 25° C. | 13 vol DMF | | CTC Resin; 0.71 |
| | | | | | mmol/g loading |
| 3 | Fmoc-L- | 20% PIP/DMF | AA/Oxyma/DIC = | 4 h | |
| | Phe—OH | 3 × 30 min, | 3.0/3.0/3.3 | | |
| | | 25° C. | 13 vol DMF | | |
| 4 | Fmoc-L- | 20% doc/DMF | AA/Oxyma/DIC = | 4 h | |
| | Thr(tBu)—OH | 3 × 30 min, | 3.0/3.0/3.3 | | |
| | | 25° C. | 13 vol DMF | | |

Four crystalline solid forms of the T-F-T-S tetramer were identified: Form A, Form B, Form C, and Form D. T-F-T-S Form A is a crystalline material generated from organic solvent/heptane mixtures such as IPA/heptane, EtOAc/heptane, MEK/heptane, iPrOAc/heptane, and THF/heptane, etc. It represents a family of iso-structural solvates. T-F-T-S Form B is a semi-disordered crystalline material that is generated from MTBE. T-F-T-S Form C is a semi-disordered crystalline material and is generated from ACN or 1:1 ACN/H₂O. T-F-T-S Form D is crystalline and a desolvated or partially desolvated material from T-F-T-S Form A, with variable amounts of solvent(s) in the unit cell.

XRPD patterns of crystalline T-F-T-S tetramers were obtained on a Bruker D8 Endeavor X-ray powder diffractometer, equipped with a CuKα (1.5418 Å) source and a Linxeye detector, operating at 40 kV and 40 mA. The sample was scanned between 4 and 30 2θ°, with a step size of 0.009 2θ° and a scan rate of 0.25 seconds/step, and using 0.3° primary slit opening, and 3.9° PSD opening. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### T-F-T-S Form A

In one experiment, T-F-T-S Form A was prepared by adding 0.5 mL of 1:1 vol/vol IPA/heptane to 104.8 mg of amorphous T-F-T-S followed by addition of another 0.5 mL heptane. The sample was stirred overnight at ambient conditions resulting a thin slurry. The sample was uncapped and left at ambient conditions for 3 days to evaporate the solvents off. The obtained white solids were consistent with T-F-T-S Form A.

In another experiment, T-F-T-S Form A was prepared by stirring 62.1 mg of amorphous T-F-T-S in 300 μL of 10:90 vol/vol MEK/heptane at 5° C. A thick white slurry was obtained. After 5 days, the sample was centrifuged at ambient conditions, the liquid phase was decanted, and the resulting white solids were consistent with T-F-T-S Form A.

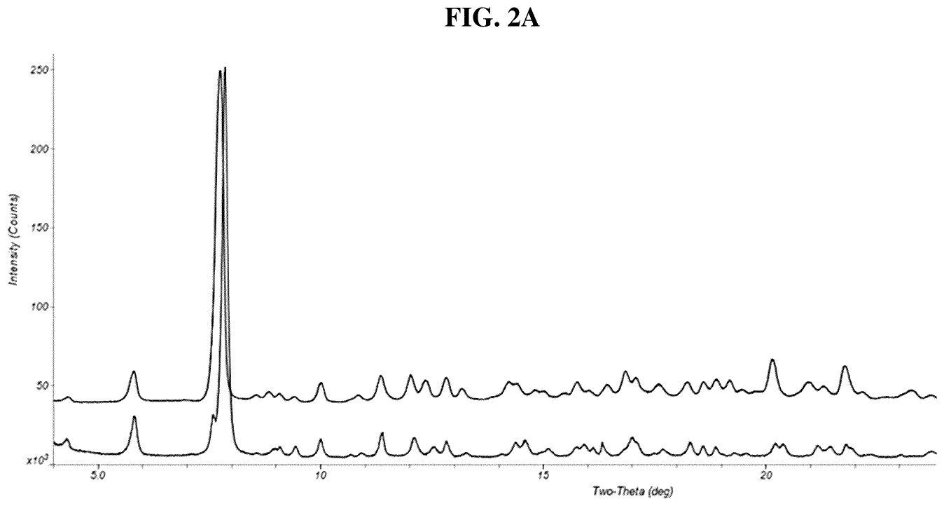

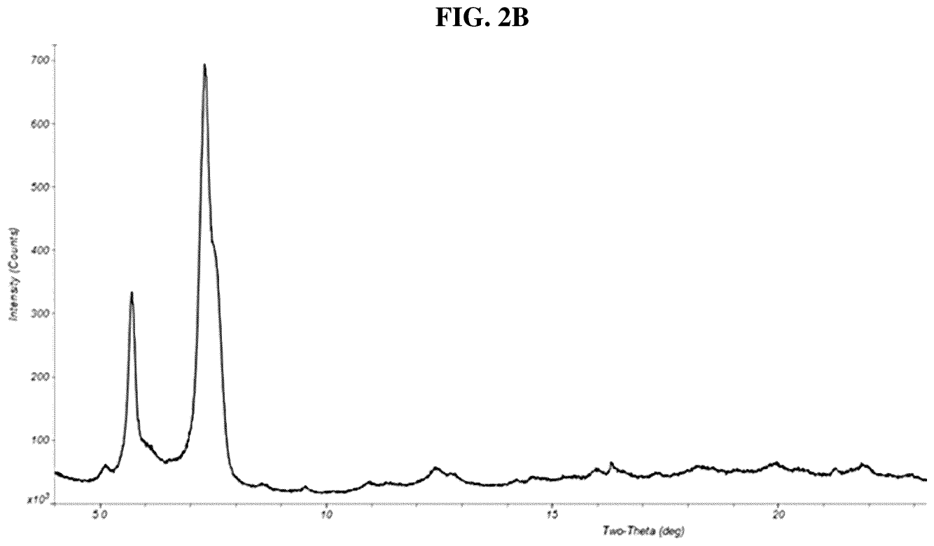

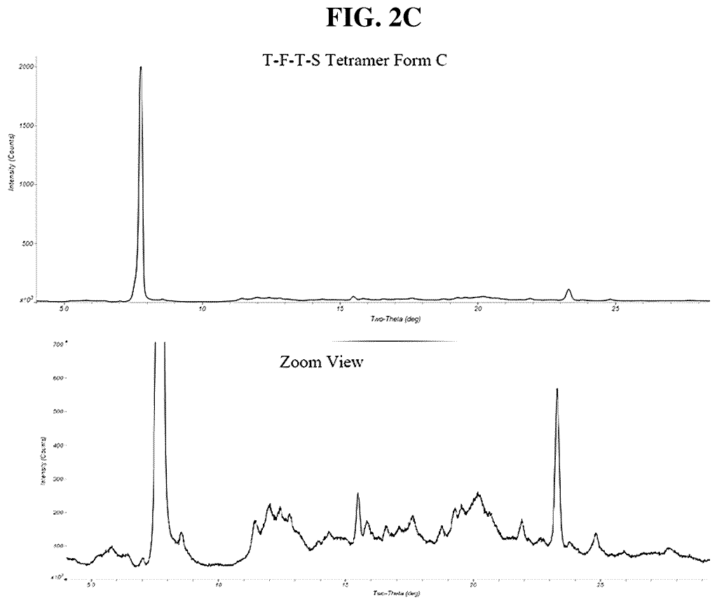

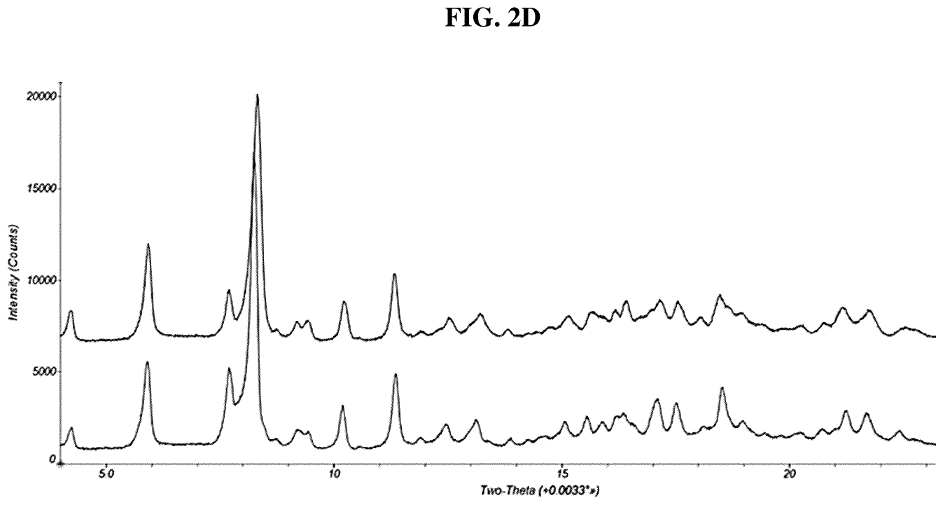

Prepared samples of the T-F-T-S Form A were characterized by an XRPD pattern using CuKα radiation as having diffraction peaks (2-theta values) as described in Table 4 below, and in particular having peaks at 4.3 and 12.8 °2-Theta, in combination with one or more of the peaks selected from 5.8, 7.7-7.9, 10.0, 10.8-10.9, 11.3-11.4, 12.0-12.1, and 21.8 °2-Thea; with a tolerance for the diffraction angles of 0.2 degrees. A representative XRPD pattern of T-F-T-S Form A is shown in FIG. 2A.

TABLE 4
X-ray powder diffraction peaks of T-F-T-S Form A
T-F-T-S Form A

| | Angle | Relative Intensity |
|---|---|---|
| Peak | (°2-Theta) +/− 0.2° | (% of most intense peak) |
| 1 | 4.3 | 1.2%-2.4% |
| 2 | 5.8 | 9.1%-10.0% |
| 3 | 7.7-7.9 | 100.0% |
| 4 | 8.5-8.6 | 0.4%-0.8% |
| 5 | 8.8-8.9 | 1.5%-1.7% |
| 6 | 9.1 | 1.3%-2.1% |
| 7 | 9.4 | 1.2%-2.5% |
| 8 | 10.0 | 4.7%-5.8% |
| 9 | 10.8-10.9 | 0.8%-1.6% |
| 10 | 11.3-11.4 | 6.1%-7.3% |
| 11 | 12.0-12.1 | 4.4%-7.4% |
| 12 | 12.3-12.6 | 1.7%-4.0% |
| 13 | 12.8 | 4.0%-5.7% |
| 14 | 13.2-13.3 | 1.1%-2.5% |
| 15 | 14.2-14.4 | 5.3%-3.6% |
| 16 | 14.4-14.6 | 3.6%-3.7% |
| 17 | 15.7-15.8 | 1.5%-4.2% |
| 18 | 16.3-16.4 | 2.7% |
| 19 | 16.8-17.0 | 4.7%-6.3% |
| 20 | 17.6-17.7 | 1.7%-2.8% |
| 21 | 18.2-18.3 | 3.3%-3.6% |
| 22 | 18.6 | 2.4% |
| 23 | 18.9 | 2.6%-2.3% |
| 24 | 20.1-20.2 | 3.4%-10.8% |
| 25 | 20.9-21.1 | 2.8%-3.0% |
| 26 | 21.8 | 1.8%-8.9% |

### T-F-T-S Tetramer Form B

2×0.2 ml, of MTBE was added to 100.2 mg of amorphous T-F-T-S and the sample was capped and stirred/vortexed at ambient conditions resulting a thick white slurry. The wet solids from the slurry were consistent with T-F-T-S Form B.

A prepared sample of T-F-T-S Form B was characterized by an XRPD pattern using CuKα radiation as having diffraction peaks (2-theta values) as described in Table 5 below, and in particular having peaks at 5.7 and 7.3 °2-Theta, in combination with one or more of the peaks selected from 5.1, 7.6, 9.5, and 12.4 °2-Theta; with a tolerance for the diffraction angles of 0.2 degrees. A representative XRPD pattern of T-F-T-S tetramer Form B is shown in FIG. 2B.

TABLE 5
X-ray powder diffraction peaks of T-F-T-S Form B
T-F-T-S Form B

| | Angle | Relative Intensity |
|---|---|---|
| Peak | (°2-Theta) +/− 0.2° | (% of most intense peak) |
| 1 | 5.1 | 3.0% |
| 2 | 5.7 | 43.5% |
| 3 | 7.3 | 100.0% |
| 4 | 7.6 | 56.9% |
| 5 | 9.5 | 1.4% |
| 6 | 10.9 | 1.9% |
| 7 | 12.4 | 3.8% |
| 8 | 12.8 | 2.1% |
| 9 | 14.2 | 1.0% |
| 11 | 17.3 | 1.2% |
| 12 | 18.4 | 1.7% |
| 13 | 20.0 | 2.5% |
| 14 | 21.3 | 1.4% |
| 15 | 21.9 | 2.3% |

### T-F-T-S Form C

In one experiment, 0.25 mL of 1:1 vol/vol ACN/H₂O was added to 89.6 mg of amorphous T-F-T-S and the sample was capped and stirred at ambient conditions overnight resulting a thick white slurry. The slurry was centrifuged at ambient conditions for approximate 5 minutes, and the liquid phase was decanted; the obtained wet solids were consistent with T-F-T-S Form C.

In another experiment, 3×0.1 mL of ACN was added to 56.6 mg of amorphous T-F-T-S and the sample was capped and stirred at ambient conditions for approximate 3 hours. Wet solids from the obtained thick white slurry were consistent with T-F-T-S Form C.

A prepared sample of T-F-T-S Form C was characterized by an XRPD pattern using CuKα radiation as having diffraction peaks (2-theta values) as described in Table 6 below, and in particular having a peak at 7.8 °2-Theta, in combination with one or more of the peaks selected from 8.5, 12.0, 15.5, 20.2, and 23.3 °2-Theta; with a tolerance for the diffraction angles of 0.2 degrees. A representative XRPD pattern of T-F-T-S Form C is shown in FIG. 2C.

TABLE 6
X-ray powder diffraction peaks of T-F-T-S Form C
T-F-T-S Form C

| | Angle | Relative Intensity |
|---|---|---|
| Peak | (°2-Theta) +/− 0.2° | (% of most intense peak) |
| 1 | 5.8 | 0.5% |
| 2 | 7.8 | 100.0% |
| 3 | 8.5 | 1.0% |
| 4 | 11.5 | 1.3% |
| 5 | 12.0 | 1.8% |
| 6 | 12.4 | 0.4% |
| 7 | 12.8 | 1.5% |
| 8 | 14.3 | 1.0% |
| 9 | 15.5 | 2.1% |
| 11 | 16.6 | 0.3% |
| 12 | 17.1 | 0.2% |
| 13 | 17.6 | 0.5% |
| 14 | 18.7 | 0.4% |
| 15 | 19.2 | 0.4% |
| 16 | 19.5 | 0.3% |
| 17 | 20.2 | 1.3% |
| 18 | 21.9 | 0.6% |
| 19 | 23.3 | 4.7% |

### T-F-T-S Form D

Solids of T-F-T-S Form A were air dried at ambient conditions for 7 days or longer to obtain T-F-T-S Form D.

A prepared sample of the T-F-T-S Form D was characterized by an XRPD pattern using CuKα radiation as having diffraction peaks (2-theta values) as described in Table 7 below, and in particular having peaks at 4.2 and 8.2-8.3 °2-Theta, in combination with one or more of the peaks selected from 5.9, 7.7, 9.2, 10.2, 11.3, 13.8-13.9, 15.5-15.7, 17.1 and 18.5 °2-Theta; with a tolerance for the diffraction angles of 0.2 degrees. A representative XRPD pattern of T-F-T-S Form D is shown in FIG. 2D.

TABLE 7
X-ray powder diffraction peaks of T-F-T-S Form D
T-F-T-S Form D

| | Angle | Relative Intensity |
|---|---|---|
| Peak | (°2-Theta) +/− 0.2° | (% of most intense peak) |
| 1 | 4.2 | 6.5%-11.0% |
| 2 | 5.9 | 29.0%-38.6% |
| 3 | 7.7 | 19.2%-26.1% |
| 4 | 8.2-8.3 | 100.0% |
| 5 | 9.2 | 3.6%-5.7% |
| 6 | 9.4 | 5.3%-8.4% |
| 7 | 10.2 | 14.5%-16.3% |
| 8 | 11.3 | 24.6%-26.5% |
| 9 | 12.5 | 6.0%-6.7% |
| 10 | 13.1-13.2 | 8.0%-8.5% |
| 11 | 13.8-13.9 | 2.2%-2.7% |
| 12 | 15.1 | 5.4%-6.1% |
| 13 | 15.5-15.7 | 5.6%-5.8% |
| 14 | 16.2 | 3.2%-5.2% |
| 15 | 16.3-16.4 | 6.2%-7.0% |
| 16 | 17.1 | 7.5%-10.8% |
| 17 | 17.5-17.6 | 7.7%-9.6% |
| 18 | 18.5 | 12.8%-14.1% |
| 19 | 18.9-19.0 | 2.9%-6.2% |
| 20 | 20.2-20.3 | 1.9%-2.3% |
| 21 | 20.7 | 2.2%-2.4% |
| 22 | 21.2 | 6.8%-7.3% |
| 23 | 21.7 | 7.1%-10.3% |

### Example 3: Pro-Ser(tBu)-Ser(tBu)-Gly-NH₂

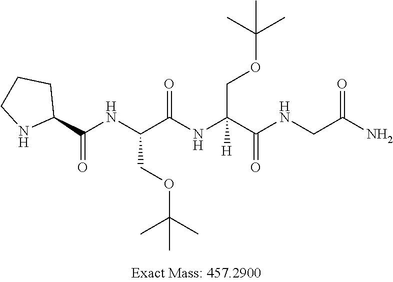 [SMILES: CC(C)(C)OC[C@H](NC(=O)[C@H](COC(C)(C)C)NC(=O)[C@@H]1CCCN1)C(=O)NCC(N)=O]

Example 3 was prepared substantially by the procedure described for Example 4, below.

Swelling of the resin: The resin (0.500 mmol) was charged to the reactor and swelled with DMF (3×10 mL×20 min).

Washing after DeFmoc: Following deprotection, the resin was washed with DMF (5×10 mL×2 min).

Washing after coupling: Following coupling, the resin was washed with DMF (5×10 mL×2 min).

Washing and drying of the resin: Following final coupling or deprotection, the resin was washed with DMF (5×10 mL×2 min), followed by DCM (5×10 mL×2 min), and drain dried under N₂ atmosphere to constant weight.

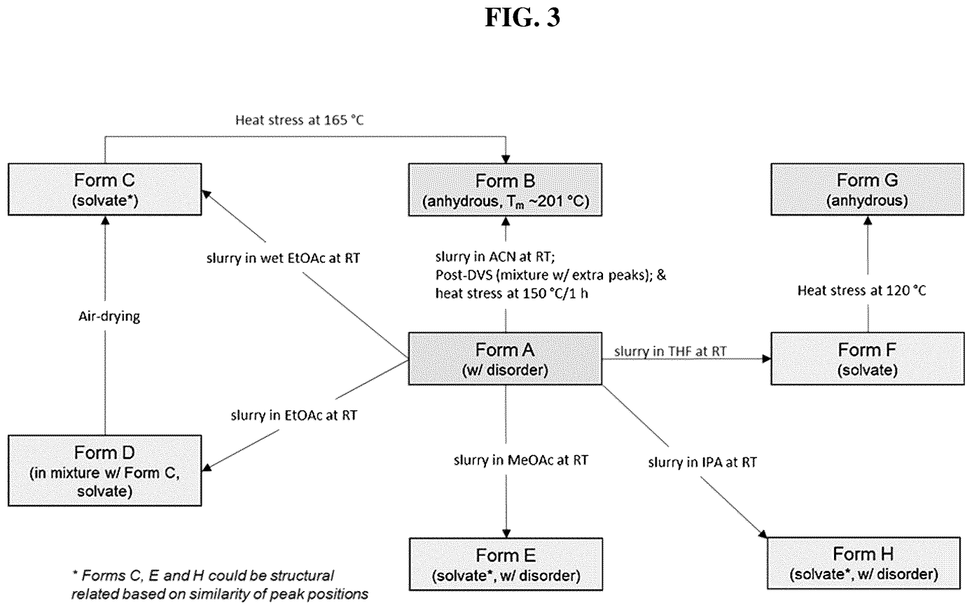

A total of seven crystalline solid forms of P-S-S-G-NH₂ were identified, including P-S-S-G-NH₂ Forms A through H. A polymorph map to describe their relationship is shown in the scheme of FIG. 3.

Among these forms, P-S-S-G-NH₂ Forms B and G are anhydrous/unsolvated while C, D, E, F, and H are solvated. Forms A, E and H are disordered crystalline materials.

The XRPD patterns of crystalline P-S-S-G-NH₂ were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. In some cases, the sample was scanned between 4 and 30 2θ° with a scan rate of 0.25 seconds/step. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### P-S-S-G-NH₂ Form A

12.7549 g of resin of the solid-phase peptide synthesis for P-S-S-G-NH₂ tetramer was charged to a 150 mL fritted reactor equipped with an overhead stirred. 128 mL of 5% TFA/DCM cleavage cocktail (6.4 mL TFA, 121.6 mL DCM) was charged to the reactor and the resultant solution was stirred for 30 minutes. The reactor was drained and washed with DCM (2×120 mL). MTBE was added to the filtrate to precipitate the tetramer. The resultant precipitate was filtered through a buchner funnel and allowed to air-dry under vacuum suction for overnight, then dried in the vacuum oven at 35° C. overnight to remove residual solvents. The obtained solids were consistent with P-S-S-G-NH₂ Form A.

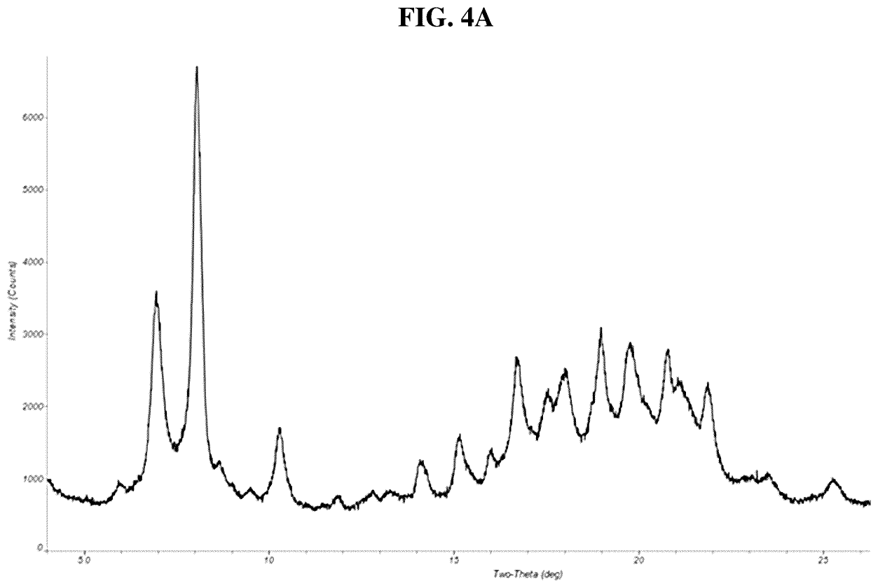

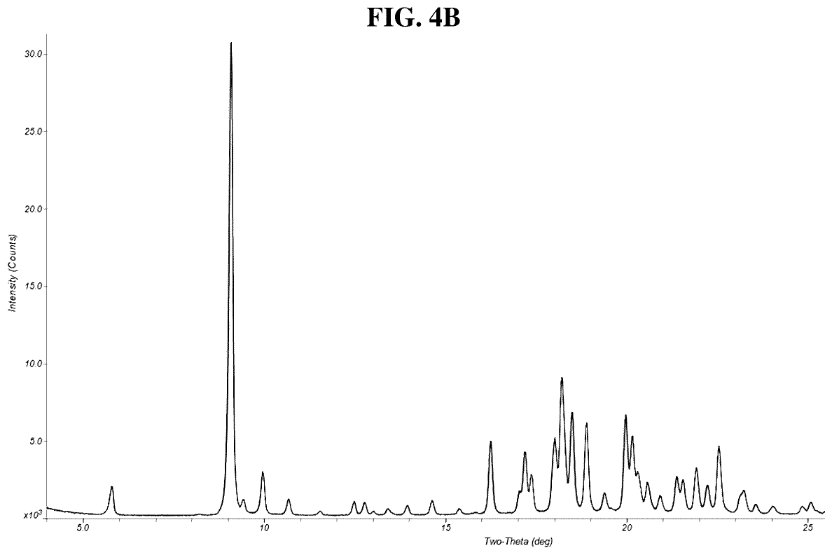

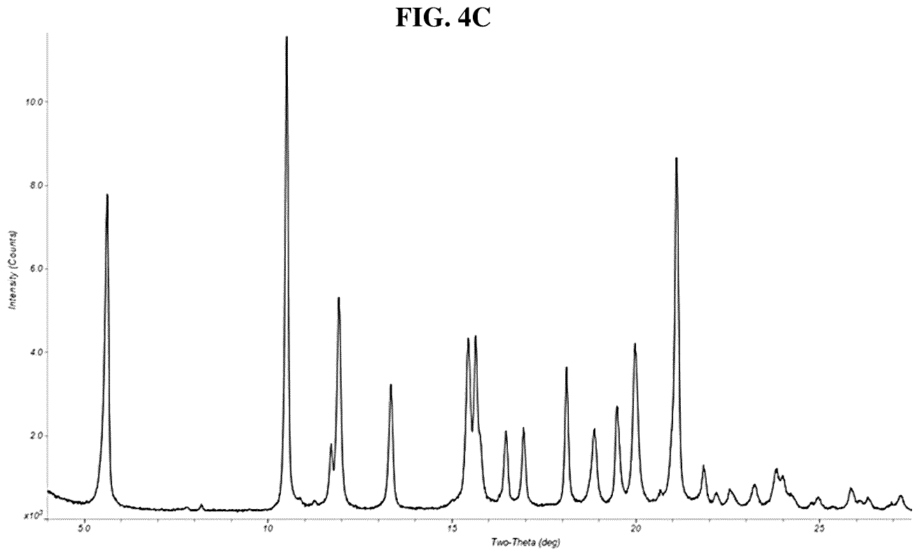

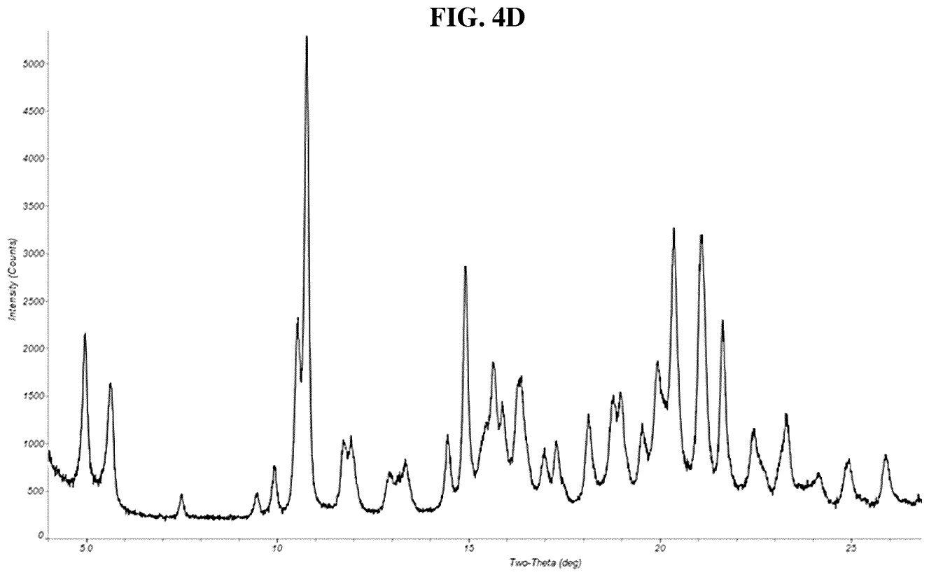

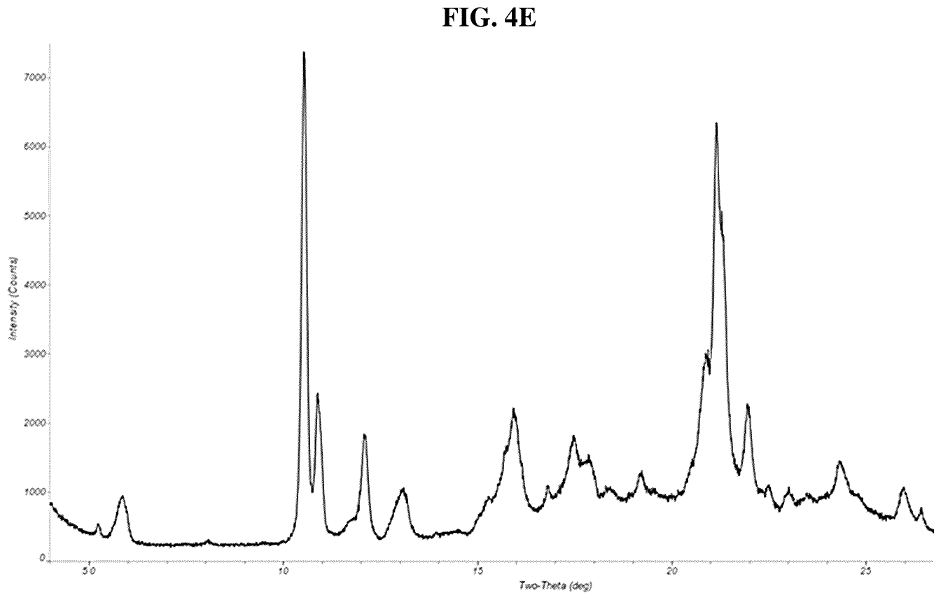

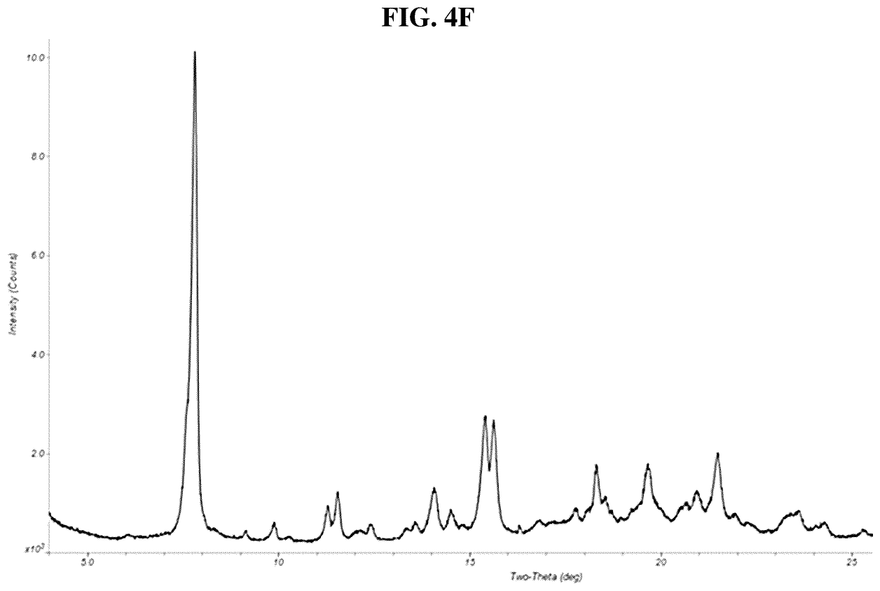

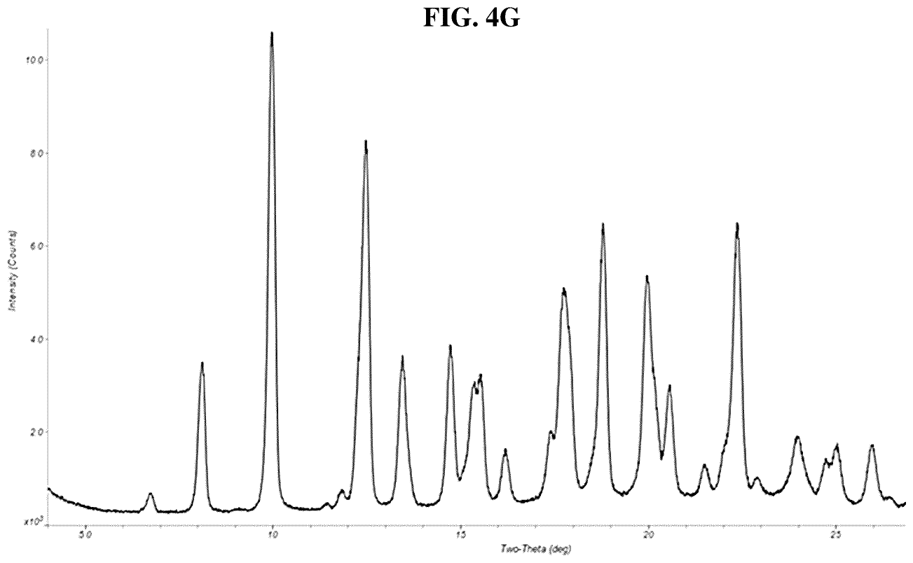

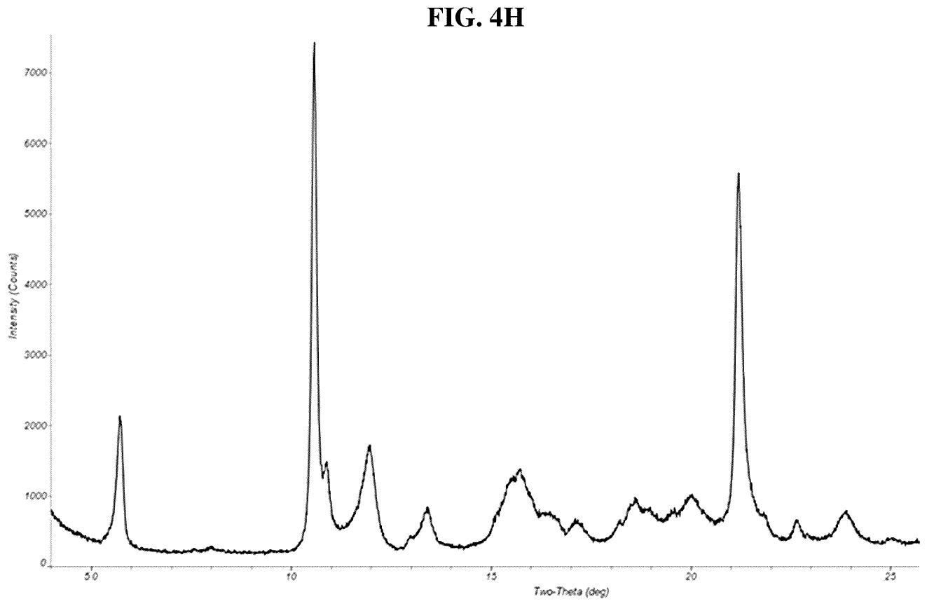

A prepared sample of the P-S-S-G-NH₂ Form A was characterized by an XRPD pattern using CuKα radiation as having diffraction peaks (2-theta values) as described in Table 8 below, and in particular having peaks at 7.0 and 8.0 °2-Theta, in combination with one or more of the peaks selected from 10.3, 14.1, 16.7, and 19.0 °2-Theta; with a tolerance for the diffraction angles of 0.2 degrees. A representative XRPD pattern of P-S-S-G-NH₂ Form A is shown in FIG. 4A.

TABLE 8
X-ray powder diffraction peaks of P—S—S-G-NH₂ Form A
P—S—S-G-NH₂ Form A

| | Angle | Relative Intensity |
|---|---|---|
| Peak | (°2-Theta) +/− 0.2° | (% of most intense peak) |
| 1 | 7.0 | 46.7% |
| 2 | 8.0 | 100.0% |
| 3 | 10.3 | 16.5% |
| 4 | 14.1 | 8.2% |
| 5 | 15.2 | 11.8% |
| 6 | 16.0 | 4.2% |
| 7 | 16.7 | 28.6% |
| 8 | 17.5 | 5.4% |
| 9 | 18.0 | 11.7% |
| 10 | 19.0 | 21.8% |
| 11 | 19.7 | 17.0% |
| 12 | 20.8 | 16.7% |
| 13 | 21.1 | 9.6% |
| 14 | 21.9 | 22.6% |

### P-S-S-G-NH₂ Form B

In one experiment, 80.9 mg solids of P-S-S-G-NH₂ Form A were stirred in 0.8 mL ACN at ambient conditions resulting a white suspension. The slurry was isolated after 8 days by centrifuging the mixture at ambient conditions for 5 minutes using a centrifuge tube filter. The obtained white solids were consistent with P-S-S-G-NH₂ Form B.

In another experiment, 5.3 mg solids of P-S-S-G-NH₂ Form A were added to a clean TGA pan, heated to 150° C. on TGA and held at the temperature for 60 minutes. The obtained off-white solids were consistent with P-S-S-G-NH₂ Form B.

In yet another experiment, 7.0 mg solids of P-S-S-G-NH₂ Form C were added to a clean TGA pan, heated to 165° C. on TGA and held at the temperature for 5 minutes. The obtained white solids were consistent with P-S-S-G-NH₂ Form B.

A prepared sample of the P-S-S-G-NH₂ Form B was characterized by an XRPD pattern using CuKα radiation as having diffraction peaks (2-theta values) as described in Table 9 below, and in particular having a peak at 9.0 °2-Theta, in combination with one or more of the peaks selected from 5.7, 9.9, 16.2 18.1, and 18.4 °2-Theta; with a tolerance for the diffraction angles of 0.2 degrees. A representative XRPD pattern of P-S-S-G-NH₂ Form B is shown in FIG. 4B.

TABLE 9
X-ray powder diffraction peaks of P—S—S-G-NH₂ Form B
P—S—S-G-NH₂ Form B

| | Angle | Relative Intensity |
|---|---|---|
| Peak | (°2-Theta) +/− 0.2° | (% of most intense peak) |
| 1 | 5.7 | 6.7% |
| 2 | 9.0 | 100.0% |
| 3 | 9.3 | 2.8% |
| 4 | 9.9 | 8.6% |
| 5 | 10.6 | 3.4% |
| 6 | 12.4 | 2.8% |
| 7 | 12.7 | 2.6% |
| 8 | 13.9 | 2.0% |
| 9 | 14.6 | 3.2% |
| 10 | 16.2 | 16.7% |
| 11 | 17.1 | 15.3% |
| 12 | 17.3 | 9.3% |
| 13 | 17.9 | 17.1% |
| 14 | 18.1 | 34.3% |
| 15 | 18.4 | 24.6% |
| 16 | 18.8 | 21.2% |
| 17 | 19.3 | 4.2% |
| 18 | 19.9 | 24.6% |
| 19 | 20.1 | 18.3% |
| 20 | 20.8 | 3.4% |
| 21 | 21.3 | 9.1% |
| 22 | 21.5 | 7.6% |
| 23 | 21.8 | 10.3% |
| 24 | 22.1 | 5.8% |
| 25 | 22.5 | 17.4% |

### P-S-S-G-NH₂ Form C

In one experiment, 83.6 mg solids of P-S-S-G-NH₂ Form A were stirred in 0.8 mL wet EtOAc at ambient conditions resulting a white suspension. The slurry was isolated after 8 days by centrifuging the mixture at ambient conditions for 5 minutes using a centrifuge tube filter. The obtained white solids were consistent with P-S-S-G-NH₂ Form C.

In another experiment, a mixture containing P-S-S-G-NH₂ Form C and Form D was air dried at ambient conditions for overnight resulting a single phase of P-S-S-G-NH₂ Form C.

A prepared sample of the P-S-S-G-NH₂ Form C was characterized by an XRPD pattern using CuKα radiation as having diffraction peaks (2-theta values) as described in Table 10 below, and in particular having peaks at 5.6 and 10.5 °2-Theta, in combination with one or more of the peaks selected from 11.9, 13.3, and 21.1 °2-Theta; with a tolerance for the diffraction angles of 0.2 degrees. A representative XRPD pattern of P-S-S-G-NH₂ Form C is shown in FIG. 4C.

TABLE 10
X-ray powder diffraction peaks of P—S—S-G-NH₂ Form C
P—S—S-G-NH₂ Form C

| | Angle | Relative Intensity |
|---|---|---|
| Peak | (°2-Theta) +/− 0.2° | (% of most intense peak) |
| 1 | 5.6 | 65.6% |
| 2 | 10.5 | 100.0% |
| 3 | 11.7 | 12.8% |
| 4 | 11.9 | 44.4% |
| 5 | 13.3 | 26.2% |
| 6 | 15.4 | 35.1% |
| 7 | 15.6 | 34.9% |
| 8 | 16.4 | 14.6% |
| 9 | 16.9 | 15.6% |
| 10 | 18.1 | 28.7% |
| 11 | 18.8 | 15.5% |
| 12 | 19.4 | 20.3% |
| 13 | 19.9 | 32.2% |
| 14 | 21.1 | 72.1% |
| 15 | 21.8 | 7.5% |

### P-S-S-G-NH₂ Form D

76.0 mg solids of P-S-S-G-NH₂ Form A were stirred in 0.8 mL EtOAc at ambient conditions resulting a thick white slurry. Additional 0.5 mL EtOAc was added the next day and the sample was stirred at ambient for another 7 days. The mixture was then transferred to a centrifuge tube filter and centrifuged at ambient conditions for 5 minutes and solids were left at ambient conditions for air drying for about 4 hours. A mixture of white solids and translucent chunks were obtained. By XRPD, the sample was composed of Form C and Form D.

A prepared sample of the P-S-S-G-NH₂ Form D was characterized by an XRPD pattern using CuKα radiation as having diffraction peaks (2-theta values) as described in Table 11 below, and in particular having a peak at 4.9 °2-Theta, in combination with one or more of the peaks selected from 10.7, 14.8, and 20.3 °2-Theta; with a tolerance for the diffraction angles of 0.2 degrees. A representative XRPD pattern of P-S-S-G-NH₂ Form D is shown in FIG. 4D.

TABLE 11
X-ray powder diffraction peaks of P—S—S-G-NH₂ Form D
P—S—S-G-NH₂ tetramer Form D

| | Angle | Relative Intensity |
|---|---|---|
| Peak | (°2-Theta) +/− 0.2° | (% of most intense peak) |
| 1 | 4.9 | 31.7% |
| 2 | 7.4 | 5.1% |
| 3 | 9.4 | 4.2% |
| 4 | 9.8 | 9.3% |
| 5 | 10.7 | 100.0% |
| 6 | 12.8 | 5.2% |
| 7 | 14.4 | 15.5% |
| 8 | 14.8 | 45.6% |
| 9 | 20.3 | 51.0% |
| 10 | 21.5 | 33.0% |

### P-S-S-G-NH₂ Form E

66.9 mg solids of P-S-S-G-NH₂ Form A were stirred in 0.8 mL MeOAc at ambient conditions resulting a white suspension. The slurry was isolated after 8 days by centrifuging the mixture at ambient conditions for 5 minutes using a centrifuge tube filter. Solids were left at ambient for air drying for about 4 hours resulting a mixture of white solids and translucent chunks displaying an XRPD pattern that was consistent with P-S-S-G-NH₂ Form E.

A prepared sample of the P-S-S-G-NH₂ Form E was characterized by an XRPD pattern using CuKα radiation as having diffraction peaks (2-theta values) as described in Table 12 below, and in particular having peaks at 13.1 and 17.5 °2-Theta, in combination with one or more of the peaks selected from 5.9, 10.5, 10.9, and 15.9 °2-Theta; with a tolerance for the diffraction angles of 0.2 degrees. A representative XRPD pattern of P-S-S-G-NH₂ Form E is shown in FIG. 4E.

TABLE 12
X-ray powder diffraction peaks of P—S—S-G-NH₂ Form E
P—S—S-G-NH₂ Form E

| | Angle | Relative Intensity |
|---|---|---|
| Peak | (°2-Theta) +/− 0.2° | (% of most intense peak) |
| 1 | 5.9 | 10.2% |
| 2 | 10.5 | 100.0% |
| 3 | 10.9 | 30.9% |
| 4 | 12.1 | 20.9% |
| 5 | 13.1 | 10.3% |
| 6 | 15.9 | 24.8% |
| 7 | 17.5 | 14.4% |
| 8 | 19.2 | 5.1% |
| 9 | 20.9 | 25.7% |
| 10 | 21.1 | 72.6% |
| 11 | 21.9 | 21.2% |
| 12 | 24.3 | 8.4% |

### P-S-S-G-NH₂ Form F

71.1 mg solids of P-S-S-G-NH₂ Form A were stirred in 0.8 mL THF at ambient conditions resulting a white suspension. The slurry was isolated after 8 days by centrifuging the mixture at ambient conditions for 5 minutes using a centrifuge tube filter and the material was left at ambient for air drying for about 4 hours. The obtained white solids were consistent with P-S-S-G-NH₂ Form F.

A prepared sample of the P-S-S-G-NH₂ Form F was characterized by an XRPD pattern using CuKα radiation as having diffraction peaks (2-theta values) as described in Table 13 below, and in particular having a peak at 7.8 °2-Theta, in combination with one or more of the peaks selected from 11.3, 11.5, 15.4, and 15.6 °2-Theta; with a tolerance for the diffraction angles of 0.2 degrees. A representative XRPD pattern of P-S-S-G-NH₂ Form F is shown in FIG. 4F.

TABLE 13
X-ray powder diffraction peaks of P—S—S-G-NH₂ Form F
P—S—S-G-NH₂ Form F

| | Angle | Relative Intensity |
|---|---|---|
| Peak | (°2-Theta) +/− 0.2° | (% of most intense peak) |
| 1 | 7.8 | 100.0% |
| 2 | 11.3 | 7.3% |
| 3 | 11.5 | 9.3% |
| 4 | 14.0 | 8.3% |
| 5 | 14.5 | 3.7% |
| 6 | 15.4 | 23.1% |
| 7 | 15.6 | 22.6% |
| 8 | 18.3 | 11.1% |
| 9 | 19.6 | 11.7% |
| 10 | 21.5 | 15.2% |

### P-S-S-G-NH₂ Form G

12.2 mg solids of P-S-S-G-NH₂ Form F were added to a clean TGA pan, heated to 125° C. on TGA and held at the temperature for 5 minutes. The obtained white solids were consistent with P-S-S-G-NH₂ Form G.

A prepared sample of the P-S-S-G-NH₂ Form G was characterized by an XRPD pattern using CuKα radiation as having diffraction peaks (2-theta values) as described in Table 14 below, and in particular having peaks at 10.0 and 12.5 °2-Theta, in combination with one or more of the peaks selected from 8.1, 14.7, 18.8, and 22.4 °2-Theta; with a tolerance for the diffraction angles of 0.2 degrees. A representative XRPD pattern of P-S-S-G-NH₂ Form G is shown in FIG. 4G.

TABLE 14
X-ray powder diffraction peaks of P—S—S-G-NH₂ Form G
P—S—S-G-NH₂ Form G

| | Angle | Relative Intensity |
|---|---|---|
| Peak | (°2-Theta) +/− 0.2° | (% of most intense peak) |
| 1 | 6.7 | 4.0% |
| 2 | 8.1 | 31.1% |
| 3 | 10.0 | 100.0% |
| 4 | 12.5 | 76.6% |
| 5 | 13.5 | 30.7% |
| 6 | 14.7 | 33.3% |
| 7 | 15.3 | 11.1% |
| 8 | 15.5 | 25.7% |
| 9 | 16.2 | 10.4% |
| 10 | 17.8 | 44.8% |
| 11 | 18.8 | 55.0% |
| 12 | 20.0 | 45.6% |
| 13 | 20.6 | 23.2% |
| 14 | 21.5 | 5.6% |
| 15 | 22.4 | 55.7% |

### P-S-S-G-NH₂ Form H

71.6 mg solids of P-S-S-G-NH₂ Form A were stirred in 0.8 mL IPA at ambient conditions resulting a thick white slurry. Additional 0.5 mL IPA was added the next day and the sample was stirred at ambient for another 7 days. The mixture was then transferred to a centrifuge tube filter and centrifuged at ambient conditions for 5 minutes and solids were left at ambient conditions for air drying for about 4 hours. A mixture of white solids and translucent chunks were obtained which displays an XRPD pattern that was consistent with P-S-S-G-NH₂ Form H.

A prepared sample of the P-S-S-G-NH₂ Form H was characterized by an XRPD pattern using CuKα radiation as having diffraction peaks (2-theta values) as described in Table 15 below, and in particular having peaks at 10.8 and 21.1 °2-Theta, in combination with one or more of the peaks selected from 5.6, 10.5, 11.9, and 15.4 °2-Theta; with a tolerance for the diffraction angles of 0.2 degrees. A representative XRPD pattern of P-S-S-G-NH₂ Form H is shown in FIG. 4H.

TABLE 15
X-ray powder diffraction peaks of P—S—S-G-NH₂ Form H
P—S—S-G-NH₂ Form H

| | Angle | Relative Intensity |
|---|---|---|
| Peak | (°2-Theta) +/− 0.2° | (% of most intense peak) |
| 1 | 5.6 | 11.1% |
| 2 | 10.5 | 67.8% |
| 3 | 10.8 | 12.0% |
| 4 | 11.9 | 17.0% |
| 5 | 13.3 | 5.4% |
| 6 | 15.4 | 11.8% |
| 7 | 16.4 | 5.3% |
| 8 | 17.0 | 2.6% |
| 9 | 18.5 | 5.6% |
| 10 | 18.8 | 4.4% |
| 11 | 19.9 | 6.2% |
| 12 | 21.1 | 100.0% |
| 13 | 22.5 | 4.5% |
| 14 | 23.8 | 10.8% |

### Example 4: Gly-Pro-Ser(tBu)-Ser(tBu)-Gly-NH₂

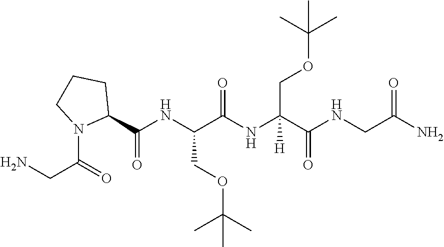 [SMILES: CC(C)(C)OC[C@H](NC(=O)[C@H](COC(C)(C)C)NC(=O)[C@@H]1CCCN1C(=O)CN)C(=O)NCC(N)=O]

Swelling of the resin: The resin (0.500 mmol) was charged to the reactor and swelled with DMF (3×10 mL×20 min).

Washing after DeFmoc: Following deprotection, the resin was washed with DMF (5×10 mL×2 min).

Washing after coupling: Following coupling, the resin was washed with DMF (5×10 mL×2 min).

Washing and drying of the resin: Following final coupling or deprotection, the resin was washed with DMF (5×10 mL×2 min), followed by DCM (5×10 mL×2 min), and drain dried under N₂ atmosphere to constant weight.

Preparation 1 Procedure

| | | H₂N-G-P-S(tBu)-S(tBu)-G-NH₂ | | | |
|---|---|---|---|---|---|
| | | DeFmoc | | | |
| | | reagents & | | | |
| | Fmoc- | DeFmoc | Coupling | Coupling | |
| Cycle | AA-OH | condition | reagents | Time | Comments |
| 1 | Fmoc- | 20% PIP/DMF | AA/Oxyma/DIC = | 4 h | Start with Sieber |
| | Gly-OH | 5 + 20 + 30 | 3.0/3.0/3.3 | | Amide Resin; |
| | | min, 25° C. | 13 vol DMF | | 0.75 mmol/g |
| | | | | | loading |
| 2 | Fmoc-L- | 20% PIP/DMF | AA/Oxyma/DIC = | 4 h | |
| | Ser(tBu)- | 5 + 20 + 30 | 3.0/3.0/3.3 | | |
| | OH | min, 25° C. | 13 vol DMF | | |
| 3 | Fmoc-L- | 20% PIP/DMF | AA/Oxyma/DIC = | 4 h | |
| | Ser(tBu)- | 5 + 20 + 30 | 3.0/3.0/3.3 | | |
| | OH | min, 25° C. | 13 vol DMF | | |
| 4 | Fmoc-L- | 20% PIP/DMF | AA/Oxyma/DIC = | 4 h | |
| | Pro-OH | 5 + 20 + 30 | 3.0/3.0/3.3 | | |
| | | min, 25° C. | 13 vol DMF | | |
| 5 | Fmoc- | 20% PIP/DMF | AA/Oxyma/DIC = | 8 h | Following |
| | Gly-OH | 5 + 20 + 30 | 3.0/3.0/3.3 | | coupling, Fmoc |
| | | min, 25° C. | 13 vol DMF | | is removed using |
| | | | | | 20% PIP/DMF, |
| | | | | | 5 + 20 + 30 |
| | | | | | min, 25° C. |

Cleavage from the Resin:

5% TFA/DCM (10 vol) was added to the resin and the reactor was stirred for 30 minutes. The reactor was drained and the resin washed with DCM (2×5 vol). The filtrate was added to pre-cooled MTBE:Heptane (1:1, 10 vol with respect to cleavage solution) and then centrifuged (3000 rpm×10 min). The supernatant was discarded and fresh cold MTBE:Heptane (5 vol) was added and the mixture centrifuged (3000 rpm×5 min). The supernatant was discarded and the process was repeated once more with fresh MTBE:Heptane. The supernatant was discarded and the resultant material was placed in a vacuum oven for 14 hours at 34° C. to afford Preparation 1.

Two crystalline solid forms of G-P-S-S-G-NH₂ pentamer were identified, including G-P-S-S-G-NH₂ Form A and Form B. G-P-S-S-G-NH₂ Form A is a crystalline solvate which physically is not stable and converts to Form B easily during isolation or upon air-drying. G-P-S-S-G-NH₂ Form B is a crystalline anhydrous/unsolvated form.

The XRPD patterns of crystalline G-P-S-S-G-NH₂ pentamers were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. For cases of G-P-S-S-G-NH₂ Form A, the sample was scanned between 4 and 25 200 with a scan rate of 0.1 seconds/step. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### G-P-S-S-G-NH₂ Form A

G-P-S-S-G-NH₂ Form A was prepared in methanol (MeOH)/tetrahydrofuran (THF). 71.0 mg amorphous G-P-S-S-G-NH₂ was dissolved in 0.2 mL MeOH at ambient temperature; 3×0.2 mL of THF was added to the solution and the sample was stirred at ambient conditions overnight result soft white solids that were consistent with G-P-S-S-G-NH₂ Form A when analyzed wet.

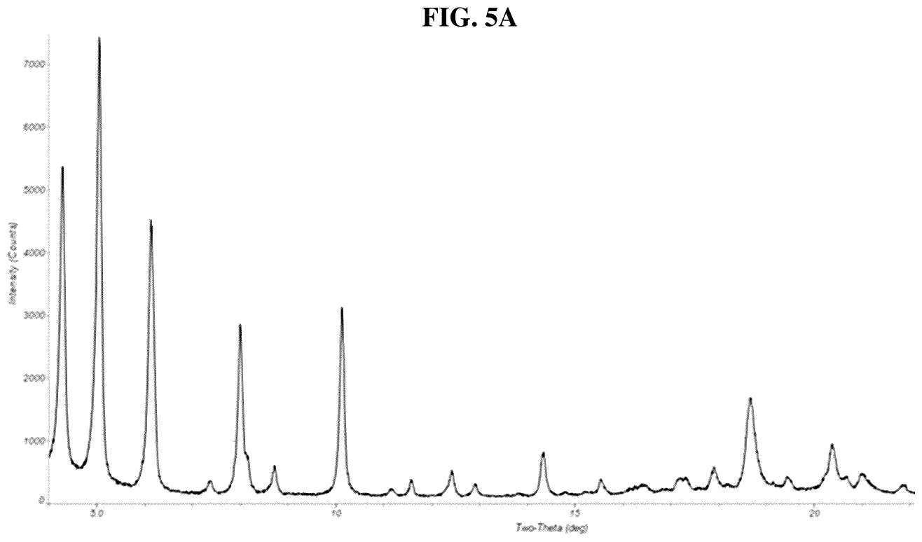

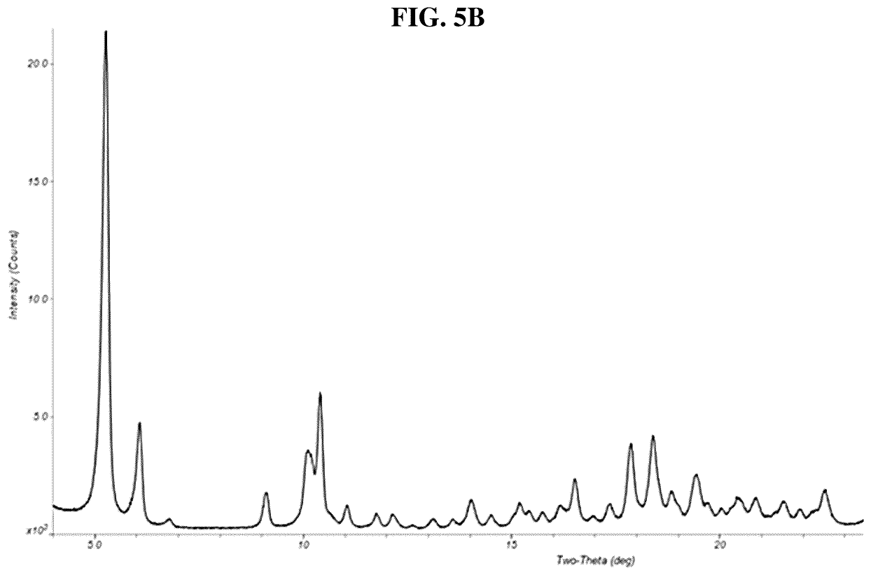

A prepared sample of the G-P-S-S-G-NH₂ Form A was characterized by an XRPD pattern using CuKα radiation as having diffraction peaks (2-theta values) as described in Table 16 below, and in particular having a peak at 4.3 °2-Theta, in combination with one or more of the peaks selected from the group consisting of 5.1, 6.1, 8.0, 10.1, and 18.7 °2-Theta; with a tolerance for the diffraction angles of 0.2 degrees. A representative XRPD pattern of G-P-S-S-G-NH₂ Form A is shown in FIG. 5A.

TABLE 16
X-ray powder diffraction peaks of G-P—S—S-G-NH₂ Form A
G-P—S—S-G-NH₂ Form A

| | Angle | Relative Intensity |
|---|---|---|
| Peak | (°2-Theta) +/− 0.2° | (% of most intense peak) |
| 1 | 4.3 | 67.7% |
| 2 | 5.1 | 100.0% |
| 3 | 6.1 | 62.0% |
| 4 | 8.0 | 38.5% |
| 5 | 8.7 | 6.0% |
| 6 | 10.1 | 43.2% |
| 7 | 11.6 | 3.4% |
| 8 | 12.4 | 5.5% |
| 9 | 12.9 | 2.7% |
| 10 | 14.3 | 9.6% |
| 11 | 15.6 | 3.2% |
| 12 | 17.9 | 4.8% |
| 13 | 18.7 | 20.4% |
| 14 | 20.4 | 9.9% |
| 15 | 21.0 | 3.0% |

### G-P-S-S-G-NH₇ Form B

In the above example for G-P-S-S-G-NH₂ Form A, additional 3×0.2 mL of THF and 1×0.2 mL of MeOH were added to the sample; the white slurry was stirred at ambient conditions for 9 days and then isolated by centrifuge using a centrifuge tube filter. The obtained solids were consistent with G-P-S-S-G-NH₂ Form B and remained as Form B after vacuum drying at 32-33° C. for 1 day.

A prepared sample of the G-P-S-S-G-NH₂ Form B was characterized by an XRPD pattern using CuKα radiation as having diffraction peaks (2-theta values) as described in Table 17 below, and in particular having peaks at 5.2 and 9.0 °2-Theta, in combination with one or more of the peaks selected from the group consisting of 6.7, 10.0, 10.3, 16.4, 17.8, 18.3, and 19.4 °2-Theta; with a tolerance for the diffraction angles of 0.2 degrees. A representative XRPD pattern of G-P-S-S-G-NH₂ Form B is shown in FIG. 5B.

TABLE 17
X-ray powder diffraction peaks of G-P—S—S-G-NH₂ Form B
G-P—S—S-G-NH₂ pentamer Form B

| | Angle | Relative Intensity |
|---|---|---|
| Peak | (°2-Theta) +/− 0.2° | (% of most intense peak) |
| 1 | 5.2 | 100.0% |
| 2 | 6.0 | 21.2% |
| 3 | 6.7 | 1.4% |
| 4 | 9.0 | 8.0% |
| 5 | 10.0 | 18.5% |
| 6 | 10.3 | 31.7% |
| 7 | 11.0 | 4.5% |
| 8 | 11.7 | 3.1% |
| 9 | 12.1 | 3.0% |
| 10 | 13.0 | 2.2% |
| 11 | 14.0 | 6.7% |
| 12 | 14.4 | 3.0% |
| 13 | 15.1 | 7.0% |
| 14 | 16.1 | 5.0% |
| 15 | 16.4 | 12.7% |
| 16 | 17.3 | 5.1% |
| 17 | 17.8 | 24.6% |
| 18 | 18.3 | 23.7% |
| 19 | 18.8 | 6.3% |
| 20 | 19.4 | 14.6% |
| 21 | 20.4 | 5.7% |
| 22 | 20.8 | 5.8% |
| 23 | 21.4 | 6.9% |
| 24 | 22.4 | 14.0% |

### Example 5 Fmoc-Thr(tBu)-Phe-Thr(tBu)-Ser(tBu)-Asp(OtBu)-OH

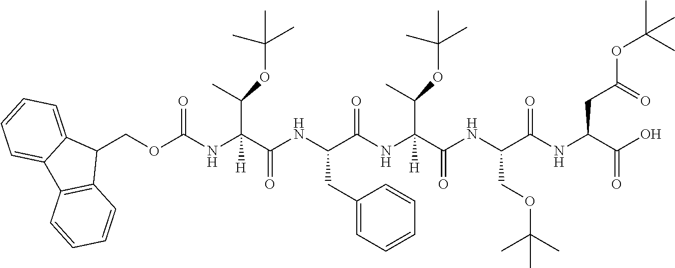 [SMILES: C[C@@H](OC(C)(C)C)[C@H](NC(=O)OCC1c2ccccc2-c2ccccc21)C(=O)N[C@@H](Cc1ccccc1)C(=O)N[C@H](C(=O)N[C@@H](COC(C)(C)C)C(=O)N[C@@H](CC(=O)OC(C)(C)C)C(=O)O)[C@@H](C)OC(C)(C)C]

Swelling of the resin: The resin (0.500 mmol) was charged to the reactor and swelled with DMF (3×10 mL×20 min).

Washing after DeFmoc: Following deprotection, the resin was washed with DMF (5×10 mL×2 min).

Washing after coupling: Following coupling, the resin was washed with DMF (5×10 mL×2 min).

Washing and drying of the resin: Following final coupling or deprotection, the resin was washed with DMF (5×10 mL×2 min), followed by DCM (5×10 mL×2 min), and drain dried under N₂ atmosphere to constant weight.

Preparation 35 Procedure

| | | T(tBu)-F-T(tBu)-S(tBu)-D(tBu) | | | |
|---|---|---|---|---|---|
| | | DeFmoc | | | |
| | | reagents & | | | |
| | Fmoc- | DeFmoc | Coupling | Coupling | |
| Cycle | AA-OH | condition | reagents | Time | Comments |
| 1 | Fmoc-L- | N/A | CTC resin | 16 h | Loading of |
| | Asp(tBu)- | | AA eq/DIEA | | Fmoc-L- |
| | OH | | eq = 0.8/3.0 | | Asp(tBu)-OH on |
| | | | 7 vol DCM | | CTC resin, |
| | | | | | measure resin |
| | | | | | loading by qNMR |
| 2 | Fmoc-L- | 20% PIP/DMF | AA/Oxyma/DIC = | 4 h | Start with Fmoc- |
| | Ser(tBu)- | 3 × 30 min, | 3.0/3.0/3.3 | | L-Asp(tBu) on |
| | OH | 25° C. | 13 vol DMF | | CTC Resin; 0.67 |
| | | | | | mmol/g loading |
| 3 | Fmoc-L- | 20% PIP/DMF | AA/Oxyma/DIC = | 4 h | |
| | Thr(tBu)- | 3 × 30 min, | 3.0/3.0/3.3 | | |
| | OH | 25° C. | 13 vol DMF | | |
| 4 | Fmoc-L- | 20% PIP/DMF | AA/Oxyma/DIC = | 4 h | |
| | Phe-OH | 3 × 30 min, | 3.0/3.0/3.3 | | |
| | | 25° C. | 13 vol DMF | | |
| 5 | Fmoc-L- | 20% PIP/DMF | AA/Oxyma/DIC = | 4 h | |
| | Thr(tBu)- | 3 × 30 min, | 3.0/3.0/3.3 | | |
| | OH | 25° C. | 13 vol DMF | | |

Two crystalline solid forms of T-F-T-S-D pentamer were identified, including T-F-T-S-D Form A and Form B. T-F-T-S-D Form B is a semi-disordered crystalline material that was observed from various solvent conditions. It represents a family of iso-structural solvates. T-F-T-S-D Form A is also a semi-disordered crystalline material which was generated by desolvation or partial desolvation of T-F-T-S-D Form B, and thus represents another family of desolvated or partially desolvated isostructural forms.

The XRPD patterns of crystalline T-F-T-S-D pentamers were obtained on a Bruker D8 Endeavor X-ray powder diffractometer, equipped with a CuKα (1.5418 Å) source and a Linxeye detector, operating at 40 kV and 40 mA. The sample was scanned between 4 and 30 2θ°, with a step size of 0.009 2θ° and a scan rate of 0.25 seconds/step, and using 0.3° primary slit opening, and 3.9° PSD opening. In some cases, a scan range of 4 and 25 2θ°, with a step size of 0.009 2θ° and a scan rate of 0.1 seconds/step was used. The sample was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of t 0.2 200 is presumed to take into account these potential variations without hindering the unequivocal identification of the indicated crystal form. Confirmation of a crystal form may be made based on any unique combination of distinguishing peaks.

### T-F-T-S-D Form A

In one experiment, 171.5 mg of amorphous T-F-T-S-D pentamer was dissolved in 0.7 mL EtOAc at ambient conditions; with stirring, 0.7 mL heptane was slowly added to the sample resulting immediate precipitation. After addition of another 0.7 mL heptane, the sample was stirred at ambient conditions for 4 days resulting a white slurry. The white solids isolated from the sample were consistent with T-F-T-S-D Form A.

In another experiment, 45.7 mg of amorphous T-F-T-S-D pentamer was dissolved in 100 μL MEK into a clear solution, and 200 μL n-propyl ether was added to the solution, and the sample was then stirred at ambient conditions for 4 days, resulting a white slurry. T-F-T-S-D Form A was produced by removing a wet aliquot from the slurry and air dried it at ambient conditions for 1 day.

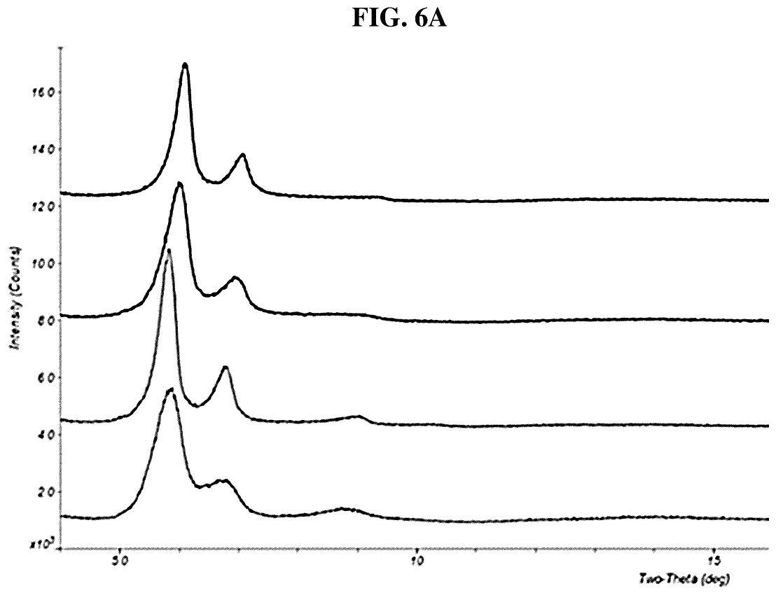

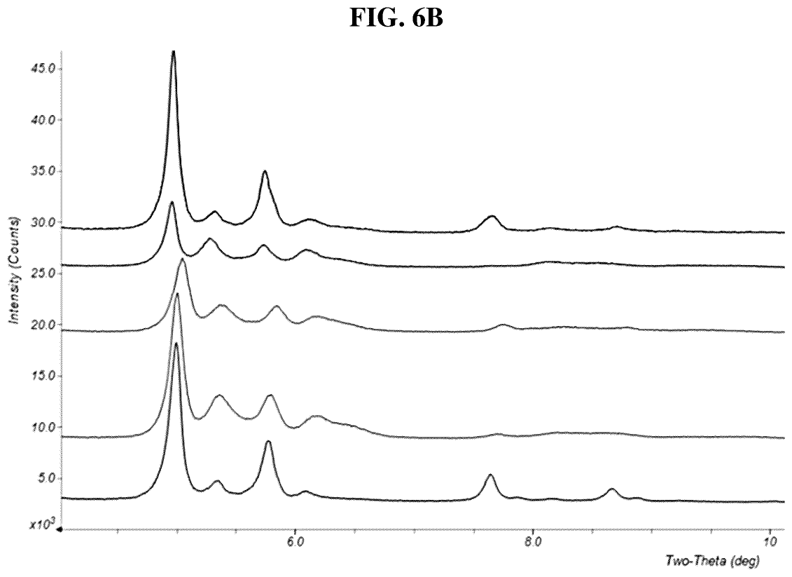

Prepared samples of the T-F-T-S-D Form A were characterized by an XRPD pattern using CuKα radiation as having diffraction peaks (2-theta values) as described in Table 18 below, and in particular having a peak at 6.7-7.1 °2-Theta, in combination with one or more of the peaks selected from the group consisting of 5.8-6.1 and 8.8-9.0 °2-Theta; with a tolerance for the diffraction angles of 0.2 degrees. A representative XRPD pattern of T-F-T-S-D Form A is shown in FIG. 6A.

TABLE 18
X-ray powder diffraction peaks of T-F-T-S-D Form A
T-F-T-S-D Form A

| | Angle | Relative Intensity |
|---|---|---|
| Peak | (°2-Theta) +/− 0.2° | (% of most intense peak) |
| 1 | 5.8-6.1 | 100.0% |
| 2 | 6.7-7.1 | 27.4% |
| 3 | 8.8-9.0 | 0-10.0% |

### T-F-T-S-D Form B

In one experiment, 195.8 mg of amorphous T-F-T-S-D pentamer was dissolved in 0.4 mL THF at ambient conditions; with stirring, 0.4 mL heptane was slowly added to the sample resulting immediate precipitation. The sample was stirred at ambient conditions for 4 days and a white slurry was observed. The wet solids from the slurry were consistent with T-F-T-S-D Form B.

In another experiment, 45.7 mg of amorphous T-F-T-S-D pentamer was dissolved in 100 μL MeOAc into a clear solution, and 100 μL heptane was added to the solution resulting white precipitates immediately. The sample was stirred at ambient conditions for 4 days, and the wet sample from the resulting white slurry was consistent with T-F-T-S-D Form B.

A prepared sample of the T-F-T-S-D Form B was characterized by an XRPD pattern using CuKα radiation as having diffraction peaks (2-theta values) as described in Table 19 below, and in particular having a peak at 5.0-5.2 °2-Theta, in combination with one or more of the peaks selected from the group consisting of 5.3-5.4, 5.7-6.0, 7.6-7.9, and 8.7-9.1 °2-Theta; with a tolerance for the diffraction angles of 0.2 degrees. A representative XRPD pattern of T-F-T-S-D Form B is shown in FIG. 6B.

TABLE 19
X-ray powder diffraction peaks of T-F-T-S-D Form B
T-F-T-S-D Form B

| | Angle | Relative Intensity |
|---|---|---|
| Peak | (°2-Theta) +/− 0.2° | (% of most intense peak) |
| 1 | 5.0-5.2 | 100.0% |
| 2 | 5.3-5.4 | 0-36.0% |
| 3 | 5.7-6.0 | 22.2%-34.6% |
| 4 | 6.1-6.2 | 0-22.1% |
| 5 | 6.3-6.5 | 0-8.8% |
| 6 | 7.6-7.9 | 0-27.5% |
| 7 | 8.1-8.2 | 0-2.8% |
| 8 | 8.7-9.1 | 2.4%-9.3% |

### Example 6: Boc-Tyr(tBu)-Aib-Gln(trt)-Gly-OH

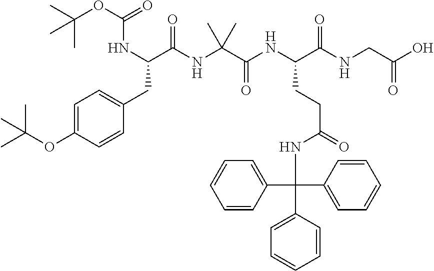 [SMILES: CC(C)(C)OC(=O)N[C@@H](Cc1ccc(OC(C)(C)C)cc1)C(=O)NC(C)(C)C(=O)N[C@@H](CCC(=O)NC(c1ccccc1)(c1ccccc1)c1ccccc1)C(=O)NCC(=O)O]

### Traditional SPPS

The tetramer is synthesized via traditional SPPS.

### Loading of Fmoc-Gly-OH on CTC Resin

The CTC resin was swelled with DMF and Fmoc-Gly-OH was loaded on the CTC resin by use of 1.5 molar equivalents of Gly and 4.0 equivalents of DIEA with respect to the loading factor of the CTC resin. The loading was done at 25° C. for 4 hours and after washing the resin, the unreacted sites on the resin were capped with a solution of 0.5 vol of MeOH, 1.6 vol of DIEA, and 8 vol of DMF with respect to the weight of resin used.

Coupling with Fmoc-L-Gln(Trt)-OH

The Fmoc group was removed by treatment with 10 vol 20% piperidine/DMF for one hour and the resin was washed with isopropyl acetate until residual piperidine was less than 500 ppm. A solution was prepared of Fmoc-L-Gln(trt)-OH (2.0 eq) and Oxyma (2.0 eq) in 5.3 vol of DMF. DIC (2.2 eq) was added and the amino acid was allowed to pre-activate for 90 minutes at 20° C. The activated ester solution was added to the resin and the reaction mixture was stirred for 12 hours at 20° C., then drained. The resin was washed with isopropyl acetate and forward processed to the next coupling.

Coupling with Fmoc-Aib-OH

The Fmoc group was removed by treatment with 10 vol 20% piperidine/DMF for one hour and the resin was washed with isopropyl acetate until residual piperidine was less than 500 ppm. A solution was prepared of Fmoc-Aib-OH (2.0 eq) and Oxyma (2.0 eq) in 5.3 vol of DMF. DIC (2.2 eq) was added and the amino acid was allowed to pre-activate for 15 minutes at 20 C. The activated ester solution was added to the resin and the reaction mixture was stirred for 12 hours at 20° C., then drained. The resin was washed with isopropyl acetate and forward processed to the next coupling.

Coupling with Boc-L-Tyr(tBu)-OH

The Fmoc group was removed by treatment with 10 vol 20% piperidine/DMF for one hour and the resin was washed with isopropyl acetate until residual piperidine was less than 500 ppm. A solution was prepared of Boc-L-Tyr(tBu)-OH (2.0 eq) and Oxyma (2.0 eq) in 5.3 vol of DMF. DIC (2.2 eq) was added and the amino acid was allowed to pre-activate for 15 minutes at 20 C. The activated ester solution was added to the resin and the reaction mixture was stirred for 12 hours at 20° C., then drained. The resin was washed with isopropyl acetate and dried at 35° C. and under vacuum to constant weight.

### Cleavage

All cleavage operations were conducted under 15-25° C.; the tetramer on resin material was placed in a cleavage filter reactor, 5 L DCM/kg resin was added, the material was stirred for not less than 10 min and the solvent was drained to waste; then 5 L 0.5 vol % TFA in DCM/kg resin was added to the cleavage filter reactor, and the mixture was stirred for not more than 60 min, the product solution was drained to the product collection container, and neutralized with 1 eq. of pyridine; then 5 L 0.5 vol % TFA in DCM/kg resin was added to the cleavage filter reactor, and stirred for not more than 60 min. The product solution was drained to the product collection container, and neutralized with 1 eq. of pyridine. Then 5 L DCM/kg was added to the cleavage filter reactor, and it was stirred for not more than 10 min. The product solution was drained to the product collection container and 5 L DCM/kg was added to the cleavage filter reactor, and it was stirred for not more than 10 min. The product solution was drained to the product collection container.

### Workup

The product solution was concentrated to 5 L/kg resin under not more than 0.4 bar pressure and 15-25° C. jacket temperature; 10 L pure water/kg resin was added and the mixture was stirred for not less than 30 min and allowed to settle until clear. The organic layer in the bottom was collected and a Karl Fischer (KF) titration, and pyridine and TFA in-process specification were taken. If the KF was greater than 0.35 wt. %, 0.4 kg Na₂SO₄/kg resin was added for re-slurry drying. If pyridine was greater than 0.2 wt. %, the corresponding equivalents of 0.5% TFA in DCM was added to titrate the excess pyridine. The mixture was stirred for not less than 10 min and 10 L water/kg resin was added and the mixture stirred for not less than 30 min. The organic layer was collected. Once the in-process specifications were passed, 2 L pentyl acetate/kg resin was added and a 40° C. jacket and 0.1 bar was added and the mixture was concentrated to 2.5 L solution/kg resin. Solution IPC was checked on DCM less than 7 wt. %; another 2 L pentyl acetate/kg resin was added.

### LPPS

Alternatively, the tetramer is synthesized via LPPS.

### Step 1:

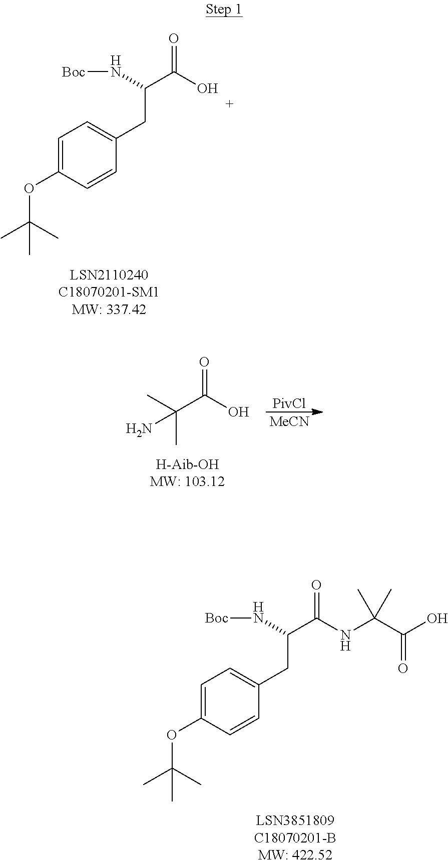

Aib Protection—In one vessel, charge H-Aib-OH (1.3 eq. to Boc-Tyr(But)-OH) and MeCN (3V), then add bis(trimethylsilyl)acetamide (BSA) (1 eq.) slowly. Stir the mixture at 20-30° C. for 16 hr.

Coupling Reaction—In another vessel, charge Boc-Tyr(But)-OH, MeCN (7V), and 2,6-lutidine (3 eq.). Adjust the temperature of the vessel to −25° C. Stir the mixture for 0.5 hr. Charge PivCl (1.1 eq.) into the vessel slowly. Stir the mixture for 2 hr. Analyze sample for complete activation. Charge the contents of the first vessel (Aib Protection) into this reaction vessel. Stir at −25° C. for 16 hr. Analyze sample for reaction completion.

Quench and Workup—Add acetic acid (2 eq) and water (10V). Concentrate to remove MeCN. Extract with EtOAc (10V) 2×. Wash organic layer with 5% citric acid (10V), and water (10V). Concentrate organic layer to 2V. Add heptane (16V) slowly, then filter the solids. Re-dissolve the solids in EtOAc (6V) and heat up to 40° C. Add heptane (24V) slowly then cool to 20° C. slowly and mix for 16 hr. Filter and dry the wet cake under vacuum at 35-45° C. for 24 hr.

### Step 2:

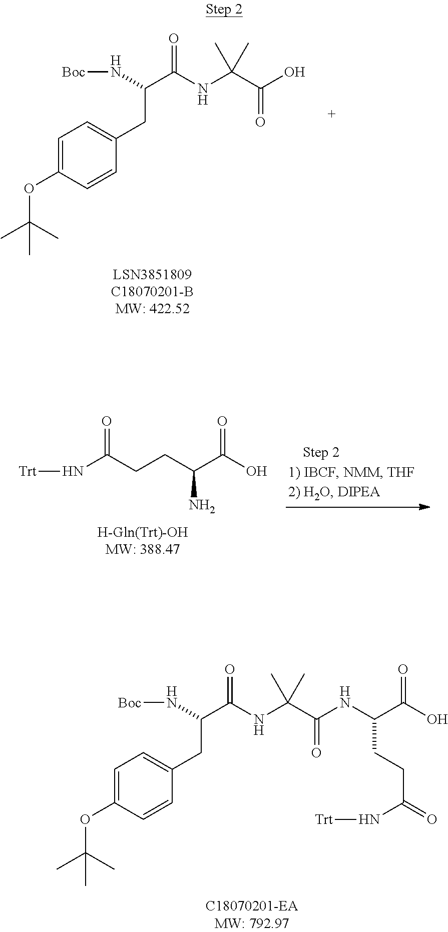

Coupling Reaction—Add a solution of Dimer Intermediate (C18070201-B) in THF (3V) and NMM (1.35 eq.) to a solution of IBCF (1.05 eq.) in THF (7V) at 0° C. Stir at 0° C. for 4-6 hr. Check sample for complete activation. In another reaction vessel dissolve H-Gln(Trt)-OH (1.4 eq.) in THF (10V) and water (3V) and stir for 0.5 hr, then add to the solution containing Dimer Intermediate B at 0° C. Add DIPEA (3.0 eq.) and stir at 0° C. for 4-6 hr. Raise the temperature to 20° C. over 3-4 hr, then stir for 12-20 hr. Check sample for reaction completion. Concentrate to 1-2V under vacuum below 45° C. Add MTBE (20V), then wash the organic layer 4× with a 10:1 solution of 5% KHSO₄ aqueous:DMF (15V). Wash the organic layer 4× with a 10:1 solution of 5% Na₂CO₃ aqueous:DMF (10:1). Wash the organic layer 1× with 5% KHSO4 aqueous (10V). Concentrate to 1-2V

Salt Formation Purification—Add EtOAc (2.67V) to the solution and heat to 50° C. Add (1S)-1-phenylpropan-1-amine (1.5 eq.), then add MeCN (5.33V) slowly. Stir for 2 hr at 50° C., then cool to 25° C. slowly. Filter and wash the cake with 1:2 EtOAc:MeCN (1V). Add MTBE (20V), then wash 2× with 5% KHSO₄. Concentrate to dryness and take sample for analysis. If necessary, repeat the salt formation purification.

Isolation—Add MTBE (1.2V) to the solids. Add heptane (6V) and stir for 16 hr at 25° C. Filter and wash the wet cake with heptane (2V). Dry the wet cake at 40° C. for 18 hr.

### Step 3:

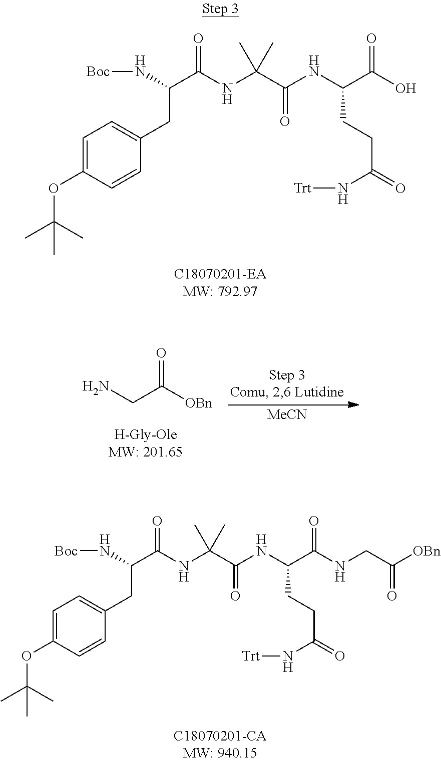

Add Trimer Intermediate (C18070201-EA), H-Gly-OBzl (1.3 eq.), and MeCN (10V) to the reaction vessel and lower the temperature to −20° C. Add 2,6-lutidine (3.0 eq.) and COMU (1.3 eq.) and stir at −20° C. for 2-4 hr. Analyze a sample to test for reaction completion. Concentrate to IV below 46° C. Add EtOAc (5V) and stir for 0.5-1 hr. Add MTBE (5V), then filter the mixture. Wash the cake with 1:1 EtOAc:MTBE (5V). Wash the filtrate 4× with 5% NaHCO₃ (10V), 4× with KHSO₄ (10V), then 1× with water (10V). Concentrate the organic layer to 1-2V below 45° C. Add DMF (2.5V) and stir to form a clear solution at 15-20° C. Add water (7.5V) slowly. Stir the mixture for 2-6 hr at 15-20° C. Filter and wash the cake with water (2-3V). Reslurry the cake with water (10V) for 2-6 hr at 15-20° C. and filter. Wash the cake with water (2-3V) then dry for 16-48 hr at 45° C. Check residual DMF and water. Reslurry with water again or dry further if needed.

### Step 4:

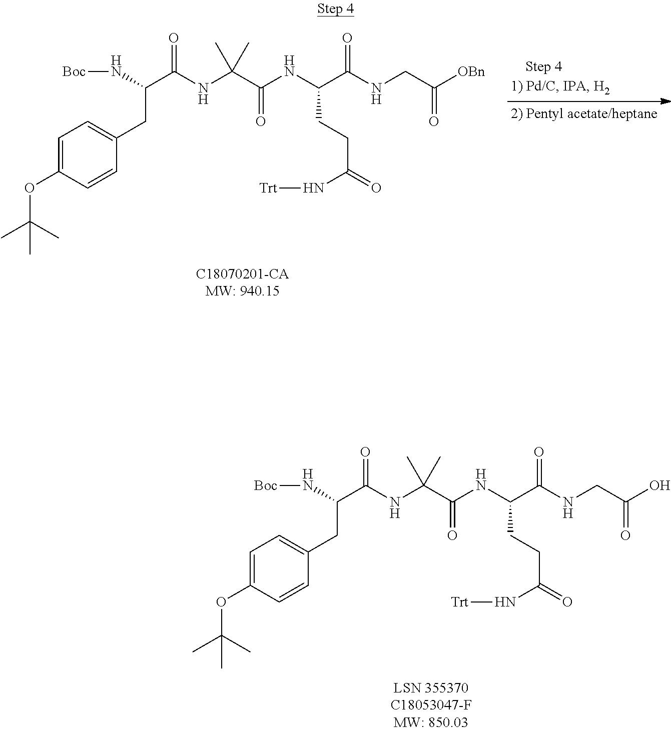

Benzyl Deprotection—Add Tetramer Ester Intermediate (C18070201-CA) and IPA (10V) into the reaction vessel. Add Pd/C (0.1×, 50 wt %) into the reaction vessel and exchange atmosphere with Argon 3×. Exchange atmosphere with Hydrogen 3×. Adjust the pressure of the vessel to 45 psi with Hydrogen and heat the reactor to 40° C. Stir the reaction at 45 psi and 40° C. for 16-20 hr. Take a sample for analysis. Cool the reaction vessel to 20-30° C. and exchange the atmosphere with Argon 3×. Filter the mixture and wash the wet cake (Pd/C) with IPA (2V). Concentrate the filtrate to IV below 45° C.

Crystallization—Charge pentyl acetate (5) and then concentrate to IV below 45° C. Repeat this process for a total of 3 times. Charge pentyl acetate (5V) and stir for 0.5-1 hr at 20° C. Charge heptane (1V) over 1 hr. Stir the mixture for 3-5 hr at 20° C. Apply thermal cycle (heat to 35° C. over 1 hr, stir for 3-5 hr, cool to 20° C. over 1 hr, stir for 3-5 hr at 20° C.) 2×. Charge 5V heptane over 1 hr. Stir for 3-5 hr at 20° C. Filter the solids and wash the wet cake with heptane (2V). Dry the wet cake under vacuum for 16-24 hr at 40-50° C. Pull sample and determine if it is necessary for a reslurry.

### Crystallization

After solvent swap via distillation, the tetramer was in 5 vol pentyl acetate (L/kg resin, tetramer concentration 80-100 mg/mL). The solution was held at 20° C. for 12 hours for primary nucleation. If solids were observed, the mixture proceeded to thermal cycles. If solids were not observed, the mixture was cooled to 5° C. and held for 12 hours. If solids were observed, the mixture proceeded to thermal cycles. If solids were not observed, 1 vol heptane was added and the mixture was held for 12 hours. If solids were observed, the mixture proceeded to thermal cycles. If solids were not observed, 1 vol heptane was added and the process was repeated.

Thermal cycles were used to convert from amorphous to crystalline form and to grow crystalline particles. The mixture was heated to 30° C., held for 1 hr, then cooled to 20° C. and held for 1 hr. Thermal cycles were repeated five times.

After thermal cycles, 5 vol heptane were added to lower the solubility and improve the yield. Then, the slurry was filtered. It was washed once with 5 vol 1:1 heptane: pentyl acetate to remove impurities. It was washed twice with 5 vol heptane in a re-slurry wash to remove pentyl acetate. Finally the solids were dried at 50° C.

Two solid forms of the Y-Aib-Q-G tetramer were identified, including Y-Aib-Q-G Form A and Form B. Y-Aib-Q-G Form A is a crystalline form. A solvated form was initially produced from pentyl acetate or mixtures containing pentyl acetate (such as pentyl acetate/ETBE, pentyl acetate/TAME, pentyl acetate/heptane, etc.).

Y-Aib-Q-G Form A represents a family of iso-structural solvates and could also be generated from other solvent conditions such as EtOAc and 2-Me THF/TAME.

Y-Aib-Q-G Form B is a semi-disordered crystalline material which was generated by desolvation or partial desolvation of Y-Aib-Q-G Form A and thus represents another family of desolvated or partially desolvated isostructural forms.

### Preparation of Y-Aib-O-G Form A

Y-Aib-Q-G Form A was obtained by adding 1 mL pentyl acetate to 210.0 mg of amorphous Y-Aib-Q-G tetramer. The sample was stirred at ambient conditions resulting in a clear solution. 3 mL of ETBE (t-butyl ethyl ether) was added into the solution, and the sample was capped and stirred at ambient conditions. A thick white slurry was obtained and an additional 2 mL of ETBE was added to the slurry after the sample had been stirred for 2 days. The sample was stirred at ambient conditions for another 3 days, and the wet sample from the resulting white slurry was consistent with Y-Aib-Q-G Form A.

Y-Aib-Q-G Form A was also obtained by dissolving 55.5 mg of amorphous Y-Aib-Q-G tetramer in 250 μL pentyl acetate. 0.5 mL of heptane was added to the solution resulting in a white suspension. The sample was stirred at ambient conditions in a capped vial for 1 day and a white slurry was obtained. The XRPD of the wet sample from the slurry was consistent with Y-Aib-Q-G Form A.

### Preparation of Y-Aib-O-G Form B

Form B solids were obtained from drying of Y-Aib-Q-G Form A. The wet solids in the first example above were isolated by vacuum filtration, and the resulting material was rinsed on the filter with 0.5 mL ETBE, air dried on the filter under continued vacuum for about 5 minutes, collected, and then dried at approximately 30° C. under vacuum for 1 day. The XRPD of the obtained white solids were consistent with Y-Aib-Q-G Form B.

The XRPD patterns of crystalline Y-Aib-Q-G tetramers were obtained on a Bruker D8 Endeavor X-ray powder diffractometer, equipped with a CuKα (1.5418 Å) source and a Linxeye detector, operating at 40 kV and 40 mA. The sample was scanned between 4 and 30 2θ°, with a step size of 0.009 2θ° and a scan rate of 0.25 seconds/step, and using 0.3° primary slit opening, and 3.9° PSD opening. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### XRPD of Y-Aib-O-G Form A

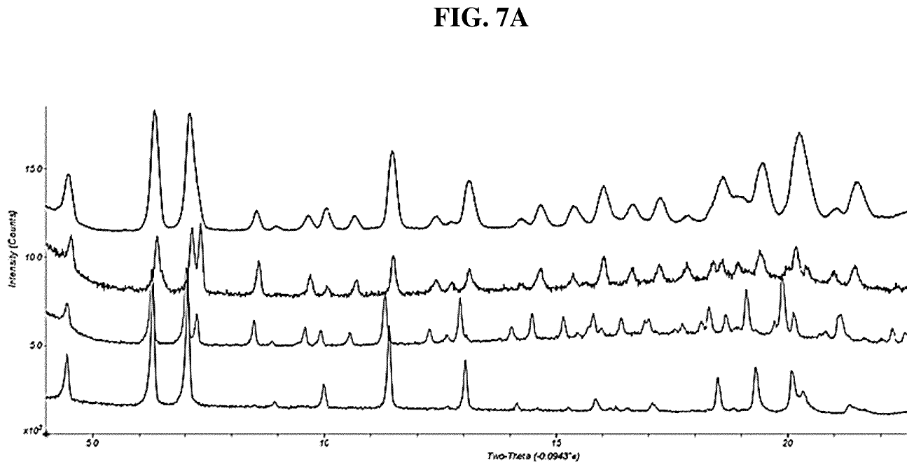

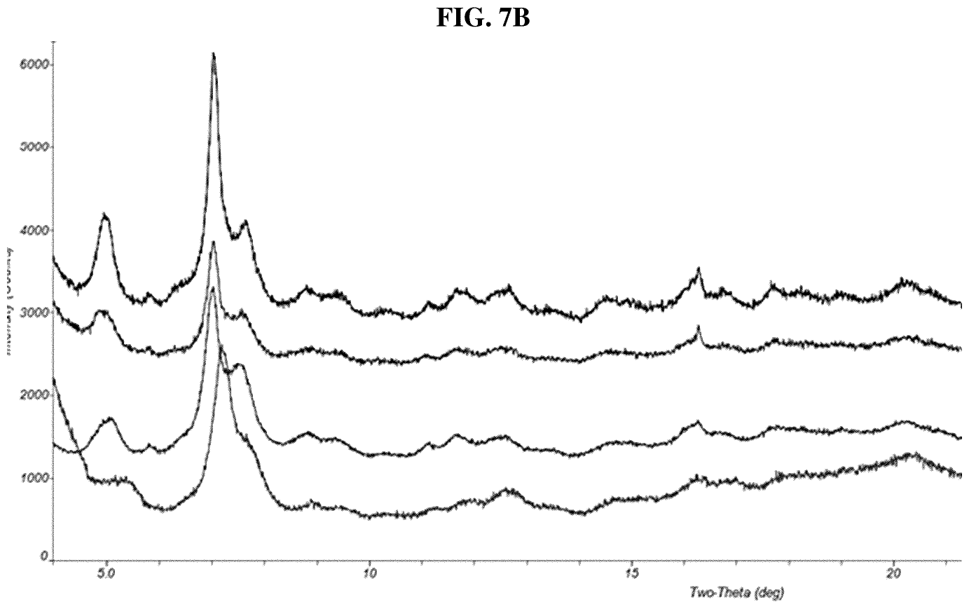

Prepared samples of the Y-Aib-Q-G Form A were characterized by XRPD patterns using CuKα radiation as having diffraction peaks (2-theta values) as described in Table 20 below, and in particular having a peak at 6.3-6.4 °2-Theta, in combination with one or more of the peaks selected from the group consisting of 4.5, 7.1, 13.0-13.1, 15.9-16.0, and 18.4-18.6 °2-Theta; with a tolerance for the diffraction angles of 0.2 degrees. A representative XRPD patter of Y-Aib-Q-G Form A is shown in FIG. 7A.

TABLE 20
X-ray powder diffraction peaks of Y-Aib-Q-G Form A
Y-Aib-Q-G tetramer Form A

| | Angle | Relative Intensity |
|---|---|---|
| Peak | (°2-Theta) +/− 0.2° | (% of most intense peak) |
| 1 | 4.5 | 25.7%-34.1% |
| 2 | 6.3-6.4 | 94.6%-100% |
| 3 | 7.1 | 96.3%-100% |
| 4 | 10.0-10.1 | 16.2%-20.6% |
| 5 | 11.4-11.5 | 48.1%-81.8% |
| 6 | 13.0-13.1 | 38.3%-48.6% |
| 7 | 14.1-14.3 | 5.1%-15.2% |
| 8 | 15.9-16.0 | 8.8-54.9% |
| 9 | 17.1-17.2 | 5.0%-33.9% |
| 10 | 18.4-18.6 | 13.5%-41.0% |
| 11 | 20.0-20.3 | 15.1%-68.7% |
| 12 | 21.2-21.5 | 3.7%-45.1% |

### XRPD of Y-Aib-O-G Form B

Prepared samples of the Y-Aib-Q-G Form B were characterized by an XRPD pattern using CuKα radiation as having diffraction peaks (2-theta values) as described in Table 21 below, and in particular having a peak at 7.0-7.2 °2-Theta, in combination with one or more of the peaks selected from the group consisting of 5.0-5.4, 7.6-7.7, 8.8-8.9, 9.4-9.5, and 12.5-12.7 °2-Theta; with a tolerance for the diffraction angles of 0.2 degrees. A representative XRPD pattern of Y-Aib-Q-G Form B is shown in FIG. 7B.

TABLE 21
X-ray powder diffraction peaks of Y-Aib-Q-G Form B
Y-Aib-Q-G tetramer Form B

| | Angle | Relative Intensity |
|---|---|---|
| Peak | (°2-Theta) +/− 0.2° | (% of most intense peak) |
| 1 | 5.0-5.4 | 19.6%-31.4% |
| 2 | 7.0-7.2 | 100.0% |
| 3 | 7.6-7.7 | 33.0%-52.1% |
| 4 | 8.8-8.9 | 4.7%-9.8% |
| 5 | 9.4-9.5 | 3.4%-7.9% |
| 5 | 11.7-11.8 | 6.3%-9.0% |
| 6 | 12.5-12.7 | 3.8%-14.2% |

### Example 7: (Boc)-His(dnp)-Aib-Gln(trt)-Gly

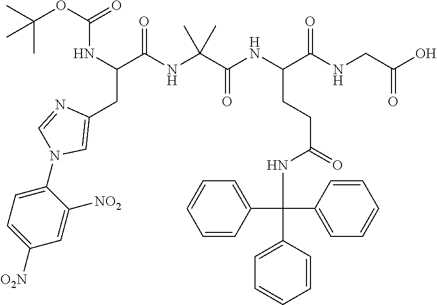 [SMILES: CC(C)(C)OC(=O)NC(Cc1cn(-c2ccc([N+](=O)[O-])cc2[N+](=O)[O-])cn1)C(=O)NC(C)(C)C(=O)NC(CCC(=O)NC(c1ccccc1)(c1ccccc1)c1ccccc1)C(=O)NCC(=O)O]

Three crystalline solid forms of the H(DNP)-Aib-Q-G tetramer were identified, including H(DNP)-Aib-Q-G Form A, Form B, and Form C.

H(DNP)-Aib-Q-G Form A is a crystalline labile solvate, and converts to H(DNP)-Aib-Q-G Form C easily (during isolation or upon drying).

H(DNP)-Aib-Q-G Form B is a crystalline material generated from multiple solvent conditions. It represents a family of iso-structural solvates.

H(DNP)-Aib-Q-G Form C is a semi-disordered crystalline material observed from multiple solvent conditions.

### H(DNP)-Aib-O-G Form A

H(DNP)-Aib-Q-G Form A was prepared from acetonitrile (ACN)/methyl tert-butyl ether (MTBE). Approximately 50 mg of amorphous H(DNP)-Aib-Q-G tetramer was dissolved in 0.2 mL 1:5 (vol/vol) ACN/MTBE at ambient conditions into a clear yellow solution. With stirring, 0.1 mL of MTBE was added into the solution resulting a light-yellow slurry. The sample was stirred at ambient conditions for 5 days, and the wet solids were consistent with H(DNP)-Aib-Q-G Form A.

### H(DNP)-Aib-O-G Form B

Approximately 50 mg of amorphous H(DNP)-Aib-Q-G tetramer was dissolved in 0.2 mL methyl acetate (MeOAc) at ambient conditions into a clear yellow solution. 5×0.2 mL of cyclopentyl methyl ether (CPME) was added into the sample. The solution was stirred at ambient conditions overnight and then placed into refrigerator at 2-8° C. for about 6 weeks. White solids were produced from the solution and were consistent with H(DNP)-Aib-Q-G Form B.

Following a similar procedure, H(DNP)-Aib-Q-G Form B was also produced from 1:10 (vol/vol) nitromethane/MTBE solution at 2-8° C.

Approximately 50 mg of amorphous H(DNP)-Aib-Q-G tetramer was dissolved in 0.2 mL ethyl acetate (EtOAc) at ambient conditions into a clear yellow solution. With stirring, 0.2 mL of heptane was added into the solution producing yellow sticky material. The sample was stirred at 36° C. for 7 days resulting a light-yellow slurry. The obtained solids were consistent with H(DNP)-Aib-Q-G Form B.

### H(DNP)-Aib-O-G Form C

H(DNP)-Aib-Q-G Form C was prepared from tetrahydrofuran (THF)/MTBE. Approximately 50 mg of amorphous H(DNP)-Aib-Q-G tetramer was dissolved in 0.1 mL THF at ambient conditions into a clear yellow solution. With stirring, 5×0.1 mL of MTBE was added, and the sample was stirred at ambient conditions overnight then 36° C. for 7 days resulting a light-yellow slurry. The sample was isolated by centrifuge at ambient conditions using a centrifuge tube filter. The obtained solids were consistent with H(DNP)-Aib-Q-G Form C.

Following a similar procedure, H(DNP)-Aib-Q-G Form C can be produced from different solvent systems containing MTBE, such as EtOAc/MTBE, methyl ethyl ketone (MEK)/MTBE, etc.

The XRPD patterns of crystalline H(DNP)-Aib-Q-G tetramers were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. For cases of Form A, the sample was scanned between 4 and 25 2θ° with a scan rate of 0.1 seconds/step. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### XRPD of H(DNP)-Aib-O-G Form A

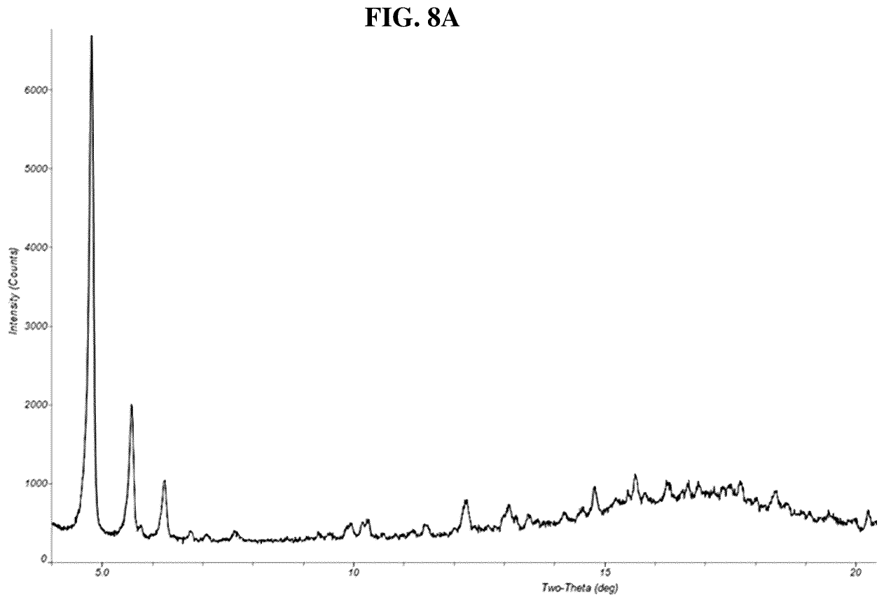

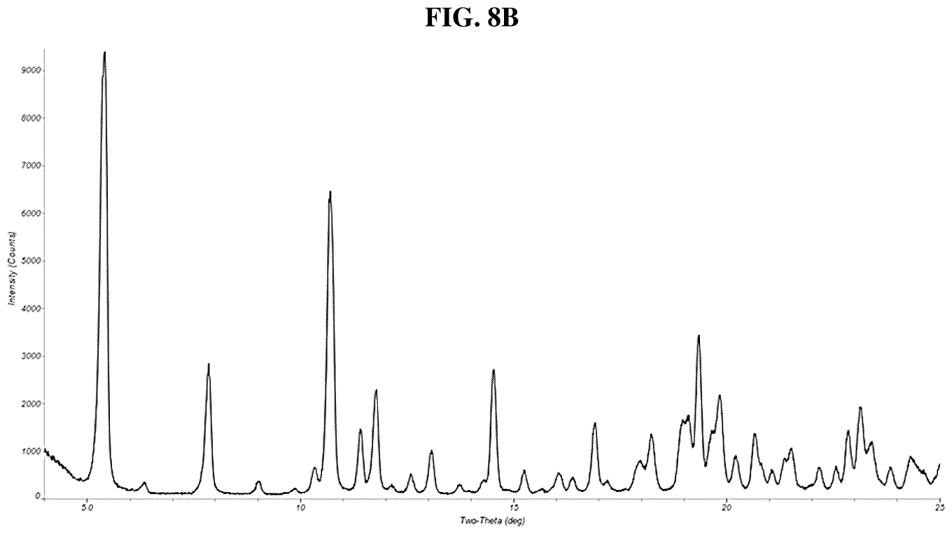

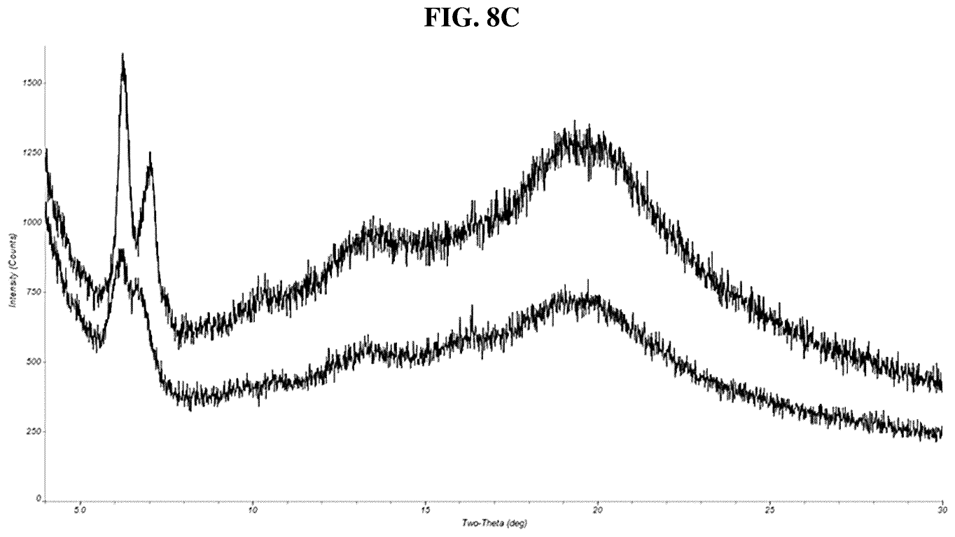

Prepared samples of the H(DNP)-Aib-Q-G Form A were characterized by an XRPD pattern using CuKα radiation as having diffraction peaks (2-theta values) as described in Table 22 below, and in particular having a peak at 4.8 °2-Theta, in combination with one or more of the peaks selected from the group consisting of 5.6, 6.2, 14.8, and 15.6 °2-Theta; with a tolerance for the diffraction angles of 0.2 degrees. A representative XRPD pattern of H(dnp)-Aib-Q-G Form A is shown in FIG. 8A.

TABLE 22
X-ray powder diffraction peaks of H(DNP)-Aib-Q-G Form A
H(DNP)-Aib-Q-G Form A

| | Angle | Relative Intensity |
|---|---|---|
| Peak | (°2-Theta) +/− 0.2° | (% of most intense peak) |
| 1 | 4.8 | 100.0% |
| 2 | 5.6 | 26.7% |
| 3 | 6.2 | 11.7% |
| 4 | 10.3 | 3.1% |
| 5 | 12.2 | 6.4% |
| 6 | 13.1 | 4.4% |
| 7 | 14.8 | 5.1% |
| 8 | 15.6 | 6.1% |
| 9 | 16.2 | 3.9% |
| 10 | 18.4 | 3.6% |

### XRPD of H(DNP)-Aib-O-G Form B

A prepared sample of the H(DNP)-Aib-Q-G Form B was characterized by an XRPD pattern using CuKα radiation as having diffraction peaks (2-theta values) as described in Table 23 below, and in particular having a peak at 5.3 °2-Theta, in combination with one or more of the peaks selected from the group consisting of 7.7, 10.5, 11.3, 11.6, and 14.4 2-Theta; with a tolerance for the diffraction angles of 0.2 degrees. A representative XRPD pattern of H(dnp)-Aib-Q-G Form B is shown in FIG. 8B.

TABLE 23
X-ray powder diffraction peaks of H(DNP)-Aib-Q-G Form B
H(DNP)-Aib-Q-G Form B

| | Angle | Relative Intensity |
|---|---|---|
| # | (°2-Theta) +/− 0.2° | (% of most intense peak) |
| 1 | 5.3 | 100.0% |
| 2 | 6.2 | 2.6% |
| 3 | 7.7 | 28.8% |
| 4 | 8.9 | 2.8% |
| 5 | 10.2 | 6.1% |
| 6 | 10.5 | 69.3% |
| 7 | 11.3 | 13.6% |
| 8 | 11.6 | 23.4% |
| 9 | 12.4 | 4.1% |
| 10 | 12.9 | 9.8% |
| 11 | 14.4 | 28.4% |
| 12 | 15.1 | 4.9% |
| 13 | 16.8 | 15.7% |
| 14 | 17.8 | 6.9% |
| 15 | 18.1 | 12.7% |
| 16 | 18.8 | 16.2% |
| 17 | 19.2 | 35.2% |
| 18 | 19.7 | 21.9% |
| 19 | 20.5 | 12.4% |

### XRPD of H(DNP)-Aib-O-G Form C

A prepared sample of the H(DNP)-Aib-Q-G Form C was characterized by an XRPD pattern using CuKα radiation as having diffraction peaks (2-theta values) at 6.2 and 6.9 °2-Theta with a tolerance for the diffraction angles of 0.2 degrees. A representative XRPD pattern of H(dnp)-Aib-Q-G Form C is shown in FIG. 8C.

### Example 8: (Boc)His(trt)-Aib-Gln (trt)-Gly

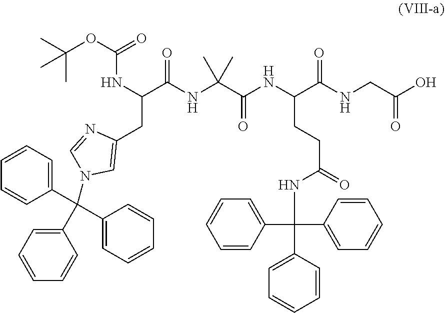 [SMILES: CC(C)(C)OC(=O)NC(Cc1cn(C(c2ccccc2)(c2ccccc2)c2ccccc2)cn1)C(=O)NC(C)(C)C(=O)NC(CCC(=O)NC(c1ccccc1)(c1ccccc1)c1ccccc1)C(=O)NCC(=O)O]

Two crystalline solid forms of the H(trt)-Aib-Q-G tetramer were identified, including H(trt)-Aib-Q-G Form A and Form B.

H(trt)-Aib-Q-G Form A is a crystalline material generated from multiple organic mixtures. It represents a family of iso-structural solvates with variable amounts of solvent(s) content in the unit cell.

H(trt)-Aib-Q-G Form B is a crystalline material generated from EtOH/H₂O. It is a solvated form.

### H(trt)-Aib-O-G Form A

H(trt)-Aib-Q-G Form A was prepared from tetrahydrofuran (THF)/methyl tert-butyl ether (MTBE). 50.3 mg of amorphous H(trt)-Aib-Q-G tetramer was stirred in 0.2 mL of 1:3 (vol/vol) THF/MTBE at ambient conditions for 5 days resulting a thick white slurry; 0.2 mL of 1:3 (vol/vol) THF/MTBE was added, and the sample was stirred at ambient conditions for another 3 days before isolation. The white solids from the slurry, either wet or isolated, were consistent with H(trt)-Aib-Q-G Form A.

Following similar procedures, H(trt)-Aib-Q-G Form A can also be prepared from other organic solvent mixtures such as THF/heptane and 1,4-dioxane/H₂O, acetonitrile (ACN)/MTBE, and ethyl acetate/MTBE, etc.

### H(trt)-Aib-O-G Form B

50.3 mg of amorphous H(trt)-Aib-Q-G tetramer was stirred in 0.2 mL of 1:1 (vol/vol) ethanol/H2O at ambient conditions resulting a mixture of white particles and light-yellow gel after 5 days. 50 μL aliquot from this slurry was added as seeds into a sample containing 311.2 mg of amorphous H(trt)-Aib-Q-G tetramer in 1 mL 1:1 (vol/vol) ethanol/H₂O. The sample was stirred at ambient conditions for 5 days. The un-dried solids isolated from the sample were consistent with H(trt)-Aib-Q-G Form B.

The XRPD patterns of crystalline H(trt)-Aib-Q-G tetramers were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. In some cases, the sample was scanned between 4 and 30 2θ° with a scan rate of 0.25 seconds/step. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### XRPD of H(trt)-Aib-O-G Form A

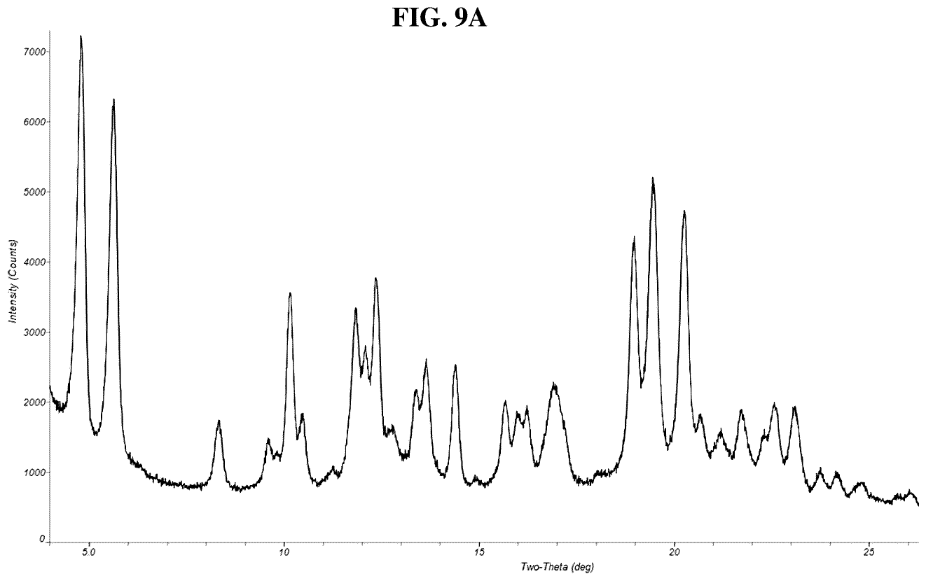

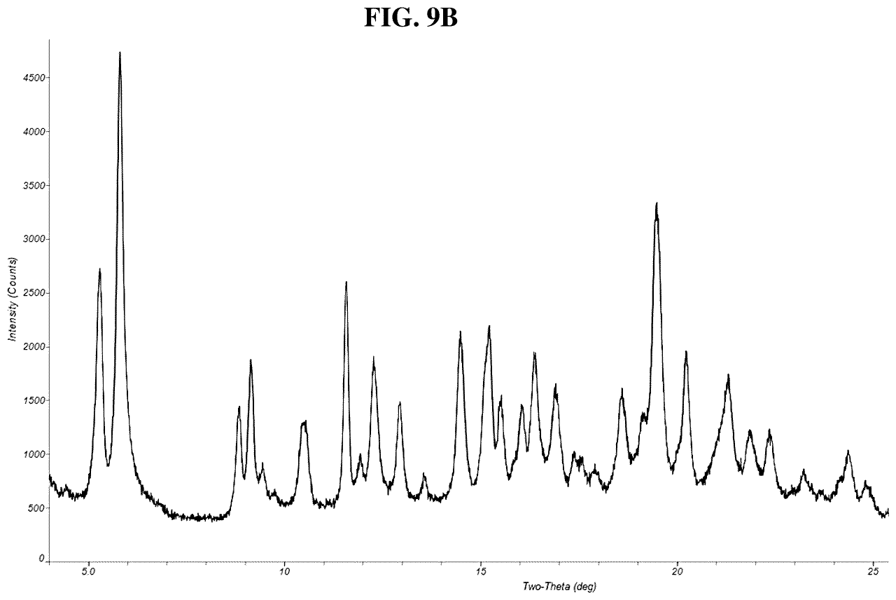

Prepared samples of the H(trt)-Aib-Q-G Form A were characterized by an XRPD pattern using CuKα radiation as having diffraction peaks (2-theta values) as described in Table 24 below, and in particular having a peak at 4.7 °2-Theta, in combination with one or more of the peaks selected from the group consisting of 5.5, 8.2, 10.1, 11.8, 13.3, 13.6, and 18.9 °2-Theta; with a tolerance for the diffraction angles of 0.2 degrees. A representative XRPD patter of H(trt)-Aib-Q-G Form A is shown in FIG. 9A.

TABLE 24
X-ray powder diffraction peaks of H(trt)-Aib-Q-G Form A
H(trt)-Aib-Q-G tetramer Form A

| | Angle | Relative Intensity |
|---|---|---|
| Peak | (°2-Theta) +/− 0.2° | (% of most intense peak) |
| 1 | 4.7 | 100.0% |
| 2 | 5.5 | 96.9% |
| 3 | 8.2 | 17.5% |
| 4 | 9.5 | 12.7% |
| 5 | 10.1 | 38.8% |
| 6 | 10.4 | 17.7% |
| 7 | 11.8 | 45.6% |
| 8 | 12.0 | 36.0% |
| 9 | 12.3 | 53.6% |
| 10 | 13.3 | 17.4% |
| 11 | 13.6 | 29.2% |
| 12 | 14.3 | 29.3% |
| 13 | 15.6 | 21.5% |
| 14 | 15.9 | 18.5% |
| 15 | 16.1 | 20.3% |
| 16 | 16.8 | 22.1% |
| 17 | 18.9 | 61.0% |
| 18 | 19.4 | 69.0% |
| 19 | 20.2 | 61.6% |
| 20 | 21.6 | 12.7% |
| 21 | 22.5 | 14.9% |
| 22 | 23.0 | 16.8% |

### XRPD of H(trt)-Aib-O-G Form B

A prepared sample of the H(trt)-Aib-Q-G Form B was characterized by an XRPD pattern using CuKα radiation as having diffraction peaks (2-theta values) as described in Table 25 below, and in particular having a peak at 5.8 °2-Theta, in combination with one or more of the peaks selected from the group consisting of 5.3, 8.9, 9.2, 15.2, 18.6, and 19.5 °2-Theta; with a tolerance for the diffraction angles of 0.2 degrees. A representative XRPD pattern of H(trt)-Aib-Q-G Form B is shown in FIG. 9B.

TABLE 25
X-ray powder diffraction peaks of H(trt)-Aib-Q-G Form B
H(trt)-Aib-Q-G tetramer Form B

| | Angle | Relative Intensity |
|---|---|---|
| Peak | (°2-Theta) +/− 0.2° | (% of most intense peak) |
| 1 | 5.3 | 45.9% |
| 2 | 5.8 | 100.0% |
| 3 | 8.9 | 25.1% |
| 4 | 9.2 | 26.2% |
| 5 | 10.6 | 19.1% |
| 6 | 11.6 | 46.3% |
| 7 | 12.3 | 27.0% |
| 8 | 13.0 | 20.2% |
| 9 | 14.5 | 35.8% |
| 10 | 15.2 | 28.8% |
| 11 | 15.5 | 11.8% |
| 12 | 16.1 | 10.7% |
| 13 | 16.4 | 21.8% |
| 14 | 16.9 | 17.4% |
| 15 | 18.6 | 17.1% |
| 16 | 19.5 | 57.7% |
| 17 | 20.2 | 28.0% |
| 18 | 21.3 | 21.6% |
| 19 | 21.9 | 8.9% |
| 20 | 22.4 | 10.7% |

### Example 9: (Fmoc)Asn(t-Bu)-Tyr(t-Bu)-Ser(t-Bu)-Lys

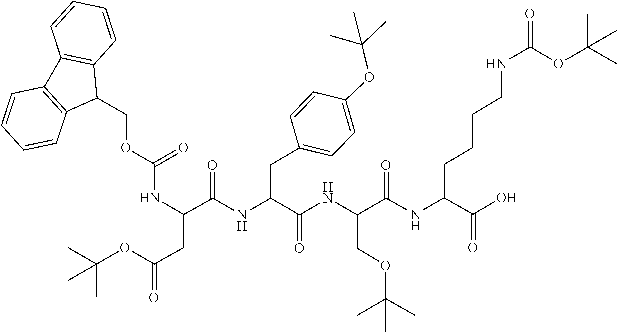 [SMILES: CC(C)(C)OCC(NC(=O)C(Cc1ccc(OC(C)(C)C)cc1)NC(=O)C(CC(=O)OC(C)(C)C)NC(=O)OCC1c2ccccc2-c2ccccc21)C(=O)NC(CCCCNC(=O)OC(C)(C)C)C(=O)O]

Three crystalline solid forms of OXM D-Y-S-K tetramer were identified, including D-Y-S-K Forms A, B, and C.

D-Y-S-K Form A is a crystalline material generated from multiple organic mixtures. It is solvated form that exists at wet conditions.

D-Y-S-K Form B is a crystalline material that is anhydrous/unsolvated.

D-Y-S-K Form C is a semi-disordered crystalline material and generated from MTBE/ETBE mixture. It is a solvated material.

### D-Y-S-K Form A

D-Y-S-K Form A was prepared from methyl acetate (MeOAc)/dibutyl ether. 0.2 mL MeOAc was added to 40.8 mg of amorphous D-Y-S-K tetramer resulting a clear solution; 3×0.2 mL of dibutyl ether was added to the solution and a white gel was developed after ambient slurry for overnight. The gel was broken into a viscous liquid and stirred at ambient conditions for 1 day and then at about 36° C. for 5 days resulting a thick white slurry. The wet solids from the slurry were consistent with D-Y-S-K Form A.

Following similar procedures, D-Y-S-K Form A can also be prepared from other organic solvent mixtures such as acetone/dibutyl ether, acetonitrile (ACN)/dibutyl ether, ethyl acetate (EtOAc)/dibutyl ether, MeOAc/heptane, and methyl ethyl ketone (MEK)/dibutyl ether, etc.

### D-Y-S-K Form B

D-Y-S-K Form B was prepared from methyl acetate (MeOAc)/ethyl tert-butyl ether (ETBE). 0.2 mL MeOAc was added to 40.8 mg of amorphous D-Y-S-K tetramer resulting a clear solution; 3×0.2 mL of ETBE was added to the solution followed by stirring at ambient conditions for a total of 7 days. A white gel was observed during the stirring which slowly converted to a thick white slurry. The solids from the slurry, either wet or isolated, were consistent with D-Y-S-K Form B.

Following similar procedures, D-Y-S-K Form B can also be prepared from other organic solvent mixtures such as ethyl acetate (EtOAc)/ETBE, methyl ethyl ketone (MEK)/ETBE, MEK/heptane, and pentyl acetate/ETBE, etc.

D-Y-S-K Form B was also observed from slurries of D-Y-S-K Form A when solids were isolated using centrifuge tube filters followed by air drying at ambient conditions for 4 hours or longer.

### D-Y-S-K Form C

40.4 mg of amorphous D-Y-S-K solids were dissolved in 0.2 mL MTBE by briefly heating the mixture at 62° C. 3×0.2 mL of ETBE was added to the clear solution at ambient conditions and a white gel was developed after ambient slurry for overnight. The gel was broken into a viscous liquid and stirred at ambient conditions for another 6 days resulting a thick white slurry. The wet solids from the slurry were consistent with D-Y-S-K Form C.

The XRPD patterns of crystalline T-F-T-S tetramers were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. For cases of Form A and Form C, the sample was scanned between 4 and 25 2θ° with a scan rate of 0.1 seconds/step. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### XRPD of D-Y-S-K Form A

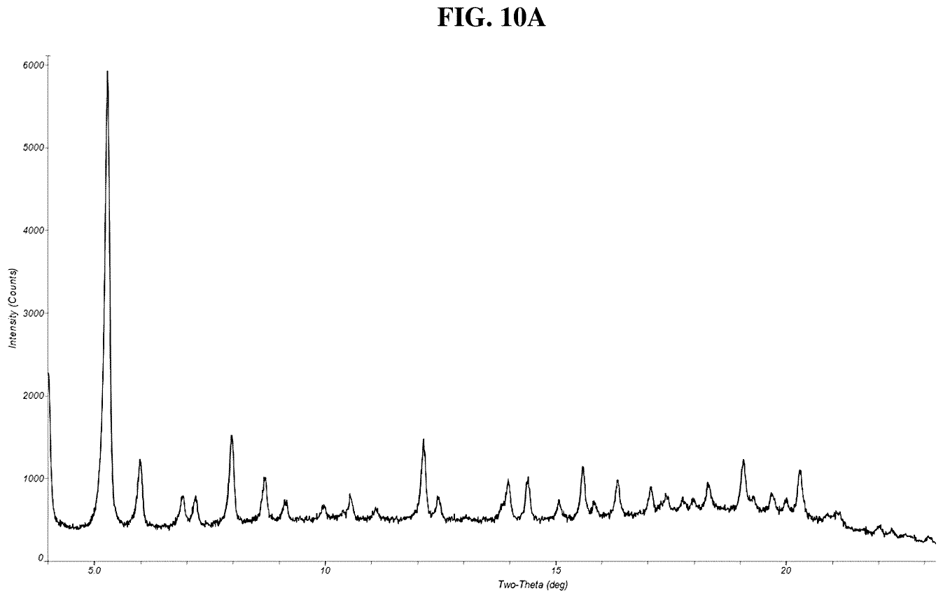

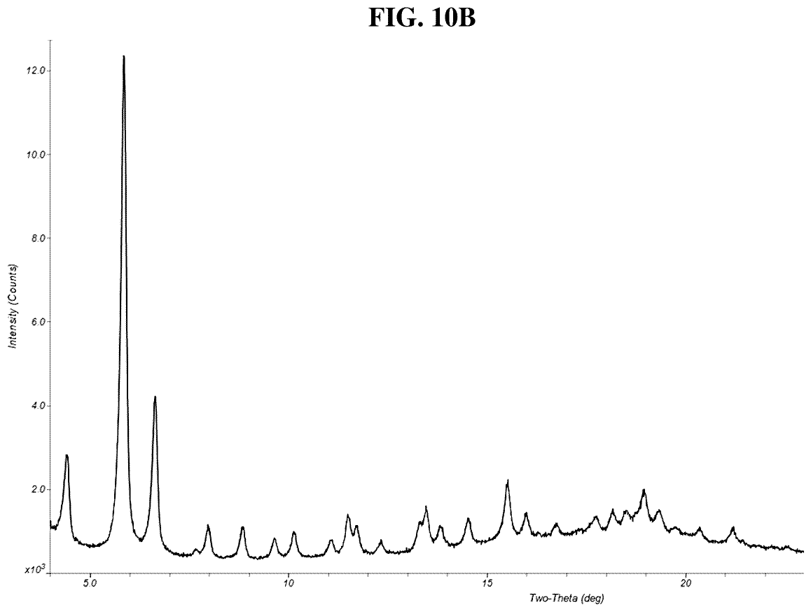

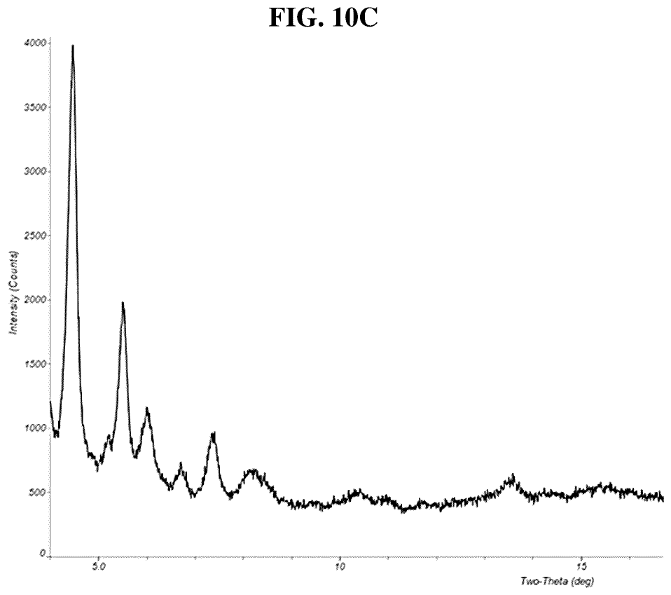

Prepared samples of the D-Y-S-K Form A were characterized by an XRPD pattern using CuKα radiation as having diffraction peaks (2-theta values) as described in Table 26 below, and in particular having a peak at 5.3 °2-Theta, in combination with one or more of the peaks selected from the group consisting of 6.0, 6.9, 7.2, 8.0, 12.2, and 15.6 °2-Theta; with a tolerance for the diffraction angles of 0.2 degrees. A representative XRPD pattern of D-Y-S-K Form A is shown in FIG. 10A.

TABLE 26
X-ray powder diffraction peaks of D-Y-S-K Form A
D-Y-S-K tetramer Form A

| | Angle | Relative Intensity |
|---|---|---|
| Peak | (°2-Theta) +/− 0.2° | (% of most intense peak) |
| 1 | 5.3 | 100.0% |
| 2 | 6.0 | 14.4% |
| 3 | 6.9 | 5.4% |
| 4 | 7.2 | 5.8% |
| 5 | 8.0 | 19.2% |
| 6 | 8.7 | 9.4% |
| 7 | 10.6 | 5.4% |
| 8 | 12.2 | 17.0% |
| 9 | 12.5 | 4.7% |
| 10 | 14.0 | 8.9% |
| 11 | 14.4 | 9.5% |
| 12 | 15.1 | 3.9% |
| 13 | 15.6 | 11.1% |
| 14 | 16.4 | 7.9% |
| 15 | 17.1 | 5.0% |
| 16 | 18.3 | 6.0% |
| 17 | 19.1 | 11.3% |
| 18 | 20.3 | 10.1% |

### XRPD of D-Y-S-K Form B

A prepared sample of the D-Y-S-K Form B was characterized by an XRPD pattern using CuKα radiation as having diffraction peaks (2-theta values) as described in Table 27 below, and in particular having a peak at 5.8 °2-Theta, in combination with one or more of the peaks selected from the group consisting of 4.4, 6.6, 10.1, 11.4, 13.4, and 15.5 °2-Theta; with a tolerance for the diffraction angles of 0.2 degrees. A representative XRPD pattern of D-Y-S-K Form B is shown in FIG. 10B.

TABLE 27
X-ray powder diffraction peaks of D-Y-S-K Form B
D-Y-S-K tetramer Form B

| | Angle | Relative Intensity |
|---|---|---|
| Peak | (°2-Theta) +/− 0.2° | (% of most intense peak) |
| 1 | 4.4 | 16.4% |
| 2 | 5.8 | 100.0% |
| 3 | 6.6 | 32.9% |
| 4 | 7.9 | 6.4% |
| 5 | 8.8 | 6.4% |
| 6 | 9.6 | 3.7% |
| 7 | 10.1 | 4.8% |
| 8 | 11.0 | 2.6% |
| 9 | 11.4 | 7.5% |
| 10 | 11.7 | 5.8% |
| 11 | 12.3 | 2.5% |
| 12 | 13.4 | 9.2% |
| 13 | 13.8 | 5.5% |
| 14 | 14.5 | 5.3% |
| 15 | 15.5 | 10.7% |
| 16 | 16.0 | 4.5% |
| 17 | 17.7 | 2.4% |
| 18 | 18.1 | 2.8% |
| 19 | 18.9 | 5.8% |
| 20 | 19.3 | 3.0% |
| 21 | 20.3 | 2.0% |

### XRPD of D-Y-S-K Form C

A prepared sample of the D-Y-S-K Form C was characterized by an XRPD pattern using CuKα radiation as having diffraction peaks (2-theta values) as described in Table 28 below, and in particular having peaks at 4.5 and 5.5 °2-Theta, in combination with one or more of the peaks selected from the group consisting of 6.0 and 7.3 °2-Theta; with a tolerance for the diffraction angles of 0.2 degrees. A representative XRPD pattern of D-Y-S-K Form C is shown in FIG. 10C.

TABLE 28
X-ray powder diffraction peaks of D-Y-S-K Form C
D-Y-S-K tetramer Form C

| | Angle | Relative Intensity |
|---|---|---|
| Peak | (°2-Theta) +/− 0.2° | (% of most intense peak) |
| 1 | 4.5 | 100.0% |
| 2 | 5.5 | 39.0% |
| 3 | 6.0 | 13.9% |
| 4 | 6.7 | 6.6% |
| 5 | 7.3 | 15.1% |
| 6 | 8.3 | 7.6% |

### Example 10: (Fmoc)Tyr-Ser(t-Bu)-Lys(Boc)-Tyr(t-Bu)

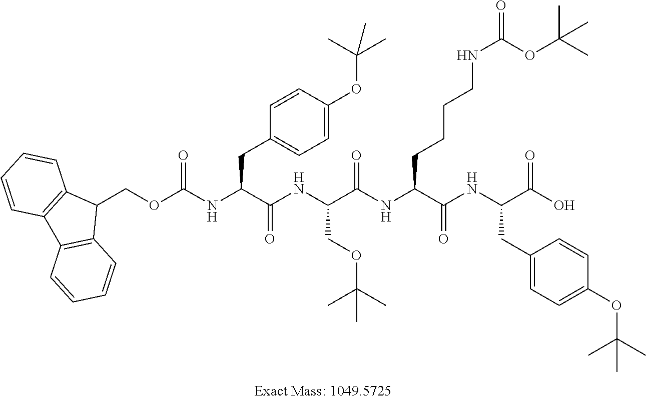 [SMILES: CC(C)(C)OC[C@H](NC(=O)[C@H](Cc1ccc(OC(C)(C)C)cc1)NC(=O)OCC1c2ccccc2-c2ccccc21)C(=O)N[C@@H](CCCCNC(=O)OC(C)(C)C)C(=O)N[C@@H](Cc1ccc(OC(C)(C)C)cc1)C(=O)O]

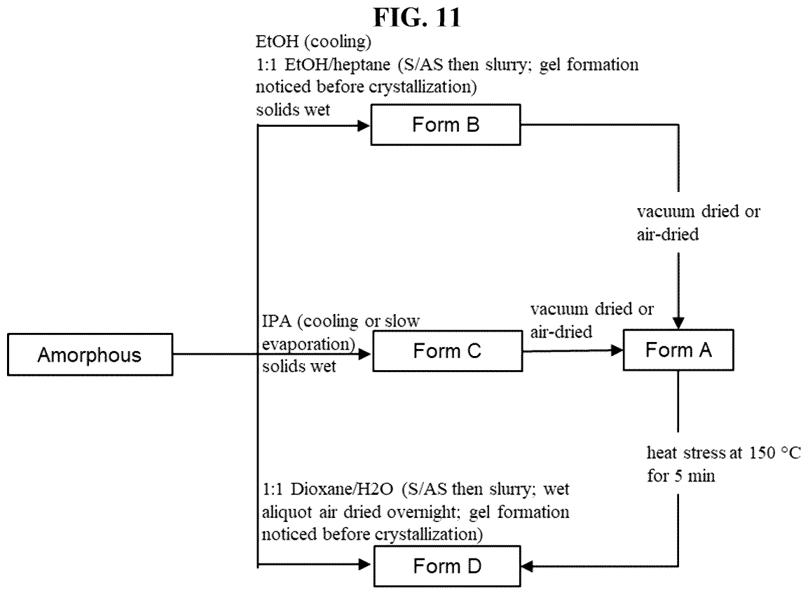

Multiple crystalline solid forms of OXM Y-S-K-Y tetramer were identified, including Y-S-K-Y Forms A through E. A polymorph map to describe their relationship is shown in FIG. 11. It is a desolvated solvate produced from solvated form came out of EtOH (Form B) or IPA (Form C). Form A converts to Form D upon heating to 150° C.; Form D was also observed from MEK.

### Y-S-K-Y Form A

50.3 mg of Y-S-K-Y amorphous solids were dissolved in 0.8 mL EtOH at 50° C. The solution was then removed from 50° C. heating plate and placed into a refrigerator. Solids were observed the next day, which were isolated, and air dried at ambient conditions. The obtained white solids are consistent with Y-S-K-Y Form A.

50.4 mg of Y-S-K-Y amorphous solids were dissolved in 0.75 mL IPA at 50° C. The solution was then removed from 50° C. heating plate and placed into a refrigerator. Solids were observed the next day, which were isolated, and air dried at ambient conditions. The obtained white solids are consistent with Y-S-K-Y Form A.

### Y-S-K-Y Form B

297.0 mg of Y-S-K-Y amorphous solids were dissolved in 2.5 mL EtOH at 54° C. yielding a slightly hazy solution. With stirring, the solution was allowed to slowly cool to ambient temperature on the heating plate with the heater turned off. A white suspension was obtained, and the solids are consistent with Y-S-K-Y Form B when analyzed wet.

### Y-S-K-Y Form C

50.4 mg of Y-S-K-Y amorphous solids were stirred in 1 mL IPA at ambient conditions for overnight resulting in a clear solution. The solution was allowed to evaporate at ambient conditions from an uncapped vial covered w/ perforated Al foil. The obtained damp solids were consistent with Y-S-K-Y Form C.

223.6 mg of Y-S-K-Y amorphous solids were dissolved in 1 mL IPA at 54° C. into a slightly hazy solution. With stirring, the solution was allowed to slowly cool to ambient temperature on the heating plate with the heater turned off. White solids were obtained in solution and were consistent with Y-S-K-Y Form C when analyzed wet.

### Y-S-K-Y Form D

11.1 mg of Y-S-K-Y Form A solids were heated to 150° C. on TGA and kept at the temperature for 5 minutes. The sample was then removed from the TGA instrument and analyzed by XRPD at ambient conditions. The obtained sample is white solids and consistent with Y-S-K-Y Form D.

50.4 mg of Y-S-K-Y amorphous solids were dissolved in 0.5 mL dioxane at ambient temperature into a clear solution. With stirring, 2×0.25 mL of water was added into the solution yielding a gel. The sample was vortexed to break the gel and the sample was stirred at ambient conditions for one day. An aliquot was removed from the white slurry to a clean silicon XRPD sample holder and air-dried on the sample holder at ambient conditions for overnight. The obtained solids were consistent with Y-S-K-Y Form D.

The XRPD patterns of crystalline Y-S-K-Y tetramers were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. Samples could also be scanned between 4 and 30 2θ° with a scan rate of 0.25 seconds/step, or between 4 and 25 2θ° with a scan rate of 0.1 seconds/step. The powder was packed on a silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of t 0.2 2θ° is presumed to take into account these potential variations without hindering the unequivocal identification of the indicated crystal form. Confirmation of a crystal form may be made based on any unique combination of distinguishing peaks.

### XRPD of Y-S-K-Y Form A

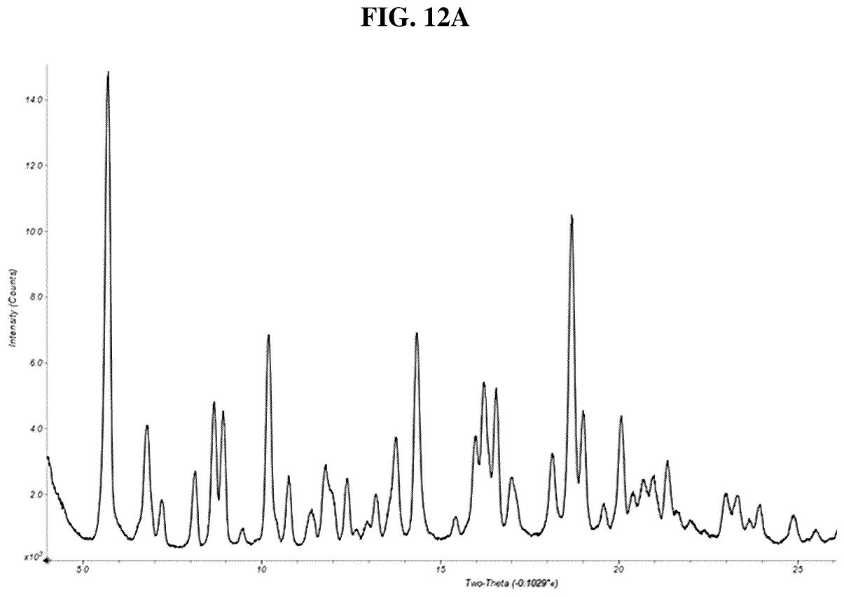

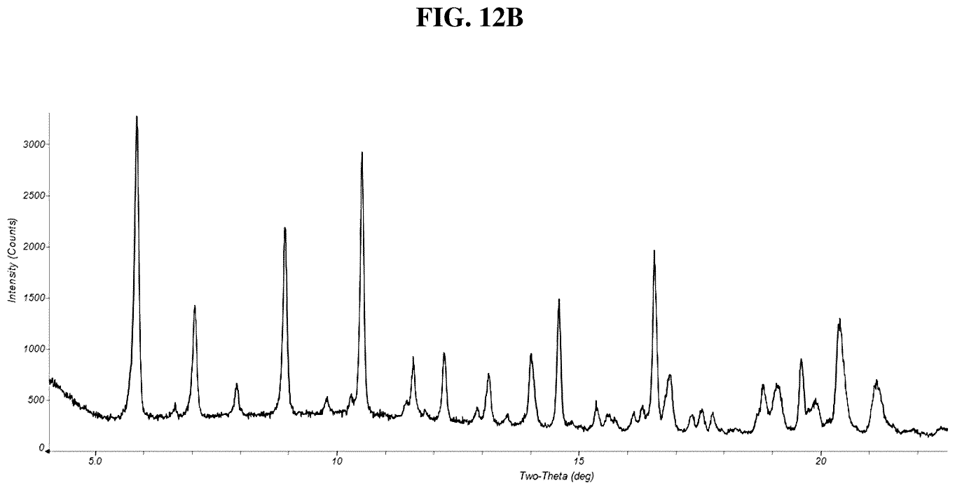

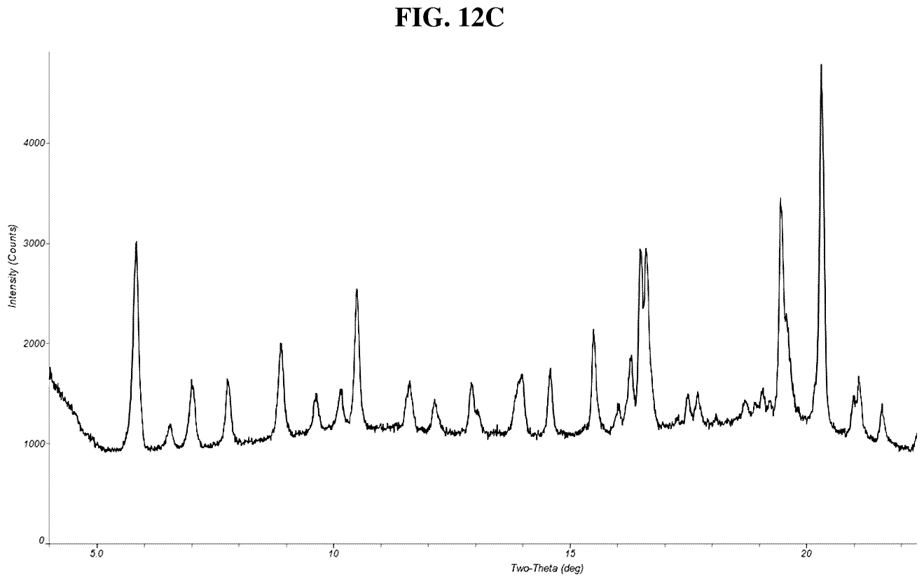

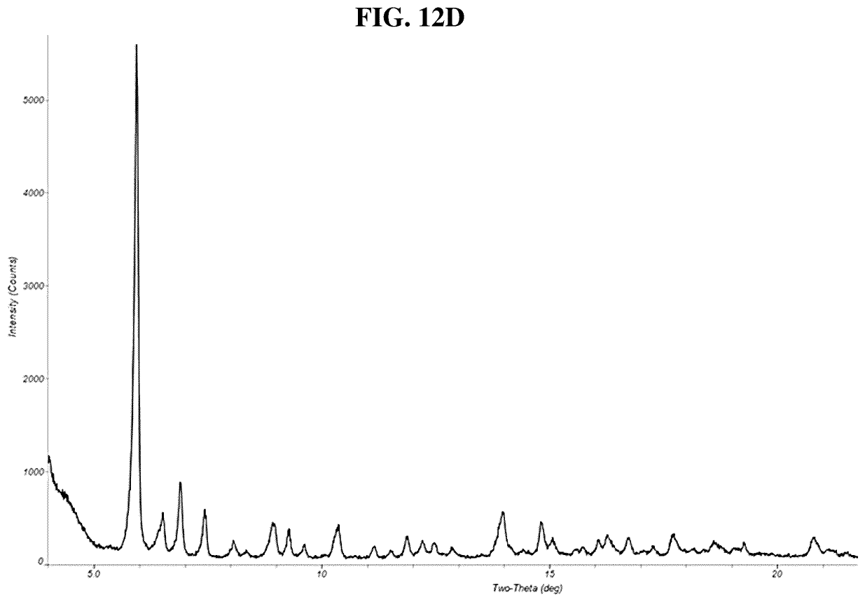

A prepared sample of the Y-S-K-Y Form A was characterized by an XRPD pattern using CuKα radiation as having diffraction peaks (2-theta values) as described in Table 29 below, and in particular having peaks at 18.1 and 18.7 °2-Theta, in combination with one or more of the peaks selected from the group consisting of 5.7, 8.7, 13.7, 14.3, 15.9, and 16.2 °2-Thea; with a tolerance for the diffraction angles of 0.2 degrees. A representative XRPD pattern of Y-S-K-Y Form A is shown in FIG. 12A.

TABLE 29
X-ray powder diffraction peaks of Y-S-K-Y Form A
Y-S-K-Y tetramer Form A

| | Angle | Relative Intensity |
|---|---|---|
| Peak | (°2-Theta) +/− 0.2° | (% of most intense peak) |
| 1 | 5.7 | 81.3% |
| 2 | 6.8 | 20.0% |
| 3 | 7.2 | 7.9% |
| 4 | 8.1 | 14.6% |
| 5 | 8.7 | 26.0% |
| 6 | 8.9 | 25.0% |
| 7 | 10.2 | 42.2% |
| 8 | 10.7 | 15.2% |
| 9 | 11.7 | 17.0% |
| 10 | 12.4 | 13.2% |
| 11 | 13.2 | 10.4% |
| 12 | 13.7 | 22.9% |
| 13 | 14.3 | 48.4% |
| 14 | 15.4 | 5.4% |
| 15 | 15.9 | 26.0% |
| 16 | 16.2 | 38.3% |
| 17 | 16.5 | 38.5% |
| 18 | 17.0 | 14.9% |
| 19 | 18.1 | 25.8% |
| 20 | 18.7 | 100.0% |
| 21 | 19.0 | 41.2% |
| 22 | 19.5 | 7.9% |
| 23 | 20.0 | 31.4% |
| 24 | 20.3 | 6.5% |
| 25 | 20.6 | 8.1% |
| 26 | 20.9 | 13.0% |
| 27 | 21.3 | 21.5% |

### XRPD of Y-S-K-Y Form B

A prepared sample of the Y-S-K-Y Form B was characterized by an XRPD pattern using CuKα radiation as having diffraction peaks (2-theta values) as described in Table 30 below, and in particular having peaks at 5.9 and 10.5 °2-Theta, in combination with one or more of the peaks selected from the group consisting of 7.1, 8.9, 14.6, and 16.6 °2-Theta; with a tolerance for the diffraction angles of 0.2 degrees. A representative XRPD pattern of Y-S-K-Y Form B is shown in FIG. 12B.

TABLE 30
X-ray powder diffraction peaks of Y-S-K-Y Form B
Y-S-K-Y tetramer Form B

| | Angle | Relative Intensity |
|---|---|---|
| Peak | (°2-Theta) +/− 0.2° | (% of most intense peak) |
| 1 | 5.9 | 100.0% |
| 2 | 7.1 | 36.7% |
| 3 | 7.9 | 10.7% |
| 4 | 8.9 | 61.9% |
| 5 | 9.8 | 5.6% |
| 6 | 10.5 | 86.7% |
| 7 | 11.6 | 20.3% |
| 8 | 12.2 | 22.4% |
| 9 | 13.1 | 16.5% |
| 10 | 14.0 | 23.9% |
| 11 | 14.6 | 42.6% |
| 12 | 16.6 | 58.7% |
| 13 | 16.9 | 18.7% |
| 14 | 18.8 | 14.8% |
| 15 | 19.1 | 12.2% |
| 16 | 19.6 | 22.8% |
| 17 | 20.4 | 37.6% |
| 18 | 21.2 | 17.5% |

### XRPD of Y-S-K-Y Form C

A prepared sample of the Y-S-K-Y Form C was characterized by an XRPD pattern using CuKα radiation as having diffraction peaks (2-theta values) as described in Table 31 below, and in particular having peaks at 7.8 and 20.3 °2-Theta, in combination with one or more of the peaks selected from the group consisting of 5.8, 15.5, and 19.5 °2-Theta; with a tolerance for the diffraction angles of 0.2 degrees. A representative XRPD pattern of Y-S-K-Y Form C is shown in FIG. 12C.

TABLE 31
X-ray powder diffraction peaks of Y-S-K-Y Form C
Y-S-K-Y tetramer Form C

| | Angle | Relative Intensity |
|---|---|---|
| Peak | (°2-Theta) +/− 0.2° | (% of most intense peak) |
| 1 | 5.8 | 57.6% |
| 2 | 6.5 | 6.7% |
| 3 | 7.0 | 18.7% |
| 4 | 7.8 | 18.4% |
| 5 | 8.9 | 26.2% |
| 6 | 9.6 | 10.2% |
| 7 | 10.1 | 9.6% |
| 8 | 10.5 | 37.1% |
| 9 | 11.6 | 13.3% |
| 10 | 12.1 | 8.5% |
| 11 | 12.9 | 14.4% |
| 12 | 14.0 | 16.9% |
| 13 | 14.6 | 18.5% |
| 14 | 15.5 | 28.8% |
| 15 | 16.3 | 12.7% |
| 16 | 16.5 | 42.2% |
| 17 | 16.6 | 49.2% |
| 18 | 19.5 | 59.3% |
| 19 | 20.3 | 100.0% |
| 20 | 21.1 | 16.9% |

### XRPD of Y-S-K-Y Form D

A prepared sample of the Y-S-K-Y Form D was characterized by an XRPD pattern using CuKα radiation as having diffraction peaks (2-theta values) as described in Table 32 below, and in particular having peaks at 5.9 and 7.4 °2-Theta, in combination with one or more of the peaks selected from the group consisting of 6.5, 6.9, and 14.8 02-Theta; with a tolerance for the diffraction angles of 0.2 degrees. A representative XRPD pattern of Y-S-K-Y Form D is shown in FIG. 12D.

TABLE 32
X-ray powder diffraction peaks of Y-S-K-Y Form D
Y-S-K-Y tetramer Form D

| | Angle | Relative Intensity |
|---|---|---|
| Peak | (°2-Theta) +/− 0.2° | (% of most intense peak) |
| 1 | 5.9 | 100.0% |
| 2 | 6.5 | 7.3% |
| 3 | 6.9 | 14.4% |
| 4 | 7.4 | 9.6% |
| 5 | 8.1 | 2.7% |
| 6 | 8.9 | 6.3% |
| 7 | 9.3 | 5.1% |
| 8 | 9.6 | 2.3% |
| 9 | 10.4 | 6.7% |
| 10 | 11.9 | 3.8% |
| 11 | 14.0 | 9.2% |
| 12 | 14.8 | 6.4% |
| 13 | 15.1 | 2.8% |
| 14 | 16.1 | 2.0% |
| 15 | 16.3 | 2.2% |
| 16 | 16.7 | 2.9% |
| 17 | 17.7 | 3.5% |
| 18 | 18.6 | 2.1% |
| 19 | 20.8 | 3.7% |

### Example 11: Fmoc-G-P-S-S-G-NH₂ Pentamer

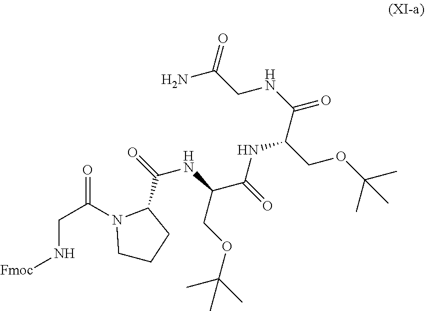

### Fmoc-G-P-S-S-G-NH₂ Crystalline Forms

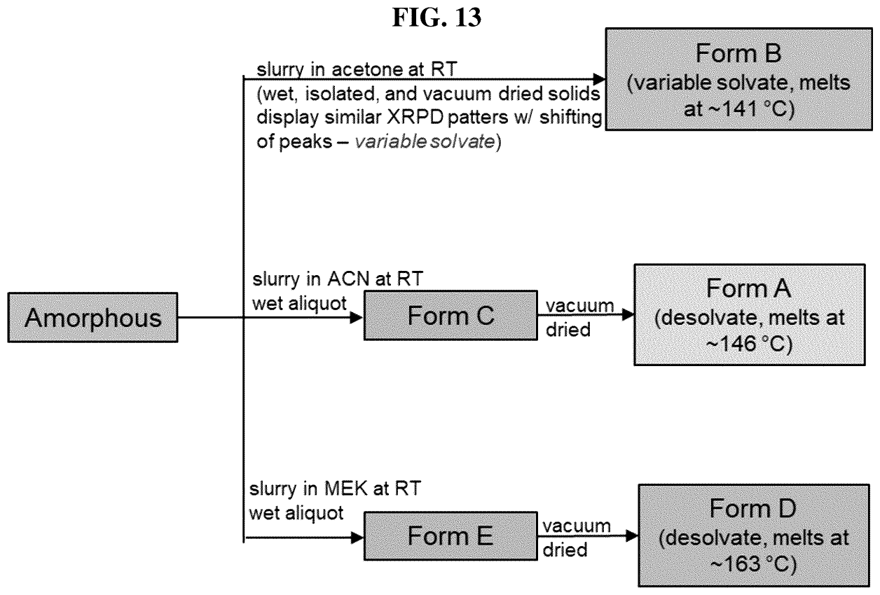

Multiple crystalline solid forms of OXM Fmoc protected G-P-S-S-G-NH₂ pentamer were identified, including Fmoc-G-P-S-S-G-NH₂ Forms A through E. A polymorph map to describe their relationship is shown in FIG. 13. Among them, Form B is a variable solvate produced from acetone, while Form A is a desolvated solvate came out of acetonitrile, and Form D is desolvated solvate came out of MEK.

### Fmoc-G-P-S-S-G-NH₂ Form A and Form C

Fmoc-G-P-S-S-G-NH₂ Form C was prepared from acetonitrile (ACN). 159.9 mg solids of amorphous Fmoc-G-P-S-S-G-NH₂ were stirred in 0.5 mL ACN at ambient conditions yielding a gel formation. Additional 0.5 mL of ACN was added to the sample to facilitate the stirring, and a white slurry was observed the next day. Additional 0.8 mL of ACN was added, and the sample was stirred at ambient conditions for a total of 3 weeks. The produced white solids were consistent with Fmoc-G-P-S-S-G-NH₂ Form C when analyzed wet.

Above white slurry was transferred to a 0.45 μm nylon centrifuge tube filter and centrifuged at ambient temperature for 5 minutes to separate solids and liquid. The isolated solids were dried in a vacuum oven at ambient temperature for overnight. The obtained white solids were consistent with Fmoc-G-P-S-S-G-NH₂ Form A.

### Fmoc-G-P-S-S-G-NH₂ Form B

Fmoc-G-P-S-S-G-NH₂ Form B was prepared from acetone. 150.1 mg solids of amorphous Fmoc-G-P-S-S-G-NH₂ were stirred in 0.5 mL acetone at ambient conditions yielding a gel formation. Additional 0.5 mL of acetone was added to the sample to facilitate the stirring, and a white slurry was observed the next day. Additional 0.3 mL of acetone was added, and the sample was stirred at ambient conditions for a total of 3 weeks. The produced white solids, either wet or dried, belong to Fmoc-G-P-S-S-G-NH₂ Form B.

### Fmoc-G-P-S-S-G-NH₂ Form D and Form E

Fmoc-G-P-S-S-G-NH₂ Form E was prepared from methyl ethyl ketone (MEK). 110.4 mg solids of amorphous Fmoc-G-P-S-S-G-NH₂ were stirred in 0.5 mL MEK at ambient conditions yielding a gel formation. Additional 2×0.5 mL of MEK was added to the sample to facilitate the stirring, which produced a mixture of white solids and gel in the sample the next day. The sample was sonicated in water bath and stirred manually by spatula to break the gel, and a white slurry was obtained. The sample was stirred at ambient conditions for a total of 3 weeks. The produced white solids were consistent with Fmoc-G-P-S-S-G-NH₂ Form E when analyzed wet.

The above white slurry was transferred to a 0.45 μm nylon centrifuge tube filter and centrifuged at ambient temperature for 5 minutes to separate solids and liquid. The isolated solids were dried in a vacuum oven at ambient temperature for overnight. The obtained white solids were consistent with Fmoc-G-P-S-S-G-NH₂ Form D.

The XRPD patterns of crystalline Fmoc-G-P-S-S-G-NH₂ pentamers were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. For Fmoc-G-P-S-S-G-NH₂ Form C and Form E, samples were scanned between 4 and 25 200 with a scan rate of 0.1 seconds/step. The powder was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### XRPD of Fmoc-G-P-S-S-G-NH₂ Form A

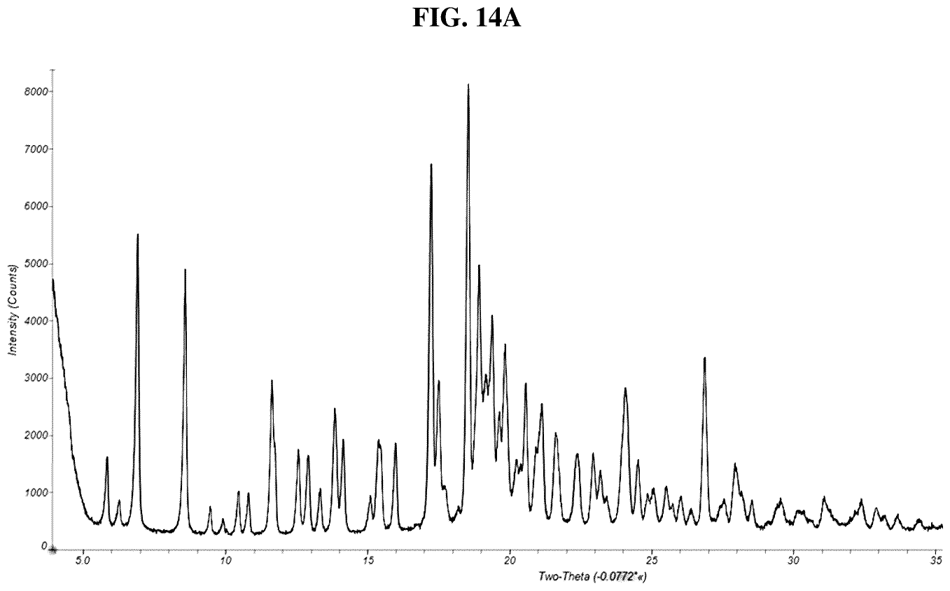

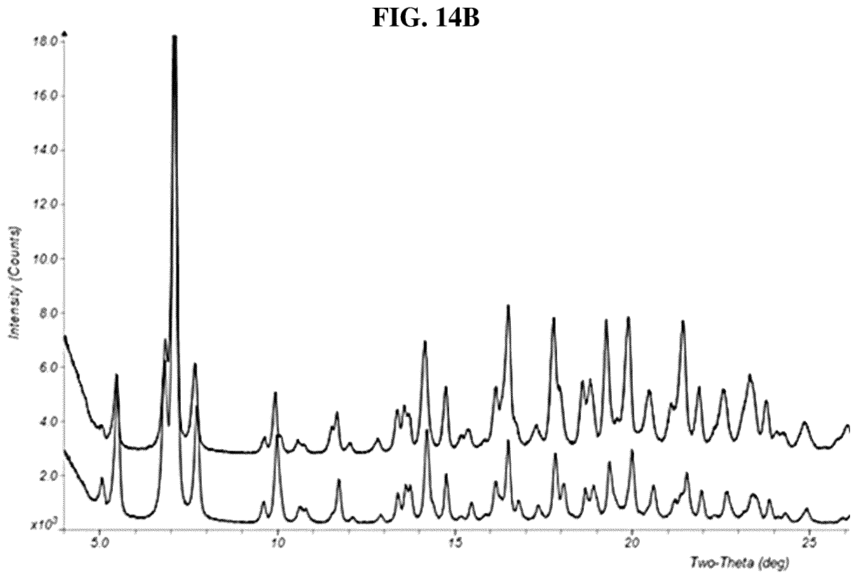

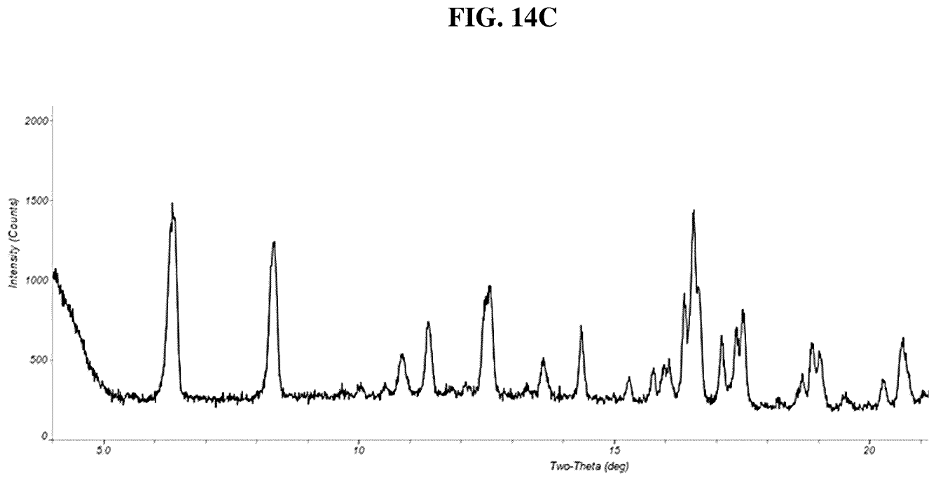

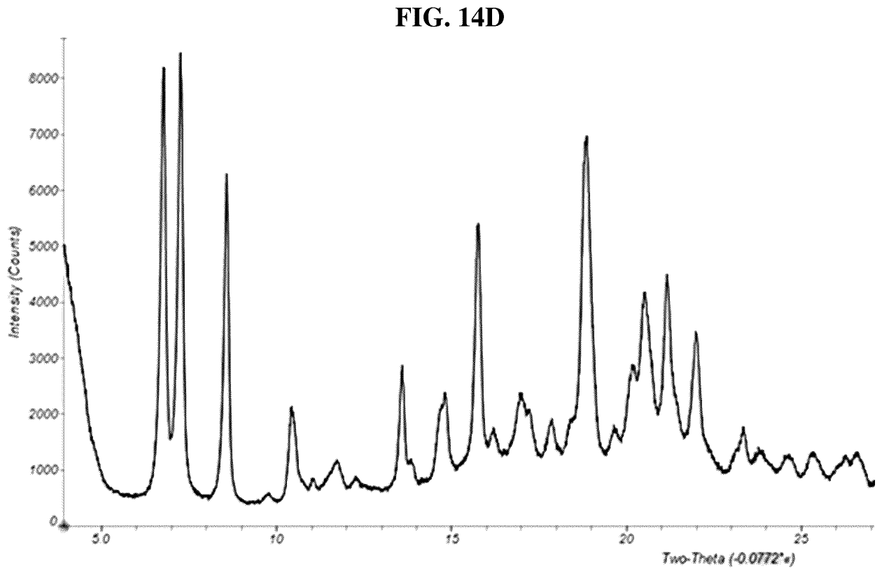

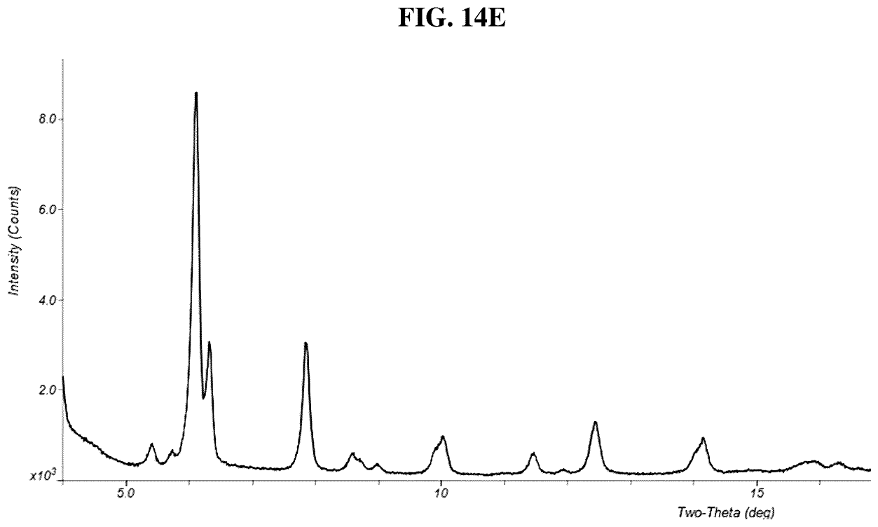

A prepared sample of the Fmoc-G-P-S-S-G-NH₂ Form A was characterized by an XRPD pattern using CuKα radiation as having diffraction peaks (2-theta values) as described in Table 33 below, and in particular having peaks at 5.8 and 18.5 °2-Theta, in combination with one or more of the peaks selected from the group consisting of 8.6, 9.4, 12.9, 13.8, 17.2, and 19.4 °2-Theta; with a tolerance for the diffraction angles of 0.2 degrees. A representative XRPD pattern of Fmoc-G-P-S-S-G-NH₂ Form A is shown in FIG. 14A.

TABLE 33
X-ray powder diffraction peaks of Fmoc-G-P-S-S-G-NH₂ Form A
Fmoc-G-P-S-S-G-NH₂ pentamer Form A

| | Angle | Relative Intensity |
|---|---|---|
| # | (°2-Theta) +/− 0.2° | (% of most intense peak) |
| 1 | 5.8 | 15.5% |
| 2 | 6.3 | 5.7% |
| 3 | 6.9 | 67.3% |
| 4 | 8.6 | 60.4% |
| 5 | 9.4 | 6.1% |
| 6 | 9.9 | 3.5% |
| 7 | 10.5 | 9.4% |
| 8 | 10.8 | 9.2% |
| 9 | 11.6 | 35.1% |
| 10 | 12.5 | 19.1% |
| 11 | 12.9 | 16.2% |
| 12 | 13.3 | 9.6% |
| 13 | 13.8 | 28.1% |
| 14 | 14.1 | 21.2% |
| 15 | 15.1 | 8.3% |
| 16 | 15.4 | 18.8% |
| 17 | 16.0 | 19.5% |
| 18 | 17.2 | 83.0% |
| 19 | 17.5 | 32.1% |
| 20 | 18.5 | 100.0% |
| 21 | 18.9 | 45.9% |
| 22 | 19.1 | 20.9% |
| 23 | 19.4 | 32.8% |
| 24 | 19.6 | 10.7% |
| 25 | 19.8 | 34.0% |
| 26 | 20.6 | 27.9% |
| 27 | 20.9 | 14.8% |
| 28 | 21.1 | 25.5% |
| 29 | 21.6 | 19.8% |
| 30 | 22.4 | 15.6% |
| 31 | 22.9 | 16.0% |
| 32 | 23.2 | 11.8% |
| 33 | 24.1 | 29.3% |
| 34 | 24.5 | 12.8% |
| 35 | 25.5 | 8.0% |
| 36 | 26.0 | 5.9% |
| 37 | 26.9 | 37.8% |

### XRPD of Fmoc-G-P-S-S-G-NH₂ Form B

A prepared sample of the Fmoc-G-P-S-S-G-NH₂ Form B was characterized by an XRPD pattern using CuKα radiation as having diffraction peaks (2-theta values) as described in Table 34 below, and in particular having peaks at 7.0-7.1, and 7.5-7.7 2 Theta, in combination with one or more of the peaks selected from the group consisting of 5.3-5.4, 9.7-9.9, and 14.7-14.9 °2-Theta; with a tolerance for the diffraction angles of 0.2 degrees. A representative XRPD pattern of Fmoc-G-P-S-S-G-NH₂ Form B is shown in FIG. 14B.

TABLE 34
X-ray powder diffraction peaks of Fmoc-G-P-S-S-G-NH₂ Form B
Fmoc-G-P-S-S-G-NH₂ pentamer Form B

| | Angle | Relative Intensity |
|---|---|---|
| Peak | (°2-Theta) +/− 0.2° | (% of most intense peak) |
| 1 | 5.3-5.4 | 2.3%-25.8% |
| 2 | 6.7-6.8 | 0-28.8% |
| 3 | 7.0-7.1 | 100.0% |
| 4 | 7.5-7.7 | 13.9%-20.7% |
| 5 | 9.7-9.9 | 2.0%-15.2% |
| 6 | 11.5-11.7 | 0.6%-8.3% |
| 7 | 14.0-14.1 | 8.2%-22.7% |
| 8 | 14.7-14.9 | 1.7%-13.0% |
| 9 | 16.1-16.2 | 7.0%-12.9% |
| 10 | 16.4-16.6 | 3.5%-30.5% |
| 11 | 17.6-17.8 | 3.6%-27.1% |
| 12 | 18.5-18.6 | 1.6%-13.4% |
| 13 | 19.2-19.3 | 3.5%-23.8% |
| 14 | 19.8-19.9 | 2.4%-24.1% |
| 15 | 21.0-21.1 | 3.0%-11.6% |

### XRPD of Fmoc-G-P-S-S-G-NH₂ Form C

A prepared sample of the Fmoc-G-P-S-S-G-NH₂ Form C was characterized by an XRPD pattern using CuKα radiation as having diffraction peaks (2-theta values) as described in Table 35 below, and in particular having peak at 8.3 °2-Theta, in combination with one or more of the peaks selected from the group consisting of 6.3, 11.4, 14.3, and 16.6 °2-Theta; with a tolerance for the diffraction angles of 0.2 degrees. A representative XRPD pattern of Fmoc-G-P-S-S-G-NH₂ Form C is shown in FIG. 14C.

TABLE 35
X-ray powder diffraction peaks of Fmoc-G-P-S-S-G-NH₂ Form C
Fmoc-G-P-S-S-G-NH₂ pentamer Form C

| | Angle | Relative Intensity |
|---|---|---|
| Peak | (°2-Theta) +/− 0.2° | (% of most intense peak) |
| 1 | 6.3 | 100.0% |
| 2 | 8.3 | 81.1% |
| 3 | 10.8 | 20.7% |
| 4 | 11.4 | 36.8% |
| 5 | 12.6 | 55.8% |
| 6 | 13.6 | 19.0% |
| 7 | 14.3 | 37.7% |
| 8 | 15.3 | 11.9% |
| 9 | 15.8 | 12.4% |
| 10 | 16.1 | 18.8% |
| 11 | 16.4 | 53.0% |
| 12 | 16.6 | 97.7% |
| 13 | 17.1 | 29.3% |
| 14 | 17.4 | 22.3% |
| 15 | 17.5 | 50.5% |
| 16 | 18.7 | 10.4% |
| 17 | 18.9 | 33.7% |
| 18 | 19.0 | 25.9% |

### XRPD of Fmoc-G-P-S-S-G-NH₂ Form D

A prepared sample of the Fmoc-G-P-S-S-G-NH₂ Form D was characterized by an XRPD pattern using CuKα radiation as having diffraction peaks (2-theta values) as described in Table 36 below, and in particular having peak at 7.2 °2-Theta, in combination with one or more of the peaks selected from the group consisting of 6.8, 8.6, 15.8, and 18.9 °2-Theta; with a tolerance for the diffraction angles of 0.2 degrees. A representative XRPD pattern of Fmoc-G-P-S-S-G-NH₂ Form D is shown in FIG. 14D.

TABLE 36
X-ray powder diffraction peaks of Fmoc-G-P-S-S-G-NH₂ Form D
Fmoc-G-P-S-S-G-NH2 pentamer Form D

| | Angle | Relative Intensity |
|---|---|---|
| Peak | (°2-Theta) +/− 0.2° | (% of most intense peak) |
| 1 | 6.8 | 96.3% |
| 2 | 7.2 | 100.0% |
| 3 | 8.6 | 72.9% |
| 4 | 10.4 | 21.6% |
| 5 | 11.7 | 6.1% |
| 6 | 13.6 | 26.1% |
| 7 | 14.8 | 17.7% |
| 8 | 15.8 | 51.0% |
| 9 | 16.2 | 4.4% |
| 10 | 17.0 | 13.6% |
| 11 | 17.9 | 7.6% |
| 12 | 18.9 | 70.9% |
| 13 | 20.1 | 11.2% |
| 14 | 20.5 | 21.5% |
| 15 | 21.2 | 31.8% |
| 16 | 22.0 | 31.2% |
| 17 | 23.3 | 8.5% |

### XRPD of Fmoc-G-P-S-S-G-NH₂ Form E

A prepared sample of the Fmoc-G-P-S-S-G-NH₂ Form E was characterized by an XRPD pattern using CuKα radiation as having diffraction peaks (2-theta values) as described in Table 37 below, and in particular having peak at 6.1 °2-Theta, in combination with one or more of the peaks selected from the group consisting of 6.3, 7.8, 10.0, and 12.4 °2-Theta; with a tolerance for the diffraction angles of 0.2 degrees. A representative XRPD pattern of Fmoc-G-P-S-S-G-NH₂ Form E is shown in FIG. 14E.

TABLE 37
X-ray powder diffraction peaks of Fmoc-G-P-S-S-G-NH₂ Form E
Fmoc-G-P-S-S-G-NH₂ pentamer Form E

| | Angle | Relative Intensity |
|---|---|---|
| Peak | (°2-Theta) +/− 0.2° | (% of most intense peak) |
| 1 | 5.4 | 5.3% |
| 2 | 6.1 | 100.0% |
| 3 | 6.3 | 34.0% |
| 4 | 7.8 | 34.2% |
| 5 | 8.6 | 4.3% |
| 6 | 9.0 | 2.3% |
| 7 | 10.0 | 9.9% |
| 8 | 11.5 | 5.3% |
| 9 | 11.9 | 0.8% |
| 10 | 12.4 | 14.0% |
| 11 | 14.1 | 9.4% |

### Example 12: Fmoc-Gly-Pro-Ser(tBu)-Ser(tBu)-Gly-OH

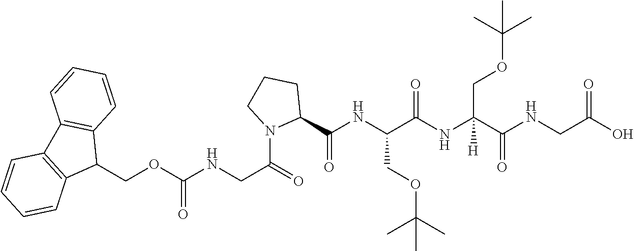 [SMILES: CC(C)(C)OC[C@H](NC(=O)[C@H](COC(C)(C)C)NC(=O)[C@@H]1CCCN1C(=O)CNC(=O)OCC1c2ccccc2-c2ccccc21)C(=O)NCC(=O)O]

A crystallization screen was performed on Fmoc-G-P-S-S-G-OH free form using various solvents and solvent mixtures. Different crystallization methods were employed including solvent-based techniques such as slurry, cooling, ambient and sub-ambient temperatures holding, solvent/anti-solvent addition, or combination of techniques, and non-solvent-based techniques such as heat stress.

Two solid forms were identified: Fmoc-G-P-S-S-G-OH Form A and Fmoc-G-P-S-S-G-OH Form B. Form A was a solvated form produced from 1-propanol (1-PrOH) or mixtures containing 1-PrOH (such as 1-PrOH/heptane). Form B was a desolvating product of Form A.

### Fmoc-G-P-S-S-G-OH Form A

### Preparation 1:

Fmoc-G-P-S-S-G-OH Form A was prepared in 1-propanol (1-PrOH). 16 mL 1-PrOH was added to 4.06 gram of amorphous solids of Fmoc-G-P-S-S-G-OH and the sample was stirred at ambient conditions which yielded a red-orange solution. The solution was seeded with 2.8 mg of Fmoc-G-P-S-S-G-OH Form A and then left at ambient conditions to continue stirring for 2 days. A light orange suspension was obtained, and solids were isolated by vacuum filtration using a 10 μm disposable filter, rinsed on the filter w/ 0.5 mL fresh 1-PrOH twice, and then collected and dried at 30° C. under vacuum for about 3-4 hours. The resulting white solids (3.2 gram) were consistent with Fmoc-G-P-S-S-G-OH Form A.

### Preparation 2:

Fmoc-G-P-S-S-G-OH Form A was prepared in 1-propanol (1-PrOH) and heptane. Approximately 50 mg of amorphous Fmoc-G-P-S-S-G-OH was dissolved in 0.6 mL 1-PrOH to form a clear yellow solution. The solution, in 30 μL aliquots, was added into 0.6 mL heptane yielding a clear light yellow solution. With stirring, additional 0.6 mL heptane was added to the solution and the sample was capped and stirred at ambient conditions for 2 days. A suspension was obtained, and solids were consistent with Fmoc-G-P-S-S-G-OH Form A.

### Preparation 3:

Fmoc-G-P-S-S-G-OH Form A was prepared in 1-propanol (1-PrOH). Approximately 10 mg of amorphous Fmoc-G-P-S-S-G-OH was dissolved in 0.1 mL 1-PrOH to form a clear yellow solution. The solution was stored at ambient conditions in capped vial for 1 day, then moved to freezer for 3 days. Solids observed in solution were consistent with Fmoc-G-P-S-S-G-OH Form A.

### Fmoc-G-P-S-S-G-OH Form B

Solids of Fmoc-G-P-S-S-G-OH Form A were loaded to a clean TGA pan, heated to 120° C. and held at the temperature for 3 minutes in TGA furnace. The resulting white solids were consistent with Fmoc-G-P-S-S-G-OH Form B.

### XRPD of Fmoc-G-P-S-S-G-OH Form A

The XRPD pattern of crystalline Fmoc-G-P-S-S-G-OH Form A was obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. The powder was packed on a quartz sample holder and a smooth surface as obtained using a glass slide. The diffraction patterns were collected at ambient temperature and relative humidity. Crystal peak positions were determined in MDI-Jade v7.9.9.

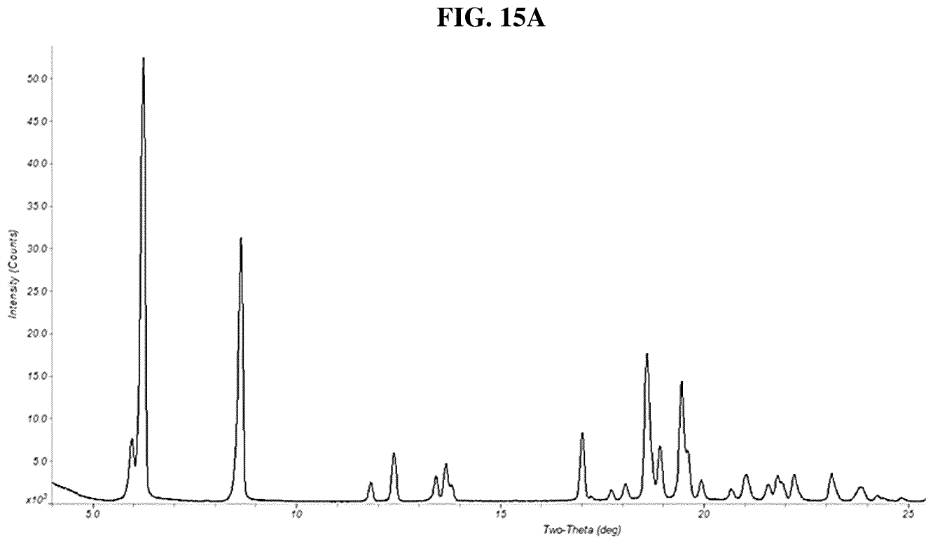

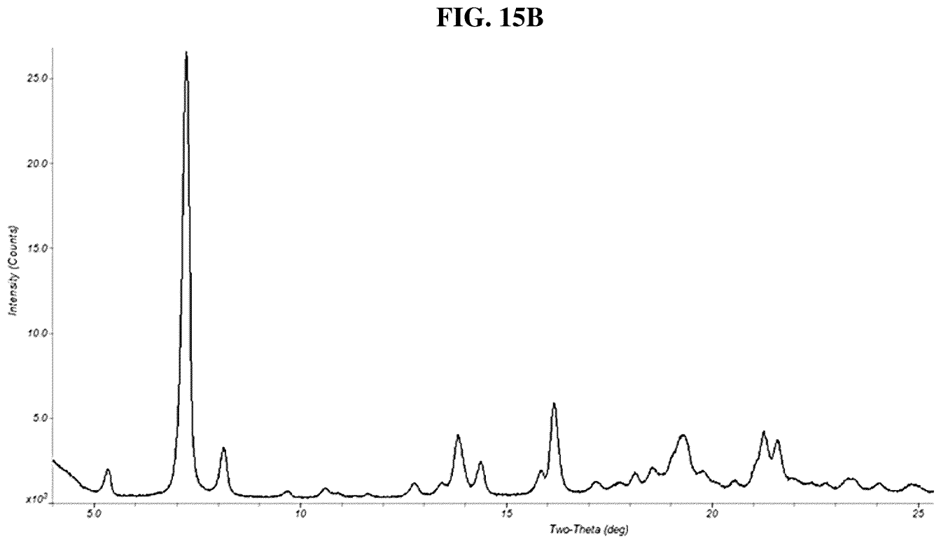

A prepared sample of Fmoc-G-P-S-S-G-OH Form A was characterized by an XRPD pattern using CuKα radiation as having diffraction peaks (2-theta values) as described in Table 38 below, and in particular having a peak at 6.1 °2-Theta in combination with one or more of the peaks selected from 8.5, 11.7, 12.3, and 16.9° 2 Theta; with a tolerance for the diffraction angles of 0.2 degrees. A representative XRPD patter of Y-Aib-Q-G Form A is shown in FIG. 15A.

TABLE 38
X-ray powder diffraction peaks of Fmoc-G-P-S-S-G-OH Form A
Fmoc-G-P-S-S-G-OH Form A

| | Angle | Relative Intensity |
|---|---|---|
| Peak | (°2-Theta) +/− 0.2° | (% of most intense peak) |
| 1 | 5.8 | 12.7% |
| 2 | 6.1 | 100.0% |
| 3 | 8.5 | 63.8% |
| 4 | 11.7 | 6.9% |
| 5 | 12.3 | 10.2% |
| 6 | 13.3 | 7.8% |
| 7 | 13.5 | 10.5% |
| 8 | 16.9 | 15.5% |
| 9 | 18.0 | 7.4% |
| 10 | 18.5 | 28.6% |
| 11 | 18.8 | 20.9% |
| 12 | 19.3 | 23.3% |
| 13 | 19.5 | 8.2% |
| 14 | 19.8 | 7.3% |
| 15 | 20.9 | 11.0% |
| 16 | 21.7 | 6.5% |
| 17 | 22.1 | 6.3% |
| 18 | 23.0 | 9.9% |

### XRPB of Fmoc-G-P-S-S-G-OH Form B

The XRPD pattern of crystalline Fmoc-G-P-S-S-G-OH Form B was obtained using the same procedures as Fmoc-G-P-S-S-G-OH Form A, but the Fmoc-GPSSG-OH Form B sample was scanned between 4 and 30 200 with a scan rate of 0.25 seconds/step.

A prepared sample of the Fmoc-G-P-S-S-G-OH Form B was characterized by an XRPD pattern using CuKα radiation as having diffraction peaks (2-theta values) as described in Table 39 below, and in particular having a peak at 7.2 °2-Theta, in combination with one or more of the peaks selected from the group consisting of 5.3, 8.1, 14.4, and 16.2 °2-Theta; with a tolerance for the diffraction angles of 0.2 degrees. A representative XRPD pattern of Fmoc-G-P-S-S-G-OH Form B is shown in FIG. 15B.

TABLE 39
X-ray powder diffraction peaks of Fmoc-G-P-S-S-G-OH Form B
Fmoc-G-P-S-S-G-OH Form B

| | Angle | Relative Intensity |
|---|---|---|
| Peak | (°2-Theta) +/− 0.2° | (% of most intense peak) |
| 1 | 5.3 | 5.3% |
| 2 | 7.2 | 100.0% |
| 3 | 8.1 | 11.0% |
| 4 | 12.8 | 2.7% |
| 5 | 13.8 | 13.4% |
| 6 | 14.4 | 7.4% |
| 7 | 15.8 | 5.5% |
| 8 | 16.2 | 19.7% |
| 9 | 19.3 | 11.6% |
| 10 | 21.2 | 12.1% |
| 11 | 21.6 | 10.1% |

### Example 13: GGG Side Chain

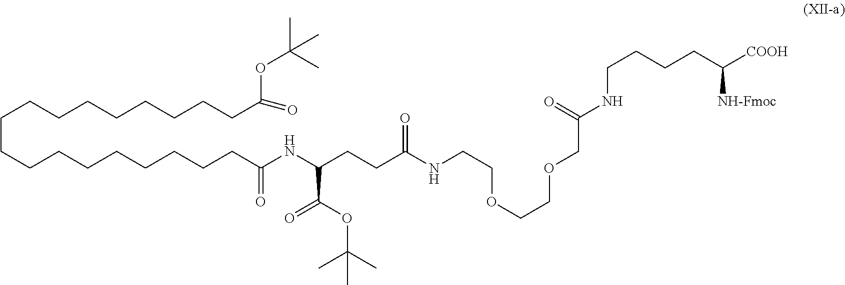

### Method of Making, Crystallization, and Use

2-(2-(2-Aminoethoxy)ethoxy)acetic acid (1.16 eq) and 5 vol acetonitrile were charged in a jacketed reactor. N-Methyl-N-trimethylsilylacetamide (2.56 eq) was added to it slowly. It was allowed to stir at 20-25° C. for about 2-3 hours. The reaction mixture was clear and 1 eq of (O1-tert-butyl 05-(2,5-dioxopyrrolidin-1-yl) (2S)-2-[(20-tert-butoxy-20-oxo-icosanoyl)amino]pentanedioate) was added to the reaction mixture. The reaction mixture was allowed to stir for 4-5 hours at about 20-25° C. About 9 vol of 2-methyltetrahydrofuran (2-Me-THF) were added to the reaction mixture, followed by extraction with 3×4 vol of aqueous 2% KHSO₄ and 1% NaCl solution. The organic solution was then washed with 4×4 vol of aqueous 2% NaCl solution. The organic solution was concentrated under vacuum to reduce acetonitrile (<0.1%) and water (KF<0.5%). One vol was DMF was charged to make the DMF solution of (2-[2-[2-[[(4S)-5-tert-butoxy-4-[(20-tert-butoxy-20-oxo-icosanoyl)amino]-5-oxo-pentanoyl]amino]ethoxy]ethoxy]acetic acid. The organic mixture was concentrated under vacuum to reduce 2-Me-THF below 5%. A DMF feed solution of (2-[2-[2-[[(4S)-5-tert-butoxy-4-[(20-tert-butoxy-20-oxo-icosanoyl)amino]-5-oxo-pentanoyl]amino]ethoxy]ethoxy]acetic acid with 2.4 eq of DIEA; a DMF solution of TNTU, and a DMF solution of (((9H-fluoren-9-yl)methoxy)carbonyl)-L-lysine hydrochloride were prepared. Feed tanks of 2-MeTHF, and aqueous 3% KHSO₄ and 5% NaCl were also prepared. The flow rates of (2-[2-[2-[[(4S)-5-tert-butoxy-4-[(20-tert-butoxy-20-oxo-icosanoyl)amino]-5-oxo-pentanoyl]amino]ethoxy]ethoxy]acetic acid (containing 2.4 eq DIEA) solution and TNTU solution were adjusted to charge 0.97 eq of TNTU with respect to (2-[2-[2-[[(4S)-5-tert-butoxy-4-[(20-tert-butoxy-20-oxo-icosanoyl)amino]-5-oxo-pentanoyl]amino]ethoxy]ethoxy]acetic acid. The reaction mixture was warmed in a reactor coil at around 30° C., which then was introduced to a stream of DMF solution of (((9H-fluoren-9-yl)methoxy)carbonyl)-L-lysine hydrochloride to react with 1.15 eq of (((9H-fluoren-9-yl)methoxy)carbonyl)-L-lysine hydrochloride at around 30° C. in a reactor coil to give the GGG side chain product. Then, the reaction solution was diluted with 30 vol of 2-MeTHF and quenched with 20 vol of aqueous 3% KHSO₄ and 5% NaCl solution. The organic solution was then cooled to 10-20° C. and aqueous phase was separated. Concentrated the organic solution to 11-13 vol, charged 2.3-2.7 vol DMF, and then extracted with 5 vol of aqueous 3% KHSO₄ and 5% NaCl solution. The organic phase was diluted with 2.3-2.7 vol DMF and extracted with 5 vol of aqueous 3% KHSO₄ and 5% NaCl solution. Then, 2.5-3.2 vol 2-MeTHF and 2.3-2.7 vol DMF were charged and extracted the organics with 5 vol of aqueous 3% KHSO₄ and 5% NaCl solution. The cycle of diluting with DMF, extraction with 3% KHSO₄ and 5% NaCl solution, then diluting with 2-MeTHF and DMF followed by extraction with 3% KHSO₄ and 5% NaCl solution was repeated once more. 2.5-3.2 vol of 2-MeTHF was then charged, and the organic solution was extracted 4 times with 5% aqueous NaCl solution. The 2-MeTHF solution was then concentrated, and 2-MeTHF was swapped with acetonitrile through repeated addition of acetonitrile and concentration of the solution under vacuum to bring down 2-MeTHF below 10%.

### Crystallization

After solvent swap via distillation, ACN was added to 30 vol. The solution was heated to 55° C. to fully dissolve all sidechain material. The solution was cooled to 5° C. over 4 hours and then held for 4 hours for primary nucleation. Primary nucleation reliably occurred during the 5° C. hold and resulted in a mix of amorphous and crystalline material. This was undesirable so the next steps were designed to convert to crystalline material.

The solution was warmed to 30° C. and held for 8 hours to dissolve amorphous material. Next, 6 vol of MEK were added and the solution was held for an additional 3 hours to dissolve additional amorphous material and then cooled to 20° C. Four thermal cycles were employed to convert from amorphous to crystalline: In each cycle, the mixture was heated to 30° C., held 3 hours, then cooled to 20° C. and held 3 hours.

After thermal cycles, the mixture was cooled to 5° C. over 1 hour and held 1 hour. A sample of the supernatant was collected and tested by HPLC for sidechain potency. If the sidechain concentration was 5 mg/mL or less, it suggested that the solid form was the low solubility crystalline form. The slurry was then filtered, rinsed three times with 10 vol ACN at 5° C. and dried at 30° C.

### GGG Side Chain Crystalline Forms

Three crystalline solid forms of the, GGG side chain were identified, including GGG SC Form A, Form B and Form C.

Form A is a crystalline anhydrous/unsolvated form and observed from various solvent conditions at ambient temperature.

Form B is a crystalline material and was observed in water.

Form C is a crystalline anhydrous/unsolvated form and was observed at elevated temperature.

### GGG SC Form A

241.2 mg solids of GGG side chain were added to 2 mL of 1:3 vol/vol MEK/ACN and the mixture was heated at about 75° C. for 10 minutes resulting a clear solution. The solution remained clear after it slowly cooled to ambient temperature.

About half volume of the above solution was seeded with disordered Form A and then stirred at ambient condition which produced gel particles first then a thick white paste with no flowable liquid. After addition of another 0.5 mL of 1:3 vol/vol MEK/ACN, the sample was stirred at ambient conditions for 2 days resulting a thick white slurry. The white solids isolated from the sample by vacuum filtration were consistent with GGG side chain Form A.

GGG side chain Form A was also obtained by stirring the above solution at ambient conditions without seeding. The sample also went through formation of gel particles and thick white paste that required adding of additional volume of 1:3 vol/vol MEK/ACN for slurry.

### GGG Side Chain Form B

Approximately 20 mg of disordered GGG side chain solids were slurried in water at 60° C. resulting birefringent white particles. The sample was isolated and dried under nitrogen stream on Whatman filter. And the obtained white solids were consistent with GGG side chain Form B.

### GGG Side Chain Form C

GGG side chain Form A was heated to 93° C. and obtained solids were consistent with GGG side chain Form C.

The XRPD patterns of crystalline GGG side chain (Form A or Form B) were obtained on a Bruker D8 Endeavor X-ray powder diffractometer, equipped with a CuKα (1.5418 Å) source and a Linxeye detector, operating at 40 kV and 40 mA. The sample was scanned between 4 and 42 2θ°, with a step size of 0.009 2θ° and a scan rate of 0.5 seconds/step, and using 0.3° primary slit opening, and 3.9° PSD opening. In some cases, a scan range of 4 and 30 2θ°, with a step size of 0.009 2θ° and a scan rate of 0.25 seconds/step was used. The sample was packed on a quartz or silicon sample holder and a smooth surface was obtained using a glass slide. The crystal form diffraction patterns were collected at ambient temperature and relative humidity. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

The XRPD patterns of crystalline GGG side chain Form C were obtained with a PANalytical X'Pert PRO MPD diffractometer using an incident beam of Cu Kα radiation produced using a long, fine-focus source and a nickel filter. The diffractometer was configured using the symmetric Bragg-Brentano geometry. Data were collected and analyzed using Data Collector software v. 2.2b. Prior to the analysis, a silicon specimen (NIST SRM 640e) was analyzed to verify the observed position of the Si 111 peak is consistent with the NIST-certified position. A specimen of the sample was packed in a nickel-coated copper well. Antiscatter slits (SS) were used to minimize the background generated by air scattering. Soller slits for the incident and diffracted beams were used to minimize broadening from axial divergence. Diffraction patterns were collected using a scanning position-sensitive detector (X'Celerator) located 240 mm from the sample. The data acquisition was from 3.5°-30° 2θ with a step size of 0.017 2θ°. Crystal peak positions were determined in MDI-Jade v7.9.9. It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of f 0.2 200 is presumed to take into account these potential variations without hindering the unequivocal identification of the indicated crystal form. Confirmation of a crystal form may be made based on any unique combination of distinguishing peaks.

### XRPD of GGG Side Chain, Form A

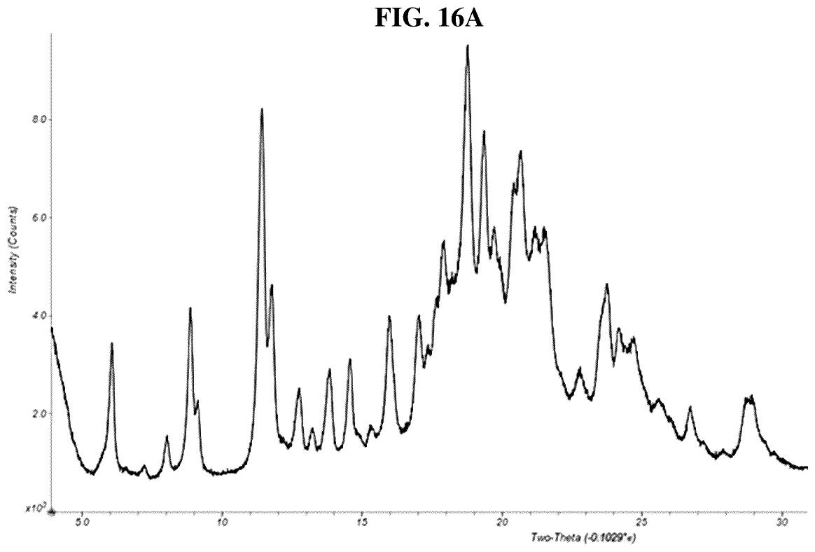

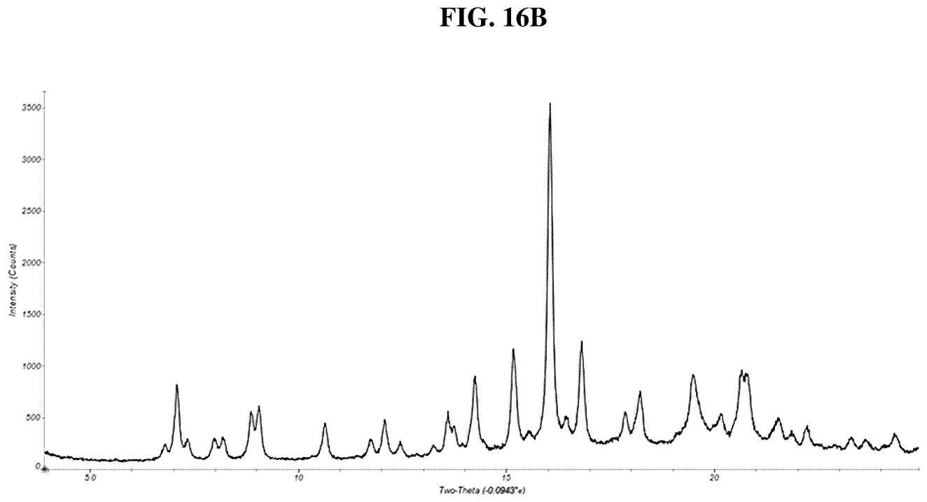

Prepared samples of the GGG side chain, Form A were characterized by an XRPD pattern using CuKα radiation as having diffraction peaks (2-theta values) as described in Table 40 below, and in particular having a peak at 11.4 °2-Theta, in combination with one or more of the peaks selected from the group consisting of 6.0, 8.9, 12.7, 13.6, 14.6, 17.0 and 18.8 °2-Theta; with a tolerance for the diffraction angles of 0.2 degrees. A representative XRPD pattern of GGG side chain Form A is shown in FIG. 16A.

TABLE 40
X-ray powder diffraction peaks of GGG side chain Form A
GGG side chain Form A

| | Angle | Relative Intensity |
|---|---|---|
| Peak | (°2-Theta) +/− 0.2° | (% of most intense peak) |
| 1 | 6.0 | 36.2% |
| 2 | 7.2 | 3.0% |
| 3 | 8.0 | 9.3% |
| 4 | 8.9 | 43.9% |
| 5 | 9.1 | 18.8% |
| 6 | 11.4 | 100.0% |
| 7 | 11.8 | 51.6% |
| 8 | 12.7 | 15.9% |
| 9 | 13.2 | 5.6% |
| 10 | 13.8 | 22.0% |
| 11 | 14.6 | 24.6% |
| 12 | 15.3 | 3.9% |
| 13 | 16.0 | 32.5% |
| 14 | 17.0 | 31.9% |
| 15 | 17.9 | 33.4% |
| 16 | 18.8 | 63.6% |
| 17 | 19.3 | 34.6% |
| 18 | 19.7 | 8.2% |
| 19 | 20.4 | 28.0% |
| 20 | 20.6 | 33.4% |
| 21 | 21.2 | 12.6% |
| 22 | 21.5 | 3.7% |
| 23 | 22.8 | 7.3% |
| 24 | 23.8 | 26.9% |
| 25 | 24.2 | 14.9% |
| 26 | 24.7 | 11.1% |

### XRPD of GGG Side Chain Form B

A prepared sample of the GGG side chain, Form B was characterized by an XRPD pattern using CuKα radiation as having diffraction peaks (2-theta values) as described in Table 41 below, and in particular having a peak at 10.6 °2-Theta, in combination with one or more of the peaks selected from the group consisting of 7.1, 12.1, 13.6, 14.2, 15.2, 16.0, and 16.8 °2-Theta; with a tolerance for the diffraction angles of 0.2 degrees. A representative XRPD pattern of GGG side chain Form B is shown in FIG. 16B.

TABLE 41
X-ray powder diffraction peaks of GGG side chain Form B
GGG side chain Form B

| | Angle | Relative Intensity |
|---|---|---|
| Peak | (°2-Theta) +/− 0.2° | (% of most intense peak) |
| 1 | 6.8 | 3.5% |
| 2 | 7.1 | 22.4% |
| 3 | 7.3 | 5.5% |
| 4 | 8.0 | 5.5% |
| 5 | 8.2 | 5.7% |
| 6 | 8.9 | 14.1% |
| 7 | 9.0 | 16.1% |
| 8 | 10.6 | 10.7% |
| 9 | 11.7 | 5.7% |
| 10 | 12.1 | 9.7% |
| 11 | 12.4 | 4.1% |
| 12 | 13.6 | 13.4% |
| 13 | 14.2 | 21.4% |
| 14 | 15.2 | 28.0% |
| 15 | 16.0 | 100.0% |
| 16 | 16.8 | 28.6% |
| 17 | 17.8 | 6.7% |
| 18 | 18.2 | 13.7% |
| 19 | 19.5 | 18.4% |
| 20 | 20.1 | 5.6% |
| 21 | 20.7 | 19.9% |
| 22 | 20.8 | 18.9% |
| 23 | 21.6 | 4.7% |
| 24 | 21.8 | 2.6% |
| 25 | 22.2 | 6.2% |
| 26 | 24.3 | 5.5% |

### XRPD of GGG Side Chain Form C

A prepared sample of the GGG side chain, Form C was characterized by an XRPD pattern using CuKα radiation as having diffraction peaks (2-theta values) as described in Table 42 below, and in particular having peaks at 10.1 and 15.5 °2-Theta, in combination with one or more of the peaks selected from the group consisting of 6.1, 8.7, 11.4, 16.6, and 19.2 °2-Theta; with a tolerance for the diffraction angles of 0.2 degrees. A representative XRPD pattern of GGG side chain Form C is shown in FIG. 16C.

TABLE 42
X-ray powder diffraction peaks of GGG side chain Form C
GGG side chain Form C

| | Angle | Relative Intensity |
|---|---|---|
| Peak | (°2-Theta) +/− 0.2° | (% of most intense peak) |
| 1 | 6.1 | 60.4% |
| 2 | 7.2 | 6.5% |
| 3 | 7.9 | 12.7% |
| 4 | 8.7 | 59.2% |
| 5 | 11.1 | 100.0% |
| 6 | 11.4 | 31.9% |
| 7 | 11.8 | 12.5% |
| 8 | 12.4 | 24.2% |
| 9 | 13.2 | 16.7% |
| 10 | 13.7 | 13.9% |
| 11 | 14.4 | 18.4% |
| 12 | 15.5 | 52.9% |
| 13 | 15.9 | 22.3% |
| 14 | 16.6 | 24.8% |
| 15 | 17.6 | 26.4% |
| 16 | 17.9 | 24.0% |
| 17 | 18.3 | 33.5% |
| 18 | 18.7 | 22.6% |
| 19 | 18.9 | 15.8% |
| 20 | 19.2 | 51.8% |
| 21 | 19.5 | 68.0% |
| 22 | 19.7 | 79.2% |
| 23 | 20.3 | 7.7% |
| 24 | 20.9 | 42.5% |
| 25 | 21.3 | 20.7% |
| 26 | 21.9 | 16.8% |
| 27 | 23.0 | 27.9% |
| 28 | 23.7 | 10.8% |
| 29 | 24.0 | 32.8% |
| 30 | 24.7 | 25.5% |

### Example 14: H-Ala-Pro-Pro-Pro-Ser(tBu)-NH₂

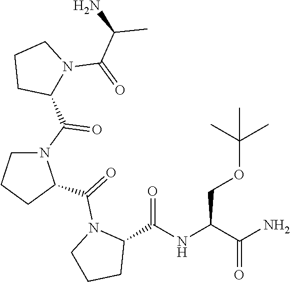 [SMILES: C[C@H](N)C(=O)N1CCC[C@H]1C(=O)N1CCC[C@H]1C(=O)N1CCC[C@H]1C(=O)N[C@@H](COC(C)(C)C)C(N)=O]

Crystallization studies for H-Ala-Pro-Pro-Pro-Ser(tBu)-NH₂ (H-A-P-P-P-S-NH₂) were performed on the free form (free base) and through salt/cocrystal formation.

Crystallization on the free form was attempted in various solvents and solvent mixtures. Different techniques were employed, such as slurry, cooling, and ambient and sub-ambient temperatures holding, etc. A crystalline H-A-P-P-P-S-NH₂ was not identified under the examined conditions.

Crystallization through salt formation was also performed. A total of 96 conditions, including 12 counterions, with each in 8 different solvents, were examined at an approximate 1:1.2 mol/mol (peptide/acid) ratio. Counterions and solvents employed in the screen are listed Table 43, below. Crystalline mono-HCl salt was observed in ACN (only at wet condition) and acetone.

TABLE 43
Counterions and solvents

| Counterions | Solvents |
|---|---|
| benzenesulfonic acid | water |
| citric acid | ACN |
| ethane-1,2-disulfonic acid | 95% Ethanol |
| ethanesulfonic acid | IPA |
| hydrochloric acid | Acetone |
| L-malic acid | 88% acetone |
| methanesulfonic acid | ethyl acetate |
| phosphoric acid | CPME |
| saccharin | |
| sulfuric acid | |
| L-tartaric acid | |
| p-toluenesulfonic acid | |

Crystallization through cocrystal formation was performed in CPME. Amorphous H-A-P-P-P-S-NH₂ solids were stirred in CPME solutions saturated with co-formers at ambient conditions overnight. 96 co-formers listed in Table 44, below, were examined. A crystalline H-A-P-P-P-S-NH₂ cocrystal was not identified under the examined conditions.

TABLE 44
Amorphous H-A-P-P-P-S-NH₂ solids

| Well | Co-former | MW |
|---|---|---|
| A1 | 2,3-dihydroxybenzoic acid | 154.12 |
| A2 | 2,4-dihydroxybenzoic acid | 154.12 |
| A3 | 2,5-dihydroxybenzoic acid | 154.12 |
| A4 | 2-amino-5-methylbenzoic acid | 151.17 |
| A5 | 2,6-dihydroxybenzoic acid | 154.12 |
| A6 | 3,4-dihydroxybenzoic acid | 154.12 |
| A7 | 3,5-dihydroxybenzoic acid | 154.12 |
| A8 | 3-hydroxybenzoic acid | 138.12 |
| A9 | 4-aminobenzoic acid | 137.13 |
| A10 | 4-hydroxybenzoic acid | 138.12 |
| A11 | acesulfame K | 201.24 |
| A12 | acetaminophen | 151.16 |
| B1 | acetylsalicylic acid | 180.16 |
| B2 | adipic acid | 146.14 |
| B3 | alanine (L) | 89.09 |
| B4 | apigenin | 270.24 |
| B5 | arginine (L) | 174.2 |
| B6 | ascorbic acid | 176.12 |
| B7 | aspartic acid (L) | 133.1 |
| B8 | azelaic acid | 188.22 |
| B9 | benzamide | 121.14 |
| B10 | benzoic acid | 122.12 |
| B11 | betaine | 135.16 |
| B12 | D-biotin | 244.31 |
| C1 | caffeic acid | 180.16 |
| C2 | caffeine | 194.19 |
| C3 | (1R,3S)-(+)-camphoric acid | 200.23 |
| C4 | caprolactam | 113.16 |
| C5 | D-(+)-10-camphorsulfonic acid | 144.21 |
| C6 | cinnamic acid (trans) | 148.16 |
| C7 | citric acid | 192.12 |
| C8 | D-glucuronic acid | 194.14 |
| C9 | EDTA | 292.24 |
| C10 | ethyl 4-hydroxybenzoate (paraben) | 166.18 |
| C11 | fructose (D) | 180.16 |
| C12 | fumaric acid | 116.08 |
| D1 | glutamic acid (L) | 147.13 |
| D2 | glutamine (L) | 146.14 |
| D3 | glutaric acid | 132.11 |
| D4 | gallic acid | |
| D5 | glycine | 75.07 |
| D6 | glycolic acid | 76.05 |
| D7 | hesperetin | 302.28 |
| D8 | hippuric acid | 179.17 |
| D9 | hydrocinnamic acid | 150.17 |
| D10 | imidazole | 68.08 |
| D11 | inositol (myo) | 180.16 |
| D12 | ketoglutaric acid (α) | 146.11 |
| E1 | lactobionic acid | 358.3 |
| E2 | lactose monohydrate (α) | 360.3 |
| E3 | leucine (L) | 131.2 |
| E4 | lysine (L) | 146.2 |
| E5 | maleic acid | 116.1 |
| E6 | meglumine | 195.2 |
| E7 | malic acid (L) | 134.1 |
| E8 | malonic acid | 104.1 |
| E9 | maltitol | 344.3 |
| E10 | mandelic acid (R) | 152.2 |
| E11 | mannitol | 182.2 |
| E12 | methionine (D or L) | 149.2 |
| F1 | methyl paraben | 152.2 |
| F2 | mucic acid | 210.1 |
| F3 | nicotinamide | 122.1 |
| F4 | nicotinic acid | 123.1 |
| F5 | orotic acid | 156.1 |
| F6 | oxalic acid | 90.03 |
| F7 | palmitic acid | 256.4 |
| F8 | phenylalanine (L) | 165.2 |
| F9 | phthalamide | 147.1 |
| F10 | pimelic acid (heptanedioic) | 160.2 |
| F11 | piperazine | 86.14 |
| F12 | proline (L) | 115.1 |
| G1 | propyl gallate | 212.2 |
| G2 | propyl paraben | 212.2 |
| G3 | pyrazine | 80.08 |
| G4 | L-Pyroglutamic acid | 129.1 |
| G5 | resorcinol | 110.1 |
| G6 | riboflavin | 376.4 |
| G7 | ribose (D) | 150.1 |
| G8 | saccharin | 183.2 |
| G9 | sebacic acid | 202.3 |
| G10 | salicylic acid | 138.1 |
| G11 | D-sorbitol | 182.2 |
| G12 | suberic acid | 174.2 |
| H1 | succinic acid | 118.1 |
| H2 | tartaric acid (L) | 150.1 |
| H3 | theophylline | 180.2 |
| H4 | thymidine | 242.2 |
| H5 | p-toluenesulfonic acid (monohydrate) | 190.2 |
| H6 | triphenylacetic acid | 288.4 |
| H7 | tryptophan (L) | 204.2 |
| H8 | tyrosine (L) | 181.2 |
| H9 | urea | 60.06 |
| H10 | vanillin | 152.2 |
| H11 | CONTROL | — |
| H12 | xylitol | 152.2 |

Two crystalline solid forms of the HCl salt of the H-A-P-P-P-S-NH₂ tetramer were identified, including H-A-P-P-P-S-NH₂ HCl Form A and Form B.

H-A-P-P-P-S-NH₂ HCl Form A is generated by salt reaction in acetonitrile which becomes amorphous upon isolation/drying. H-A-P-P-P-S-NH₂ HCl Form B is generated by salt reaction in acetone. Both are mono-HCl salts based on Ion chromatography analysis.

### H-A-P-P-P-S-NH₂ HCl Form A

### Lot 1

1 mL acetonitrile (ACN) was added to 79.4 mg of amorphous solids of H-A-P-P-P-S-NH₂ which produced a clear colorless solution at ambient conditions. With stirring, 183 μL 1N HCl in EtOAc (~1:1.2 mol/mol of peptide:HCl) was added to the peptide solution. The sample was stirred at ambient conditions and quickly the entire sample was full of white solids which appear as thin hairlike birefringent needles under polarized microscopy. An additional 3 mL of ACN was added to the sample, resulting a white slurry. The wet solids from the slurry were consistent with H-A-P-P-P-S-HCl Form A, which had lose crystallinity upon isolation/drying.

### Lot 2

3.1 gram of amorphous -A-P-P-P-S-NH₂ TFA salt was dissolved in 50 mL ACN into a clear yellow solution. With stirring, 8 mL IN HCl in EtOAc was added to the peptide solution. The sample was stirred at ambient conditions and white precipitates appeared shortly after stirring, until the entire sample became an off-white thick slurry. An additional 150 mL of ACN was added to the sample and stirred at ambient conditions for 2 days. The wet solids from the slurry were consistent with H-A-P-P-P-S-HCl Form A.

Solids from the slurry were isolated by vacuum filtration with N₂ flow on top of the filter, rinsed on the filter with fresh ACN, and then collected and dried at 30° C. under vacuum overnight. The resulting white solids were amorphous.

### H-A-P-P-P-S-NH₂ HCl Form B

1 mL acetone was added to 89.3 mg of amorphous solids of H-A-P-P-P-S-NH₂ which produced a clear colorless solution at ambient conditions. With stirring, 205 μL 1N HCl in EtOAc (~1:1.2 mol/mol of peptide:HCl) was added to the peptide solution which resulted a white thick slurry. An additional 2 mL of acetone was added and the sample was stirred at ambient conditions for 2 days. Solids from the slurry were isolated by vacuum filtration with N₂ flow on top of the filter, and the resulting white solids (55 mg) were consistent with H H-A-P-P-P-S-HCl Form B.

### XRPD of H-A-P-P-P-S-NH₂ HCl Form A

The XRPD pattern of crystalline H-A-P-P-P-S-NH₂ HCl was obtained on a wet sample using a PANalytical Empyrean diffractometer equipped with CuKα (1.5418 Å) source and a PIXcel3D 1×1 detector, operating at 45 kV and 40 mA. Samples were scanned between 2 and 40 2θ°, with step size of 0.0065652 2θ° for a total of 5788 steps over 3725 seconds. The sample was sandwiched between Etnom film in a sample holder to prevent solvent evaporation and analyzed in transmission geometry. The diffraction pattern was collected at ambient temperature.

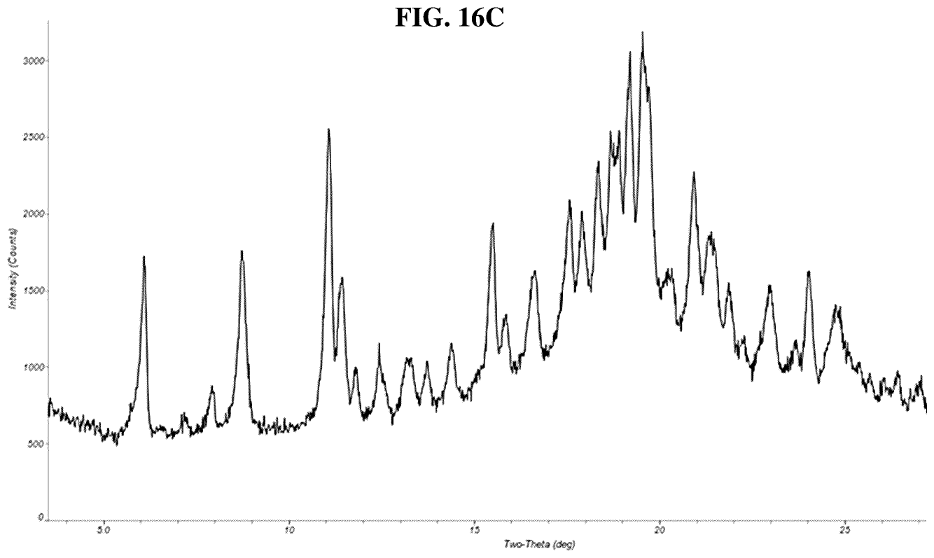

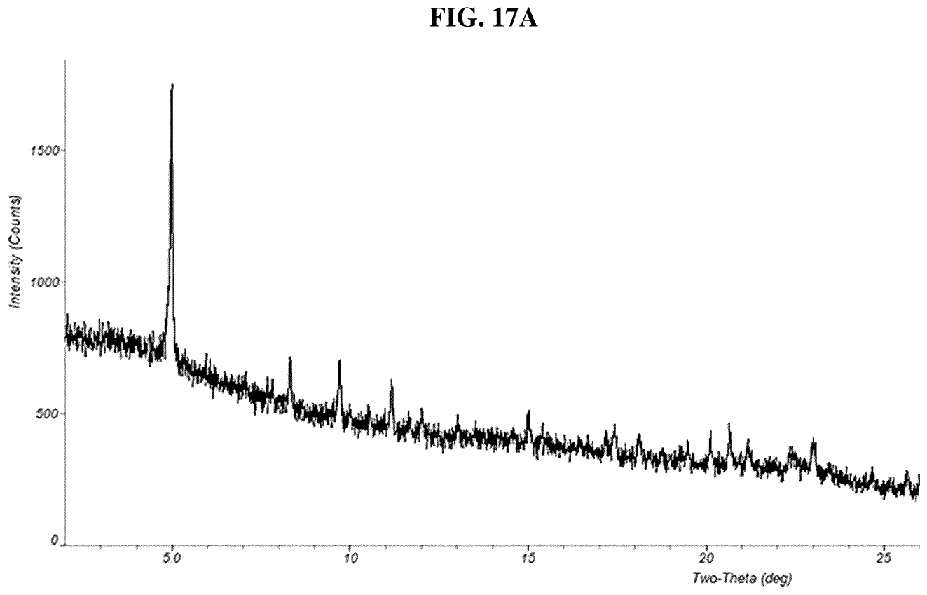

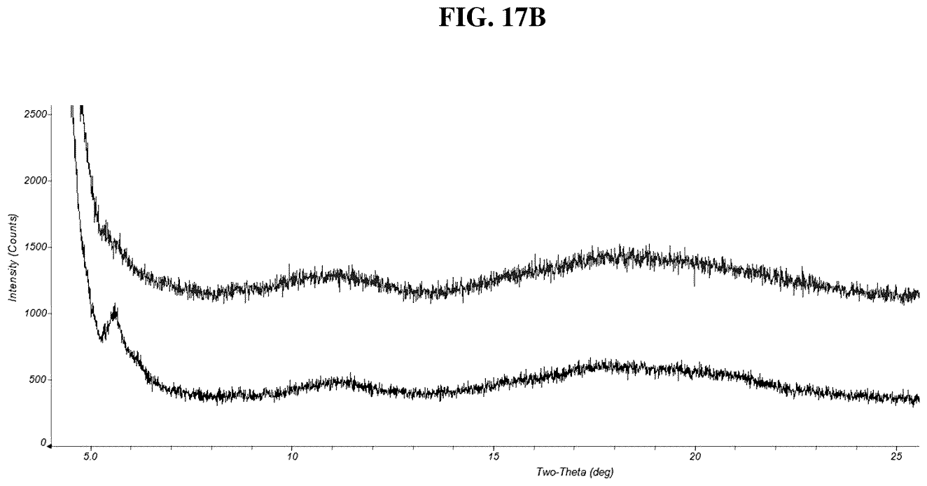

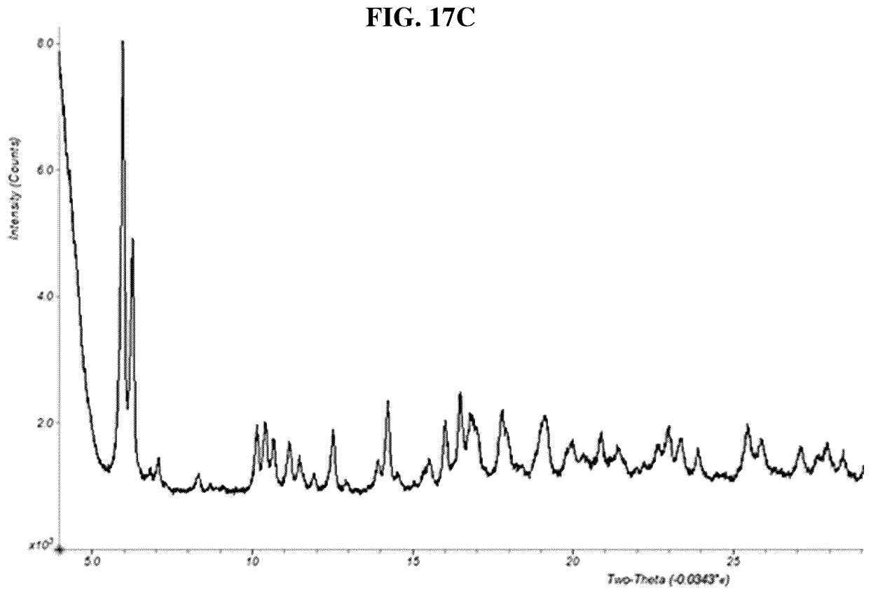

A prepared sample of H-A-P-P-P-S-NH₂ HCl Form A was characterized by an XRPD pattern using CuKα radiation as described above. The sample had diffraction peaks (2-theta values) as described in Table 45 and shown in FIG. 17A, and in particular had a peak at 5.0 °2-Theta in combination with one or more of the peaks selected from 8.3, 9.7, and 11.2 °2-Theta; with a tolerance for the diffraction angles of 0.2 degrees.

TABLE 45
X-ray powder diffraction peaks of H-A-P-P-P-S-NH2 HCl Form A
H-A-P-P-P-S-NH2 HCl Form A

| | Angle | Relative Intensity |
|---|---|---|
| Peak | (°2-Theta) +/− 0.2° | (% of most intense peak) |
| 1 | 5.0 | 100.0% |
| 2 | 8.3 | 18.1% |
| 3 | 9.7 | 19.7% |
| 4 | 11.2 | 18.5% |
| 5 | 12.0 | 8.8% |
| 6 | 15.0 | 9.2% |
| 7 | 17.2 | 7.2% |
| 8 | 17.4 | 9.7% |
| 9 | 18.1 | 9.4% |
| 10 | 19.5 | 7.9% |
| 11 | 20.1 | 12.8% |
| 12 | 20.7 | 14.8% |
| 13 | 21.2 | 7.4% |
| 14 | 23.0 | 12.1% |

XRPD of Dried Solids from H-A-P-P-P-S-NH₂ HCl Form A

H-A-P-P-P-S-NH₂ HCl Form A loses crystallinity upon isolation/drying. The dried sample displayed XRPD patterns with broad halos with or without broad peaks at low angle, as shown in FIG. 17B.

### XRPD of H-A-P-P-P-S-NH₂ HCl Form B

A prepared sample of H-A-P-P-P-S-NH₂ HCl Form B was characterized by an XRPD pattern using CuKα radiation as described above. The sample had diffraction peaks (2-theta values) as described in Table 46 and shown in FIG. 17C, and in particular had a peak at 7.2 °2-Theta in combination with one or more of the peaks selected from 5.3, 8.1, 14.4, and 16.2 °2-Theta; with a tolerance for the diffraction angles of 0.2 degrees.

TABLE 46
X-ray powder diffraction peaks of H-A-P-P-P-S-NH₂ HCl Form B
H-A-P-P-P-S-NH2 HCl Form B

| | Angle | Relative Intensity |
|---|---|---|
| Peak | (°2-Theta) +/− 0.2° | (% of most intense peak) |
| 1 | 6.0 | 100.0% |
| 2 | 6.2 | 56.1% |
| 3 | 7.1 | 6.9% |
| 4 | 10.1 | 15.4% |
| 5 | 10.4 | 10.4% |
| 6 | 10.7 | 10.1% |
| 7 | 12.5 | 13.3% |
| 8 | 14.2 | 16.8% |
| 9 | 16.0 | 11.4% |
| 10 | 16.5 | 13.7% |
| 11 | 16.8 | 13.8% |
| 12 | 17.8 | 15.0% |
| 13 | 19.2 | 13.6% |
| 14 | 20.0 | 6.8% |
| 15 | 20.9 | 7.1% |
| 16 | 23.0 | 6.0% |
| 17 | 23.3 | 6.1% |
| 18 | 25.4 | 12.0% |
| 19 | 27.1 | 6.2% |

### Example 15: Fmoc-Ser(tBu)-Ser(tBu)-Gly-Ala-Pro-Pro-Pro-Ser(tBu)-NH₂

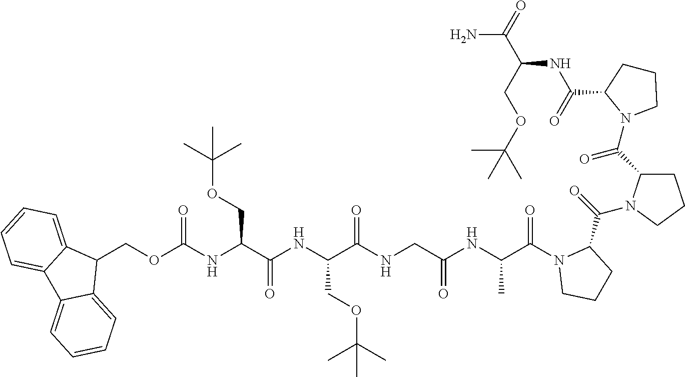 [SMILES: C[C@H](NC(=O)CNC(=O)[C@H](COC(C)(C)C)NC(=O)[C@H](COC(C)(C)C)NC(=O)OCC1c2ccccc2-c2ccccc21)C(=O)N1CCC[C@H]1C(=O)N1CCC[C@H]1C(=O)N1CCC[C@H]1C(=O)N[C@@H](COC(C)(C)C)C(N)=O]

Crystallization studies for Fmoc-Ser(tBu)-Ser(tBu)-Gly-Ala-Pro-Pro-Pro-Ser(tBu)-NH₂ (Fmoc-S-S-G-A-P-P-P-S-NH₂) were performed through salt/cocrystal formation. One solid form, Fmoc-S-S-G-A-P-P-P-S-NH₂ Form A was identified. Form A represents a family of isostructural solvates and can be generated from multiple solvent conditions such as acetone, acetonitrile (ACN), methyl acetate (MeOAc) and methyl ethyl ketone (MEK).

### Lot 1

0.4 mL acetone was added to approximately 60 mg of amorphous Fmoc-S-S-G-A-P-P-P-S-NH₂ solids resulting a clear yellow solution. The solution was stirred at ambient conditions for 2 days which yielded a white slurry. Solids isolated from the slurry were consistent with Fmoc-S-S-G-A-P-P-P-S-NH₂ Form A. Following similar procedures, Fmoc-S-S-G-A-P-P-P-S-NH₂ Form A was also be prepared from other organic solvents including as acetonitrile (ACN), methyl acetate (MeOAc) and methyl ethyl ketone (MEK).

### Lot 2

3.09 gram of amorphous solids of Fmoc-S-S-G-A-P-P-P-S-NH₂ was dissolved in 15 mL MeOAc. The solution was seeded with Fmoc-S-S-G-A-P-P-P-S-NH₂ Form A solids and stirred at ambient conditions for 2 days. Additional 12 mL of MeOAc was added during the process to facilitate the stirring. White solids were isolated from the slurry by vacuum filtration using a whatman paper filter, rinsed on the filter w/fresh MeOAc, 3 mL, for four times, and then collected and dried at 30° C. under vacuum for about 3 hours. The resulting white solids (2.4 gram) were consistent with Fmoc-S-S-G-A-P-P-P-S-NH₂ Form A.

### XRPD of Fmoc-S-S-G-A-P-P-P-S-NH₂ Form A

The XRPD pattern of crystalline Fmoc-S-S-G-A-P-P-P-S-NH₂ Form A was obtained on a Bruker D8 Endeavor X-ray powder diffractometer, equipped with a CuKα (1.5418 Å) source and a Linxeye detector, operating at 40 kV and 40 mA. The sample is scanned between 4 and 42 2θ°, with a step size of 0.009 2θ° and a scan rate of 0.5 seconds/step, and using 0.30 primary slit opening, and 3.9° PSD opening. The powder is packed on a quartz sample holder and a smooth surface is obtained using a glass slide. The diffraction patterns are collected at ambient temperature and relative humidity. Crystal peak positions are determined in MDI-Jade v7.9.9.

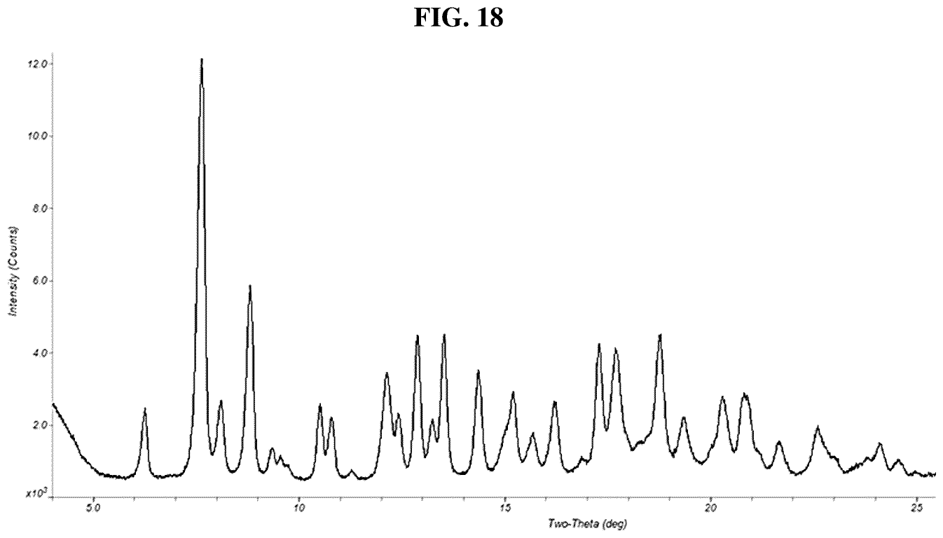

A prepared sample of Fmoc-S-S-G-A-P-P-P-S-NH₂ Form A was characterized by an XRPD pattern using CuKα radiation as described above. The sample had diffraction peaks (2-theta values) as described in Table 47 and shown in FIG. 18, and in particular had a peak at 7.5 °2-Theta in combination with one or more of the peaks selected from 6.1, 8.7, 10.6, 15.0, 16.1, and 18.6 °2-Theta; with a tolerance for the diffraction angles of 0.2 degrees.

TABLE 47
X-ray powder diffraction peaks of Fmoc-S-S-G-A-P-P-P-S-NH2 Form A
Fmoc-S-S-G-A-P-P-P-S-NH2 Form A

| | Angle | Relative Intensity |
|---|---|---|
| Peak | (°2-Theta) +/− 0.2° | (% of most intense peak) |
| 1 | 6.1 | 9.5% |
| 2 | 7.5 | 100.0% |
| 3 | 8.0 | 25.8% |
| 4 | 8.7 | 50.9% |
| 5 | 10.3 | 24.2% |
| 6 | 10.6 | 16.7% |
| 7 | 12.0 | 38.1% |
| 8 | 12.7 | 38.7% |
| 9 | 13.3 | 46.8% |
| 10 | 14.2 | 38.7% |
| 11 | 15.0 | 27.0% |
| 12 | 15.5 | 9.7% |
| 13 | 16.1 | 23.6% |
| 14 | 17.1 | 46.9% |
| 15 | 17.5 | 34.3% |
| 16 | 18.6 | 51.2% |
| 17 | 19.1 | 15.5% |
| 18 | 20.0 | 23.6% |
| 19 | 20.6 | 32.5% |
| 20 | 21.5 | 12.6% |
| 21 | 22.4 | 19.1% |

### Example 16: Liquid Phase Peptide Synthesis (LPPS) of Tirzepatide (TZP) Fragment 1

Tirzepatide Fragment 1 (TZP fragment 1) is a peptide that consist of ten amino acids (GPSSGAPPPS). Liquid-phase peptide synthesis (LPPS) strategies for preparing TZP fragment 1 were studied using the TFA salt of the compound (TFA NH₂-G-P-S-S-G-A-P-P-S-CONH₂). LPPS synthesis of TZP fragment 1 was achieved via coupling Fmoc-GP-OH and NH₂-G-P-S-S-G-A-P-P-S-CONH₂, followed by deprotecting the Fmoc group and precipitating the product out with known amount of trifluoroacetic acid.

### Materials

Fmoc-GP-OH, EDC·HCl, oxyma, diisopropyethylether (DIPEA), acetonitrile (ACN), isopropylacetate (IPAc), trifluoroacetic acid (TFA), ethylacetate (EtOAc), sodium bicarbonate (NaHCO₃), diethylamine (DEA), methyl-tertbutylether (MTBE), heptane, and sodium sulfate were purchased and used without any purification. NH₂-G-P-S-S-G-A-P-P-S-CONH₂ was synthesized by solid-phase peptide synthesis (SPPS).

### Synthesis of Fmoc-G-P-S-S-G-A-P-P-S-CONH₂—Fmoc Coupling

Synthesis of Fmoc-G-P-S-S-G-A-P-P-S-CONH₂ (the protected TZP fragment 1) was achieved by coupling Fmoc-GP-OH to NH₂-G-P-S-S-G-A-P-P-S-CONH₂ in acetonitrile/isopropyl acetate (ACN/IPAc).

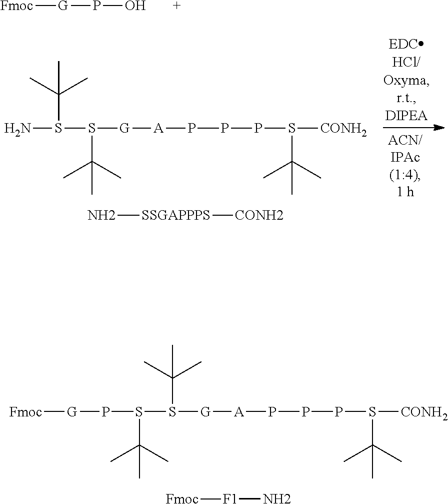

To a round-bottom flask containing a stir bar was added 20 ml (20 V) of ACN/IPAc (1:3), Fmoc-GP-OH (1.00 eq, 1.16 mmol, 456 mg), and oxyma (1.12 eq, 1.29 mmol, 184 mg) at room temperature while stirring. EDC·HCl (1.26 eq, 1.46 mmol, 279 mg) and DIPEA (1.0 eq, 1.16 mmol, 199 μL) was added and stirred for 10 minutes at room temperature for pre-activation (reaction turns yellow). Reaction flask was sonicated for 1 minutes before NH₂-G-P-S-S-G-A-P-P-S-CONH₂ (1.00 eq, 1.16 mmol, 1.00 g) was added in one portion and stir for 1 hour. The pH of the reaction was kept between 3.5-5.5. Upon completion, the crude mixture was transferred to a separatory funnel, the flask was rinsed with 2 ml of ethylacetate (EtOAc) and transfer to the separatory funnel. The mixture was washed with 7 ml (7V) of 1M aqueous HCl 2× and the aqueous layer was separated into an Erlenmeyer flask1. This was followed by 2× base washes each with 7 ml (7V) of saturated aqueous NaHCO₃, and the aqueous layer was drained into an Erlenmeyer flask2. Then 2× washes with 7 ml DI water and the aqueous layer drained to Erlenmeyer flask2. The organic layer was transferred to an Erlenmeyer flask3.

20 ml (20V) of fresh ethyl acetate was added to the separatory funnel to back-extract the aqueous acid and the base layers. The organic layers were combined, dried with Na₂SO₄, and filtered to a flask. Solvent was removed via rotary evaporation to obtain Fmoc-G-P-S-S-G-A-P-P-S-CONH₂ as a solid 1.032 g (70% yield).

### Synthesis of F1-NH₂·TFA-Fmoc Deprotection and Subsequent TFA Salting

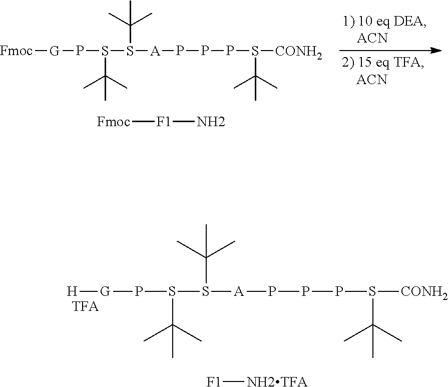

To a flask charged with a stir bar was dissolved Fmoc-G-P-S-S-G-A-P-P-S-CONH₂ (1.0 eq, 0.79 mmol, 0.98 g) in ACN (3V, 2.92 ml) at room temperature. Diethylamine (10 eq, 0.812 ml) was added and stirred for 1 h at room temperature. Solvent was reduced via rotary evaporation to 2 ml (~2V) and 15 ml (~15V) of cooled 1:1 MTBE: Heptane was added slowly while swirling to precipitate out the product, which was then filtered. The flask was rinsed with more cooled 1:1 MTBE:Heptane and filtered.

The combined solid was dissolved in 2 ml ACN and transferred into a vial and completely dried by rotary evaporation. 2 ml of ACN and a stir bar were added to the material in a vial and trifluoroacetic acid (15 eq, 11.8 mmol, 0.904 ml) was added in one portion at room temperature. Reaction was allowed to stir for 50 minutes, stir bar was removed and 15 ml of cooled 1:1 MTBE:Heptane was slowly added while swirling to obtain a suspension which was filtered to obtain the solid product. The combine solid was dried via rotary evaporation to obtain F1-NH₂·TFA as a whitish solid, 0.87 g (77% yield).

### Screening Coupling Agents

Different coupling agents were screened for the synthesis of Fmoc-G-P-S-S-G-A-P-P-S-CONH₂ via coupling Fmoc-GP-OH to NH₂-G-P-S-S-G-A-P-P-S-CONH₂. A summary of the coupling agent screened for reaction is presented in Table 48.

TABLE 48

| | 1 | 2 | 3 | 4 | 5 | 6 |
|---|---|---|---|---|---|---|
| A | EEDQ | TPTU | HATU | CDMT | TCTU | HBTU |
| B | HDMA | PyBOP | T3P | HOPO/EDCI | TATU | TNTU |
| C | TOTU | PyOxim | TCFH | COMU | PyAOP | TSTU |
| D | TBTU | PyClock | DEPBT | TOTT | HCTU | CITU |

In the experiments, a total of 24 coupling agents were screened for the reaction to investigate which coupling agent(s) is/are best for complete conversion of starting materials to product within minimal time, reduced impurity formation, ease of impurity rejection via extraction, cost effectiveness and minimal health hazard.

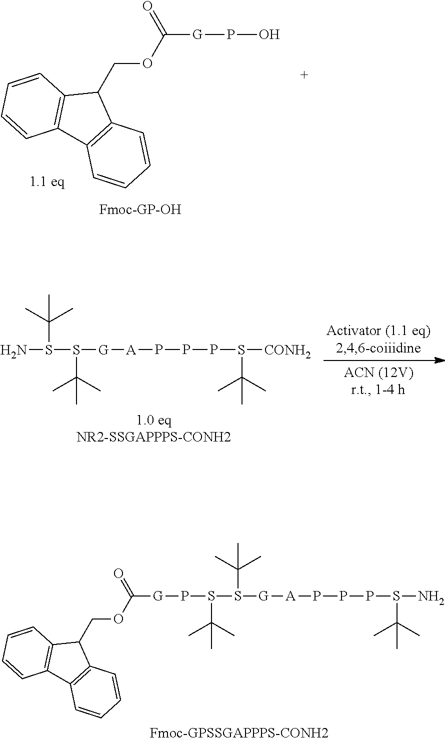

Coupling agent screening was carried out using the ‘Dreadnought’ Equipment Set. Fmoc-GP-OH and each coupling agent was dispersed into the appropriate well using Quantos. Stock solutions of 2,4,6-collidine in ACN, DEPBT/2,4,6-collidine in ACN, and T3P/2,4,6-collidine in ACN were prepared and dispense in the appropriate wells in a glove box. The plate was sealed and removed from the glove box, stirred for 30 minutes at 800 rpm on a poly block and moved back to the glove box where the stock solution of NH₂-G-P-S-S-G-A-P-P-S-CONH₂ was prepared and 1.1 eq was dispense to each vial.

The plate was removed from the glove box and allowed to stir at room temperature for four hours. LCMS analytical samples were harvested from each vial at 1 hour and at 4-hour intervals respectively, diluted and analyzed by LCMS for percentage conversion at 1-hour and 4-hour marks. Results are shown in FIG. 19.

Out of the 24 coupling agents screened for the reaction, 15 coupling agents showed 100% product formation (100% conversion of limiting starting material to product) within the 1-hour interval, while 2 coupling agents showed 100% product formation only at 4 hour intervals. 7 of the coupling agents did not achieve full conversion of limiting starting material to product even at 4 hours of reaction time.

### Example 17: Solvent Screening Study

Peptide samples were screened for crystal stability in several organic solvents. The following four peptide tetramers were used for the study: (i) Boc-Y(tBu)-Aib-E(Me)-G-COOH; (ii) Boc-Y(tBu)-Aib-E(All)-G-COOH; (iii) Boc-Y(tBu)-Aib-E(cHx)-G-COOH; and (iv) Boc-Y(tBu)-Aib-E(Bzl)-G-COOH. The following organic solvents were used for the study: Acetone, ACN, MeOH, EtOH, IPA, BuOH, EtOAc, MTBE, THF, MEK, toluene, and water.

30 mg samples of each peptide tetramer were dispensed into sample vials using an automated dispenser (one vial of each peptide for testing with each solvent). Solvent was then added to the vials and vortexed, with solvent being added until peptide dissolution was observed or until solution was still cloudy with 1000 μL of solvent. Samples were then allowed to stand for 24 hours or more. Results are shown in Tables 49-52, including indication of which solvents formed crystals for each peptide sample.

TABLE 49
Boc-Y(tBu)-Aib-E(Me)-G-COOH

| | | Total | Solubility | Crystal |
|---|---|---|---|---|
| wt (mg) | Solvent | volume (mL) | (mg/mL) | Formed |
| 30.02 | Acetone | 0.03 | 1001 | No |
| 30.21 | ACN | 0.03 | 1007 | No |
| 30.66 | EtOH | 0.03 | 1022 | No |
| 30.21 | EtOAc | 0.03 | 1007 | No |
| 30.28 | MTBE | 0.3 | 101 | Yes |
| 30.29 | BuOH | 0.06 | 505 | No |
| 29.92 | MeOH | 0.03 | 997 | No |
| 35.22 | THF | 0.06 | 587 | No |
| 31.63 | IPA | 0.09 | 351 | No |
| 36.29 | MEK | 0.06 | 605 | No |
| 31.53 | toluene | 0.075 | 420 | No |
| 30.14 | H2O | 0.63 | 48 | No |

TABLE 50
Boc-Y(tBu)-Aib-E(All)-G-COOH

| | | Total | Solubility | Crystal |
|---|---|---|---|---|
| wt (mg) | Solvent | volume (mL) | (mg/mL) | Formed |
| 30.055 | Acetone | 0.03 | 1002 | No |
| 30.06 | ACN | 0.03 | 1002 | No |
| 30.04 | EtOH | 0.045 | 668 | No |
| 30.045 | EtOAc | 0.15 | 200 | Yes |
| 30.045 | MTBE | 0.09 | 334 | Yes |
| 30.035 | BuOH | 0.075 | 400 | No |
| 30.025 | MeOH | 0.03 | 1001 | No |
| 30.04 | THF | 0.045 | 668 | No |
| 30.06 | IPA | 0.06 | 501 | No |
| 30.05 | MEK | 0.03 | 1002 | Yes |
| 30.05 | toluene | 0.075 | 401 | No |
| 30.035 | H2O | 0.3 | 100 | No |

TABLE 51
Boc-Y(tBu)-Aib-E(cHx)-G-COOH

| | | Total | Solubility | Crystal |
|---|---|---|---|---|
| wt (mg) | Solvent | volume (mL) | (mg/mL) | Formed |
| 27.53 | Acetone | 0.03 | 918 | No |
| 27.225 | ACN | 0.03 | 908 | No |
| 27.23 | EtOH | 0.03 | 908 | No |
| 27.225 | EtOAc | 0.03 | 908 | No |
| 27.19 | MTBE | 0.06 | 453 | No |
| 27.23 | BuOH | 0.06 | 454 | No |
| 27.185 | MeOH | 0.03 | 906 | No |
| 27.23 | THF | 0.03 | 908 | No |
| 27.245 | IPA | 0.06 | 454 | No |
| 27.225 | MEK | 0.03 | 908 | No |
| 27.21 | toluene | 0.03 | 907 | No |
| 27.845 | H2O | 0.6 | 46 | No |

TABLE 52
Boc-Y(tBu)-Aib-E(Bzl)-G-COOH

| | | Total | Solubility | Crystal |
|---|---|---|---|---|
| wt (mg) | Solvent | volume (mL) | (mg/mL) | Formed |
| 29.825 | Acetone | 0.03 | 994 | Yes |
| 21.33 | ACN | 0.03 | 711 | Yes |
| 29.985 | EtOH | 0.03 | 1000 | No |
| 28.23 | EtOAc | 0.03 | 941 | Yes |
| 30.03 | MTBE | 0.06 | 501 | Yes |
| 30.04 | BuOH | 0.06 | 501 | No |
| 30.04 | MeOH | 0.03 | 1001 | No |
| 30.04 | THF | 0.03 | 1001 | Yes |
| 30.025 | IPA | 0.06 | 500 | No |
| 30.04 | MEK | 0.03 | 1001 | Yes |
| 30.03 | toluene | 0.045 | 667 | Yes |
| 30.02 | H2O | 0.6 | 50 | No |

### Example 18: Fmoc-Tyr (tBu)-Aib-Glu(tBu)-Gly-COOH

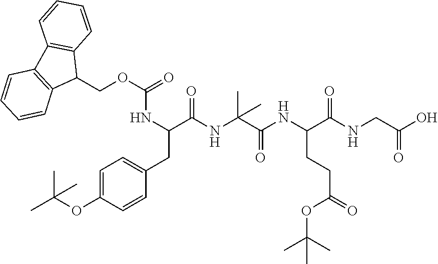 [SMILES: CC(C)(C)OC(=O)CCC(NC(=O)C(C)(C)NC(=O)C(Cc1ccc(OC(C)(C)C)cc1)NC(=O)OCC1c2ccccc2-c2ccccc21)C(=O)NCC(=O)O]

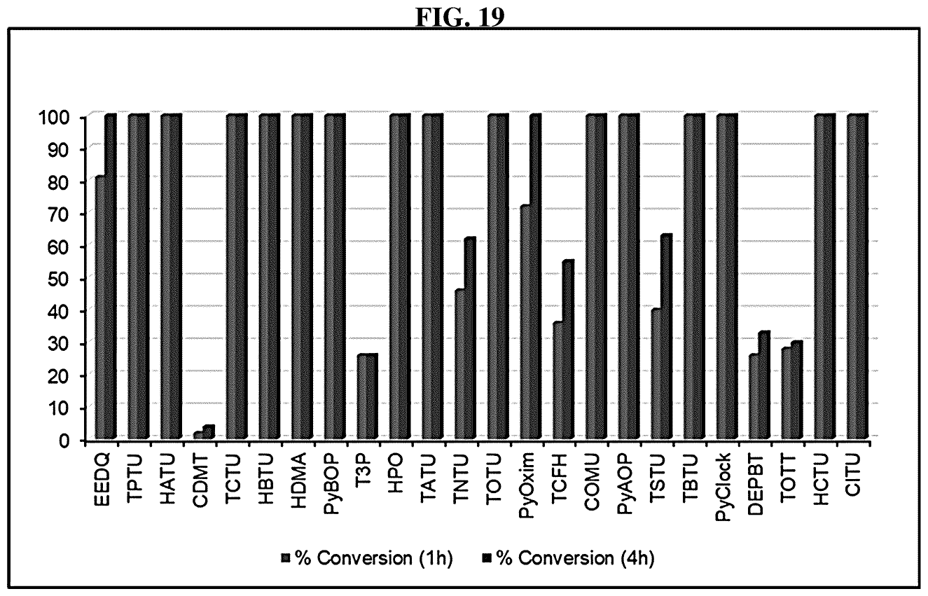

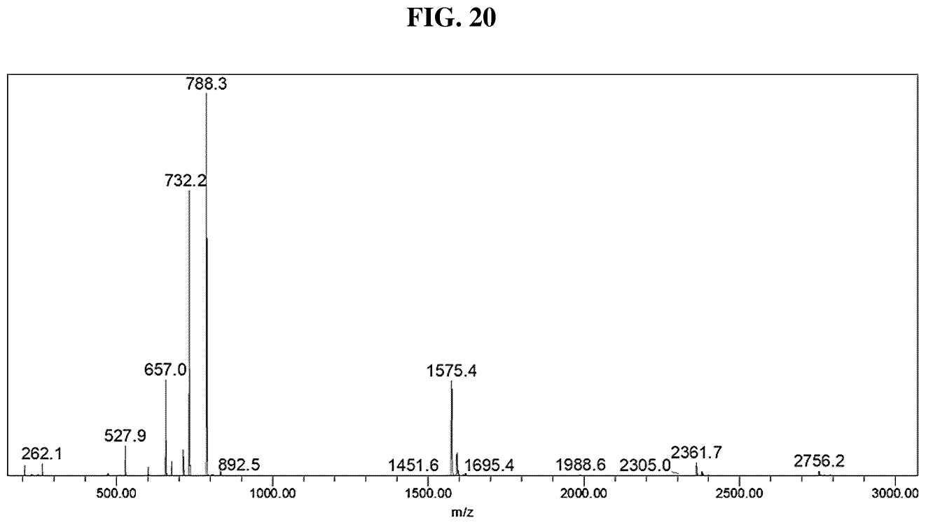

Fmoc-Y(tBu)-Aib-E(tBu)-G-COOH was synthesized using Fmoc solid-phase synthesis with Oxyma/DIC protocols on glycine pre-loaded CTC resin with a Symphony peptide synthesizer. Coupling were as follows: (i) E-G: 6 hours for E(tBu); (ii) Aib-E: 12 hours; and (iii) Y-Aib: 18 hours. Peptides were detached from the resins without deprotecting sidechains and N-terminals by treating the peptides on the CTC resins with 30% HFIP in DCM for 2 hours. The cleavage solution was rotavated, and then the residue was dissolved in acetonitrile and rotavated to make it foam form. The foam form of solids was dried overnight in vacuum chamber, and then analyzed by UPLC-MS for purity and mass. UPLC-MS results are shown in FIG. 20.

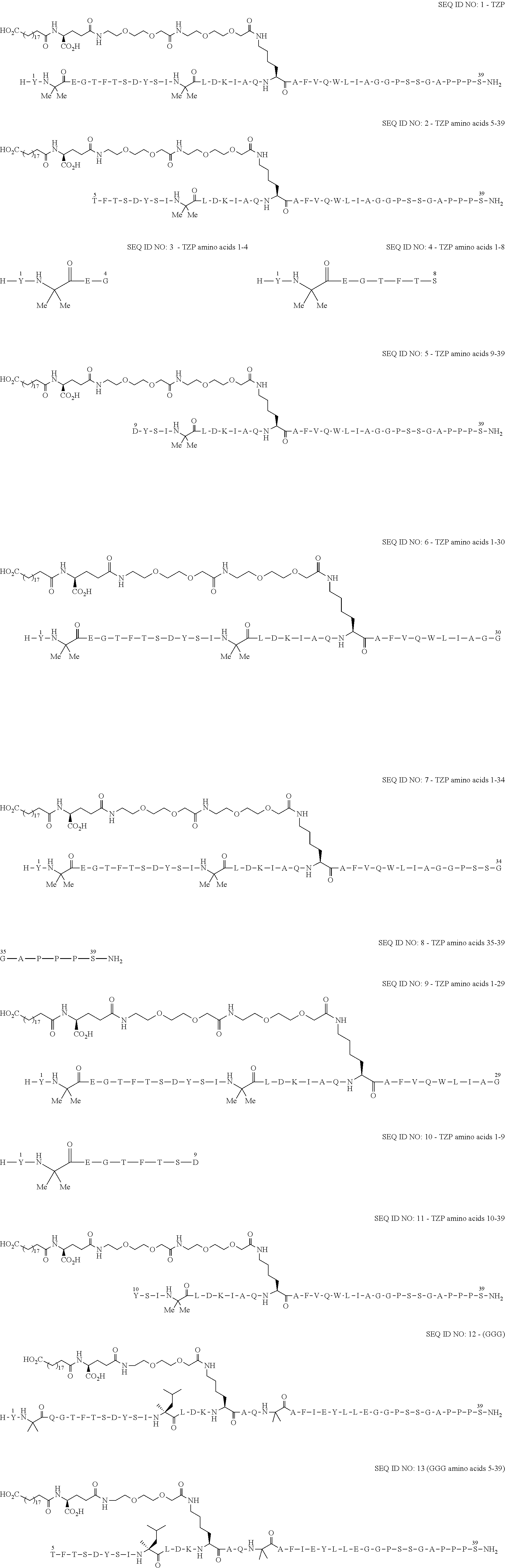

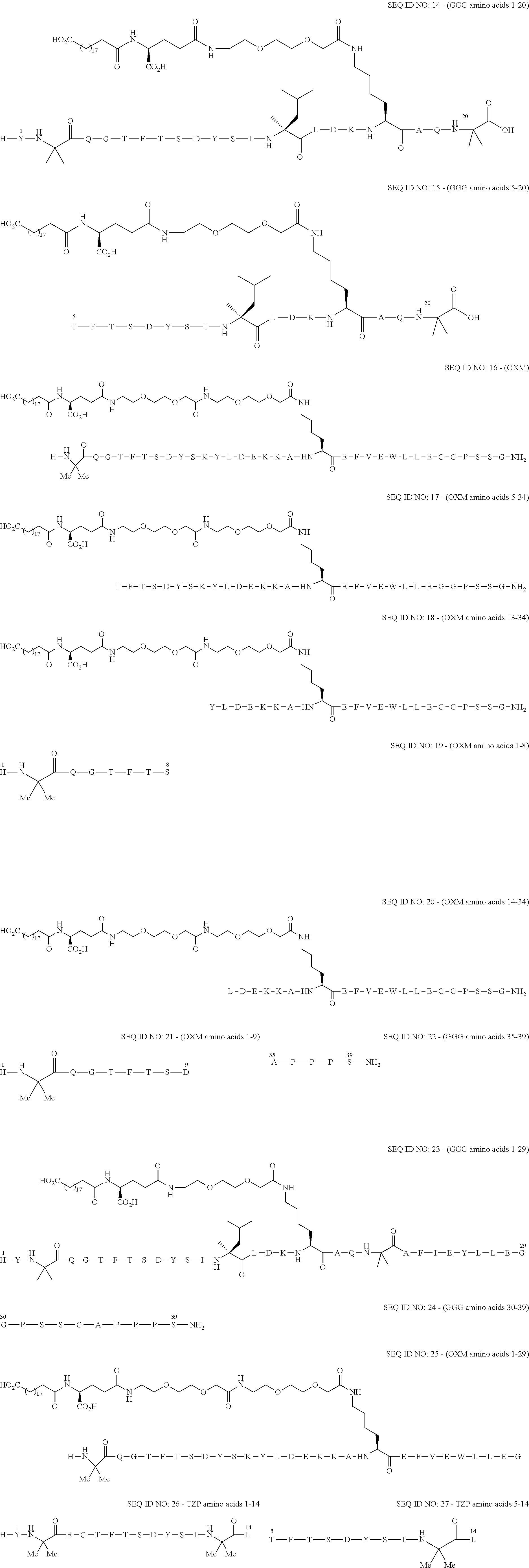

What is claimed:

1-99. (canceled)

100. A compound comprising the amino acid sequence of SEQ ID NO: 2, wherein the compound is optionally substituted with one or more protecting groups independently chosen from Boc, Fmoc, tert-butyl, and trityl groups.

101. A composition comprising the compound of claim 100.

102. A composition comprising:
(a) the compound of claim 100; and
(b) a compound comprising the amino acid sequence of SEQ ID NO: 1.

103. A method of making a compound comprising the amino acid sequence of SEQ ID NO: 1, wherein the method comprises attaching a C-terminus of a compound of claim 100 to a N-terminus of the compound of SEQ ID NO: 3.

104. A compound comprising the amino acid sequence of SEQ ID NO: 11, wherein the compound is optionally substituted with one or more protecting groups independently chosen from Boc, Fmoc, tert-butyl, and trityl groups.

105. A composition comprising the compound of claim 104.

106. A composition comprising:
(a) the compound of claim 104; and
(b) a compound comprising the amino acid sequence of SEQ ID NO: 1.

107. A method of making a compound comprising the amino acid sequence of SEQ ID NO: 1, wherein the method comprises attaching a C-terminus of a compound of claim 104 to a N-terminus of the compound of SEQ ID NO:10.
